(19) United States
(12) Patent Application Publication
(10) Pub. No.: US 2019/0300918 A1
(43) Pub. Date: Oct. 3, 2019

(54) MICROORGANISMS AND METHODS FOR THE PRODUCTION OF BIOSYNTHESIZED TARGET PRODUCTS HAVING REDUCED LEVELS OF BYPRODUCTS

(71) Applicant: Genomatica, Inc., San Diego, CA (US)

(72) Inventors: Robin E. OSTERHOUT, San Diego, CA (US); Priti PHARKYA, San Diego, CA (US)

(21) Appl. No.: 15/579,118

(22) PCT Filed: Jun. 22, 2016
(86) PCT No.: PCT/US16/38647

§ 371 Date: Mar. 15, 2018

Related U.S. Application Data
(60) Provisional application No. 62/183,620, filed on Jun. 23, 2015.

(51) Int. Cl. C12P 17/10; C12N 15/52; C12P 13/00; C12P 7/40; C12P 7/44; C12P 7/18; C12N 9/04; C12N 9/02; C12P 17/08; C12N 9/06; C12N 9/10; C12N 9/16; C12N 9/00

(52) U.S. Cl. CPC C12P 17/10; C12Y 603/03004; C12P 13/001; C12P 7/40; C12P 13/005; C12P 7/44; C12P 7/18; C12N 9/0006; C12Y 101/01035; C12N 9/0008; C12N 9/001; C12P 17/08; C12Y 101/01037; C12Y 101/01027; C12Y 101/01172; C12Y 101/0103; C12Y 103/01031; C12N 9/0016; C12Y 104/01009; C12N 9/1029; C12Y 203/01174; C12N 9/1096; C12Y 206/01019; C12N 9/13; C12Y 208/03006; C12N 9/16; C12Y 301/02004; C12N 9/93; C12N 15/52

(57) ABSTRACT

Provided herein are non-naturally occurring microbial organisms having biosynthetic pathways for production of target products and one or more genetic modifications that reduce a byproduct of the biosynthetic pathway. Compositions of target products from such cells and methods of using such cells are provided.

### CROSS REFERENCE TO RELATED APPLICATIONS

This application claims priority to U.S. Provisional Application No. 62/183,620, filed Jun. 23, 2015, the entirety of which is incorporated herein by reference and for all purposes.

### BACKGROUND

Byproducts and impurities generated in biosynthetic pathways for producing chemicals of interest are wasted carbon not used to make the desired product. Such compounds can be toxic to the cell, or may impart an undesirable property to final products and as color, odor, instability, degradation, and inhibition of polymerization in such reaction. Such byproducts and impurities therefore increase burden, cost, and complexity of biosynthesizing compounds and can decrease efficiency or yield of downstream purification.

Caprolactone (ε-Caprolactone) is a cyclic ester with a seven-membered ring having the formula (CH₂)₅CO₂. This colorless liquid is miscible with most organic solvents. It is produced as a precursor to caprolactam. The caprolactone monomer is used in the manufacture of highly specialized polymers because of its ring-opening potential. Ring-opening polymerization, for example, results in the production of polycaprolactone. Caprolactone is typically prepared by oxidation of cyclohexanone with peracetic acid.

Caprolactone undergoes reactions typical for primary alcohols. Downstream applications of these product groups include protective and industrial coatings, polyurethanes, cast elastomers, adhesives, colorants, pharmaceuticals and many more. Other useful properties of caprolactone include high resistance to hydrolysis, excellent mechanical properties, and low glass transition temperature.

Adipic acid, a dicarboxylic acid, has a molecular weight of 146.14. It can be used is to produce polyamide 6,6, a linear polyamide made by condensing adipic acid with hexamethylenediamine. This is employed for manufacturing different kinds of fibers. Other uses of adipic acid include its use in plasticizers, unsaturated polyesters, and polyester polyols. Additional uses include for production of polyurethane, lubricant components, and as a food ingredient as a flavorant and gelling aid.

In addition to hexamethylenediamine (HMD) being used in the production of polyamide-6,6 as described above, it is also utilized to make hexamethylene diisocyanate, a monomer feedstock used in the production of polyurethane. The diamine also serves as a cross-linking agent in epoxy resins. HMD can be produced by the hydrogenation of adiponitrile.

Caprolactam is an organic compound which is a lactam of 6-aminohexanoic acid (ε-aminohexanoic acid, 6-aminocaproic acid). It can alternatively be considered cyclic amide of caproic acid. One use of caprolactam is as a monomer in the production of nylon-6. Caprolactam can be synthesized from cyclohexanone via an oximation process using hydroxylammonium sulfate followed by catalytic rearrangement using the Beckmann rearrangement process step.

Non-naturally occurring microorganisms for producing target products such as those described above are known in the art. However, these non-naturally occurring microorganisms can have byproducts produced during biosynthesis as a result of undesired enzymatic activity on pathway intermediates and final products. Accordingly, there is a need in the art to develop cells and methods for effectively producing commercial quantities of compounds such as hexamethylenediamine, 6-aminocaproic acid, adipic acid, 1,6-hexanediol, levulinic acid, caprolactone, and caprolactam with reduced byproducts and impurities. Provided herein, inter alia, are solutions to these and other problems in the art.

### BRIEF SUMMARY

The present invention relates generally to biosynthetic processes, and more specifically to organisms having capability to biosynthesize target products with less byproduct.

Provided herein are genetically modified cells capable of producing a target product described herein. In one aspect is a genetically modified cell capable of producing a target product, where the target product includes hexamethylenediamine (HMD), levulinic acid (LVA), 6-aminocaproic acid (6ACA), caprolactam (CPL), caprolactone (CPO), adipic acid (ADA), or 1,6-hexanediol (HDO) or a combination thereof, where the genetically modified cell includes one or more genetic modifications selected from: (a) a genetic modification that decreases activity of an enzyme selected from an Oxidoreductase acting on an aldehyde or oxo moiety (A1); Oxidoreductase acting on a acyl-CoA moiety (A2); Oxidoreductase acting on an aldehyde moiety (A3); Oxidoreductase acting on an aldehyde or acyl-CoA moiety (A4); Aldehyde oxidase acting on an aldehyde moiety (A5); Oxidoreductase acting on an alkene or alkane moiety (A6); Oxidoreductase acting on an amine moiety (A7); Amine N-methyltransferase acting on an amine moiety (A8); Carbamoyl transferase acting on an amine moiety (A9); Acyltransferase acting on an acyl-CoA moiety (A10); Acyltransferase acting on an amine or acyl-CoA moiety (A11); N-propylamine synthase acting on an amine moiety (A12); Aminotransferase acting on an amine or aldehyde moiety (A13); CoA transferase acting on an acyl-CoA or an acid moiety (A14); Thioester hydrolase acting on an acyl-CoA moiety (A15); Decarboxylase acting on an oxoacid moiety (A16); Dehydratase acting on a hydroxyacid moiety (A17); Ammonia-lyase acting on an amine moiety (A18); CoA ligase acting on an acyl-CoA or acid moiety (A19); glutamyl:amine ligase acting on an amine moiety (A20); Amine hydroxylase acting on an amine moiety (A21); Oxidoreductase acting on an acyl-CoA moiety (A22); Amine oxidase acting on an amine moiety (A23); short chain diamine exporter acting on a diamine moiety (A24); and putrescine permease acting on a diamine moiety (A25); (b) a genetic modification that increases activity of an enzyme selected from Amide hydrolase or amidase acting on an amide moiety (B1); Cyclic amide hydrolase or lactamase acting on a cyclic amide moiety (B2); CoA ligase acting on an acid moiety (B3); Diamine transporter (longer chain diamines) acting on an amine moiety (B4); and diamine permease acting on an amine moiety (B5); and (c) a combination of two or more, three or more, four or more, five or more, six or more, seven or more, eight or more, nine or more, ten or more, or all of the genetic modifications of (a) and (b); wherein the cell produces a reduced amount of one or more byproducts when compared to a cell without the one or more genetic modifications.

Also provided herein is a non-naturally occurring microbial organism, that includes a hexamethylenediamine (HMD) pathway and is capable of producing HMD, wherein the non-naturally occurring microbial organism further includes: (a) a genetic modification selected from: (i) a genetic modification that decreases activity of an enzyme selected from A1, A2, A3, A4, A5, A6, A7, A8, A9, A10, A11, A12, A13, A14, A15, A16, A17, A18, A19, A20, A21, A22, A23, A24, or A25; (ii) a genetic modification that increases activity of an enzyme selected from B1, B2, B3, B4, or B5; and (iii) a combination of two or more, three or more, four or more, five or more, six or more, seven or more, eight or more, or all of the genetic modifications of (i) and (ii); and (b) a HMD pathway as described herein that includes at least one exogenous nucleic acid encoding a HMD pathway enzyme.

In another aspect is a non-naturally occurring microbial organism that includes a levulinic acid (LVA) pathway and is capable of producing LVA, wherein the non-naturally occurring microbial organism further includes: (a) a genetic modification selected from: (i) a genetic modification that decreases activity of an enzyme selected from A1, A2, A3, A4, A5, A6, A7, A8, A9, A10, A11, A12, A13, A14, A15, A16, A17, A18, A19, A20, A21, A22, A23, A24, or A25; (ii) a genetic modification that increases activity of an enzyme selected from B 1, B2, B3, B4, or B5; and (iii) a combination of two or more, three or more, four or more, five or more, six or more, seven or more, eight or more, or all of the genetic modifications of (i) and (ii); and (b) a LVA pathway described herein that includes at least one exogenous nucleic acid encoding a LVA pathway enzyme.

In yet another aspect is a non-naturally occurring microbial organism, that includes a caprolactone (CPO) pathway and is capable of producing CPO, wherein the non-naturally occurring microbial organism further includes: (a) a genetic modification selected from: (i) a genetic modification that decreases activity of an enzyme selected from A1, A2, A3, A4, A5, A6, A7, A8, A9, A10, A11, A12, A13, A14, A15, A16, A17, A18, A19, A20, A21, A22, A23, A24, or A25; (ii) a genetic modification that increases activity of an enzyme selected from B1, B2, B3, B4, or B5; and (iii) a combination of two or more, three or more, four or more, five or more, six or more, seven or more, eight or more, or all of the genetic modifications of (i) and (ii); and a CPO pathway described herein that includes at least one exogenous nucleic acid encoding a CPO pathway enzyme.

In still another aspect is a non-naturally occurring microbial organism that includes a 1,6-hexanediol (HDO) pathway and is capable of producing HDO, wherein the non-naturally occurring microbial organism further includes: (a) a genetic modification selected from: (i) a genetic modification that decreases activity of an enzyme selected from A1, A2, A3, A4, A5, A6, A7, A8, A9, A10, A11, A12, A13, A14, A15, A16, A17, A18, A19, A20, A21, A22, A23, A24, or A25; (ii) a genetic modification that increases activity of an enzyme selected from B1, B2, B3, B4, or B5; and (iii) a combination of two or more, three or more, four or more, five or more, six or more, seven or more, eight or more, or all of the genetic modifications of (i) and (ii); and a HDO pathway described herein that includes at least one exogenous nucleic acid encoding a HDO pathway enzyme.

In another aspect is a non-naturally occurring microbial organism that includes a 1,6-hexanediol (HDO) pathway and at least one exogenous nucleic acid encoding a HDO pathway enzyme expressed in a sufficient amount to produce HDO, where the HDO pathway includes: a 6-aminocaproyl-CoA transferase or synthetase catalyzing conversion of 6ACA to 6-aminocaproyl-CoA (4A); a 6-aminocaproyl-CoA reductase catalyzing conversion of 6-aminocaproyl-CoA to 6-aminocaproate semialdehyde (4B); a 6-aminocaproate semialdehyde reductase catalyzing conversion of 6-aminocaproate semialdehyde to 6-aminohexanol (4C); a 6-aminocaproate reductase catalyzing conversion of 6ACA to 6-aminocaproate semialdehyde (4D); an adipyl-CoA reductase adipyl-CoA to adipate semialdehyde (4E); an adipate semialdehyde reductase catalyzing conversion of adipate semialdehyde to 6-hydroxyhexanoate (4F); a 6-hydroxyhexanoyl-CoA transferase or synthetase catalyzing conversion of 6-hydroxyhexanoate to 6-hydroxyhexanoyl-CoA (4G); a 6-hydroxyhexanoyl-CoA reductase catalyzing conversion of 6-hydroxyhexanoyl-CoA to 6-hydroxyhexanal (4H); a 6-hydroxyhexanal reductase catalyzing conversion of 6-hydroxyhexanal to HDO (4I); a 6-aminohexanol aminotransferase or oxidoreductases catalyzing conversion of 6-aminohexanol to 6-hydroxyhexanal (4J); a 6-hydroxyhexanoate reductase catalyzing conversion of 6-hydroxyhexanoate to 6-hydroxyhexanal (4K); an adipate reductase catalyzing conversion of ADA to adipate semialdehyde (4L); or an adipyl-CoA transferase, hydrolase or synthase catalyzing conversion of adipyl-CoA to ADA (4M).

Further provided herein are methods of producing a target product described herein. In one aspect is a method of producing a target product selected from HMD, 6ACA, ADA, CPL, CPO, LVA, and HDO the method includes culturing cells as described herein under conditions and for a sufficient period of time to produce the target product.

Provided herein are target product produced according to the methods described herein. In one aspect is HMD according to the methods described herein. In another aspect is 6ACA according to the methods described herein. In another aspect is ADA according to the methods described herein. In another aspect is CPL according to the methods described herein. In another aspect is CPO according to the methods described herein. In another aspect is LVA according to the methods described herein. In another aspect is HDO according to the methods described herein.

Provided herein are target products produced using the cells described herein. In one aspect is HMD produced from a cell described herein. In another aspect is 6ACA produced from a cell described herein. In another aspect is ADA produced from a cell described herein. In another aspect is CPL produced from a cell described herein. In another aspect is CPO produced from a cell described herein. In another aspect is LVA produced from a cell described herein. In another aspect is HDO produced from a cell described herein.

Also provided herein are compositions of target products. In one aspect is a composition that includes a target product described herein and a byproduct selected from Table 10 or Table 11. In another aspect is a composition that includes a target product selected from LVA, 6ACA, CPL, CPO, ADA, HMD or HDO and a byproduct selected from Table 10 or Table 11.

In another aspect is a biobased product that includes one or more target products described herein. In yet another aspect is a molded product obtained by molding a biobased product described herein.

In yet another aspect is a method for producing polyamide derived from renewable resources. In one aspect, the method includes initiating polymerization of HMD, ADA, or CPL in a starting composition that includes HMD, ADA, or CPL described herein; allowing the polymerization of the HMD, ADA, or CPL to continue thereby producing a polyamide; terminating the polymerization; and isolating the produced polyamide, thereby producing polyamide from a renewable source.

### BRIEF DESCRIPTION OF THE DRAWINGS

FIG. 1 illustrates exemplary pathways from succinyl-CoA and acetyl-CoA to hexamethylenediamine (HMD), caprolactam or levulinic acid. Pathways for the production of for example adipate, 6-aminocaproate, caprolactam, hexamethylenediamine and levulinic acid from succinyl-CoA and acetyl-CoA are depicted. The enzymes are designated as follows: A) 3-oxoadipyl-CoA thiolase, B) 3-oxoadipyl-CoA reductase, C) 3-hydroxyadipyl-CoA dehydratase, D) 5-carboxy-2-pentenoyl-CoA reductase, E) 3-oxoadipyl-CoA/acyl-CoA transferase, F) 3-oxoadipyl-CoA synthase, G) 3-oxoadipyl-CoA hydrolase, H) 3-oxoadipate reductase, I) 3-hydroxyadipate dehydratase, J) 5-carboxy-2-pentenoate reductase, K) adipyl-CoA/acyl-CoA transferase, L) adipyl-CoA synthase, M) adipyl-CoA hydrolase, N) adipyl-CoA reductase (aldehyde forming), O) 6-aminocaproate transaminase, P) 6-aminocaproate dehydrogenase, Q) 6-aminocaproyl-CoA/acyl-CoA transferase, R) 6-aminocaproyl-CoA synthase, S) amidohydrolase, T) spontaneous cyclization, U) 6-aminocaproyl-CoA reductase (aldehyde forming), V) HMDA transaminase, W) HMDA dehydrogenase, X) adipate reductase, Y) adipate kinase, Z) adipylphosphate reductase, and AA) 3-oxoadipate decarboxylase.

FIG. 2 illustrates exemplary biosynthetic pathways leading to hexanoyl-CoA using NADH-dependent enzymes and with acetyl-CoA as a central metabolite. A) is an Acetyl-CoA carboxylase (EC 6.4.1.2); B) is a Beta-ketothiolase (EC 2.3.1.9; such as atoB, phaA, bktB); C) is an Acetoacetyl-CoA synthase (EC 2.3.1.194); D) is a 3-hydroxyacyl-CoA dehydrogenase or an Acetoacetyl-CoA reductase (EC 1.1.1.35 or 1.1.1.157; such as fadB, hbd or phaB); E) is an Enoyl-CoA hydratase (EC 4.2.1.17 or 4.2.1.119, such as crt or phaJ); F) is a Trans-2-enoy-CoA reductase (EC 1.3.1.8, 1.3.1.38 or 1.3.1.44, such as Ter or tdter); G) is a Beta-ketothiolase (EC 2.3.1.16, such as bktB); H) is a 3-hydroxyacyl-CoA dehydrogenase or Acetoacetyl-CoA reductase (EC 1.1.1.35 or 1.1.1.157, such as fadB, hbd, phaB, or FabG); J) is an Enoyl-CoA hydratase (EC 4.2.1.17 or 4.2.1.119, such as crt or phaJ); K) is a Trans-2-enoy-CoA reductase (EC 1.3.1.8, 1.3.1.38, or 1.3.1.44, such as Ter or tdter); L) is a Butanal dehydrogenase (EC 1.2.1.57); M) is an Aldehyde dehydrogenase (EC 1.2.1.4); and N) is a thioesterase (EC 3.2.1, such as YciA, tesB, or Acot13).

FIG. 3 illustrates exemplary biosynthetic pathway leading to 6-aminhexanoate using hexanoate as a central precursor and a schematic of an exemplary biosynthetic pathway leading to caprolactam from 6-aminohexanoate. P) is a Monooxygenase (EC 1.14.15.1, such as CYP153A, ABE47160.1, ABE47159.1, ABE47158.1, CAH04396.1, CAH04397.1, CAH04398.1, or ACJ06772.1); Q) is an Alcohol dehydrogenase (EC 1.1.1.2 or 1.1.1.258, such as CAA90836.1, YMR318c, cpnD, gabD, or ChnD); R) is a ω-transaminase (EC 2.6.1.18, 2.6.1.19, 2.6.1.29, 2.6.1.48, or 2.6.1.82, such as AA59697.1, AAG08191.1, AAY39893.1, ABA81135.1, AEA39183.1); and S) is a lactamase (EC 3.5.2).

FIG. 4 illustrates exemplary biosynthetic pathways leading to 1,6-hexanediol. A) is a 6-aminocaproyl-CoA transferase or synthetase catalyzing conversion of 6ACA to 6-aminocaproyl-CoA; B) is a 6-aminocaproyl-CoA reductase catalyzing conversion of 6-aminocaproyl-CoA to 6-aminocaproate semialdehyde; C) is a 6-aminocaproate semialdehyde reductase catalyzing conversion of 6-aminocaproate semialdehyde to 6-aminohexanol; D) is a 6-aminocaproate reductase catalyzing conversion of 6ACA to 6-aminocaproate semialdehyde; E) is an adipyl-CoA reductase adipyl-CoA to adipate semialdehyde; F) is an adipate semialdehyde reductase catalyzing conversion of adipate semialdehyde to 6-hydroxyhexanoate; G) is a 6-hydroxyhexanoyl-CoA transferase or synthetase catalyzing conversion of 6-hydroxyhexanoate to 6-hydroxyhexanoyl-CoA; H) is a 6-hydroxyhexanoyl-CoA reductase catalyzing conversion of 6-hydroxyhexanoyl-CoA to 6-hydroxyhexanal; I) is a 6-hydroxyhexanal reductase catalyzing conversion of 6-hydroxyhexanal to HDO; J) is a 6-aminohexanol aminotransferase or oxidoreductases catalyzing conversion of 6-aminohexanol to 6-hydroxyhexanal; K) is a 6-hydroxyhexanoate reductase catalyzing conversion of 6-hydroxyhexanoate to 6-hydroxyhexanal; L) is an adipate reductase catalyzing conversion of ADA to adipate semialdehyde; and M) is an adipyl-CoA transferase, hydrolase or synthase catalyzing conversion of adipyl-CoA to ADA.

FIG. 5 illustrates exemplary pathways from adipate or adipyl-CoA to caprolactone. Enzymes are A). adipyl-CoA reductase, B) adipate semialdehyde reductase, C) 6-hydroxyhexanoyl-CoA transferase or synthetase, D) 6-hydroxyhexanoyl-CoA cyclase or spontaneous cyclization, E) adipate reductase, F) adipyl-CoA transferase, synthetase or hydrolase, G) 6-hydroxyhexanoate cyclase, H) 6-hydroxyhexanoate kinase, I) 6-hydroxyhexanoyl phosphate cyclase or spontaneous cyclization, and J) phosphotrans-6-hydroxyhexanoylase.

### DETAILED DESCRIPTION

Unless defined otherwise, all technical and scientific terms used herein have the same meaning as commonly understood by those of ordinary skill in the art to which the invention belongs. Any methods, devices and materials similar or equivalent to those described herein can be used in the practice of this invention. The following definitions are provided to facilitate understanding of certain terms used frequently herein and are not meant to limit the scope of the present disclosure. All references referred to herein are incorporated by reference in their entirety.

As used herein, the phrases “non-naturally occurring” and “genetically modified cell” are used interchangeably and refer to a microbial organism having at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a biosynthetic pathway capable of producing hexamethylenediamine (HMD); levulinic acid (LVA), 6-aminocaproic acid (6ACA), caprolactam (CPL), caprolactone (CPO), adipic acid (ADA), or 1,6-hexanediol (HDO) or a combination thereof. Thus, in certain instances the biosynthetic pathway is one producing HMD (or an intermediate thereof). In another example is a biosynthetic pathway that produces HDO.

A “hexamethylenediamine (HMD) pathway” refers to polypeptides, including enzymes or proteins in a biosynthetic pathway capable of producing HMD. A “levulinic acid (LVA) pathway” refers to polypeptides, including enzymes or proteins in a biosynthetic pathway capable of producing LVA. A “caprolactone (CPO) pathway” refers to polypeptides, including enzymes or proteins in a biosynthetic pathway capable of producing HMD. A “1,6-hexanediol (HDO) pathway” refers to polypeptides, including enzymes or proteins in a biosynthetic pathway capable of producing HDO. Pathways described herein can include genetic disruptions as described herein that can result in increased product yield as well as include genetic modifications described herein which result in decreased levels of byproducts compared to production without such genetic disruptions.

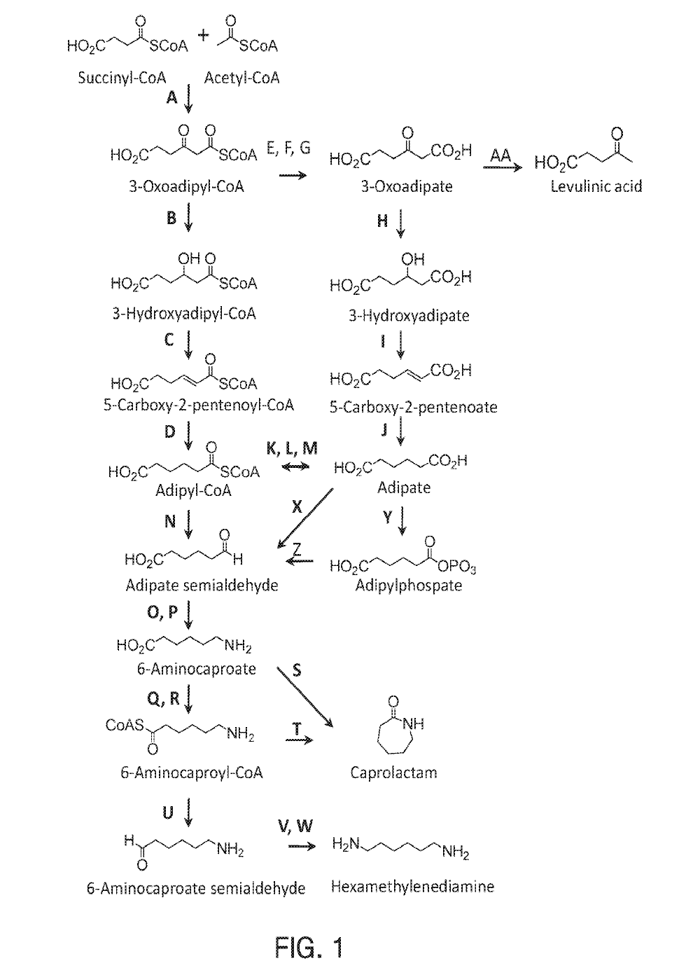

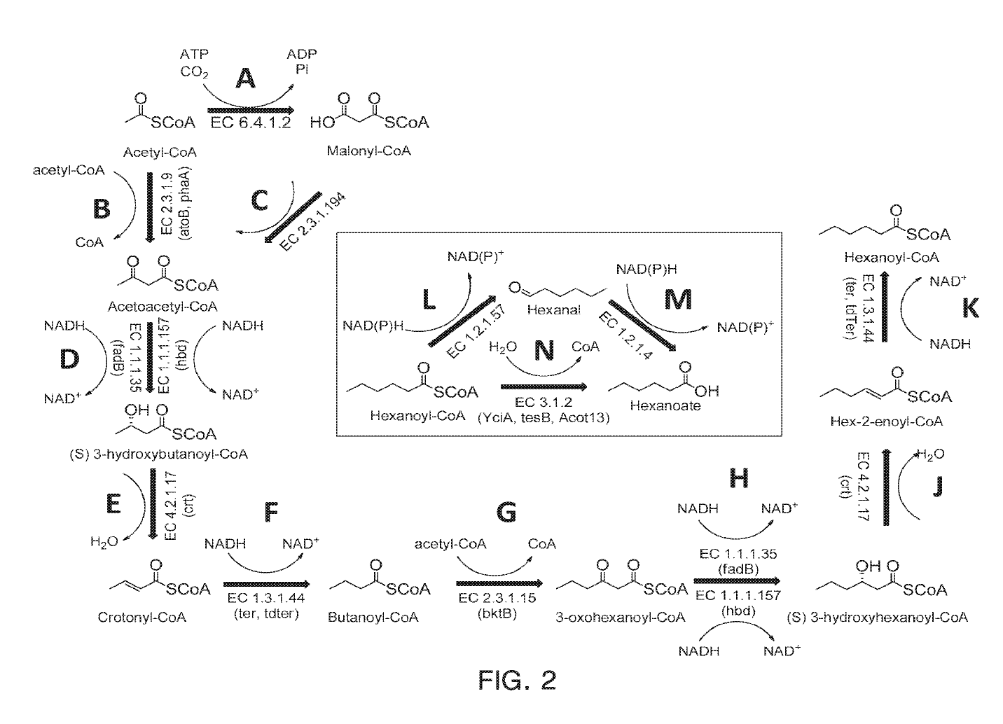

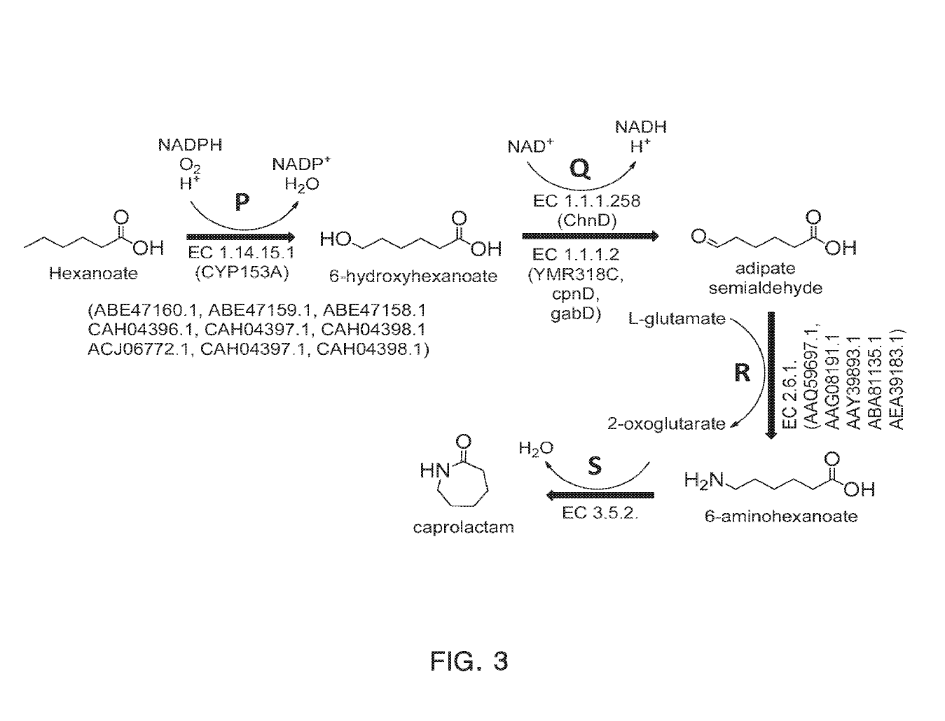

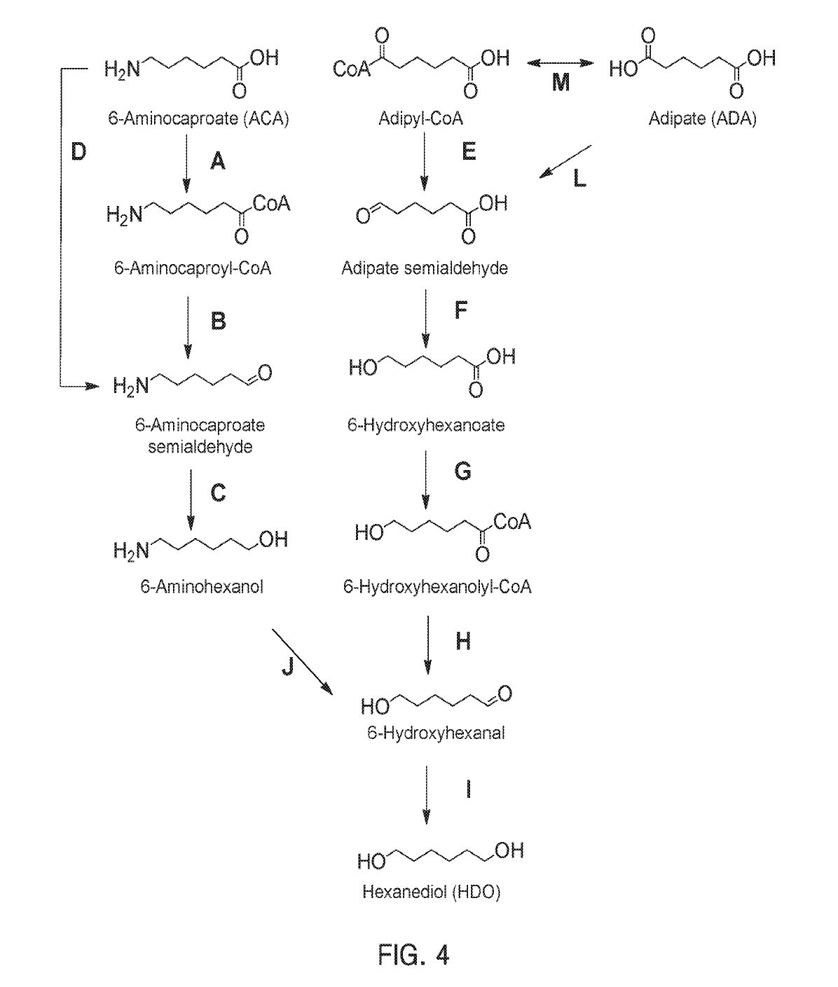

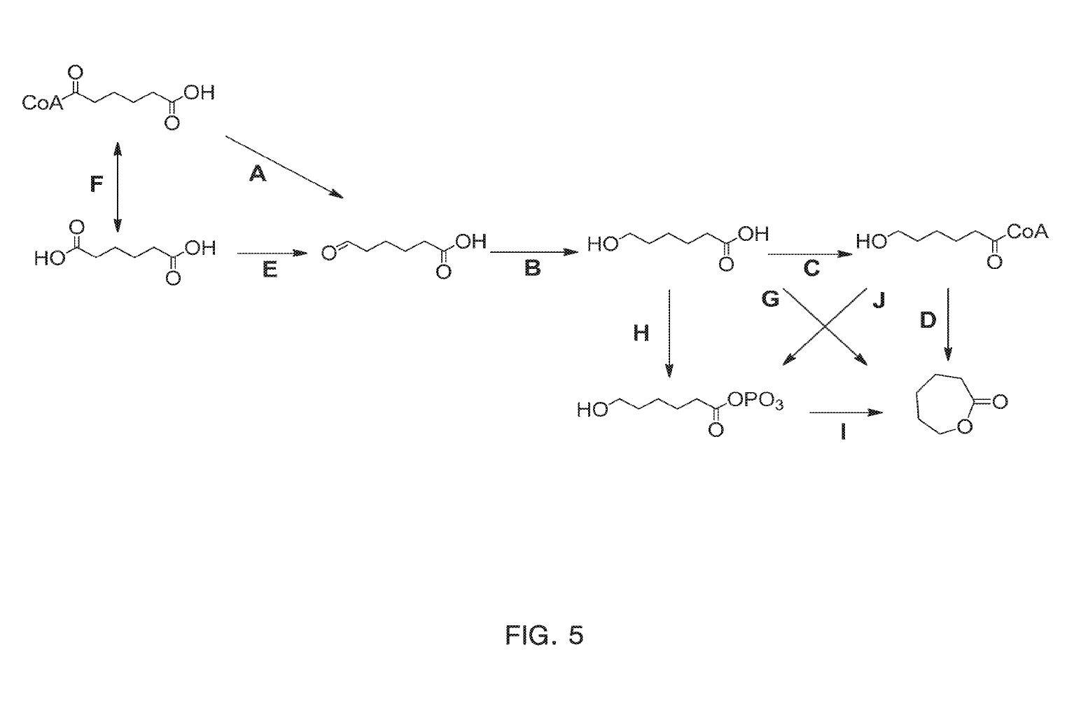

As used herein a “target product” refers to a product or compound synthesized using a biosynthetic pathway described herein (e.g. HMD biosynthesized using a HMD pathway described herein). The phrase typically refers to an “end product” of the biosynthetic pathway that is the terminal compound of a biosynthetic pathway described herein. Thus, a target product can refer to a compound present in a biosynthetic pathway described herein where the biosynthetic pathway terminates at that compound. Accordingly, intermediate compounds set forth in the biosynthetic pathways described herein can be target products in embodiments described herein. Exemplary target products include HMD, LVA, 6ACA, CPL, CPO, ADA, and HDO and the intermediate compounds within biosynthetic pathways described herein to biosynthesize such target products as exemplified, for example, in FIG. 1, FIG. 2, FIG. 3, FIG. 4, and FIG. 5.

“Byproduct” as used herein refers to compounds biosynthesized in a biosynthetic pathway described herein which lower target product purity (e.g. are present in combination with the final target product) or otherwise decrease target product yields. A byproduct can be an intermediate of a compound along the pathway. That is, a byproduct can be an intermediate compound itself (as shown in for example FIG. 1, FIG. 2, FIG. 3, FIG. 4, and FIG. 5). A byproduct can also be a compound resulting from catalytic activity of a compound set forth in a biosynthetic pathway described herein.

Enzymes can react or catalyze reactions on pathway intermediates which can subsequently draw reactants away from biosynthesis of a selected target product. In such instances, the yield, titer, or rate of production of a desired target product can be reduced. Such byproducts also need not be present in the final target product composition. That is, byproducts arising from, for example, catalysis of intermediates within a biosynthetic pathway described herein may not be found in detectable amounts within a final target product composition described herein. Accordingly, a byproduct can be a compound which is a result of undesired catalysis on pathway intermediates or final products described herein optionally present in the final composition. Furthermore, byproducts described herein can result from catalysis of other byproducts. Thus, in certain instances described herein, a byproduct is 2 or more steps removed from a biosynthetic pathway described herein One skilled in the art would readily understand that such enzymes can react in a “cascade” such that generating one byproduct from a pathway intermediate can lead to generation of multiple other byproducts which can subsequently catalyze reactions on each independent byproduct in the chain. Likewise, attenuation of enzymes resulting in a particular byproduct can reduce production of other byproducts which result from catalysis on the particular byproduct.

A metabolic modification refers to a biochemical reaction that is altered from its naturally occurring state. Therefore, non-naturally occurring microorganisms can have genetic modifications to nucleic acids encoding metabolic polypeptides, or functional fragments thereof. Exemplary metabolic modifications are disclosed herein.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood, as disclosed herein, that such more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

As used herein, the term “gene disruption,” “genetic modification” or grammatical equivalents thereof, is intended to mean a genetic alteration that renders the encoded gene product inactive or attenuated. The genetic alteration can be, for example, deletion of the entire gene, deletion of a regulatory sequence required for transcription or translation, deletion of a portion of the gene which results in a truncated gene product, or by any of various mutation strategies that inactivate or attenuate the encoded gene product. One particularly useful method of gene disruption is complete gene deletion because it reduces or eliminates the occurrence of genetic reversions in the non-naturally occurring microorganisms of the invention. A gene disruption also includes a null mutation, which refers to a mutation within a gene or a region containing a gene that results in the gene not being transcribed into RNA and/or translated into a functional gene product. Such a null mutation can arise from many types of mutations including, for example, inactivating point mutations, deletion of a portion of a gene, entire gene deletions, or deletion of chromosomal segments.

As used herein, the term “growth-coupled” when used in reference to the production of a target product is intended to mean that the biosynthesis of the referenced target product is produced during the growth phase of a microorganism. In a particular embodiment, the growth-coupled production can be obligatory, meaning that the biosynthesis of the referenced biochemical is an obligatory product produced during the growth phase of a microorganism.

As used herein, the term “attenuate,” or grammatical equivalents thereof, is intended to mean to weaken, reduce or diminish the activity or amount of an enzyme or protein. Attenuation of the activity or amount of an enzyme or protein can mimic complete disruption if the attenuation causes the activity or amount to fall below a critical level required for a given pathway to function. However, the attenuation of the activity or amount of an enzyme or protein that mimics complete disruption for one pathway, can still be sufficient for a separate pathway to continue to function. For example, attenuation of an endogenous enzyme or protein can be sufficient to mimic the complete disruption of the same enzyme or protein for production of a target product described herein, but the remaining activity or amount of enzyme or protein can still be sufficient to maintain other pathways, such as a pathway that is critical for the host microbial organism to survive, reproduce or grow. Attenuation of an enzyme or protein can also be weakening, reducing or diminishing the activity or amount of the enzyme or protein in an amount that is sufficient to increase yield of a target product described herein, but does not necessarily mimic complete disruption of the enzyme or protein.

The non-naturally occurring microbial organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

In the case of gene disruptions and genetic modifications described herein, a particularly useful stable genetic alteration is a gene deletion. The use of a gene deletion to introduce a stable genetic alteration is particularly useful to reduce the likelihood of a reversion to a phenotype prior to the genetic alteration. For example, stable growth-coupled production of a biochemical can be achieved, for example, by deletion of a gene encoding an enzyme catalyzing one or more reactions within a set of metabolic modifications. The stability of growth-coupled production of a biochemical can be further enhanced through multiple deletions, significantly reducing the likelihood of multiple compensatory reversions occurring for each disrupted activity.

Those skilled in the art will understand that the genetic alterations, including those exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An “ortholog” is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, “paralogs” are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having biosynthetic capability to produce a target product described herein and one or more genetic modifications described herein, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. Similarly for a gene disruption, evolutionally related genes can also be disrupted or deleted in a host microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

Provided herein, inter alia, are genetically modified cells (e.g. non-naturally occurring microorganisms) capable of producing a target product (e.g. HMD, LVA, 6ACA, CPL, CPO, ADA, or HDO), where the genetically modified cell includes one or more genetic modifications selected from:

(a) a genetic modification that decreases activity of an enzyme selected from Oxidoreductase (oxo to alcohol) (A1); Oxidoreductase (acyl-CoA to alcohol) (A2); Oxidoreductase (aldehyde to acid) (A3); Oxidoreductase (acyl-CoA to aldehyde) (A4); Aldehyde oxidase (aldehyde to acid) (A5); Oxidoreductase (alkene to alkane) (A6); Oxidoreductase (amine to oxo) (A7); Amine N-methyltransferase (amine to methylamine) (A8); Carbamoyl transferase (amine to carbamoylamine) (A9); Acyltransferase (acyl-CoA and acetyl-CoA to 3-oxoacyl-CoA) (A10); Acyltransferase (N-acyltransferase) (A11); N-propylamine synthase (amine to N-propylamine) (A12); Aminotransferase (pyrroline forming) (A13); CoA transferase (acyl-CoA to acid) (A14); Thioester hydrolase (acyl-CoA to acid) (A15); Decarboxylase acting on 3-oxoacids (A16); Dehydratase (hydroxyacid to alkene) (A17); Ammonia-lyase (aminoacid to alkene) (A18); CoA ligase (acyl-CoA to acid) (A19); glutamyl:amine ligase (A20); Amine hydroxylase (amine to hydroxylamine) (A21); Oxidoreductase (alkane to alkene, irreversible) (A22); Amine oxidase (amine to aldehyde, irreversible) (A23); Short-chain diamine exporter (A24); and Putrescine permease (A25);

(b) a genetic modification that increases activity of an enzyme selected from Amide hydrolase or amidase (B1); Cyclic amide hydrolase or lactamase (B2); CoA ligase (B3); Diamine transporter (longer chain diamines) (B4); Diamine permease (B5); and

(c) a combination of two or more, three or more, four or more, five or more, six or more, seven or more, eight or more, nine or more, ten or more, or all of the genetic modifications of (a) and (b). The cell produces less byproduct than a cell without such one or more genetic modifications.

Further provided herein are genetically modified cells capable of producing a target product, where the target product can be levulinic acid (LVA), 6-aminocaproic acid (6ACA), caprolactam (CPL), caprolactone (CPO), adipic acid (ADA), hexamethylenediamine (HMD), or 1,6-hexanediol (HDO) or a combination thereof. In such instances the genetically modified cell includes one or more genetic modifications selected from: (a) a genetic modification that decreases activity of an enzyme selected from an Oxidoreductase acting on an aldehyde or oxo moiety (A1); Oxidoreductase acting on a acyl-CoA moiety (A2); Oxidoreductase acting on an aldehyde moiety (A3); Oxidoreductase acting on an aldehyde or acyl-CoA moiety (A4); Aldehyde oxidase acting on an aldehyde moiety (A5); Oxidoreductase acting on an alkene or alkane moiety (A6); Oxidoreductase acting on an amine moiety (A7); Amine N-methyltransferase acting on an amine moiety (A8); Carbamoyl transferase acting on an amine moiety (A9); Acyltransferase acting on an acyl-CoA moiety (A10); Acyltransferase acting on an amine or acyl-CoA moiety (A11); N-propylamine synthase acting on an amine moiety (A12); Aminotransferase acting on an amine or aldehyde moiety (A13); CoA transferase acting on an acyl-CoA or an acid moiety (A14); Thioester hydrolase acting on an acyl-CoA moiety (A15); Decarboxylase acting on an oxoacid moiety (A16); Dehydratase acting on a hydroxyacid moiety (A17); Ammonia-lyase acting on an amine moiety (A18); CoA ligase acting on an acyl-CoA or acid moiety (A19); glutamyl:amine ligase acting on an amine moiety (A20); Amine hydroxylase acting on an amine moiety (A21); Oxidoreductase acting on an acyl-CoA moiety (A22); Amine oxidase acting on an amine moiety (A23); short chain diamine exporter acting on a diamine moiety (A24); and putrescine permease acting on a diamine moiety (A25); (b) a genetic modification that increases activity of an enzyme selected from Amide hydrolase or amidase acting on an amide moiety (B1); Cyclic amide hydrolase or lactamase acting one a cyclic amide moiety (B2); CoA ligase acting on an acid moiety (B3); Diamine transporter (longer chain diamines) acting on an amine moiety (B4); and diamine permease acting on an amine moiety (B5); and a combination of two or more, three or more, four or more, five or more, six or more, seven or more, eight or more, nine or more, ten or more, or all of the genetic modifications of (a) and (b), where the cell produces a reduced amount of one or more byproducts described herein when compared to a cell without the one or more genetic modifications.

In certain instances cells described herein include a combination of 2, 3, 4, 5, 6, 7, 8, 9, or 10, or more, or all of the genetic modifications of (a) and (b) where such a cell produces less byproduct than a cell without such one or more genetic modifications. Cells described herein are capable of synthesizing target products described herein, including pathway intermediates therein as shown, for example, in FIGS. 1-5. Thus, in certain instances, pathways described herein can be modified as described herein to biosynthesize a particular intermediate compound within a described pathway. Such modifications are understood by those in the art to prevent or reduce conversion of such a pathway intermediate to another downstream compound, such as for example HMD or HDO.

The genetic modifications described herein are useful for biosynthetically producing target products with reduced or eliminated byproducts. Such genetic modifications can include modifications that decrease activity of an enzyme. Thus, a cell described herein can include a genetic modification of an enzyme selected from A1-A25 (e.g., A1, A2, A3, A4, A5, A6, A7, A8, A9, A10, A11, A12, A13, A14, A15, A16, A17, A18, A19, A20, A21, A22, A23, A24, A25) of Table 3 where A1 is an oxidoreductase (aldehyde or oxo to alcohol); A2 is an oxidoreductase (2 step, acyl-CoA to alcohol); A3 is an Oxidoreductase (aldehyde to acid); A4 is an Oxidoreductase (acyl-CoA to aldehyde); A5 is an Aldehyde oxidase (aldehyde to acid); A6 is an Oxidoreductase (alkene to alkane); A7 is an Oxidoreductase (amine to oxo); A8 is an Amine N-methyltransferase (amine to methylamine); A9 is a Carbamoyl transferase (amine to carbamoylamine); A10 is an Acyltransferase (N-acyltransferase); A11 is an N-propylamine synthase (amine to N-propylamine); A12 is an N-propylamine synthase (amine to N-propylamine); A13 is an Aminotransferase (pyrroline forming); A14 is a CoA transferase (acyl-CoA to acid); A15 is a thioester hydrolase (acyl-CoA to acid); A16 is a Decarboxylase acting on 3-oxoacids; A17 is a Dehydratase (hydroxyacid to alkene); A18 is an Ammonia-lyase (aminoacid to alkene); A19 is a CoA ligase (acyl-CoA to acid); A20 is a gluyamyl:amine ligase; A21 is an Amine hydroxylase (amine to hydroxylamine); A22 is an Oxidoreductase (alkane to alkene, other e-acceptor); A23 is an Amine oxidase (amine to aldehyde, irreversible); A24 is an Short-chain diamine exporter; A25 is an Putrescine permease; B1 is amide hydrolase or amidase; B2 is an Cyclic amide hydrolase or lactamase; B3 is a CoA ligase; B4 is a Diamine transporter (longer chain diamines; and B5 is an Diamine permease.

In certain instances, the cell produces less byproduct when the cell includes a combination of two or more genetic modification of enzymes selected from A1-A25 than a cell lacking such genetic modifications as described herein. Thus, cells described herein can include a combination of 2, 3, 4, or more genetic modifications of enzymes selected from A1-A25. In such instances, the cells can produce HMD, LVA, 6ACA, CPL, CPO, ADA, or HDO having less byproduct than a cell lacking such genetic modifications. In certain instances, the cells can produce HMD, LVA, 6ACA, CPL, CPO, ADA, or HDO at a greater amount when the cells have one or more genetic modifications described herein. When enzyme activity is decreased using a genetic modification described herein, the decreased activity can reduce or eliminate production of a byproduct set forth in any one of Tables 10, 11, or 12.

The genetic modification can be one that increases activity of an enzyme in a cell intended to produce a target product. In such instances, the genetic modification can be an enzyme selected from B1-B5 (e.g., B1, B2, B3, B4, B5) of Table 3 where B1-B5 are as described above. The genetically modified cell having such a genetic modification can produce less byproduct than a cell lacking such modifications. In certain instances, the cells can produce HMD, LVA, 6ACA, CPL, CPO, ADA, or HDO at a greater amount when the cells have one or more genetic modifications described herein. Accordingly, in certain instances, a genetically modified cell can include a genetic modification of an enzyme selected from B1-B5 as described herein, where the genetically modified cell is capable of producing a target product described herein. The target product can be HMD, LVA, 6ACA, CPL, CPO, ADA, or HDO. The genetic modification can be two or more enzymes selected from B1-B5 as described herein. Thus, cells described herein can include a combination of 2, 3, 4, or 5 genetic modifications of enzymes selected from B1 to B5. In such instances, the cells can produce a target product described herein (e.g. HMD, LVA, 6ACA, CPL, CPO, ADA, or HDO) having less byproducts than a cell lacking such genetic modifications. When enzyme activity is decreased using a genetic modification described herein, the decreased activity can reduce or eliminate production of a byproduct set forth in Table 10.

Those of skill in the art would readily recognize that combinations of genetic modifications set forth in Table 3 and Table 4 are useful for reducing byproducts described herein. For example, each of A1-A25 can be combined with one of B1-B5. In another example each of A1-A25 can be combined with each of B1-B5. In yet another example each of A1-A25 can be combined with two, three, or four of B1-B5 (e.g. A1 combined with B1B2, B1B3, B1B4, etc. . . . ). Alternatively, each of B1-B5 can be combined with one of A1-A25. In another example each of B1-B5 can be combined with each of A1-A25. In yet another each of B1-B5 can be combined with two, three, or four or more of A1-A25 (e.g. B1 combined with A1A2, A1A3, A1A4, etc. . . . ). One skilled in the art will understand combinations of A1-A25 set forth in Table 1 can combined with the combinations of B1-B5 set forth in Table 2 to make combinations of A1-A25 and B1-B5 useful for reducing levels of byproducts in target products synthesized using the biosynthetic pathways described herein. Thus, provided herein are genetically modified cells where the cell has a combination of genetic modifications as described above or as exemplified by the combinations set forth in Tables 1 and 2. Accordingly, in all such instances, a cell having any such a genetic modification can be capable of producing HMD, LVA, 6ACA, CPL, CPO, ADA, or HDO.

TABLE 1
combinations of A1-A25

| | A1 | A2 | A3 | A4 | A5 | A6 | A7 | A8 | A9 | A10 | A11 | A12 | A13 |
|---|---|---|---|---|---|---|---|---|---|---|---|---|---|
| A1′ | X | ± | ± | ± | ± | ± | ± | ± | ± | ± | ± | ± | ± |
| A2′ | ± | X | ± | ± | ± | ± | ± | ± | ± | ± | ± | ± | ± |
| A3′ | ± | ± | X | ± | ± | ± | ± | ± | ± | ± | ± | ± | ± |
| A4′ | ± | ± | ± | X | ± | ± | ± | ± | ± | ± | ± | ± | ± |
| A5′ | ± | ± | ± | ± | X | ± | ± | ± | ± | ± | ± | ± | ± |
| A6′ | ± | ± | ± | ± | ± | X | ± | ± | ± | ± | ± | ± | ± |
| A7′ | ± | ± | ± | ± | ± | ± | X | ± | ± | ± | ± | ± | ± |
| A8′ | ± | ± | ± | ± | ± | ± | ± | X | ± | ± | ± | ± | ± |
| A9′ | ± | ± | ± | ± | ± | ± | ± | ± | X | ± | ± | ± | ± |
| A10 | ± | ± | ± | ± | ± | ± | ± | ± | ± | X | ± | ± | ± |
| A11 | ± | ± | ± | ± | ± | ± | ± | ± | ± | ± | X | ± | ± |
| A12 | ± | ± | ± | ± | ± | ± | ± | ± | ± | ± | ± | X | ± |
| A13 | ± | ± | ± | ± | ± | ± | ± | ± | ± | ± | ± | ± | X |
| A14 | ± | ± | ± | ± | ± | ± | ± | ± | ± | ± | ± | ± | ± |
| A15 | ± | ± | ± | ± | ± | ± | ± | ± | ± | ± | ± | ± | ± |
| A16 | ± | ± | ± | ± | ± | ± | ± | ± | ± | ± | ± | ± | ± |
| A17 | ± | ± | ± | ± | ± | ± | ± | ± | ± | ± | ± | ± | ± |
| A18 | ± | ± | ± | ± | ± | ± | ± | ± | ± | ± | ± | ± | ± |
| A19 | ± | ± | ± | ± | ± | ± | ± | ± | ± | ± | ± | ± | ± |
| A20 | ± | ± | ± | ± | ± | ± | ± | ± | ± | ± | ± | ± | ± |
| A21 | ± | ± | ± | ± | ± | ± | ± | ± | ± | ± | ± | ± | ± |
| A22 | ± | ± | ± | ± | ± | ± | ± | ± | ± | ± | ± | ± | ± |
| A23 | ± | ± | ± | ± | ± | ± | ± | ± | ± | ± | ± | ± | ± |
| A24 | ± | ± | ± | ± | ± | ± | ± | ± | ± | ± | ± | ± | ± |
| A25 | ± | ± | ± | ± | ± | ± | ± | ± | ± | ± | ± | ± | ± |

combinations of A1-A25

| | A14 | A15 | A16 | A17 | A18 | A19 | A20 | A21 | A22 | A23 | A24 | A25 |
|---|---|---|---|---|---|---|---|---|---|---|---|---|
| A1′ | ± | ± | ± | ± | ± | ± | ± | ± | ± | ± | ± | ± |
| A2′ | ± | ± | ± | ± | ± | ± | ± | ± | ± | ± | ± | ± |
| A3′ | ± | ± | ± | ± | ± | ± | ± | ± | ± | ± | ± | ± |
| A4′ | ± | ± | ± | ± | ± | ± | ± | ± | ± | ± | ± | ± |
| A5′ | ± | ± | ± | ± | ± | ± | ± | ± | ± | ± | ± | ± |
| A6′ | ± | ± | ± | ± | ± | ± | ± | ± | ± | ± | ± | ± |
| A7′ | ± | ± | ± | ± | ± | ± | ± | ± | ± | ± | ± | ± |
| A8′ | ± | ± | ± | ± | ± | ± | ± | ± | ± | ± | ± | ± |
| A9′ | ± | ± | ± | ± | ± | ± | ± | ± | ± | ± | ± | ± |
| A10 | ± | ± | ± | ± | ± | ± | ± | ± | ± | ± | ± | ± |
| A11 | ± | ± | ± | ± | ± | ± | ± | ± | ± | ± | ± | ± |
| A12 | ± | ± | ± | ± | ± | ± | ± | ± | ± | ± | ± | ± |
| A13 | ± | ± | ± | ± | ± | ± | ± | ± | ± | ± | ± | ± |
| A14 | X | ± | ± | ± | ± | ± | ± | ± | ± | ± | ± | ± |
| A15 | ± | X | ± | ± | ± | ± | ± | ± | ± | ± | ± | ± |
| A16 | ± | ± | X | ± | ± | ± | ± | ± | ± | ± | ± | ± |
| A17 | ± | ± | ± | X | ± | ± | ± | ± | ± | ± | ± | ± |
| A18 | ± | ± | ± | ± | X | ± | ± | ± | ± | ± | ± | ± |
| A19 | ± | ± | ± | ± | ± | X | ± | ± | ± | ± | ± | ± |
| A20 | ± | ± | ± | ± | ± | ± | X | ± | ± | ± | ± | ± |
| A21 | ± | ± | ± | ± | ± | ± | ± | X | ± | ± | ± | ± |
| A22 | ± | ± | ± | ± | ± | ± | ± | ± | X | ± | ± | ± |
| A23 | ± | ± | ± | ± | ± | ± | ± | ± | ± | X | ± | ± |
| A24 | ± | ± | ± | ± | ± | ± | ± | ± | ± | ± | X | ± |
| A25 | ± | ± | ± | ± | ± | ± | ± | ± | ± | ± | ± | X |

TABLE 2
combinations of B1-B5

| | B1 | B2 | B3 | B4 | B5 |
|---|---|---|---|---|---|
| B1 | X | ± | ± | ± | ± |
| B2 | ± | X | ± | ± | ± |
| B3 | ± | ± | X | ± | ± |
| B4 | ± | ± | ± | X | ± |
| B5 | ± | ± | ± | ± | X |

The genetically modified cells described herein can include a genetic modification of an enzyme selected from A1 to A25, where A1 and A25 correspond to the enzymes described above.

Enzymes described herein can also be referred to according to their EC number as set forth in Table 3 (e.g. an oxidoreductase (aldehyde or oxo to alcohol) of the EC class 1.1.1. In certain instances enzymes can be further described by their EC number where such an EC number includes a 4th tier value (e.g. 1.1.1.a., where a is 1 or 2). EC numbers for enzymes are well understood in the art. See, for example, Yu et al., Biotech. and Bioengin., Vol. 111, No. 12, December, 2014, 2580-86. For example, an enzyme of EC class 1.1.1.1 includes all oxidoreductases classified under the EC 1.1.1.1 classification. Accordingly, one skilled in the art would readily recognize enzymes listed in Table 3 and 4, for example, can be substituted or exchanged with enzymes of similar or identical function. Such enzymes can be considered redundant in a particular organism (e.g., enzymes in a cell that perform the same enzymatic reaction using the same substrate).

Enzymes described herein (e.g. A1-A25 and B1-B5) can include EC class numbers as set forth in Tables 3 and 4. In certain instances A1 is of the EC class 1.1.1; A2 is of EC class 1.1.1; A3 is of EC class 1.2.1; A4 is of EC class 1.2.1; A5 is of EC class 1.2.3; A6 is of EC class 1.3.1; A7 is of EC class 1.4.1; A8 is of EC class 2.1.1; A9 is of EC class 2.1.3; A10 is of EC class 2.3.1; A11 is of EC class 2.3.1; A12 is of EC class 2.5.1; A13 is of EC class 2.6.1; A14 is of EC class 2.8.3; A15 is of EC class 3.1.2; A16 is of EC class 4.1.1; A17 is of EC class 4.2.1; A18 is of EC class 4.3.1; A19 is of EC class 6.2.1; A20 is of EC class 6.3.1; A22 is of EC class 1.3.8; A23 is of EC class 1.4.9; A24 is of EC class 3.6.3; B1 is of EC class 3.5.1; B2 is of EC class 3.5.2; B3 is of EC class 6.2.1; B4 is of EC class 3.6.3; and/or B5 is of EC class 3.6.3.

Enzymes described herein (e.g. A1-A25 and B1-B5) can also be characterized by a corresponding EC number that includes a 4th tier value as described herein. In such instances A1 is of the EC class 1.1.1.a, wherein a is 1 or 2; A2 is of EC class 1.1.1.b, wherein b is 1; A3 is of EC class 1.2.1.c, wherein c is 3, 4, 5, 19, 31, or 79; A4 is of EC class 1.2.1.d, wherein d is 57; A5 is of EC class 1.2.3.1; A6 is of EC class 1.3.1.31; A7 is of EC class 1.4.1.18; A8 is of EC class 2.1.1.h, wherein his 17, 49, or 53; A9 is of EC class 2.1.3.i, wherein i is 2, 3, 6, 8 or 9; A10 is of EC class 2.3.1.j, wherein j is 9 or 15; A11 is of EC class 2.3.1.k, wherein k is 32 or 57; A12 is of EC class 2.5.1.16; A13 is of EC class 2.6.1.m, wherein m is 11, 13, 18, 19, 22, 29, 36, 43, 46, 48, 71, 82, or 96; A14 is of EC class 2.8.3.n, wherein n is g is 1, 4, 5, 6, or 18; A15 is of EC class 3.1.2.o, wherein o is 1, 3, 5, 18, 19, or 20; A16 is of EC class 4.1.1.4; A17 is of EC class 4.2.1.q, wherein q is 2, 10, 53, or 80; A18 is of EC class 4.3.1.1; A19 and is of EC class 6.2.1.s, wherein s is 2, 4, 5, 23, or 40; A20 is of EC class 6.3.1.t, wherein t is 6, 8, or 11; A22 is of EC class 1.3.8; A23 is of EC class 1.4.9.1; A24 is of EC class 3.6.3.31; B1 is of EC class 3.5.1.u, wherein u is 46, 53, 62, or 63; B2 and is of EC class 3.5.2.v, wherein v is 9, 11, or 12; B3 is of EC class 6.2.1.w, wherein w is 2, 3, 5, 14, or 40; B4 is of EC class 3.6.3.31; or B5 is of EC class 3.6.3.31.

Alternatively, enzymes such as those set forth in Table 3 can be a homolog, ortholog, or paralog of a protein having similar or identical function—including catalysis of similar or identical substrates. Exemplary enzymes useful for genetic modification as described herein include those set forth in Table 4. The enzyme can be an enzyme of Table 4 or a homolog, paralog, or otholog thereof. Thus, one of skill in the art could readily understand that modification of enzymes as described herein in Table 3 or 4 in a suitable host can result in target products having reduced byproducts (e.g. greater purity) than identical target products produced in a cell lacking such modifications. Enzyme A1-A25 can therefore be an enzyme set forth in Table 3 or 4. Enzyme B1-B5 can be an enzyme set forth in Table 3 or 4.

Enzymes described herein can also be described by their gene name and in certain instances, by the associated host. Thus, for example, an enzyme useful for a genetic modification described herein can be yqhD of E. coli, including homologs, paralogs, and orthologs thereof (such as those described by EC class 1.1.1.a, where a is 1 or 2 including all enzymes set forth Table 3 and 4).

TABLE 3
Exemplary enzymes

| | | Exem. | | Substrate | | | Exem. |
|---|---|---|---|---|---|---|---|
| Enyme | | EC tier | | functional | Exemplary | | Pathway |
| No. | EC | 4 | Function | group | gene | Organism | substrate |
| A1 | 1.1.1 | 1, 2 | Oxidoreductase | aldehyde | yqhD | Escherichia coli | adipsa, 6- |
| | | | (oxo to alcohol) | or oxo | | | acasa |
| A2 | 1.1.1 | 1 | Oxidoreductase | acyl-CoA | adhE | Escherichia coli | accoa, |
| | | | (acyl-CoA to | | | | succoa, |
| | | | alcohol) | | | | 3oacoa, |
| | | | | | | | 3hacoa, |
| | | | | | | | 5c2pc0a, |
| | | | | | | | adipcoa, |
| | | | | | | | 6acacoa |
| A3 | 1.2.1 | 3, 4, 5, | Oxidoreductase | aldehyde | aldB, sad, | Escherichia coli | adipsa, 6- |
| | | 19, 31, | (aldehyde to acid) | | gabD | | acasa |
| | | 79 | | | | | |
| A4 | 1.2.1 | 57 | Oxidoreductase | aldehyde; | adhE | Escherichia coli | adipsa, 6- |
| | | | (acyl-CoA to | acyl-CoA | | | acasa, |
| | | | aldehyde) | | | | accoa, |
| | | | | | | | succoa, |
| | | | | | | | 3oacoa, |
| | | | | | | | 3hacoa, |
| | | | | | | | 5c2pcoa, |
| | | | | | | | adipcoa, |
| | | | | | | | 6acacoa |
| A5 | 1.2.3 | 1 | Aldehyde oxidase | aldehyde | amms, | Methylobacillus sp. | adipsa, 6- |
| | | | (aldehyde to acid) | | ammm, | KY4400 | acasa |
| | | | | | amml | | |
| A6 | 1.3.1 | 31 | Oxidoreductase | alkene, | nemA | Escherichia coli | Byprod. |
| | | | (alkene to alkane) | alkane | | | Intermed. |
| A7 | 1.4.1 | 18 | Oxidoreductase | amine | lys9 | Methyloglobulus | 6aca, |
| | | | (amine to oxo) | | | morosus KoM1 | 6acasa, |
| | | | | | | | hmda |
| A8 | 2.1.1 | 17, 49, | Amine N- | amine | cho2 | Pichia pastoris | 6aca, |
| | | 53 | methyltransferase | | | | 6acasa, |
| | | | (amine to | | | | hmda |
| | | | methylamine) | | | | |
| A9 | 2.1.3 | 2, 3, 6, | Carbamoyl | amine | argFl | Escherichia coli | 6aca, |
| | | 8, 9 | transferase (amine | | | | 6acasa, |
| | | | to | | | | hmda |
| | | | carbamoylamine) | | | | |
| A10 | 2.3.1 | 15, 9 | Acyltransferase | acyl-CoA | atoB, fadA, | Escherichia coli | accoa, |
| | | | (acyl-CoA and | | fadl | | succoa, |
| | | | acetyl-CoA to 3- | | | | 3oacoa, |
| | | | oxoacyl-CoA) | | | | 3hacoa, |
| | | | | | | | 5c2pcoa, |
| | | | | | | | adipcoa, |
| | | | | | | | 6acacoa |
| A11 | 2.3.1 | 32, 57 | Acyltransferase (N- | amine, | speG | Escherichia coli | 6aca, |
| | | | acyltransferase) | acyl-CoA | | | 6acasa, |
| | | | | | | | hmda, |
| | | | | | | | accoa, |
| | | | | | | | succoa, |
| | | | | | | | 3oacoa, |
| | | | | | | | 3hacoa, |
| | | | | | | | 5c2pcoa, |
| | | | | | | | adipcoa, |
| | | | | | | | 6acacoa |
| A12 | 2.5.1 | 16 | N-propylamine | amine | speE | Escherichia coli | 6aca, |
| | | | synthase (amine to | | | | 6acasa, |
| | | | N-propylamine) | | | | hmda |
| A13 | 2.6.1 | 11, 13, | Aminotransferase | amine, | puuE | Escherichia coli | 6aca, |
| | | 18, 19, | (pyrroline forming) | aldehyde | | | 6acasa, |
| | | 22, 29, | | | | | hmda, |
| | | 36, 43, | | | | | adipsa |
| | | 46, 48, | | | | | |
| | | 71, 82, | | | | | |
| | | 96 | | | | | |
| A14 | 2.8.3 | 1, 4, 5, | CoA transferase | acyl-CoA, | atoAD | Escherichia coli | accoa, |
| | | 6, 18 | (acyl-CoA to acid) | acid | | | succoa, |
| | | | | | | | 3oacoa, |
| | | | | | | | 3hacoa, |
| | | | | | | | 5c2pcoa, |
| | | | | | | | adipcoa, |
| | | | | | | | 6acacoa |
| A15 | 3.1.2 | 1, 3, 5, | Thioester | acyl-CoA | yciA, tesB, | Escherichia coli | accoa, |
| | | 18, 19, | hydrolase (acyl- | | ybgC | | succoa, |
| | | 20 | CoA to acid) | | | | 3oacoa, |
| | | | | | | | 3hacoa, |
| | | | | | | | 5c2pcoa, |
| | | | | | | | adipcoa, |
| | | | | | | | 6acacoa |
| A16 | 4.1.1 | 4 | Decarboxylase | oxoacid | mdcAD | Methylobacterium | Byprod. |
| | | | acting on 3- | | | extorquens | Intermed. |
| | | | oxoacids | | | | |
| A17 | 4.2.1 | 2, 10, | Dehydratase | hydroxyacid | MexAM1_ | Methylobacterium | Byprod. |
| | | 53, 80 | (hydroxyacid to | | META1p09 | extorquens | Intermed. |
| | | | alkene) | | 70 | | |
| A18 | 4.3.1 | 1 | Ammonia-lyase | amine | aspA | Escherichia coli | Byprod. |
| | | | (aminoacid to | | | | Intermed. |
| | | | alkene) | | | | |
| A19 | 6.2.1 | 2, 4, 5, | CoA ligase (acyl- | acyl-CoA, | sucCD | Escherichia coli | accoa, |
| | | 23, 40 | CoA to acid) | acid | | | succoa, |
| | | | | | | | 3oacoa, |
| | | | | | | | 3hacoa, |
| | | | | | | | 5c2pc0a, |
| | | | | | | | adipcoa, |
| | | | | | | | 6acacoa, |
| | | | | | | | 6aca |
| A20 | 6.3.1 | 6, 8, 11 | glutamyl:amine | amine | puuA | Escherichia coli | 6aca, |
| | | | ligase | | | | 6acasa, |
| | | | | | | | hmda |
| A21 | no EC | no EC | Amine hydroxylase | amine | pubA | Shewanella | 6aca, |
| | | | (amine to | | | oneidensis | 6acasa, |
| | | | hydroxylamine) | | | | hmda |
| A22 | 1.3.* | EC 1.3.8 | Oxidoreductase | acyl-CoA | fadE | Escherichia coli | adipcoa, |
| | | | (alkane to alkene, | | | | 6acacoa |
| | | | irreversible) | | | | |
| A23 | 1.4.* | 1.4.9.1 | Amine oxidase | amine | tynA | Escherichia coli | 6aca, |
| | | | (amine to | | | | 6acasa, |
| | | | aldehyde, | | | | hmda |
| | | | irreversible) | | | | |
| A24 | 3.6.3 | 31 | Short-chain | diamine | potFGHI | Escherichia coli | 6aca, |
| | | | diamine exporter | | | | 6acasa, |
| | | | | | | | hmda |
| A25 | no EC | | Putrescine | diamine | puuP | Escherichia coli | 6aca, |
| | | | permease | | | | 6acasa, |
| | | | | | | | hmda |
| B1 | 3.5.1 | 46, 53, | Amide hydrolase | amide | aphA | Mycoplana ramosa | — |
| | | 62, 63 | or amidase | | | | |
| B2 | 3.5.2 | 9, 11, | Cyclic amide | cyclic | nylA | Flavobacterium sp. | — |
| | | 12 | hydrolase or | amide | | KI723T1 | |
| | | | lactamase | | | | |
| B3 | 6.2.1 | 2, 3, 5, | CoA ligase | acid | Msed_0394 | Metallosphaera | — |
| | | 14, 40 | | | | sedula | |
| B4 | 3.6.3 | 31 | Diamine | amine | potABCD | Escherichia coli | — |
| | | | transporter (longer | | | | |
| | | | chain diamines) | | | | |
| B5 | 3.6.3 | 31 | Diamine permease | amine | cadB | Escherichia coli | — |

Abbreviations: acetyl-CoA=accoa; succinyl-CoA=succoa; 3-oxoadipyl-CoA=3oacoa; 3-hydroxyadipyl-CoA=3hacoa; 5-carboxy-2-pentenoyl-CoA=5c2pcoa; adipyl-CoA=adipcoa; adipate semialdehyde=adipsa; 6-aminocaproate=6aca; 6-aminocaproate semialdehyde=6acasa; hexamethylene diamine=hmda; byprod=byproduct; intermed=intermediates; Exem.=exemplary

TABLE 4
Exemplary enzymes for use in methods and cells described herein

| | | Substrate | Product | | | |
|---|---|---|---|---|---|---|
| | | Functional | Functional | | | |
| EC | Function | Group | Group | Gene | GenBank | Organism |
| 1.1.1 | Oxidoreductase | aldehyde | alcohol | NZ_AFE | ZP_101314 | Bacillus |
| | (aldehyde to | | | U01000 | 90 | methanolicus |
| | alcohol) | | | 002.1:9 | | |
| | | | | 81149 . . . | | |
| | | | | 982312 | | |
| 1.1.1 | Oxidoreductase | aldehyde | alcohol | adhP | WP_011015 | Corynebacterium |
| | (aldehyde to | | | | 397 | glutamicum |
| | alcohol) | | | | | |
| 1.1.1 | Oxidoreductase | aldehyde | alcohol | yahK | P75691 | Escherichia coli |
| | (aldehyde to | | | | | |
| | alcohol) | | | | | |
| 1.1.1 | Oxidoreductase | ketone | alcohol | fdmH | P33677 | Hansenula |
| | (oxo to alcohol) | | | | | polymorpha |
| 1.1.1 | Oxidoreductase | aldehyde | alcohol | yqhD | NP_417484 | Escherichia coli |
| | (aldehyde to | | | | | |
| | alcohol) | | | | | |
| 1.1.1 | Oxidoreductase | aldehyde | alcohol | fucO | NP_417279 | Escherichia coli |
| | (aldehyde to | | | | | |
| | alcohol) | | | | | |
| 1.1.1 | Oxidoreductase | aldehyde | alcohol | adhP | NP_415995 | Escherichia coli |
| | (aldehyde to | | | | | |
| | alcohol) | | | | | |
| 1.1.1 | Oxidoreductase | ketone | alcohol | IdhA | NP_415898 | Escherichia coli |
| | (oxo to alcohol) | | | | | |
| 1.1.1 | Oxidoreductase | aldehyde | alcohol | HPODL_ | ESX01257 | Hansenula |
| | (aldehyde to | | | 00654 | | polymorpha |
| | alcohol) | | | | | |
| 1.1.1 | Oxidoreductase | aldehyde | alcohol | HPODL_ | ESW99796 | Hansenula |
| | (aldehyde to | | | 02528 | | polymorpha |
| | alcohol) | | | | | |
| 1.1.1 | Oxidoreductase | aldehyde | alcohol | HPODL_ | ESW95881 | Hansenula |
| | (aldehyde to | | | 02528 | | polymorpha |
| | alcohol) | | | | | |
| 1.1.1 | Oxidoreductase | ketone | alcohol | fdmH | CAA00531 | Hansenula |
| | (oxo to alcohol) | | | | | polymorpha |
| 1.1.1 | Oxidoreductase | aldehyde | alcohol | Adh | ACZ57808 | Pichia pastoris |
| | (aldehyde to | | | | | |
| | alcohol) | | | | | |
| 1.1.1 | Oxidoreductase | aldehyde | alcohol | MexAM | ACS41497 | Methylobacterium |
| | (aldehyde to | | | 1_MET | | extorquens |
| | alcohol) | | | A1p380 | | |
| | | | | 3 | | |
| 1.1.1 | Oxidoreductase | aldehyde | alcohol | dkgA | ACS39809 | Methylobacterium |
| | (aldehyde to | | | | | extorquens |
| | alcohol) | | | | | |
| 1.1.1 | Oxidoreductase | ketone | alcohol | mdh | AAC76268 | Escherichia coli |
| | (oxo to alcohol) | | | | | |
| 1.1.1 | Oxidoreductase (2 | acyl-CoA | alcohol | comple | ZP_101304 | Bacillus |
| | step, acyl-CoA to | | | ment(N | 43 | methanolicus |
| | alcohol) | | | Z_AFEU | | |
| | | | | 010000 | | |
| | | | | 01.1:97 | | |
| | | | | 9273 . . . 9 | | |
| | | | | 80670) | | |
| 1.1.1 | Oxidoreductase (2 | acyl-CoA | alcohol | comple | ZP_101304 | Bacillus |
| | step, acyl-CoA to | | | ment(N | 42 | methanolicus |
| | alcohol) | | | Z_AFEU | | |
| | | | | 010000 | | |
| | | | | 01.1:97 | | |
| | | | | 7194 . . . 9 | | |
| | | | | 78591) | | |
| 1.1.1 | Oxidoreductase (2 | acyl-CoA | alcohol | adhE | NP_415757 | Escherichia coli |
| | step, acyl-CoA to | | | | | |
| | alcohol) | | | | | |
| 1.1.1 | Oxidoreductase (2 | acyl-CoA | alcohol | bdh I | NP_349892 | Clostridium |
| | step, acyl-CoA to | | | | | acetobutylicum |
| | alcohol) | | | | | |
| 1.1.1 | Oxidoreductase (2 | acyl-CoA | alcohol | bdh II | NP_349891 | Clostridium |
| | step, acyl-CoA to | | | | | acetobutylicum |
| | alcohol) | | | | | |
| 1.1.1 | Oxidoreductase (2 | acyl-CoA | alcohol | adhE2 | AAK09379 | Clostridium |
| | step, acyl-CoA to | | | | | acetobutylicum |
| | alcohol) | | | | | |
| 1.2.1 | Oxidoreductase | aldehyde | acid | astD | P76217 | Escherichia coli |
| | (aldehyde to acid) | | | | | |
| 1.2.1 | Oxidoreductase | aldehyde | acid | aldB | NP_418045 | Escherichia coli |
| | (aldehyde to acid) | | | | | |
| 1.2.1 | Oxidoreductase | aldehyde | acid | ydcW | NP_415961 | Escherichia coli |
| | (aldehyde to acid) | | | | | |
| 1.2.1 | Oxidoreductase | aldehyde | acid | aldA | NP_415933 | Escherichia coli |
| | (aldehyde to acid) | | | | | |
| 1.2.1 | Oxidoreductase | aldehyde | acid | betB | NP_414846 | Escherichia coli |
| | (aldehyde to acid) | | | | | |
| 1.2.1 | Oxidoreductase | aldehyde | acid | asd CDS | EIJ81447 | Bacillus |
| | (aldehyde to acid) | | | | | methanolicus |
| 1.2.1 | Oxidoreductase | aldehyde | acid | fdhA | EIJ78226 | Bacillus |
| | (aldehyde to acid) | | | CDS | | methanolicus |
| 1.2.1 | Oxidoreductase | aldehyde | acid | FDH1 | CCA39210 | Hansenula |
| | (aldehyde to acid) | | | CDS | | polymorpha |
| 1.2.1 | Oxidoreductase | aldehyde | acid | ALD5 | CCA39155 | Pichia pastoris |
| | (aldehyde to acid) | | | CDS | | |
| 1.2.1 | Oxidoreductase | aldehyde | acid | ALD2 | CCA38525 | Pichia pastoris |
| | (aldehyde to acid) | | | CDS | | |
| 1.2.1 | Oxidoreductase | aldehyde | acid | PP7435_ | CCA37057 | Pichia pastoris |
| | (aldehyde to acid) | | | Chr1- | | |
| | | | | 0922 | | |
| 1.2.1 | Oxidoreductase | aldehyde | acid | CDS | CCA36189 | Pichia pastoris |
| | (aldehyde to acid) | | | | | |
| 1.2.1 | Oxidoreductase | aldehyde | acid | argC | ACS42527 | Methylobacterium |
| | (aldehyde to acid) | | | CDS | | extorquens |
| 1.2.1 | Oxidoreductase | aldehyde | acid | fdh2D | ACS42458 | Methylobacterium |
| | (aldehyde to acid) | | | CDS | | extorquens |
| 1.2.1 | Oxidoreductase | aldehyde | acid | ald | ACS42227 | Methylobacterium |
| | (aldehyde to acid) | | | | | extorquens |
| 1.2.1 | Oxidoreductase | aldehyde | acid | aldA | ACS41363 | Methylobacterium |
| | (aldehyde to acid) | | | CDS | | extorquens |
| 1.2.1 | Oxidoreductase | aldehyde | acid | gabD | AAC75708 | Escherichia coli |
| | (aldehyde to acid) | | | | | |
| 1.2.1 | Oxidoreductase | aldehyde | acid | sad | AAC74598. | Escherichia coli |
| | (aldehyde to acid) | | | | 2 | |
| 1.2.1 | Oxidoreductase | aldehyde | acid | feaB | AAC74467 | Escherichia coli |
| | (aldehyde to acid) | | | | | |
| 1.2.1 | Oxidoreductase | aldehyde | acid | aldH | AAC74382 | Escherichia coli |
| | (aldehyde to acid) | | | | | |
| 1.2.1 | Oxidoreductase | aldehyde | acid | ALD | AAA83769 | Hansenula |
| | (aldehyde to acid) | | | CDS | | polymorpha |
| 1.2.1 | Oxidoreductase | acyl-CoA | aldehyde | comple | ZP_101304 | Bacillus |
| | (acyl-CoA to | | | ment(N | 43 | methanolicus |
| | aldehyde) | | | Z_AFEU | | |
| | | | | 010000 | | |
| | | | | 01.1:97 | | |
| | | | | 9273 . . . 9 | | |
| | | | | 80670) | | |
| 1.2.1 | Oxidoreductase | acyl-CoA | aldehyde | comple | ZP_101304 | Bacillus |
| | (acyl-CoA to | | | ment(N | 42 | methanolicus |
| | aldehyde) | | | Z_AFEU | | |
| | | | | 010000 | | |
| | | | | 01.1:97 | | |
| | | | | 7194 . . . 9 | | |
| | | | | 78591) | | |
| 1.2.1 | Oxidoreductase | acyl-CoA | aldehyde | adhE | NP_415757 | Escherichia coli |
| | (acyl-CoA to | | | | | |
| | aldehyde) | | | | | |
| 1.2.1 | Oxidoreductase | acyl-CoA | aldehyde | PB1_02 | EIJ81770 | Bacillus |
| | (acyl-CoA to | | | 485 | | methanolicus |
| | aldehyde) | | | | | |
| 1.2.1 | Oxidoreductase | acyl-CoA | aldehyde | hmg1 | CCA37938 | Pichia pastoris |
| | (acyl-CoA to | | | | | |
| | aldehyde) | | | | | |
| 1.2.1 | Oxidoreductase | acid | aldehyde | car | YP_001070 | Mycobacterium sp. |
| | (acid to aldehyde) | | | | 587 | strain JLS |
| 1.2.1 | Oxidoreductase | acid | aldehyde | npt | YP_001070 | Mycobacterium sp. |
| | (acid to aldehyde) | | | | 355 | strain JLS |
| 1.2.1 | Oxidoreductase | acid | aldehyde | LYS5 | P50113 | Saccharomyces |
| | (acid to aldehyde) | | | | | cerevisiae |
| 1.2.1 | Oxidoreductase | acid | aldehyde | Lys2 | EIJ81770 | Bacillus |
| | (acid to aldehyde) | | | | | methanolicus |
| 1.2.1 | Oxidoreductase | acid | aldehyde | Lys2 | CCA37057 | Pichia pastoris |
| | (acid to aldehyde) | | | | | |
| 1.2.1 | Oxidoreductase | acid | aldehyde | Lys2 | ACS41990 | Methylobacterium |
| | (acid to aldehyde) | | | | | extorquens |
| 1.2.1 | Oxidoreductase | acid | aldehyde | npt | ABI83656 | Nocardia iowensis |
| | (acid to aldehyde) | | | | | |
| 1.2.1 | Oxidoreductase | acid | aldehyde | car | AAR91681 | Nocardia iowensis |
| | (acid to aldehyde) | | | | | |
| 1.2.1 | Oxidoreductase | acid | aldehyde | LYS2 | AAA34747 | Saccharomyces |
| | (acid to aldehyde) | | | | | cerevisiae |
| 1.2.3 | Aldehyde oxidase | aldehyde | acid | aomm | EIJ80428 | Bacillus |
| | (aldehyde to acid | | | | | methanolicus |
| | in presence of O2) | | | | | |
| 1.2.3 | Aldehyde oxidase | aldehyde | acid | aomm | EIJ78153 | Bacillus |
| | (aldehyde to acid | | | | | methanolicus |
| | in presence of O2) | | | | | |
| 1.2.3 | Aldehyde oxidase | aldehyde | acid | aoms | EIJ78152 | Bacillus |
| | (aldehyde to acid | | | | | methanolicus |
| | in presence of O2) | | | | | |
| 1.2.3 | Aldehyde oxidase | aldehyde | acid | AOH2 | CCA37815 | Pichia pastoris |
| | (aldehyde to acid | | | | | |
| | in presence of O2) | | | | | |
| 1.2.3 | Aldehyde oxidase | aldehyde | acid | aomm | BAC54901 | Methylobacillus sp. |
| | (aldehyde to acid | | | | | KY4400 |
| | in presence of O2) | | | | | |
| 1.2.3 | Aldehyde oxidase | aldehyde | acid | aomm | BAC54900 | Methylobacillus sp. |
| | (aldehyde to acid | | | | | KY4400 |
| | in presence of O2) | | | | | |
| 1.2.3 | Aldehyde oxidase | aldehyde | acid | aoms | BAC54899 | Methylobacillus sp. |
| | (aldehyde to acid | | | | | KY4400 |
| | in presence of O2) | | | | | |
| 1.2.3 | Aldehyde oxidase | aldehyde | acid | aomm | ACS41608 | Methylobacterium |
| | (aldehyde to acid | | | | | extorquens |
| | in presence of O2) | | | | | |
| 1.2.3 | Aldehyde oxidase | aldehyde | acid | aomm | ACS40763 | Methylobacterium |
| | (aldehyde to acid | | | | | extorquens |
| | in presence of O2) | | | | | |
| 1.2.3 | Aldehyde oxidase | aldehyde | acid | aoms | ACS40762 | Methylobacterium |
| | (aldehyde to acid | | | | | extorquens |
| | in presence of O2) | | | | | |
| 1.2.3 | Aldehyde oxidase | aldehyde | acid | MexAM | ACS38613 | Methylobacterium |
| | (aldehyde to acid | | | 1_MET | | extorquens |
| | in presence of O2) | | | A1p068 | | |
| | | | | 4 | | |
| 1.2.3 | Aldehyde oxidase | aldehyde | acid | aomm | ACS38534 | Methylobacterium |
| | (aldehyde to acid | | | | | extorquens |
| | in presence of O2) | | | | | |
| 1.2.3 | Aldehyde oxidase | aldehyde | acid | aomm | ACS38533 | Methylobacterium |
| | | | | | | extorquens |
| | (aldehyde to acid | | | | | |
| | in presence of O2) | | | | | |
| 1.2.3 | Aldehyde oxidase | aldehyde | acid | aoms | ACS38532 | Methylobacterium |
| | (aldehyde to acid | | | | | extorquens |
| | in presence of O2) | | | | | |
| 1.3.* | Oxidoreductase | acyl-CoA | enoyl-CoA | fadE | EIJ80650 | Bacillus |
| | (alkene to alkane, | | | | | methanolicus |
| | other e− acceptor) | | | | | |
| 1.3.* | Oxidoreductase | acyl-CoA | enoyl-CoA | caiA | EIJ80277 | Bacillus |
| | (alkene to alkane, | | | | | methanolicus |
| | other e− acceptor) | | | | | |
| 1.3.* | Oxidoreductase | acyl-CoA | enoyl-CoA | Pox2 | CCA37459 | Pichia pastoris |
| | (alkene to alkane, | | | | | |
| | other e− acceptor) | | | | | |
| 1.3.* | Oxidoreductase | acyl-CoA | enoyl-CoA | MexAM | ACS42290 | Methylobacterium |
| | (alkene to alkane, | | | 1_MET | | extorquens |
| | other e− acceptor) | | | A1p466 | | |
| | | | | 1 | | |
| 1.3.* | Oxidoreductase | acyl-CoA | enoyl-CoA | MexAM | ACS42125 | Methylobacterium |
| | (alkene to alkane, | | | 1_MET | | extorquens |
| | other e− acceptor) | | | A1p449 | | |
| | | | | 4 | | |
| | Oxidoreductase | | | | | |
| 1.3.* | (alkene to alkane, | acyl-CoA | enoyl-CoA | ydiO | AAC74765 | Escherichia coli |
| | other e− acceptor) | | | | | |
| | Oxidoreductase | | | | | |
| 1.3.* | (alkene to alkane, | acyl-CoA | enoyl-CoA | fadE | AAC73325 | Escherichia coli |
| | other e− acceptor) | | | | | |
| 1.3.* | Oxidoreductase | acyl-CoA | enoyl-CoA | fadE | AAC73325 | Methylobacterium |
| | (alkene to alkane, | | | | | extorquens |
| | other e− acceptor) | | | | | |
| 1.3.1 | Oxidoreductase | acyl-CoA | enoyl-CoA | fabl | POAEK4 | Escherichia coli |
| | (alkene to alkane, | | | | | |
| | other e− acceptor) | | | | | |
| 1.3.1 | Oxidoreductase | acyl-CoA | enoyl-CoA | PB1_03 | EIJ82038 | Bacillus |
| | (alkene to alkane, | | | | | methanolicus |
| | other e− acceptor) | | | 835 | | |
| 1.3.1 | Oxidoreductase | acyl-CoA | enoyl-CoA | PB1_09 | | Bacillus |
| | (alkene to alkane, | | | EIJ80650 | | methanolicus |
| | other e− acceptor) | | | 827 | | |
| 1.3.1 | Oxidoreductase | acyl-CoA | enoyl-CoA | PB1_07 | EIJ80277 | Bacillus |
| | (alkene to alkane, | | | 947 | | methanolicus |
| | other e− acceptor) | | | | | |
| 1.3.1 | Oxidoreductase | acyl-CoA | enoyl-CoA | PB1_07 | EIJ80276 | Bacillus |
| | (alkene to alkane, | | | 942 | | methanolicus |
| | other e− acceptor) | | | | | |
| 1.3.1 | Oxidoreductase | acyl-CoA | enoyl-CoA | PB1_15 | EIJ78902 | Bacillus |
| | (alkene to alkane, | | | 129 | | methanolicus |
| | other e− acceptor) | | | | | |
| 1.3.1 | Oxidoreductase | acyl-CoA | enoyl-CoA | PB1_11 | EIJ78194 | Bacillus |
| | (alkene to alkane, | | | 559 | | methanolicus |
| | other e− acceptor) | | | | | |
| 1.3.1 | Oxidoreductase | acyl-CoA | enoyl-CoA | PB1_10 | EIJ78074 | Bacillus |
| | (alkene to alkane, | | | 959 | | methanolicus |
| | other e− acceptor) | | | | | |
| 1.3.1 | Oxidoreductase | acyl-CoA | enoyl-CoA | PP7435_ | CCA37459 | Pichia pastoris |
| | (alkene to alkane, | | | Chr1- | | |
| | other e− acceptor) | | | 1341 | | |
| 1.3.1 | Oxidoreductase | acyl-CoA | enoyl-CoA | MexAM | ACS42652 | Methylobacterium |
| | (alkene to alkane, | | | 1_MET | | extorquens |
| | other e− acceptor) | | | A1p504 | | |
| | | | | 8 | | |
| 1.3.1 | Oxidoreductase | enoyl-CoA | MET | MexAM | ACS42290 | Methylobacterium |
| | (alkene to alkane, | acyl-CoA | | A1p466 | | extorquens |
| | other e− acceptor) | | | 1 | | |
| 1.3.1 | Oxidoreductase | acyl-CoA | enoyl-CoA | MexAM | ACS42125 | Methylobacterium |
| | (alkene to alkane, | | | MET | | extorquens |
| | other e− acceptor) | | | A1p449 | | |
| | | | | 4 | | |
| 1.3.1 | Oxidoreductase | acyl-CoA | enoyl-CoA | MexAM | ACS41858 | Methylobacterium |
| | (alkene to alkane, | | | 1_MET | | extorquens |
| | other e− acceptor) | | | A1p422 | | |
| | | | | 0 | | |
| 1.3.1 | Oxidoreductase | acyl-CoA | enoyl-CoA | MexAM | ACS41605 | Methylobacterium |
| | (alkene to alkane, | | | 1_MET | | extorquens |
| | other e− acceptor) | | | A1p392 | | |
| | | | | 1 | | |
| 1.3.1 | Oxidoreductase | acyl-CoA | enoyl-CoA | MexAM | ACS41438 | Methylobacterium |
| | (alkene to alkane, | | | 1_MET | | extorquens |
| | other e− acceptor) | | | A1p372 | | |
| | | | | 8 | | |
| 1.3.1 | Oxidoreductase | acyl-CoA | enoyl-CoA | MexAM | ACS41426 | Methylobacterium |
| | (alkene to alkane, | | | 1_MET | | extorquens |
| | other e− acceptor) | | | A1p371 | | |
| | | | | 6 | | |
| 1.3.1 | Oxidoreductase | acyl-CoA | enoyl-CoA | MexAM | ACS41288 | Methylobacterium |
| | (alkene to alkane, | | | 1_MET | | extorquens |
| | other e− acceptor) | | | A1p355 | | |
| | | | | 4 | | |
| 1.3.1 | Oxidoreductase | acyl-CoA | enoyl-CoA | MexAM | ACS41193 | Methylobacterium |
| | (alkene to alkane, | | | 1_MET | | extorquens |
| | other e− acceptor) | | | A1p345 | | |
| | | | | 6 | | |
| 1.3.1 | Oxidoreductase | acyl-CoA | enoyl-CoA | MexAM | ACS40016 | Methylobacterium |
| | (alkene to alkane, | | | 1_MET | | extorquens |
| | other e− acceptor) | | | A1p222 | | |
| | | | | 3 | | |
| 1.3.1 | Oxidoreductase | acyl-CoA | enoyl-CoA | MexAM | ACS38844 | Methylobacterium |
| | (alkene to alkane, | | | 1_MET | | extorquens |
| | other e− acceptor) | | | A1p094 | | |
| | | | | 6 | | |
| | | | | MexAM | | |
| 1.3.1 | Oxidoreductase | acyl-CoA | enoyl-CoA | 1_MET | ACS38823 | Methylobacterium |
| | (alkene to alkane, | | | A1p092 | | extorquens |
| | other e− acceptor) | | | 2 | | |
| 1.4.* | Amine oxidase (O2 | amine | aldehyde | mauB | Q49124 | Methylobacterium |
| | or alternate e− | | | | | extorquens |
| | acceptor) | | | | | |
| 1.4.* | Amine oxidase (O2 | amine | aldehyde | madh | P00372 | Methylobacterium |
| | or alternate e− | | | | | extorquens |
| | acceptor) | | | | | |
| 1.4.* | Amine oxidase (O2 | amine | aldehyde | tynA | NP_415904 | Escherichia coli |
| | or alternate e− | | | | | |
| | acceptor) | | | | | |
| 1.4.* | Amine oxidase (O2 | amine | aldehyde | PB1_03 | EIJ82048 | Bacillus |
| | or alternate e− | | | 885 | | methanolicus |
| | acceptor) | | | | | |
| 1.4.* | Amine oxidase (O2 | amine | aldehyde | PB1_03 | EIJ82043 | Bacillus |
| | or alternate e− | | | 860 | | methanolicus |
| | acceptor) | | | | | |
| 1.4.* | Amine oxidase (O2 | amine | aldehyde | PB1_01 | EIJ81618 | Bacillus |
| | or alternate e− | | | 715 | | methanolicus |
| | acceptor) | | | | | |
| 1.4.* | Amine oxidase (O2 | amine | aldehyde | PB1_10 | EIJ77997 | Bacillus |
| | or alternate e− | | | 524 | | methanolicus |
| | acceptor) | | | | | |
| 1.4.* | Amine oxidase (O2 | amine | aldehyde | Aoc3 | CCA40518 | Pichia pastoris |
| | or alternate e− | | | | | |
| | acceptor) | | | | | |
| 1.4.* | Amine oxidase (O2 | amine | aldehyde | Cbp1 | CCA40304 | Pichia pastoris |
| | or alternate e− | | | | | |
| | acceptor) | | | | | |
| 1.4.* | Amine oxidase (O2 | amine | aldehyde | PP7435_ | CCA39220 | Pichia pastoris |
| | or alternate e− | | | Chr3- | | |
| | acceptor) | | | 0249 | | |
| 1.4.* | Amine oxidase (O2 | amine | aldehyde | aoc3 | CCA38674 | Pichia pastoris |
| | or alternate e− | | | | | |
| | acceptor) | | | | | |
| 1.4.* | Amine oxidase (O2 | amine | aldehyde | amo | CCA37360 | Pichia pastoris |
| | or alternate e− | | | | | |
| | acceptor) | | | | | |
| 1.4.* | Amine oxidase (O2 | amine | aldehyde | AMO | CAA33209 | Hansenula |
| | or alternate e− | | | | | polymorpha |
| | acceptor) | | | | | |
| 1.4.* | Amine oxidase (O2 | amine | aldehyde | MexAM | ACS42429 | Methylobacterium |
| | or alternate e− | | | 1_MET | | extorquens |
| | acceptor) | | | A1p481 | | |
| | | | | 7 | | |
| 1.4.* | Amine oxidase (O2 | amine | aldehyde | MexAM | ACS39659 | Methylobacterium |
| | or alternate e− | | | 1_MET | | extorquens |
| | acceptor) | | | A1p181 | | |
| | | | | 7 | | |
| 1.4.* | Amine oxidase (O2 | amine | aldehyde | MexAM | ACS38343 | Methylobacterium |
| | or alternate e− | | | 1_MET | | extorquens |
| | acceptor) | | | A1p039 | | |
| | | | | 6 | | |
| 1.4.* | Amine oxidase (O2 | amine | aldehyde | mauA | AAA25379 | Methylobacterium |
| | or alternate e− | | | | | extorquens |
| | acceptor) | | | | | |
| 1.4.1 | Oxidoreductase | amine | aldehyde | Lys9 | WP_023493 | Methyloglobulus |
| | operating on | | | | 400 | morosus KoM1 |
| | amino groups | | | | | |
| 1.4.1 | Oxidoreductase | amine | aldehyde | lysDH | NP_353966 | Agrobacterium |
| | operating on | | | | | turnefaciens |
| | amino groups | | | | | |
| 1.4.1 | Oxidoreductase | amine | aldehyde | Lys9 | CCA39634 | Pichia pastoris |
| | operating on | | | | | |
| | amino groups | | | | | |
| | Oxidoreductase | | | | | |
| | Geobacillus | | | | | |
| 1.4.1 | operating on | amine | aldehyde | lysDH | BAB39707 | stearothermophilus |
| | amino groups | | | | | |
| 1.4.1 | Oxidoreductase | amine | aldehyde | lysDH | AAZ94428 | Achromobacter |
| | operating on | | | | | denitrificans |
| | amino groups | | | | | |
| 2.1.1 | Amine N- | amine | methyl | cho2 | C4QXE9 | Pichia pastoris |
| | methyltransferase | | amine | | | |
| 2.1.1 | Amine N- | amine | methyl | BANMT | AAP03058 | Limonium |
| | methyltransferase | | amine | 1 | | latifolium |
| 2.1.3 | Carbamoyl | amine | carbamoyl- | argl | NP_418675 | E. coli |
| | transferase | | amine | | | |
| 2.1.3 | Carbamoyl | amine | carbamoyl- | argF | NP_414807 | E. coli |
| | transferase | | amine | | | |
| 2.1.3 | Carbamoyl | amine | carbamoyl- | argF | EIJ81870 | Bacillus |
| | transferase | | amine | | | methanolicus |
| 2.1.3 | Carbamoyl | amine | carbamoyl- | pyrB | EIJ81566 | Bacillus |
| | transferase | | amine | CDS | | methanolicus |
| 2.1.3 | Carbamoyl | amine | carbamoyl- | ARG3 | CCA39537 | Pichia pastoris |
| | transferase | | amine | | | |
| 2.1.3 | Carbamoyl | amine | carbamoyl- | URA2 | CCA37846 | Pichia pastoris |
| | transferase | | amine | CDS | | |
| 2.1.3 | Carbamoyl | amine | carbamoyl- | argF | ACS42096 | Methylobacterium |
| | transferase | | amine | | | extorquens |
| 2.1.3 | Carbamoyl | amine | carbamoyl- | pyrB | ACS41262 | Methylobacterium |
| | transferase | | amine | CDS | | extorquens |
| 2.3.1 | Acyltransferase | acyl-CoA | acyl-CoA | fadA | YP 026272 | Escherichia coli |
| | (beta-ketothiolase) | | | | | |
| 2.3.1 | Acyltransferase | acyl-CoA | acyl-CoA | yqeF | NP_417321 | Escherichia coli |
| | (beta-ketothiolase) | | | | | |
| 2.3.1 | Acyltransferase | acyl-CoA | acyl-CoA | fadl | NP_416844 | Escherichia coli |
| | (beta-ketothiolase) | | | | | |
| 2.3.1 | Acyltransferase | acyl-CoA | acyl-CoA | atoB | NP_416728 | Escherichia coli |
| | (beta-ketothiolase) | | | | | |
| 2.3.1 | Acyltransferase | acyl-CoA | acyl-CoA | paaJ | NP_415915 | Escherichia coli |
| | (beta-ketothiolase) | | | | | |
| 2.3.1 | Acyltransferase | acyl-CoA | acyl-CoA | HPODL_ | ESX00212 | Hansenula |
| | (beta-ketothiolase) | | | 01088 | | polymorpha |
| 2.3.1 | Acyltransferase | acyl-CoA | acyl-CoA | HPODL_ | ESW98901 | Hansenula |
| | (beta-ketothiolase) | | | 004502 | | polymorpha |
| 2.3.1 | Acyltransferase | acyl-CoA | acyl-CoA | atoB | EIJ80649 | Bacillus |
| | (beta-ketothiolase) | | | | | methanolicus |
| 2.3.1 | Acyltransferase | acyl-CoA | acyl-CoA | mmgA | EIJ80274 | Bacillus |
| | (beta-ketothiolase) | | | | | methanolicus |
| 2.3.1 | Acyltransferase | acyl-CoA | acyl-CoA | atoB | EIJ79785 | Bacillus |
| | (beta-ketothiolase) | | | | | methanolicus |
| 2.3.1 | Acyltransferase | acyl-CoA | acyl-CoA | atoB | CCA37973 | Pichia pastoris |
| | (beta-ketothiolase) | | | | | |
| 2.3.1 | Acyltransferase | acyl-CoA | acyl-CoA | atoB | CCA37220 | Pichia pastoris |
| | (beta-ketothiolase) | | | | | |
| 2.3.1 | Acyltransferase | acyl-CoA | acyl-CoA | AIK858 | AIK85817 | Corynebacterium |
| | (beta-ketothiolase) | | | 17 | | glutamicum |
| 2.3.1 | Acyltransferase | acyl-CoA | acyl-CoA | CGLAR1_ | AIK84853 | Corynebacterium |
| | (beta-ketothiolase) | | | 06190 | | glutamicum |
| 2.3.1 | Acyltransferase | acyl-CoA | acyl-CoA | atoB | ACS42949 | Methylobacterium |
| | (beta-ketothiolase) | | | | | extorquens |
| 2.3.1 | Acyltransferase | acyl-CoA | acyl-CoA | phaA | ACS41411 | Methylobacterium |
| | (beta-ketothiolase) | | | | | extorquens |
| 2.3.1 | Acyltransferase | acyl-CoA | acyl-CoA | atoB | ACS41192 | Methylobacterium |
| | (beta-ketothiolase) | | | | | extorquens |
| 2.3.1 | Acyltransferase | amine, acyl- | acyl-amine | not | WP_003862 | Corynebacterium |
| | (N-acyltransferase) | CoA | | | 331 | glutamicum |
| 2.3.1 | Acyltransferase | amine, acyl- | acyl-amine | pubB | NP_718599 | Shewanella |
| | (N-acyltransferase) | CoA | | | | |
| 2.3.1 | Acyltransferase | amine, acyl- | acyl-amine | speG | NP_416101 | Escherichia coli |
| | (N-acyltransferase) | CoA | | | | |
| 2.3.1 | Acyltransferase | amine, acyl- | acyl-amine | HPODL_ | ESW99535 | Hansenula |
| | (N-acyltransferase) | CoA | | 03421 | | polymorpha |
| 2.3.1 | Acyltransferase | amine, acyl- | acyl-amine | PB1_03 | EIJ81991 | Bacillus |
| | (N-acyltransferase) | CoA | | 600 | | methanolicus |
| 2.3.1 | Acyltransferase | amine, acyl- | acyl-amine | PB1_13 | EIJ78477 | Bacillus |
| | (N-acyltransferase) | CoA | | 004 | | methanolicus |
| 2.3.1 | Acyltransferase | amine, acyl- | acyl-amine | ECO1 | CCA39230 | Pichia pastoris |
| | (N-acyltransferase) | CoA | | | | |
| 2.3.1 | Acyltransferase | amine, acyl- | acyl-amine | argJ | CAA60097 | Corynebacterium |
| | (N-acyltransferase) | CoA | | | | glutamicum |
| 2.3.1 | Acyltransferase | amine, acyl- | acyl-amine | MexAM | ACS41790 | Methylobacterium |
| | (N-acyltransferase) | CoA | | 1_MET | | extorquens |
| | | | | A1p413 | | |
| | | | | 7 | | |
| 2.3.1 | Acyltransferase | amine, acyl- | acyl-amine | speG | ACS40652 | Methylobacterium |
| | (N-acyltransferase) | CoA | | | | extorquens |
| 2.3.1 | Acyltransferase | acyl-CoA | | fabD | AAC74176 | Escherichia coli |
| | (N-acyltransferase) | | | | | |
| 2.3.1 | Acyltransferase | acyl-CoA | | fabH | AAC74175 | Escherichia coli |
| | (N-acyltransferase) | | | | | |
| 2.5.1 | Diamine synthase | amine | diamine | PB1_07 | EIJ80267 | Bacillus |
| | | | | 897 | | methanolicus |
| 2.5.1 | Diamine synthase | amine | diamine | spe4 | CCA40492 | Pichia pastoris |
| 2.5.1 | Diamine synthase | amine | diamine | spe3 | CCA38201 | Pichia pastoris |
| 2.5.1 | Acyl-ACP | amine | diamine | speE | AAC73232 | Escherichia coli |
| | thioesterase | | | | | |
| 2.6.1 | Aminotransferase | amine | aldehyde | avtA | YP_026231 | Escherichia coli |
| 2.6.1 | Aminotransferase | amine | aldehyde | avtA | YP_026231 | Escherichia coli |
| 2.6.1 | Aminotransferase | amine | aldehyde | clot | P56744 | Acinetobacter |
| | | | | | | baumanii |
| 2.6.1 | Aminotransferase | amine | aldehyde | clot | P44951 | Haemophilus |
| | | | | | | influenzae |
| 2.6.1 | Aminotransferase | amine | aldehyde | ygjG | NP_417544 | Escherichia coli |
| 2.6.1 | Diamine synthase | amine | aldehyde | gabT | NP_417148 | Escherichia coli |
| 2.6.1 | Aminotransferase | amine | aldehyde | puuE | NP_415818 | Escherichia coli |
| 2.6.1 | Aminotransferase | amine | aldehyde | aspC | NP_415448 | Escherichia coli |
| 2.6.1 | Aminotransferase | amine | aldehyde | aspC | NP_415448 | Escherichia coli |
| 2.6.1 | Aminotransferase | amine | aldehyde | serC | NP_415427 | Escherichia coli |
| 2.6.1 | Aminotransferase | amine | aldehyde | serC | NP_415427 | Escherichia coli |
| 2.6.1 | Aminotransferase | amine | aldehyde | HPODL_ | ESX02294 | Hansenula |
| | | | | 05044 | | polymorpha |
| 2.6.1 | Aminotransferase | amine | aldehyde | gabT | ESW97620 | Hansenula |
| | | | | | | polymorpha |
| 2.6.1 | Aminotransferase | amine | aldehyde | HPODL_ | ESW97476 | Hansenula |
| | | | | 01574 | | polymorpha |
| 2.6.1 | Aminotransferase | amine | aldehyde | argD | EIJ81873 | Bacillus |
| | | | | | | methanolicus |
| 2.6.1 | Aminotransferase | amine | aldehyde | patA | EIJ81692 | Bacillus |
| | | | | | | methanolicus |
| 2.6.1 | Aminotransferase | amine | aldehyde | at | EIJ81360 | Bacillus |
| | | | | | | methanolicus |
| 2.6.1 | Aminotransferase | amine | aldehyde | rocD | EIJ80718 | Bacillus |
| | | | | | | methanolicus |
| 2.6.1 | Aminotransferase | amine | aldehyde | at | EIJ80434 | Bacillus |
| | | | | | | methanolicus |
| 2.6.1 | Aminotransferase | amine | aldehyde | at | EIJ79061 | Bacillus |
| | | | | | | methanolicus |
| 2.6.1 | Aminotransferase | amine | aldehyde | ARG8 | CCA40494 | Pichia pastoris |
| 2.6.1 | Aminotransferase | amine | aldehyde | UGA1 | CCA40463 | Pichia pastoris |
| 2.6.1 | Aminotransferase | amine | aldehyde | CAR2 | CCA39756 | Pichia pastoris |
| 2.6.1 | Aminotransferase | amine | aldehyde | PP7435_ | CCA38877 | Pichia pastoris |
| | | | | Chr2- | | |
| | | | | 1202 | | |
| 2.6.1 | Aminotransferase | amine | aldehyde | lat | BAB13756 | Flavobacterium |
| | | | | | | lutescens |
| 2.6.1 | Aminotransferase | amine | aldehyde | argD | ACS42095 | Methylobacterium |
| | | | | | | extorquens |
| 2.6.1 | Aminotransferase | amine | aldehyde | MexAM | ACS40861 | Methylobacterium |
| | | | | 1_MET | | extorquens |
| | | | | A1p311 | | |
| | | | | 3 | | |
| 2.6.1 | Aminotransferase | aldehyde | amine | MexAM | ACS40262 | Methylobacterium |
| | | | | 1_MET | | extorquens |
| | | | | A1p248 | | |
| | | | | 3 | | |
| 2.6.1 | Aminotransferase | amine | aldehyde | ectB | AAZ57191 | Halobacillus |
| | | | | | | dabanensis |
| 2.6.1 | Aminotransferase | amine | aldehyde | pvdH | AAG05801 | Pseudomonas |
| | | | | | | aeruginosa |
| 2.6.1 | Aminotransferase | amine | aldehyde | spuC | AAG03688 | Pseudomonas |
| | | | | | | aeruginosa |
| 2.6.1 | Aminotransferase | amine | aldehyde | ectB | AAB57634 | Marinococcus |
| | | | | | | halophilus |
| 2.6.1 | Aminotransferase | amine | aldehyde | lat | AAA26777 | Streptomyces |
| | | | | | | clavuligenus |
| 2.8.3 | CoA transferase | acyl-CoA, | acid | atoA | P76459 | Escherichia coli |
| | | acid | | | | |
| 2.8.3 | CoA transferase | acyl-CoA, | acid | atoD | P76458 | Escherichia coli |
| | | acid | | | | |
| 2.8.3 | CoA transferase | acyl-CoA, | acid | ygfH | NP_417395 | Escherichia coli |
| | | acid | | | | |
| 2.8.3 | CoA transferase | acyl-CoA, | acid | SD36_1 | KIH72944 | Corynebacterium |
| | | acid | | 1620 | | glutamicum |
| 2.8.3 | CoA transferase | acyl-CoA, | acid | atoD | EIJ78763 | Bacillus |
| | | acid | | | | methanolicus |
| 2.8.3 | CoA transferase | acyl-CoA, | acid | atoA | EIJ78762 | Bacillus |
| | | acid | | | | methanolicus |
| 2.8.3 | CoA transferase | acyl-CoA, | acid | atoD | EIJ78548 | Bacillus |
| | | acid | | | | methanolicus |
| 2.8.3 | CoA transferase | acyl-CoA, | acid | atoA | EIJ78547 | Bacillus |
| | | acid | | | | methanolicus |
| 2.8.3 | CoA transferase | acyl-CoA, | acid | pcal | AGT06117 | Corynebacterium |
| | | acid | | | | glutamicum |
| 2.8.3 | CoA transferase | acyl-CoA, | acid | atoA | ACS40873 | Methylobacterium |
| | | acid | | | | extorquens |
| 2.8.3 | CoA transferase | acyl-CoA, | acid | atoD | ACS40872 | Methylobacterium |
| | | acid | | | | extorquens |
| 2.8.3 | CoA transferase | acyl-CoA, | acid | atoAB | ACS39856 | Methylobacterium |
| | | acid | | | | extorquens |
| 3.1.2 | CoA hydrolase | acyl-CoA | acid | paal | NP_415914 | Escherichia coli |
| 3.1.2 | CoA hydrolase | acyl-CoA | acid | yciA | NP_415769 | Escherichia coli |
| 3.1.2 | CoA hydrolase | acyl-CoA | acid | ybgC | NP_415264 | Escherichia coli |
| 3.1.2 | CoA hydrolase | acyl-CoA | acid | ybdB | NP_415129 | Escherichia coli |
| 3.1.2 | CoA hydrolase | acyl-CoA | acid | tesA | NP_415027 | Escherichia coli |
| 3.1.2 | CoA hydrolase | acyl-CoA | acid | tesB | NP_414986 | Escherichia coli |
| 3.1.2 | CoAhydrolase | acyl-CoA | acid | HPODL_ | ESW98635 | Hansenula |
| | | | | 04251 | | polymorpha |
| 3.1.2 | CoA hydrolase | acyl-CoA | acid | HPODL_ | ESW96601 | Hansenula |
| | | | | 03216 | | polymorpha |
| 3.1.2 | CoA hydrolase | acyl-CoA | acid | MGA3_ | EIJ82858 | Bacillus |
| | | | | 06520 | | methanolicus |
| 3.1.2 | CoA hydrolase | acyl-CoA | acid | tesB | CCA38431 | Pichia pastoris |
| 3.1.2 | CoA hydrolase | acyl-CoA | acid | CGLAR1_ | AIK85986 | Corynebacterium |
| | | | | 12305 | | glutamicum |
| 3.1.2 | CoA hydrolase | acyl-CoA | acid | CGLAR1_ | AIK85969 | Corynebacterium |
| | | | | 12220 | | glutamicum |
| 3.1.2 | CoA hydrolase | acyl-CoA | acid | CGLAR1_ | AIK84631 | Corynebacterium |
| | | | | 05010 | | glutamicum |
| 3.1.2 | CoA hydrolase | acyl-CoA | acid | tesB | ACS39883 | Methylobacterium |
| | | | | | | extorquens |
| 3.1.2 | CoA hydrolase | acyl-CoA | acid | entH | AAC73698 | Escherichia coli |
| 3.5.1 | Amidase | amide | amine | ACY3 | Q96HD9 | Homo sapiens |
| 3.5.1 | Amidase | amide | amine | ramA | Q75SP7 | Pseudomonas sp. |
| | | | | | | MC13434 |
| 3.5.1 | Amidase | amide | amine | aphA | Q48935 | Mycoplana ramosa |
| 3.5.1 | Amidase | amide | amine | blr3999 | NP_770639 | Bradyrhizobium |
| | | | | | | diazoefficiens |
| 3.5.1 | Amidase | amide | amine | aguB | KFL09211 | Pseudomonas |
| | | | | | | aeruginosa |
| 3.5.1 | Amidase | amide | amine | At2g27 | BAH19976 | Arabidopsis |
| | | | | 450 | | thaliana |
| 3.5.1 | Amidase | amide | amine | nylB | B22644 | Flavobacterium sp. |
| | | | | | | KI723T1 |
| 3.5.1 | Amidase | amide | amine | nylB | AKE75031 | Klebsiella |
| | | | | | | pneumoniae |
| 3.5.1 | Amidase | amide | amine | C8J_08 | ABV52489 | Campylobacter |
| | | | | 90 | | jejuni jejuni 81116 |
| 3.5.2 | Cyclic amidase | amide | amine | PP4_27 | BAN54575 | Pseudomonas |
| | | | | 220 | | putida |
| 3.5.2 | Cyclic amidase | amide | amine | oplah | AAH85330 | Rattus norvegicus |
| 3.5.2 | Cyclic amidase | amide | amine | nylA | AAA24929 | Flavobacterium sp. |
| | | | | | | KI723T1 |
| 3.5.2 | Cyclic amidase | amide | amine | A44761 | A44761 | Pseudomonas sp. |
| | | | | | | (strain NK87) |
| 3.6.3 | Diamine | amine | amine | potA | AAC74210 | Escherichia coli |
| | transporter | intracellular | extracellular | | | |
| 3.6.3 | Diamine | amine | amine | potB | AAC74209 | Escherichia coli |
| | transporter | intracellular | extracellular | | | |
| 3.6.3 | Diamine | amine | amine | potC | AAC74208 | Escherichia coli |
| | transporter | intracellular | extracellular | | | |
| 3.6.3 | Diamine | amine | amine | potD | AAC74207 | Escherichia coli |
| | transporter | intracellular | extracellular | | | |
| 3.6.3 | Diamine | amine | amine | potl | AAC73944 | Escherichia coli |
| | transporter | intracellular | extracellular | | | |
| 3.6.3 | Diamine | amine | amine | potH | AAC73943 | Escherichia coli |
| | transporter | intracellular | extracellular | | | |
| 3.6.3 | Diamine | amine | amine | potG | AAC73942 | Escherichia coli |
| | transporter | intracellular | extracellular | | | |
| 3.6.3 | Diamine | amine | amine | potF | AAC73941 | Escherichia coli |
| | transporter | intracellular | extracellular | | | |
| 4.1.1 | Decarboxylase | 3-oxoacid | 2-keto | mdcD | ACS37998 | Methylobacterium |
| | | | alkane | | | extorquens |
| 4.1.1 | Decarboxylase | 3-oxoacid | 2-keto | mdcA | ACS37996 | Methylobacterium |
| | | | alkane | | | extorquens |
| 4.2.1 | Dehydratase | dehydratase | alkene | PB1_03 | EIJ81937 | Bacillus |
| | | | | 320 | | methanolicus |
| 4.2.1 | Dehydratase | dehydratase | alkene | MexAM | ACS38865 | Methylobacterium |
| | | | | 1_MET | | extorquens |
| | | | | A1p097 | | |
| | | | | 0 | | |
| 4.3.1 | Thioester | amine | alkene | aspA | NP_418562 | Escherichia coli |
| | hydrolase | | | | | |
| 4.3.1 | Ammonia-lyase | amine | alkene | PB1_05 | EIJ79784 | Bacillus |
| | | | | 447 | | methanolicus |
| 4.3.1 | Ammonia-lyase | amine | alkene | aspA | AAC77099 | Methylobacterium |
| | | | | | | extorquens |
| | | | | | | Pyrobaculum |
| 6.2.1 | CoA ligase | acid | acyl-CoA | Pisl_02 | YP_929773 | islandicum DSM |
| | | | | 50 | | 4184 |
| 6.2.1 | CoA ligase | acid | acyl-CoA | acs | YP_003431 | Hydrogenobacter |
| | | | | | 745 | thermophilus TK-6 |
| 6.2.1 | CoA ligase | acid | acyl-CoA | Cagg_3 | YP_002465 | Chloroflexus |
| | | | | 790 | 062 | aggregans DSM |
| | | | | | | 9485 |
| 6.2.1 | CoA ligase | acid | acyl-CoA | Cour_0 | YP_001633 | Chloroflexus |
| | | | | 002 | 649 | ourantiocus J-10-fl |
| 6.2.1 | CoA ligase | acyl-CoA | acid | sucC | NP_415256 | Escherichia coli |
| 6.2.1 | CoA ligase | acid | acyl-CoA | sucC | NP_415256 | Escherichia coli |
| 6.2.1 | CoA ligase | acid | acyl-CoA | bioW | KIX83609 | Bacillus subtilis |
| 6.2.1 | CoA ligase | acyl-CoA | acid | HPODL_ | ESW96363 | Hansenula |
| | | | | 02989 | | polymorpha |
| 6.2.1 | CoA ligase | acid | acyl-CoA | HPODL_ | ESW96363 | Hansenula |
| | | | | 02989 | | polymorpha |
| 6.2.1 | CoA ligase | acyl-CoA | acid | PB1_17 | EIJ79289 | Bacillus |
| | | | | 069 | | methanolicus |
| 6.2.1 | CoA ligase | acid | acyl-CoA | PB1_17 | EIJ79289 | Bacillus |
| | | | | 069 | | methanolicus |
| 6.2.1 | CoA ligase | acyl-CoA | acid | acsA | CCA39763 | Pichia pastoris |
| 6.2.1 | CoA ligase | acid | acyl-CoA | acsA | CCA39763 | Pichia pastoris |
| 6.2.1 | CoA ligase | acyl-CoA | acid | sucD | AIE59640 | Bacillus |
| | | | | | | methanolicus |
| 6.2.1 | CoA ligase | acid | acyl-CoA | sucD | AIE59640 | Bacillus |
| | | | | | | methanolicus |
| 6.2.1 | CoA igase | acyl-CoA | acid | MexAM | ACS42955 | Methylobacterium |
| | | | | 1_MET | | extorquens |
| | | | | A2p001 | | |
| | | | | 4 | | |
| 6.2.1 | CoA ligase | acid | acyl-CoA | MexAM | ACS42955 | Methylobacterium |
| | | | | 1_MET | | extorquens |
| | | | | A2p001 | | |
| | | | | 4 | | |
| 6.2.1 | CoA ligase | acyl-CoA | acid | acs1 | ACS42661 | Methylobacterium |
| | | | | | | extorquens |
| 6.2.1 | CoA ligase | acid | acyl-CoA | acs1 | ACS42661 | Methylobacterium |
| | | | | | | extorquens |
| 6.2.1 | CoA ligase | acyl-CoA | acid | acs | ACS40309 | Methylobacterium |
| | | | | | | extorquens |
| 6.2.1 | CoA ligase | acid | acyl-CoA | acs | ACS40309 | Methylobacterium |
| | | | | | | extorquens |
| 6.2.1 | CoA ligase | acid | acyl-CoA | Tneu_0 | ACB39368 | Thermoproteus |
| | | | | 420 | | neutrophilus |
| 6.2.1 | CoA ligase | acid | acyl-CoA | Nmar_ | ABX13205 | Nitrosopumilus |
| | | | | 1309 | | maritimus |
| 6.2.1 | CoA ligase | acyl-CoA | acid | Nmar_ | ABX13205 | Nitrosopumilus |
| | | | | 1309 | | maritimus |
| 6.2.1 | CoA ligase | acid | acyl-CoA | Nmar_ | ABX12102 | Nitrosopumilus |
| | | | | 0206 | | maritimus |
| 6.2.1 | CoA ligase | acyl-CoA | acid | Nmar_ | ABX12102 | Nitrosopumilus |
| | | | | 0206 | | maritimus |
| 6.2.1 | CoA ligase | acid | acyl-CoA | Msed_1 | ABP95580 | Metallosphaera |
| | | | | 422 | | sedula |
| 6.2.1 | CoA ligase | acid | acyl-CoA | Msed_1 | ABP95511 | Metallosphaera |
| | | | | 353 | | sedula |
| 6.2.1 | CoA ligase | acid | acyl-CoA | Msed_0 | ABP94583 | Metallosphaera |
| | | | | 406 | | sedula |
| 6.2.1 | CoA ligase | acid | acyl-CoA | Msed_0 | ABP94571 | Metallosphaera |
| | | | | 394 | | sedula |
| 6.2.1 | CoA ligase | acyl-CoA | acid | acs1 | ABC87079 | Methanothermobacter |
| | | | | | | thermautotrophicus |
| 6.2.1 | CoA ligase | acid | acyl-CoA | acs1 | ABC87079 | Methanothermobacter |
| | | | | | | thermautotrophicus |
| 6.2.1 | CoA ligase | acyl-CoA | acid | sucD | AAC73823 | Escherichia coli |
| 6.2.1 | CoA ligase | acid | acyl-CoA | sucD | AAC73823 | Escherichia coli |
| 6.3.1 | Acetylglutamate | amine | glutamyl | puuA | NP_415813 | Escherichia coli |
| | synthase | | amine | | | |
| 6.3.1 | Acetylglutamate | amine | glutamyl | HPODL_ | ESX01082 | Hansenula |
| | synthase | | amine | 00487 | | polymorpha |
| 6.3.1 | Acetylglutamate | amine | glutamyl | glnA | EIJ81404 | Bacillus |
| | synthase | | amine | | | methanolicus |
| 6.3.1 | Acetylglutamate | amine | glutamyl | glnA | ACS40162 | Methylobacterium |
| | synthase | | amine | | | extorquens |
| 6.3.1 | Acetylglutamate | amine | glutamyl | MexAM | ACS39415 | Methylobacterium |
| | synthase | | amine | 1_MET | | extorquens |
| | | | | A1p155 | | |
| | | | | 3 | | |
| no EC | Putrescine | amine | amine | puuP | AAC74378 | Escherichia coli |
| | permease | intracellular | extracellular | | | |
| no EC | Cadaverine | amine | amine | cadB | AAA97032 | Escherichia coli |
| | permease | intracellular | extracellular | | | |
| None | Amine hydroxylase | amine | hydroxyl | pubA | WP_011072 | Shewanella |
| | | | amine | | 933 | oneidensis |
| None | Amine hydroxylase | amine | hydroxyl | pp7435_ | CCA36870 | Pichia pastoris |
| amine | | | | Chr1- | | |
| | | | | 0727 | | |

Target products described herein can be biosynthesized using the pathways described herein (e.g. FIG. 1). In one aspect the pathway is a HMD pathway as set forth in FIG. 1. The HMD pathway is provided in genetically modified cell described herein (e.g., a non-naturally occurring microorganism) where the HMD pathway includes at least one exogenous nucleic acid encoding a HMD pathway enzyme expressed in a sufficient amount to produce HMD where the pathway is selected from Tables 5, 6, or 7. 1A is a 3-oxoadipyl-CoA thiolase; 1B is a 3-oxoadipyl-CoA reductase; 1C is a 3-hydroxyadipyl-CoA dehydratase; 1D is a 5-carboxy-2-pentenoyl-CoA reductase; 1E is a 3-oxoadipyl-CoA/acyl-CoA transferase; 1F is a 3-oxoadipyl-CoA synthase; 1G is a 3-oxoadipyl-CoA hydrolase; 1H is a 3-oxoadipate reductase; 1I is a 3-hydroxyadipate dehydratase; 1J is a 5-carboxy-2-pentenoate reductase; 1K is an adipyl-CoA/acyl-CoA transferase; 1L is an adipyl-CoA synthase; 1M is an adipyl-CoA hydrolase; 1N is an adipyl-CoA reductase (aldehyde forming); 10 is a 6-aminocaproate transaminase; 1P is a 6-aminocaproate dehydrogenase; 1Q is a 6-aminocaproyl-CoA/acyl-CoA transferase; 1R is a 6-aminocaproyl-CoA synthase; 1S is an amidohydrolase; 1T is spontaneous cyclization; 1U is a 6-aminocaproyl-CoA reductase (aldehyde forming); 1V is a HMDA transaminase; and 1W is a HMDA dehydrogenase.

Also provided herein is a HMD pathway as set forth in FIG. 1 where the pathway includes at least 2, 3, 4, 5, 6, 8, 9, or 10 (or all) exogenous nucleic acids encoding HMD pathway enzymes expressed in a sufficient amount to produce HMD.

One skilled in the art will readily recognize the function associated with each of the above-identified enzymes and that such enzymes can catalyze reactions on more than one substrate. In such instances, one skilled in the art will recognize such enzymes can be substituted with orthologs, paralogs, and homologs of enzymes having similar or identical function as is known in art and provided for, by example, U.S. Pat. Nos. 8,377,680 and 8,940,509 which are herein incorporated in their entireties and for all purposes.

The HMD pathway can be an acyl-CoA HMD pathway as set forth in FIG. 1 and Table 5. Accordingly, an acyl-CoA HMD pathway includes at least one exogenous nucleic acid encoding a HMD pathway enzyme selected from: 1A, 1B, 1C, 1D, 1N, (1O/1P), (1Q/1R), 1U, and (1V/1W). The acyl-CoA HMD pathway described herein and useful in the microorganisms described herein for producing HMD having reduced byproducts therefore includes all possible alternatives of the referenced pathway. Thus, for example, the acyl-CoA HMD pathway includes enzymes selected from 1A, 1B, 1C, 1D, 1N, 1O, 1P, 1Q, 1R, 1U, 1V, and 1W as defined herein. The pathway can include at least 2, 3, 4, 5, 6, or all exogenous nucleic acids for encoding HMD pathway enzymes expressed in a sufficient amount to produce HMD. The acyl-CoA HMD pathway can be a pathway as shown in Table 5.

TABLE 5
acyl-CoA HMD pathway enzymes

| | |
|---|---|
| 1A-1B-1C-1D-1N-1O-1Q-1U-1V | 1A-1B-1C-1D-1N-1P-1Q-1U-1V |
| 1A-1B-1C-1D-1N-1O-1Q-1U-1W | 1A-1B-1C-1D-1N-1P-1Q-1U-1W |
| 1A-1B-1C-1D-1N-1O-1R-1U-1V | 1A-1B-1C-1D-1N-1P-1R-1U-1V |
| 1A-1B-1C-1D-1N-1O-1R-1U-1W | 1A-1B-1C-1D-1N-1P-1R-1U-1W |

The HMD pathway can alternatively be an acid HMD pathway as set forth in FIG. 1 and Table 6. The acid HMD pathway includes at least one exogenous nucleic acid encoding a HMD pathway enzyme selected from 1A, (1E/1F/1G), 1H, 1I, 1J, (1K/1L/1M), 1D, 1N, (1O/1P), (1Q/1R), 1U, (1V/1W). An acid HMD pathway as described herein and useful in the microorganisms described herein for producing HMD having lower byproducts therefore includes all possible alternatives of the referenced pathway. Thus, for example the acid HMD pathway includes enzymes selected from 1A, 1B, 1C, 1D, 1E, 1F, 1G, 1H, 1I, 1J, 1K, 1L, 1M, 1N, 1O, 1P, 1Q, 1R, 1S, 1T, 1U, 1V, and 1W as defined herein. The pathway can include at least 2, 3, 4, 5, 6, 7, 8, 9, or 10 (or more) exogenous nucleic acids encoding HMD pathway enzymes expressed in a sufficient amount to produce HMD. The acid HMD pathway can be a pathway as shown in Table 6.

TABLE 6
Acid HMD pathway enzymes

| | | |
|---|---|---|
| 1A-1E-1H-1I-1J-1K-1D-1N-1O-1Q-1U-1V | 1A-1E-1H-1I-1J-1L-1D-1N-1O-1Q-1U-1V | 1A-1E-1H-1I-1J-1M-1D-1N-1O-1Q-1U-1V |
| 1A-1E-1H-1I-1J-1K-1D-1N-1O-1Q-1U-1W | 1A-1E-1H-1I-1J-1L-1D-1N-1O-1Q-1U-1W | 1A-1E-1H-1I-1J-1M-1D-1N-1O-1Q-1U-1W |
| 1A-1E-1H-1I-1J-1K-1D-1N-1O-1R-1U-1V | 1A-1E-1H-1I-1J-1L-1D-1N-1O-1R-1U-1V | 1A-1E-1H-1I-1J-1M-1D-1N-1O-1R-1U-1V |
| 1A-1E-1H-1I-1J-1K-1D-1N-1O-1R-1U-1W | 1A-1E-1H-1I-1J-1L-1D-1N-1O-1R-1U-1W | 1A-1E-1H-1I-1J-1M-1D-1N-1O-1R-1U-1W |
| 1A-1E-1H-1I-1J-1K-1D-1N-1P-1Q-1U-1V | 1A-1E-1H-1I-1J-1L-1D-1N-1P-1Q-1U-1V | 1A-1E-1H-1I-1J-1M-1D-1N-1P-1Q-1U-1V |
| 1A-1E-1H-1I-1J-1K-1D-1N-1P-1Q-1U-1W | 1A-1E-1H-1I-1J-1L-1D-1N-1P-1Q-1U-1W | 1A-1E-1H-1I-1J-1M-1D-1N-1P-1Q-1U-1W |
| 1A-1E-1H-1I-1J-1K-1D-1N-1P-1R-1U-1V | 1A-1E-1H-1I-1J-1L-1D-1N-1P-1R-1U-1V | 1A-1E-1H-1I-1J-1M-1D-1N-1P-1R-1U-1V |
| 1A-1E-1H-1I-1J-1K-1D-1N-1P-1R-1U-1W | 1A-1E-1H-1I-1J-1L-1D-1N-1P-1R-1U-1W | 1A-1E-1H-1I-1J-1M-1D-1N-1P-1R-1U-1W |
| 1A-1F-1H-1I-1J-1K-1D-1N-1O-1Q-1U-1V | 1A-1F-1H-1I-1J-1L-1D-1N-1O-1Q-1U-1V | 1A-1F-1H-1I-1J-1M-1D-1N-1O-1Q-1U-1V |
| 1A-1F-1H-1I-1J-1K-1D-1N-1O-1Q-1U-1W | 1A-1F-1H-1I-1J-1L-1D-1N-1O-1Q-1U-1W | 1A-1F-1H-1I-1J-1M-1D-1N-1O-1Q-1U-1W |
| 1A-1F-1H-1I-1J-1K-1D-1N-1O-1R-1U-1V | 1A-1F-1H-1I-1J-1L-1D-1N-1O-1R-1U-1V | 1A-1F-1H-1I-1J-1M-1D-1N-1O-1R-1U-1V |
| 1A-1F-1H-1I-1J-1K-1D-1N-1O-1R-1U-1W | 1A-1F-1H-1I-1J-1L-1D-1N-1O-1R-1U-1W | 1A-1F-1H-1I-1J-1M-1D-1N-1O-1R-1U-1W |
| 1A-1F-1H-1I-1J-1K-1D-1N-1P-1Q-1U-1V | 1A-1F-1H-1I-1J-1L-1D-1N-1P-1Q-1U-1V | 1A-1F-1H-1I-1J-1M-1D-1N-1P-1Q-1U-1V |
| 1A-1F-1H-1I-1J-1K-1D-1N-1P-1Q-1U-1W | 1A-1F-1H-1I-1J-1L-1D-1N-1P-1Q-1U-1W | 1A-1F-1H-1I-1J-1M-1D-1N-1P-1Q-1U-1W |
| 1A-1F-1H-1I-1J-1K-1D-1N-1P-1R-1U-1V | 1A-1F-1H-1I-1J-1L-1D-1N-1P-1R-1U-1V | 1A-1F-1H-1I-1J-1M-1D-1N-1P-1R-1U-1V |
| 1A-1F-1H-1I-1J-1K-1D-1N-1P-1R-1U-1W | 1A-1F-1H-1I-1J-1L-1D-1N-1P-1R-1U-1W | 1A-1F-1H-1I-1J-1M-1D-1N-1P-1R-1U-1W |
| 1A-1G-1H-1I-1J-1K-1D-1N-1O-1Q-1U-1V | 1A-1G-1H-1I-1J-1L-1D-1N-1O-1Q-1U-1V | 1A-1G-1H-1I-1J-1M-1D-1N-1O-1Q-1U-1V |
| 1A-1G-1H-1I-1J-1K-1D-1N-1O-1Q-1U-1W | 1A-1G-1H-1I-1J-1L-1D-1N-1O-1Q-1U-1W | 1A-1G-1H-1I-1J-1M-1D-1N-1O-1Q-1U-1W |
| 1A-1G-1H-1I-1J-1K-1D-1N-1O-1R-1U-1V | 1A-1G-1H-1I-1J-1L-1D-1N-1O-1R-1U-1V | 1A-1G-1H-1I-1J-1M-1D-1N-1O-1R-1U-1V |
| 1A-1G-1H-1I-1J-1K-1D-1N-1O-1R-1U-1W | 1A-1G-1H-1I-1J-1L-1D-1N-1O-1R-1U-1W | 1A-1G-1H-1I-1J-1M-1D-1N-1O-1R-1U-1W |
| 1A-1G-1H-1I-1J-1K-1D-1N-1P-1Q-1U-1V | 1A-1G-1H-1I-1J-1L-1D-1N-1P-1Q-1U-1V | 1A-1G-1H-1I-1J-1M-1D-1N-1P-1Q-1U-1V |
| 1A-1G-1H-1I-1J-1K-1D-1N-1P-1Q-1U-1W | 1A-1G-1H-1I-1J-1L-1D-1N-1P-1Q-1U-1W | 1A-1G-1H-1I-1J-1M-1D-1N-1P-1Q-1U-1W |
| 1A-1G-1H-1I-1J-1K-1D-1N-1P-1R-1U-1V | 1A-1G-1H-1I-1J-1L-1D-1N-1P-1R-1U-1V | 1A-1G-1H-1I-1J-1M-1D-1N-1P-1R-1U-1V |
| 1A-1G-1H-1I-1J-1K-1D-1N-1P-1R-1U-1W | 1A-1G-1H-1I-1J-1L-1D-1N-1P-1R-1U-1W | 1A-1G-1H-1I-1J-1M-1D-1N-1P-1R-1U-1W |

The HMD pathway can alternatively be an acetoacetyl-CoA HMD pathway as set forth in FIG. 2 and Table 7. The acetoacetyl-CoA HMD pathway includes at least one exogenous nucleic acid encoding a HMD pathway enzyme selected from 2A an Acetyl-CoA carboxylase (EC 6.4.1.2); 2B a Beta-ketothiolase (EC 2.3.1.9; such as atoB, phaA, bktB); 2C an Acetoacetyl-CoA synthase (EC 2.3.1.194); 2D a 3-hydroxyacyl-CoA dehydrogenase or an Acetoacetyl-CoA reductase (EC 1.1.1.35 or 1.1.1.157; such as fadB, hbd or phaB); 2E an Enoyl-CoA hydratase (EC 4.2.1.17 or 4.2.1.119, such as crt or phaJ); 2F a Trans-2-enoy-CoA reductase (EC 1.3.1.8, 1.3.1.38 or 1.3.1.44, such as Ter or tdter); 2G a Beta-ketothiolase (EC 2.3.1.16, such as bktB); 2H a 3-hydroxyacyl-CoA dehydrogenase or Acetoacetyl-CoA reductase (EC 1.1.1.35 or 1.1.1.157, such as fadB, hbd, phaB, or FabG); 2J an Enoyl-CoA hydratase (EC 4.2.1.17 or 4.2.1.119, such as crt or phaJ); 2K a Trans-2-enoy-CoA reductase (EC 1.3.1.8, 1.3.1.38, or 1.3.1.44, such as Ter or tdter); 2L a Butanal dehydrogenase (EC 1.2.1.57); 2M an Aldehyde dehydrogenase (EC 1.2.1.4); 2N a thioesterase (EC 3.2.1, such as YciA, tesB, or Acot13); 3P a Monooxygenase (EC 1.14.15.1, such as CYP153A, ABE47160.1, ABE47159.1, ABE47158.1, CAH04396.1, CAH04397.1, CAH04398.1, or ACJ06772.1); 3Q an Alcohol dehydrogenase (EC 1.1.1.2 or 1.1.1.258, such as CAA90836.1, YMR318c, cpnD, gabD, or ChnD); 3R a co-transaminase (EC 2.6.1.18, 2.6.1.19, 2.6.1.29, 2.6.1.48, or 2.6.1.82, such as AA59697.1, AAG08191.1, AAY39893.1, ABA81135.1, AEA39183.1); and 3S a lactamase (EC 3.5.2). The pathway can include at least 2, 3, 4, 5, 6, 7, 8, 9, 10 (or all) exogenous nucleic acids for encoding HMD pathway enzymes expressed in a sufficient amount to produce HMD. The acetoacyl-CoA HMD pathway can be a pathway as shown in Table 7.

TABLE 7
acetoacetyl-CoA HMD enzymes

| | |
|---|---|
| 2B-2D-2E-2F-2G-2H-2J-2K-2L-2M-3P-3Q-3R-1Q-1U-1V | 2A-2C-2D-2E-2F-2G-2H-2J-2K-2L-2M-3P-3Q-3R-1Q-1U-1V |
| 2B-2D-2E-2F-2G-2H-2J-2K-2L-2M-3P-3Q-3R-1Q-1U-1W | 2A-2C-2D-2E-2F-2G-2H-2J-2K-2L-2M-3P-3Q-3R-1Q-1U-1W |
| 2B-2D-2E-2F-2G-2H-2J-2K-2L-2M-3P-3Q-3R-1R-1U-1V | 2A-2C-2D-2E-2F-2G-2H-2J-2K-2L-2M-3P-3Q-3R-1R-1U-1V |
| 2B-2D-2E-2F-2G-2H-2J-2K-2L-2M-3P-3Q-3R-1R-1U-1W | 2A-2C-2D-2E-2F-2G-2H-2J-2K-2L-2M-3P-3Q-3R-1R-1U-1W |
| 2B-2D-2E-2F-2G-2H-2J-2K-2N-3P-3Q-3R-1Q-1U-1V | 2A-2C-2D-2E-2F-2G-2H-2J-2K-2N-3P-3Q-3R-1Q-1U-1V |
| 2B-2D-2E-2F-2G-2H-2J-2K-2N-3P-3Q-3R-1Q-1U-1W | 2A-2C-2D-2E-2F-2G-2H-2J-2K-2N-3P-3Q-3R-1Q-1U-1W |
| 2B-2D-2E-2F-2G-2H-2J-2K-2N-3P-3Q-3R-1R-1U-1V | 2A-2C-2D-2E-2F-2G-2H-2J-2K-2N-3P-3Q-3R-1R-1U-1V |
| 2B-2D-2E-2F-2G-2H-2J-2K-2N-3P-3Q-3R-1R-1U-1W | 2A-2C-2D-2E-2F-2G-2H-2J-2K-2N-3P-3Q-3R-1R-1U-1W |

An acetoacetyl-CoA HMD pathway as described herein and useful in the microorganisms described herein for producing HMD having lower byproducts therefore includes all possible alternatives of the referenced pathway. As one skilled in the art will readily understand, the biosynthetic pathways described herein have overlapping and corresponding enzymatic steps. Thus, for example, conversion of adipate semialdehyde to 6ACA can be completed in a non-naturally occurring microorganism described herein using any one or combination of the HMD pathways described herein. Furthermore, the HMD pathway of FIG. 2 and FIG. 3 can be used in combination with an acyl-CoA HMD pathway or acid HMD pathways set forth in FIG. 1. For example, the pathways of FIG. 2 and FIG. 3 can be used in combination with the pathway of FIG. 1 to synthesize 6ACA which can be converted by an acyl-CoA HMD pathway or acid HMD pathway described herein to 6ACA-semialdehyde (e.g. by enzyme 1U). Such overlap and crossover is readily apparent to those of skill in the art and is included in the invention described herein.

Target products such as 6ACA, ADA and CPL including intermediates in pathways capable of producing such target products are present within the HMD pathways described herein. Accordingly, 6ACA, ADA, CPL, and other intermediates of the HMD pathways described herein can be biosynthetically derived using the enzymes described herein for a HMD pathway described herein. For example, 6ACA, ADA, and CPL can be produced from a genetically engineered cell described herein having a HMD pathway described herein modified as described herein to produce 6ACA, ADA, and CPL. In such instances, these pathways can be referred to a “6ACA pathway,” a “ADA pathway,” and a “CPL pathway” respectively. Such pathways also, while including HMD pathway enzymes, can likewise be referred to as including a “6ACA pathway enzyme,” “ADA pathway enzyme,” and a “CPL pathway enzyme” respectively.

The invention therefore includes a non-naturally occurring microbial organism that includes a HMD pathway and is capable of producing HMD, where the non-naturally occurring microbial organism further includes: (a) a genetic modification selected from: (i) a genetic modification that decreases activity of an enzyme selected from A1-A25; (ii) a genetic modification that increases activity of an enzyme selected from B1-B5; and (iii) a combination of two or more, three or more, four or more, five or more, six or more, seven or more, eight or more, or all of the genetic modifications of (i) and (ii). The non-naturally occurring microorganism also includes a HMD pathway as described herein that includes at least one exogenous nucleic acid encoding a HMD pathway enzyme described herein. Such cells can include at least two, at least three, at least four, at least five, at least six, at least seven, at least eight, at least nine or at least ten exogenous nucleic acids encoding a HMD pathway enzyme.

In another aspect is a LVA pathway as set forth in FIG. 1. The LVA pathway includes at least one exogenous nucleic acid encoding a LVA pathway enzyme selected from: 1A-1E-1AA; 1A-1F-1AA; or 1A-1G-1AA as set forth in FIG. 1, where 1A, 1E, 1F, and 1G are as defined herein and 1AA is a 3-oxoadipate decarboxylase. The pathway can include at least 2, or 3 exogenous nucleic acids for encoding LVA pathway enzymes expressed in a sufficient amount to produce LVA. In yet another aspect is a genetically modified cell described herein that includes a LVA pathway having at least one exogenous nucleic acid encoding a LVA pathway enzyme expressed in a sufficient amount to produce LVA, where the LVA pathway includes a pathway selected from: 1A-1E-1AA; 1A-1F-1AA; 1A-1G-1AA, wherein 1A is a 3-oxoadipyl-CoA thiolase, 1E is a 3-oxoadipyl-CoA/acyl-CoA transferase, 1F is a 3-oxoadipyl-CoA synthase, and 1AA is an is a 3-oxoadipate decarboxylase. Such cells can include at least two or at least three exogenous nucleic acids encoding a LVA pathway enzyme.

In still another aspect is a non-naturally occurring microbial organism that includes a LVA pathway and is capable of producing LVA, where the non-naturally occurring microbial organism further includes: (a) a genetic modification selected from: (i) a genetic modification that decreases activity of an enzyme selected from A1-A25; (ii) a genetic modification that increases activity of an enzyme selected from B1-B5; and (iii) a combination of two or more, three or more, four or more, five or more, six or more, seven or more, eight or more, or all of the genetic modifications of (i) and (ii). The non-naturally occurring microorganism also includes a LVA pathway as described herein that includes at least one exogenous nucleic acid encoding a LVA pathway enzyme described herein. Such cells can include at least two or at least three exogenous nucleic acids encoding a LVA pathway enzyme.

In yet another aspect is a CPO pathway as set forth in FIG. 5. The CPO pathway can be a pathway substantially the same as that of FIG. 5 or Table 8. In another aspect is a cell that includes a CPO pathway that includes at least one exogenous nucleic acid encoding a CPO pathway enzyme expressed in a sufficient amount to produce CPO, where the CPO pathway is a pathway selected from Table 8. and where 5A is an adipyl-CoA reductase; 5B is an adipate semialdehyde reductase; 5C is a 6-hydroxyhexanoyl-CoA transferase or synthetase; 5D is a 6-hydroxyhexanoyl-CoA cyclase or spontaneous cyclization; 5E is an adipate reductase; 5F is an adipyl-CoA transferase, synthetase or hydrolase; 5G is a 6-hydroxyhexanoate cyclase; 5H is a 6-hydroxyhexanoate kinase; 5I is a 6-hydroxyhexanoyl phosphate cyclase or spontaneous cyclization; and 5J is a phosphotrans-6-hydroxyhexanoylase. The pathway can include at least 2, 3, 4, 5, or all exogenous nucleic acids encoding CPO pathway enzymes expressed in a sufficient amount to produce CPO. Thus, such a cell can include at least two, at least three, at least four, at least five, at least six, at least seven, at least eight, at least nine or at least ten exogenous nucleic acids encoding a CPO pathway enzyme. The pathway can be a CPO pathway that includes CPO pathway enzymes 5A-5B-5C-5D of FIG. 5.

TABLE 8
CPO pathway enzymes

| | |
|---|---|
| 5A-5B-5C-5D | 5A-5B-5C-5J-5I |
| 5E-5B-5C-5D | 5E-5B-5C-5J-5I |
| 5F-5A-5B-5C-5D | 5F-5A-5B-5C-5J-5I |
| 5F-5E-5B-5C-5D | 5F-5E-5B-5C-5J-5I |
| 5A-5B-5G | 5A-5B-5H-5I |
| 5E-5B-5G | 5E-5B-5H-5I |
| 5F-5A-5B-5G | 5F-5A-5B-5H-5I |
| 5F-5E-5B-5G | 5F-5E-5B-5H-5I |

In another aspect is a non-naturally occurring microbial organism that includes a CPO pathway and is capable of producing CPO, where the non-naturally occurring microbial organism further includes: (a) a genetic modification selected from: (i) a genetic modification that decreases activity of an enzyme selected from A1-A25; (ii) a genetic modification that increases activity of an enzyme selected from B1-B5; and (iii) a combination of two or more, three or more, four or more, five or more, six or more, seven or more, eight or more, or all of the genetic modifications of (i) and (ii). The non-naturally occurring microorganism also includes a CPO pathway as described herein that includes at least one exogenous nucleic acid encoding a CPO pathway enzyme described herein. Such cells can include at least two, at least three, at least four, at least five, at least six, at least seven, at least eight, at least nine or at least ten exogenous nucleic acids encoding a CPO pathway enzyme.

Also provided herein is a pathway to HDO (i.e. an “HDO pathway”). The pathway can be a pathway substantially the same as FIG. 4. HDO can be biosynthesized starting from 6ACA, adipyl-CoA, or adipate, including intermediates thereof. The pathway includes at least one exogenous nucleic acid encoding a HDO pathway enzyme selected from Table 9, where 4A is a 6-aminocaproyl-CoA transferase or synthetase catalyzing conversion of 6ACA to 6-aminocaproyl-CoA; 4B is a 6-aminocaproyl-CoA reductase catalyzing coversion of 6-aminocaproyl-CoA to 6-aminocaproate semialdehyde; 4C is a 6-aminocaproate semialdehyde reductase catalyzing conversion of 6-aminocaproate semialdehyde to 6-aminohexanol; 4D is a 6-aminocaproate reductase catalyzing conversion of 6ACA to 6-aminocaproate semialdehyde; 4E is an adipyl-CoA reductase adipyl-CoA to adipate semialdehyde; 4F is an adipate semialdehyde reductase catalyzing conversion of adipate semialdehyde to 6-hydroxyhexanoate; 4G is a 6-hydroxyhexanoyl-CoA transferase or synthetase catalyzing conversion of 6-hydroxyhexanoate to 6-hydroxyhexanoyl-CoA; 4H is a 6-hydroxyhexanoyl-CoA reductase catalyzing conversion of 6-hydroxyhexanoyl-CoA to 6-hydroxyhexanal; 4I is a 6-hydroxyhexanal reductase catalyzing conversion of 6-hydroxyhexanal to HDO; 4J is a 6-aminohexanol aminotransferase or oxidoreductases catalyzing conversion of 6-aminohexanol to 6-hydroxyhexanal; 4K is a 6-hydroxyhexanoate reductase catalyzing conversion of 6-hydroxyhexanoate to 6-hydroxyhexanal; 4L is an adipate reductase catalyzing conversion of ADA to adipate semialdehyde; and 4M is an adipyl-CoA transferase, hydrolase or synthase catalyzing conversion of adipyl-CoA to ADA. The pathway can include at least 2, 3, 4, 5, 6, 7, 8, 9, or 10 (or all) exogenous nucleic acids for encoding HDO pathway enzymes expressed in a sufficient amount to produce HDO.

In another aspect is a cell that includes a HDO pathway described herein having at least one exogenous nucleic acid encoding a HDO pathway enzyme expressed in a sufficient amount to produce HDO, where the HDO pathway is a pathway selected from Table 9. In still another aspect is a non-naturally occurring microbial organism that includes a HDO pathway and is capable of producing HDO, where the non-naturally occurring microbial organism further includes: (a) a genetic modification selected from: (i) a genetic modification that decreases activity of an enzyme selected from A1-A25; (ii) a genetic modification that increases activity of an enzyme selected from B1-B5; and (iii) a combination of two or more, three or more, four or more, five or more, six or more, seven or more, eight or more, or all of the genetic modifications of (i) and (ii). The non-naturally occurring microorganism also includes a HDO pathway as described herein that includes at least one exogenous nucleic acid encoding a HDO pathway enzyme described herein. Such cells can include at least two, at least three, at least four, at least five, at least six, at least seven, at least eight, at least nine or at least ten exogenous nucleic acids encoding a HDO pathway enzyme. Such a cell can include at least two, at least three, at least four, at least five, at least six, at least seven, at least eight, at least nine or at least ten exogenous nucleic acids encoding a HDO pathway enzyme.

Moreover, 6ACA, adipyl-CoA, and adipate are intermediates as described above in the HMD pathways described herein. Thus, HDO can be synthesized using intermediates produced in a biosynthetic pathway described herein such as those set forth in FIG. 1 or FIG. 2 that results in subsequent enzyme catalysis to an intermediate provided in FIG. 4. Accordingly, HDO can be synthesized using any combination of a HMD pathway (e.g., FIG. 1, 2, or 3) in combination with a HDO pathway (e.g., FIG. 4) provided the HMD pathway supplies an intermediate useful in the HDO pathway. The pathway can be a HDO pathway that includes HDO pathway enzymes 4E-4F-4G-4H-4I of FIG. 4.

TABLE 9
HDO pathway enzymes

| | | |
|---|---|---|
| 4D-4C-4J-4I | 4M-4E-4F-4G-4H-4I | 4M-4L-4F-4G-4H-4I |
| 4E-4F-4G-4H-4I | 4L-4F-4G-4H-4I | 4M-4L-4F-4K-4I |
| 4E-4F-4K-4I | 4L-4F-4K-4I | 4A-4B-4C-4J-4I |
| 4M-4E-4F-4K-4I | | |

In another aspect is a non-naturally occurring microbial organism that includes a pathway described herein to produce a target product and a genetic modification of one or more enzymes selected from A1-A25 and B1-B2 as described herein. The byproduct can be a compound set forth in Table 10 or 11. Byproducts described herein can include intermediates found in the biosynthetic pathways described herein. Byproducts useful for reduction or elimination during the biosynthesis of a target products described herein include those exemplified in Table 10, Table 11, and Table 12. It should be appreciated that each byproduct may not be present in certain pathways to biosynthesize a described target product as set forth herein and in for example Table 10 and 11.

The invention provides a non-naturally occurring microbial organism having a HDO pathway and capable of producing HDO, where the non-naturally occurring microbial organism further includes a genetic modification selected from: (a) a genetic modification that decreases activity of an enzyme selected from A1-A25; (b) a genetic modification that increases activity of an enzyme selected from B1-B5; and (c) a combination of two or more, three or more, four or more, five or more, six or more, seven or more, eight or more, or all of the genetic modifications of (a) and (b); and a HDO pathway described herein that includes at least one exogenous nucleic acid encoding a HDO pathway enzyme. Such non-naturally occurring microbial organism can be grown in substantially anaerobic culture medium.

In another aspect is a non-naturally occurring microbial organism having a HDO pathway described herein and at least one exogenous nucleic acid encoding a HDO pathway enzyme as described herein expressed in a sufficient amount to produce HDO, wherein the HDO pathway includes: a 6-aminocaproyl-CoA transferase or synthetase catalyzing conversion of 6ACA to 6-aminocaproyl-CoA (4A); a 6-aminocaproyl-CoA reductase catalyzing conversion of 6-aminocaproyl-CoA to 6-aminocaproate semialdehyde (4B); a 6-aminocaproate semialdehyde reductase catalyzing conversion of 6-aminocaproate semialdehyde to 6-aminohexanol (4C); a 6-aminocaproate reductase catalyzing conversion of 6ACA to 6-aminocaproate semialdehyde (4D); an adipyl-CoA reductase adipyl-CoA to adipate semialdehyde (4E); an adipate semialdehyde reductase catalyzing conversion of adipate semialdehyde to 6-hydroxyhexanoate (4F); a 6-hydroxyhexanoyl-CoA transferase or synthetase catalyzing conversion of 6-hydroxyhexanoate to 6-hydroxyhexanoyl-CoA (4G); a 6-hydroxyhexanoyl-CoA reductase catalyzing conversion of 6-hydroxyhexanoyl-CoA to 6-hydroxyhexanal (4H); a 6-hydroxyhexanal reductase catalyzing conversion of 6-hydroxyhexanal to HDO (4I); a 6-aminohexanol aminotransferase or oxidoreductases catalyzing conversion of 6-aminohexanol to 6-hydroxyhexanal (4J); a 6-hydroxyhexanoate reductase catalyzing conversion of 6-hydroxyhexanoate to 6-hydroxyhexanal (4K); an adipate reductase catalyzing conversion of ADA to adipate semialdehyde (4L); or an adipyl-CoA transferase, hydrolase or synthase catalyzing conversion of adipyl-CoA to ADA (4M). The HDO pathway can be a HDO pathway selected from Table 9. The HDO pathway can include at least 2, 3, 4, 5, 6, 7, 8, 9, 10, 11, 12, or 13 pathway enzymes of a HDO pathway selected from Table 9. The HDO pathway can include at least 2, 3, 4, 5, 6, 7, 8, 9, 10, 11, 12, or 13 exogenous nucleic acids encoding 2, 3, 4, 5, 6, 7, 8, 9, 10, 11, 12, or 13 HDO pathway enzymes selected from 4A, 4B, 4C, 4D, 4E, 4F, 4G, 4H, 4I, 4J, 4K, 4L, and 4M.

It may be undesirable, in certain instances, to reduce or eliminate a byproduct set forth in Table 10 or 11, when such a byproduct is an intermediate compound biosynthesized in a pathway to product a target product. Thus, for example, one skilled in the art will readily recognize it may be undesirable to eliminate an intermediate of a biosynthesis pathway described herein for producing a target product (e.g. HMD, LVA, 6ACA, CPL, CPO, ADA, or HDO) where the intermediate is considered important to produce the target product in sufficient quantities as described herein. For exemplary purposes, one skilled in the art would understand deleting the enzyme that biosynthesizes 3-hydroxyadipate may reduce or eliminate yields of, for example, adipate as a target product. Similarly, and for exemplary purposes, one skilled in the art would understanding deleting the enzyme that biosynthesizes 3-oxoadipate may reduce or eliminate yields of, for example, adipate or LVA as a target product.

Further, one skilled in the art would readily understand particular byproducts listed in Table 10 may be found as intermediates in the biosynthetic pathways described herein of a target product. In such instances, when biosynthesizing a target product, it may be undesirable to genetically modify a cell expressing enzymes useful for synthesizing such target products. For example, By17 (6ACA), can be a byproduct for biosynthesis of, for example, HMD as exemplified by Table 10 and FIG. 1. Thus, in certain instances, byproducts and target products are mutually exclusive when referring to the same compound in a biosynthetic pathway. Accordingly byproducts set forth in Table 10 may have relevance to specific pathways and may not be applicable to certain other pathways. Table 12 shows exemplary byproducts of the pathways described herein to biosynthesize target products described herein.

In another aspect are cells described herein that can contain a HMD pathway described herein where such a cell is capable of producing HMD as a target product, and has one or more genetic modifications described herein resulting in a reduced level of at least one of byproducts By 1 to By66 as set forth in Table 10 and Table 11. Such genetic modifications can also reduce levels of at least one byproduct selected from IB1-IB34 of Table 11. Cells expressing a HMD pathway described herein and capable of producing HMD as a target product, and having one or more genetic modifications described herein can have reduced levels of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66 byproducts selected from By1-By67 as set forth in Table 10 and Table 12 and optionally in combination with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 byproducts selected from IB1-IB34 of Table 11. It should also be appreciated that such a cell can include a HMD pathway having at least one exogenous nucleic acid encoding a pathway enzyme expressed in a sufficient amount to produce ADA, 6ACA, or CPL (e.g. a ADA, 6ACA, or CPL pathway enzyme).

Cells described herein can contain an acetoacetyl-CoA HMD pathway described herein where such a cell is capable of producing HMD as a target product, and has one or more genetic modification described herein. Such cells can have reduced levels of least one of byproducts By8-By12, By15, By17-By38, or By40-By60 as set forth in Table 10 and Table 12 or of IB1-IB34 of Table 11. Cells expressing an acetoacetyl-CoA HMD pathway described herein capable of producing HMD as a target product, and at least one genetic modification described herein can include a reduction of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 byproducts selected from By8-By12, By15, By17-By38, or By40-By60 or IB1-IB34 as set forth in Table 10 and Table 11. It should also be appreciated that such a cell can include a HMD pathway having at least one exogenous nucleic acid encoding a pathway enzyme expressed in a sufficient amount to produce ADA, 6ACA, or CPL (e.g. a ADA, 6ACA, or CPL pathway enzyme).

HMD produced by cells described herein can include one or more byproducts as described herein. Particular byproducts may be desirable to reduce to lower levels than other byproducts produced by the same biosynthetic pathway. For example, a byproduct described herein can degrade or promote degradation of HMD. Byproducts described herein can also decrease yield of target products. HMD produced using the cells and methods described herein can include one or more byproducts selected from By1, By9, By13, By14, By17, By18, By20, By 24, By25, By27, By35, By39, or By40 or IB1-IB34 as set forth in Table 10 and Table 11. HMD produced using cells and methods described herein can include at least 2, 3, 4, 5, 6, or all of By1, By9, By13, By14, By17, By18, By20, By 24, By25, By27, By35, By39, and By40. HMD produced using the cells and methods described herein can include at least 2, 3, 4, 5, 6, or all of By1, By9, By13, By14, By17, By18, By20, By 24, By25, By27, By35, By39, and By40, where at least one of the byproducts is present at level lower than HMD produced in a cell lacking genetic modifications associated with reduction of the byproduct as described herein.

TABLE 10
Exemplary byproducts

| | | | | Relevant | Steps |
|---|---|---|---|---|---|
| | | | | EC | from |
| By# | Byproduct Name | Mode of formation | Dissimilation Pathway | classes | Pathway |
| By1 | 3-oxoadipate | From 3-oxoadipyl-CoA via non- | 3OaCoA --> | 2.8.3; | 1 |
| | | specific CoA hydrolase, ligase | 3OAdip3OAdip | 3.1.2; | |
| | | or transferase activity | | 6.2.1 | |
| By 2 | 4-oxopentanoate | Oxoadipate can be | 3OaCoA --> | 2.8.3; | 1 |
| | | decarboxylated to 4- | 3OAdip3OAdip --> | 3.1.2; | |
| | | oxopentanoate | 4OPent (LEV) | 6.2.1; | |
| | | | | 4.1.1 | |
| By 3 | 3-oxo-6-amino | From 3-oxoadipate | 3OaCoA --> 3OaSald --> | 1.2.1; | 2 |
| | hexanoate | semialdehyde (formed by a | 3O6Ahx | 1.4.1; | |
| | | non-specific ALD activity on 3- | | 2.6.1 | |
| | | oxoadipyl-CoA) and via non- | | | |
| | | specific transaminase activity | | | |
| By 4 | 3,6-diamino | From transamination of 3-oxo, | 3OACoA --> 3OaSald --> | 1.2.1; | 3 |
| | hexanoate | 6-amino hexanoate or 3-amino | 3O6Ahx --> 36DAhx | 1.4.1; | |
| | | adipate semialdehyde | | 2.6.1 | |
| By 5 | 3-oxo-6-hydroxy | From non-specific ald and adh | 3OaCoA --> 3OaSald --> | 1.1.1; | 2 |
| | hexanoate | activity on 3-oxoadipyl-CoA | 3K6Hhx | 1.2.1 | |
| By 6 | 3,6-dihydroxy | From non-specific adh activity | 3OaCoA --> 3OaSald --> | 1.1.1; | 3 |
| | hexanoate | on 3-oxo, 6-hydroxy hexanoate | 3K3Hhx --> 36DHhx | 1.2.1 | |
| | | or from non-specific adh | | | |
| | | activity on 3-hydroxyadipate | | | |
| | | semialdehyde | | | |
| By 7 | 3-amino-6-hydroxy | From 3-oxo, 6-hydroxy | 3OaCoA --> 3OaSald --> | 1.1.1; | 3 |
| | hexanoate | hexanoate via non-specific | 3K6Hhx --> 3A6Hhx | 1.2.1; | |
| | | transaminase activity | | 1.4.1; | |
| | | | | 2.6.1 | |
| By 8 | 6-hydroxyhex-2- | From non-specific dehydratase | 3OaCoA --> 3OaSald --> | 1.2.1; | 4 |
| | enoate | activity on 3,6-dihydroxy | 3K6Hhx --> 36DHhx --> | 1.4.1; | |
| | | hexanoate or deaminase | 6H2HEN | 2.6.1; | |
| | | activity on 3-amino 6-hydroxy | 3OaCoA --> 3OaSald --> | 4.2.1; | |
| | | hexanoate | 3K6Hhx --> 3A6Hhx --> | 4.3.1 | |
| | | | 6H2HEN | | |
| By 9 | 3-hydroxyadipate | From hydrolysis or CoA | 3HACoA --> 3HAdip | 2.8.3; | 1 |
| | | ligase/transferase activity on 3- | | 3.1.2; | |
| | | hydroxyadipyl-CoA | | 6.2.1 | |
| By 10 | 3-hydroxy-6-amino | From 3-hydroxyadipate | 3HACoA --> 3HAdipSA --> | 1.2.1; | 2 |
| | hexanoate | semialdehyde (result of non- | 3H6Ahx | 1.4.1; | |
| | | specific ALD) via a non-specific | 3OaCoA --> 3OaSald --> | 2.6.1; | |
| | | transaminase activity or from | 3O6Ahx --> 3H6Ahx | 1.1.1 | |
| | | 3-oxoadipate semialdehyde by | | | |
| | | non-specific transaminase and | | | |
| | | adh activity | | | |
| By 11 | 6-aminohex-2- | From 3-hydroxy, 6-amino | 3HACoA --> 3HAdipSA --> | 1.2.1; | 3 |
| | enoate | hexanoate via dehydration | 3H6Ahx --> 6AH2EN | 2.6.1; | |
| | | | | 1.4.1; | |
| | | | | 4.2.1 | |
| By 12 | 4- | From CoA ligase activity on 3- | 3HACoA --> 3HAdipSA --> | 1.2.1; | 4 |
| | hydroxypiperidin- | hydroxy,6-amino hexanoate | 3H6Ahx --> | 2.6.1; | |
| | 2-one | (the same enzyme that works | 3H6AhxCoA --> | 1.4.1; | |
| | | on 6-ACA could work here), the | 4Hpip2one | 2.8.3; | |
| | | byproduct could cyclize | | 6.2.1 | |
| By 13 | 5-carboxy-2- | From 5-carboxypentenoyl-CoA | 5C2PenCoA --> 5C2Pen | 2.8.3; | 1 |
| | pentenoate | via non-specific CoA hydrolase, | | 3.1.2; | |
| | | ligase or transferase activity | | 6.2.1 | |
| By 14 | 6-hydroxy hex-4- | From 5-carboxy 2-pentenoyl- | 5C2PenCoA --> | 1.2.1; | 2 |
| | enoate | CoA via a non-specific ald and | 5C2Penald --> 6HH4en | 1.1.1; | |
| | | adh | | 1.1.1 | |
| By 15 | 6- | Several; Non-specific enoate | 5C2PenCoA --> | 1.2.1; | 3 |
| | hydroxyhexanoate | reductases such as nemA can | 5C2Penald --> 6HH4en --> | 1.1.1; | |
| | | work on 6-hydroxyhex-4- | 6HHex | 1.3.1 | |
| | | enoate or 6-hydroxyhex-2- | AdipSA --> 6HHex | | |
| | | enoate. ADH reacts with | | | |
| | | adipate semialdehyde | | | |
| By 16 | 6-aminohex 4- | From non-specific ald and | 5C2PenCoA --> | 1.2.1; | 2 |
| | enoate | transaminase activity on 5- | 5C2Penald --> 6AH4en | 2.6.1; | |
| | | carboxy 2-pentenoyl-CoA | | 1.4.1 | |
| By 17 | 6-aminocaproic | Pathway intermediate; | HMDA --> 6acasa --> | 1.2.1; | 3 |
| | acid (6-ACA) | Backflux from HMDA by | 6ACA | 1.2.3; | |
| | | irreversible enzymes (eg amine | | 1.4* | |
| | | oxidase and aldehyde | | | |
| | | dehydrogenase or oxidase) | | | |
| By 18 | adipate | From hydrolysis of adipyl-CoA | Adipyl-CoA --> Adip | 1.2.1; | 1-2 |
| | | or non-specific CoA | AdipSA --> Adip | 3.1.2; | |
| | | ligase/transferase activity; | | 2.8.3; | |
| | | backflux from adipate | | 6.2.1; | |
| | | semialdehyde by irreversible | | 1.2.3 | |
| | | aldehyde dehyrogenase | | | |
| By 19 | caprolactam (CPL) | If 6-aminocaproyl-CoA is | 6-ACA-CoA --> CPL | — | 1 |
| | | formed, it can cyclize to form | | | |
| | | CPL | | | |
| By 20 | 6-aminohexanol | ADH can react with | 6acasa --> 6-AHexOH | 1.1.1 | 1 |
| | | 6acasaehyde | 6-ACA-CoA --> 6-ACA- | | |
| | | | OH | | |
| By 21 | N-hydroxy 6-ACA | By reaction of O2 with 6-ACA | 6-ACA--> NOH-6ACA | No EC¹ | 1 |
| By 22 | N-hydroxy | By reaction of N-hydroxy 6-ACA | 6-ACA--> NOH-6ACA --> | 2.3.1; No | 2 |
| | succinyl-6ACA | with succinyl-CoA | NOH-succ-6ACA | EC¹ | |
| By 23 | N-methyl 6-ACA | By reaction of SAM with 6-ACA | 6-ACA -> Nme-6ACA | 2.1.1 | 1 |
| By 24 | N-glutamyl-6-ACA | Via glutamyl-putrescine ligase | 6-ACA --> Nglu-6ACA | 6.3.1 | 1 |
| By 25 | N-acetyl-6-amino | By reaction of acetyl-CoA with | 6-ACA --> acetyl-6-ACA | 2.3.1 | 1 |
| | caproic acid | 6-amino caproic acid | | | |
| By 26 | N-carbamoyl-6ACA | By reaction of carbamoyl | 6-ACA --> 6-ACA-Carb | 2.1.3 | 1 |
| | | phosphate with 6-ACA | | | |
| By 27 | N-acetyl-HMDA | By reaction of acetyl-CoA with | HMDA --> Acetyl- | 2.3.1 | 1 |
| | | HMDA | HMDA | | |
| By 28 | N-carbamoyl- | By reaction of HMDA with | HMDA --> HMDA-Carb | 2.1.3 | 1 |
| | HMDA | carbamoyl phosphate | | | |
| By 29 | Tetrahydroazepine | From 6-aminocaproate | 6acasa--> | 2.6.1 | 1 |
| | | semialdehyde by putrescine | Tetrahydroazepine | | |
| | | amino transferases or | | | |
| | | spontaneous | | | |
| By By | N-hydroxy HMDA | By reaction of O2 with HMDA | HMDA -> OH-HMDA | No EC¹ | 1 |
| 30 | | | | | |
| By 31 | N-succinyl HMDA | By reaction of HMDA with | HMDA --> Succ-HMDA | 2.3.1 | 1 |
| | | succinyl-CoA | | | |
| By 32 | N-hydroxy succinyl | By reaction of N-hydroxy | HMDA --> OH-HMDA --> | No EC¹; | 2 |
| | HMDA | HMDA with succinyl-CoA | N—OH-succ-HMDA | 2.3.1 | |
| By 33 | N-methyl HMDA | By reaction of SAM with HMDA | HMDA -> ME-HMDA | 2.1.1 | 1 |
| By 34 | N,N-dimethyl | By reaction of SAM with Me- | HMDA -> ME-HMDA --> | 2.1.1 | 2 |
| | HMDA | HMDA | NN-DM-HMDA | | |
| By 35 | Glutamyl-HMDA | Via glutamyl-putrescine ligase | HMDA --> Glu-HMDA | 6.3.1 | 1 |
| By 36 | 7-carboxy-3- | The thiolase for 3-oxoadipyl- | 5C2PenCoA --> | 2.3.1; | 2 |
| | oxohept-5-enoate | CoA has been documented to | 3oxooct-4-enoyl-CoA --> | 3.1.2; | |
| | (or 3-oxo 5,6- | combine with acetyl-CoA and | 7-c-3-oxooct-4- | 2.8.3; | |
| | didehydrosuberate) | make the CoA form of this | enoate | 6.2.1 | |
| | | compound | | | |
| By 37 | N-acyl-HMDA or | By reaction of acyl-CoA with | HMDA --> acyl-HMDA | 2.3.1 | 1-2+ |
| | N1,N6-diacyl- | HMDA on one or both amines | | | |
| | HMDA | | | | |
| By 38 | N-propylamine- | HMDA can react with S-MetP | HMDA + SMet --> | 2.5.1 | 1 |
| | HMDA | | HMDA-NPA | | |
| By 39 | succinate | Via native pathways or from | SucCoA --> Succ | 3.1.2; | 1 |
| | | CoA hydrolases acting on | SucCoA --> Sucsal --> | 2.8.3; | |
| | | succinyl-CoA | Succ | 6.2.1; | |
| | | | | 1.2.1; | |
| | | | | 1.2.1; | |
| | | | | 1.2.3 | |
| By 40 | 4-aminobutyrate | Native and pathway | SuCoA --SucSal --> | 1.2.1; | 2 |
| | | transaminases can convert | GABA | 2.6.1; | |
| | | succinate semialdehyde to 4- | | 1.4.1 | |
| | | aminobutyrate | | | |
| By 41 | N-acetyl-4-amino | From reaction of 4- | SuCoA --SucSal --> | 1.2.1; | 3 |
| | butyrate | aminobutyrate with acetyl-CoA | GABA --> Ac-GABA | 2.6.1; | |
| | | | | 2.3.1; | |
| | | | | 1.4.1 | |
| By 42 | methyl-4-amino | By reaction of SAM with 4- | SuCoA --SucSal --> | 1.2.1; | 3 |
| | butyrate | amino butyrate | GABA--> Me-GABA | 2.6.1; | |
| | | | | 2.1.1; | |
| | | | | 1.4.1 | |
| By 43 | 4-aminobutanol | From 4-aminobutyrate | SuCoA --SucSal --> | 1.2.1; | 5 |
| | | | GABA --> GABA-CoA --> | 2.6.1; | |
| | | | 4ABal --> 4AB-OH | 2.8.3; | |
| | | | | 6.2.1; | |
| | | | | 1.1.1; | |
| | | | | 1.4.1 | |
| By 44 | Glutamyl | Via a glutamyl-putrescine ligase | SuCoA --SucSal --> | 1.2.1; | 6 |
| | putrescine | | GABA --> GABA-CoA --> | 1.4.1; | |
| | | | 4ABal --> Put --> Glu- | 2.6.1; | |
| | | | Put | 2.8.3; | |
| | | | | 6.2.1; | |
| | | | | 6.3.1 | |
| By 45 | putrescine | 4-aminobutyrate can be | SuCoA --SucSal --> | 1.2.1; | 5 |
| | | converted into putrescine | GABA --> GABA-CoA --> | 2.6.1; | |
| | | | 4ABal --> Put | 2.8.3; | |
| | | | | 6.2.1; | |
| | | | | 1.4.1 | |
| By 46 | N-acetyl putrescine | By reaction of acetyl-CoA wth | SuCoA --SucSal --> | 1.2.1; | 6 |
| | | putrescine | GABA --> GABA-CoA --> | 2.6.1; | |
| | | | 4ABal --> Put --> Ac-Put | 2.8.3; | |
| | | | | 6.2.1; | |
| | | | | 1.4.1; | |
| | | | | 2.3.1 | |
| By 47 | N- | By reaction of putrescine with | SuCoA --SucSal --> | 1.2.1; | 6 |
| | hydroxyputrescine | O2 | GABA --> GABA-CoA --> | 2.6.1; | |
| | | | 4ABal --> Put --> Put- | 2.8.3; | |
| | | | OH | 6.2.1; | |
| | | | | 1.4.1; No | |
| | | | | EC¹ | |
| By 48 | methyl-putrescine | By reaction of SAM with | SuCoA --SucSal --> | 1.2.1; | 6 |
| | | putrescine | GABA --> GABA-CoA --> | 2.6.1; | |
| | | | 4ABal --> Put -> Me-Put | 2.8.3; | |
| | | | | 6.2.1; | |
| | | | | 1.4.1; | |
| | | | | 2.1.1 | |
| By 49 | Pyrroline | From 4-aminobutanal by | SuCoA --SucSal --> | 1.2.1; | 5 |
| | | putrescine amino transferase | GABA --> GABA-CoA --> | 2.6.1; | |
| | | | 4ABal --> pyrroline | 2.8.3; | |
| | | | | 6.2.1; | |
| | | | | 1.4.1 | |
| By 50 | Pyrrolidone | From CoA activation of 4- | SuCoA --SucSal --> | 1.2.1; | 4 |
| | | aminobutyrate | GABA --> GABA-CoA --> | 1.4.1; | |
| | | | cycle | 2.6.1; | |
| | | | | 2.8.3; | |
| | | | | 6.2.1 | |
| By 51 | 4-hydroxybutyrate | Succinyl-CoA can be converted | SucCoA --> SucSal --> | 1.2.1; | 2 |
| | | to succinate semialdehyde (a | 4HB | 1.1.1 | |
| | | non-specific aid activity) and | | | |
| | | then a native 4HB | | | |
| | | dehydrogenase(s) could make | | | |
| | | this molecule | | | |
| By 52 | N- | Putrescine can react with | SuCoA --> SucSal --> | 1.2.1; | 6 |
| | Carbamoylputrescine | carbamoyl-phosphate by | GABA --> GABA-CoA --> | 2.6.3; | |
| | | carbamoyl transferase | 4ABal --> Put --> Cm- | 1.4.1; | |
| | | | Put | 2.8.3; | |
| | | | | 6.2.1; | |
| | | | | 2.1.3 | |
| By 53 | N- | GABA can react with | SuCoA --> SucSal --> | 1.2.1; | 3 |
| | carbamoylaminobutyrate | carbamoyl-phosphate by | GABA --> Carb-GABA | 2.6.1; | |
| | | carbamoyl transferase | | 1.4.1; | |
| | | | | 2.1.3 | |
| By 54 | N- | 4-aminobutanol can react with | SuCoA --> SucSal --> | 1.2.1; | 6 |
| | carbamoylaminobutanol | carbamoyl-phosphate by | GABA --> GABA-CoA --> | 2.6.1; | |
| | | carbamoyl transferase | 4ABal --> 4ABol --> Cm- | 2.8.3; | |
| | | | 4ABol | 6.2.1; | |
| | | | | 1.1.1; | |
| | | | | 2.1.3; | |
| | | | | 1.4.1 | |
| By 55 | N1,N4- | N-acetyltransferase reacts with | SuCoA --> SucSal --> | 1.2.1; | 7 |
| | diacetylputrescine | putrescine 2x | GABA --> GABA-CoA --> | 2.6.3; | |
| | | | 4ABal --> Put --> Ac-Put | 1.4.1; | |
| | | | -> 2Ac-Put | 2.8.3; | |
| | | | | 6.2.1; | |
| | | | | 2.3.1 | |
| By 56 | N1,N4- | N-acetyltransferase reacts with | SuCoA --> SucSal --> | 1.2.1; | 7 |
| | diacylputrescine or | putrescine and acyl-CoA | GABA --> GABA-CoA --> | 2.6.3; | |
| | N-acylputrescine | pathway intermediates (other | 4ABal --> Put --> Ac-Put | 1.4.1; | |
| | | than acetyl-CoA) | -> 2Ac-Put | 2.8.3; | |
| | | | | 6.2.1; | |
| | | | | 2.3.1 | |
| By 57 | Spermidine | Putrescine reacts with S-MetP | SuCoA --> SucSal --> | 1.2.1; | 6 |
| | | to form spermidine | GABA --> GABA-CoA --> | 2.6.3; | |
| | | | 4ABal --> Put --> Sp | 1.4.1; | |
| | | | | 2.8.3; | |
| | | | | 6.2.1; | |
| | | | | 2.5.1 | |
| By 58 | N-acetyl-6- | N-acetyltransferase reacts with | 6acasa --> 6-AHexOH --> | 1.1.1; | 2 |
| | aminohexanol | 6-aminohexanol | N-acetyl-6-AHexOH | 2.3.1 | |
| By 59 | N-hydroxy-6- | 6-Aminohexanol reacts with O2 | 6acasa --> 6-AHexOH --> | 1.1.1; No | 2 |
| | aminohexanol | | N-hydroxy-6-AHexOH | EC¹ | |
| By 60 | N-glutamyl-6- | Glutamylation of 6- | 6acasa --> 6-AHexOH --> | 1.1.1; | 2 |
| | aminohexanol | aminohexanol | N-glu-6-AHexOH | 6.3.1 | |
| By 61 | 3,5- | The thiolase for 3-oxoadipyl- | 3OACoA --> 3,5- | 2.3.1; | 2 |
| | dioxooctanedioate | CoA has been documented to | dioxooctanoyl-CoA --> | 3.1.2; | |
| | | combine with acetyl-CoA and | 3,5-dioxooctanedioate | 2.8.3; | |
| | | make the CoA form of this | | 6.2.1 | |
| | | compound | | | |
| By 62 | 3-oxooctanedioate | Thioase acts on adipyl-CoA and | Adip-CoA --> 3- | 2.3.1; | 2 |
| | | acetyl-CoA | oxooctanedioyl-CoA --> | 3.1.2; | |
| | | | 3-oxooctanedioate | 2.8.3; | |
| | | | | 6.2.1 | |
| By 63 | 5-hydroxy-3- | Thiolase acts on 3- | 3HACoA --> 5-hydroxy- | 2.3.1; | 2 |
| | oxooctanedioate | hydroxyadipyl-CoA and acetyl- | 3-oxooctanoyl-CoA --> | 3.1.2; | |
| | | CoA | 5-hydroxy-3- | 2.8.3; | |
| | | | oxooctanedioate | 6.2.1 | |
| By 64 | 3-oxooct-4- | Thiolase acts on 5-carboxy-2- | 5C2PenCoA --> | 2.3.1; | 2 |
| | enedioic acid | pentenoyl-CoA and acetyl-CoA | 3oxooct-4-enoyl-CoA --> | 3.1.2; | |
| | | | 3-oxooct-4-enoate | 2.8.3; | |
| | | | | 6.2.1 | |
| By 65 | 8-amino-3- | Thiolase acts on 6-ACA-CoA | 6-ACA-CoA --> | 2.3.1; | 2 |
| | oxooctanoate | and acetyl-CoA | 8A3OOct-CoA --> | 3.1.2; | |
| | | | 8A3OOctate | 2.8.3; | |
| | | | | 6.2.1 | |
| By 66 | N-propylamine-6- | 6-ACA reacts with S-MetP | 6-ACA --> NP-6ACA | 2.5.1 | 1 |
| | aminocaproate | | | | |
| By 67 | 4- | Levulinic acid reacts with ADH | Levulinate --> 4HP | 1.1.1 | 1 |
| | hydroxypentanoate | | | | |

TABLE 11
Acetoacetyl HMD, ACA, CPL, HDO, ADA Pathway Byproducts

| Byproduct | | | |
|---|---|---|---|
| No | Byproduct | Mode of formation | Exemplary EC classes |
| IB1 | Acetate | Hydrolysis of pathway intermediate | 2.8.3; 3.1.2; 6.2.1 |
| IB2 | Malonate | Hydrolysis of pathway intermediate | 2.8.3; 3.1.2; 6.2.1; 4.1.1 |
| IB3 | Acetoacetate | Hydrolysis of pathway intermediate | 3.2.1; 2.8.3 |
| IB4 | 3-Hydroxybutyrate | Hydrolysis of pathway intermediate | 3.2.1; 2.8.3 |
| IB5 | Crotonate | Hydrolysis of pathway intermediate | 3.2.1; 2.8.3 |
| IB6 | Butyrate | Hydrolysis of pathway intermediate | 3.2.1; 2.8.3 |
| IB7 | 3-Oxohexanoate | Hydrolysis of pathway intermediate | 3.2.1; 2.8.3 |
| IB8 | 3-hydroxyhexanoate | Hydrolysis of pathway intermediate | 3.2.1; 2.8.3 |
| IB9 | Hex-2-enoate | Hydrolysis of pathway intermediate | 3.2.1; 2.8.3 |
| IB10 | Hexanoate | Hydrolysis of pathway intermediate | 3.2.1; 2.8.3 |
| IB11 | Adipate | Reaction of adipate semialdehyde | 1.2.1 |
| | | with acid-forming dehydrogenase | |
| IB12 | 4-hydroxy-3-oxobutanoate | Reaction of acid byproducts with | 3.2.1.; 2.8.3; 1.14.13 |
| | | alkane hydroxylase | |
| IB13 | 3,4-dihydroxybutanoate | Reaction of acid byproducts with | 3.2.1.; 2.8.3; 1.14.13 |
| | | alkane hydroxylase | |
| IB14 | 4-hydroxybut-2-enoate | Reaction of acid byproducts with | 3.2.1.; 2.8.3; 1.14.13 |
| | | alkane hydroxylase | |
| IB15 | 4-hydroxybutyrate | Reaction of acid byproducts with | 3.2.1.; 2.8.3; 1.14.13 |
| | | alkane hydroxylase | |
| IB16 | 6-hydroxy-3-oxohexanoate | Reaction of acid byproducts with | 3.2.1.; 2.8.3; 1.14.13 |
| | | alkane hydroxylase | |
| IB17 | 3,6-dihydroxyhexanoate | Reaction of acid byproducts with | 3.2.1.; 2.8.3; 1.14.13 |
| | | alkane hydroxylase | |
| IB18 | 6-hydroxyhex-2-enoate | Reaction of acid byproducts with | 3.2.1.; 2.8.3; 1.14.13 |
| | | alkane hydroxylase | |
| IB19 | 4-amino-3-oxobutanoate | Reaction of acid byproducts with | 3.2.1.; 2.8.3; 1.14.13; |
| | | alkane hydroxylase, ADH, | 1.1.1; 2.6.1 |
| | | aminotransferase | |
| IB20 | 3-hydroxy-4- | Reaction of acid byproducts with | 3.2.1.; 2.8.3; 1.14.13; |
| | aminobutanoate | alkane hydroxylase, ADH, | 1.1.1; 2.6.1 |
| | | aminotransferase | |
| IB21 | 4-aminobut-2-enoate | Reaction of acid byproducts with | 3.2.1.; 2.8.3; 1.14.13; |
| | | alkane hydroxylase, ADH, | 1.1.1; 2.6.1 |
| | | aminotransferase | |
| IB22 | 4-aminobutyrate (GABA) | Reaction of acid byproducts with | 3.2.1.; 2.8.3; 1.14.13; |
| | | alkane hydroxylase, ADH, | 1.1.1; 2.6.1 |
| | | aminotransferase | |
| IB23 | 6-amino-3-oxohexanoate | Reaction of acid byproducts with | 3.2.1.; 2.8.3; 1.14.13; |
| | | alkane hydroxylase, ADH, | 1.1.1; 2.6.1 |
| | | aminotransferase | |
| IB24 | 3-hydroxy-6- | Reaction of acid byproducts with | 3.2.1.; 2.8.3; 1.14.13; |
| | aminohexanoate | alkane hydroxylase, ADH, | 1.1.1; 2.6.1 |
| | | aminotransferase | |
| IB25 | 6-aminohex-2-enoate | Reaction of acid byproducts with | 3.2.1.; 2.8.3; 1.14.13; |
| | | alkane hydroxylase, ADH, | 1.1.1; 2.6.1 |
| | | aminotransferase | |
| IB26 | 3-hydroxyadipate | Reaction of acid byproducts with | 3.2.1.; 2.8.3; 1.14.13; |
| | | alkane hydroxylase, ALD | 1.2.1 |
| IB27 | octanoate | Thiolase extending chain length | 2.3.1; 3.2.1; 2.8.3 |
| IB28 | octanol | Thiolase extending chain length | 2.3.1 |
| IB29 | 3-hydroxyoctanoate | Thiolase extending chain length | 2.3.1 |
| IB30 | 3-oxooctanoate | Thiolase extending chain length | 2.3.1 |
| IB31 | octan-2-enoate | Thiolase extending chain length | 2.3.1 |
| IB32 | 3,8-dihydroxyoctanoate | Thiolase extending chain length | 2.3.1 |
| IB33 | 3-oxo-8-hydroxyoctanoate | Thiolase extending chain length | 2.3.1 |
| IB34 | 8-hydroxyoctan-2-enoate | Thiolase extending chain length | 2.3.1 |

HMD produced using cells and methods described herein can include one or more byproducts selected from By3, By5, By6, By10, By16, By19, By21, By30, By36, By41, By44, By45, By50, By51, By61, By62, By63, By64, or By65. HMD produced using the cells and methods described herein can include at least 2, 3, 4, 5, 6, or all of By3, By5, By6, By10, By16, By19, By21, By30, By36, By41, By44, By45, By50, By51, By61, By62, By63, By64, and By65. HMD produced using the cells and methods described herein can include at least 2, 3, 4, 5, 6, or all of By3, By5, By6, By10, By16, By19, By21, By30, By36, By41, By44, By45, By50, By51, By61, By62, By63, By64, and By65 where at least one of the byproducts is present at level lower than HMD produced in a cell lacking genetic modifications associated with reduction of the byproduct as described herein. HMD produced by cells and methods described herein can include at least 2, 3, 4, 5, 6, or all of By1, By9, By13, By14, By17, By18, By20, By 24, By25, By27, By35, By39, or By40 and By3, By5, By6, By10, By16, By19, By21, By30, By36, By41, By44, By45, By50, By51, By61, By62, By63, By64, and By65 where at least one of the byproducts is present at level lower than HMD produced in a cell lacking genetic modifications associated with reduction of the byproduct as described herein.

HMD produced using cells and methods described herein can include one or more byproducts selected from IB1-IB34 of Table 11. HMD produced using the cells and methods described herein can include at least 2, 3, 4, 5, 6, or all of the byproducts IB1-IB34 of Table 11. HMD produced using the cells and methods described herein can include at least 2, 3, 4, 5, 6, or all of the byproducts IB1-IB34 of Table 11 where at least one of the byproducts is present at level lower than HMD produced in a cell lacking genetic modifications associated with reduction of the byproduct as described herein. HMD produced by cells and methods described herein can include at least 2, 3, 4, 5, 6, or all of the byproducts IB1-IB34 of Table 11 where at least two of the byproducts is present at level lower than HMD produced in a cell lacking genetic modifications associated with reduction of the byproduct as described herein.

TABLE 12
Byproducts and corresponding pathways

| | | | Acetoacetyl- | | | | | | |
|---|---|---|---|---|---|---|---|---|---|
| | | | CoA | | | | | | |
| | | | HMD | | | | | | |
| By# | Byproduct Name | HMD | pathway | LVA | ADA | HDO | CPO | 6ACA | CPL |
| By1 | 3-oxoadipate | Y | N | Y | Y | Y | Y | Y | Y |
| By2 | 4-oxopentanoate | Y | N | Y | Y | Y | Y | Y | Y |
| By3 | 3-oxo-6-amino hexanoate | Y | N | Y | Y | Y | Y | Y | Y |
| By4 | 3,6-diamino hexanoate | Y | N | Y | Y | Y | Y | Y | Y |
| By5 | 3-oxo-6-hydroxy | Y | N | Y | Y | Y | Y | Y | Y |
| | hexanoate | | | | | | | | |
| By6 | 3,6-dihydroxy hexanoate | Y | N | Y | Y | Y | Y | Y | Y |
| By7 | 3-amino-6-hydroxy | Y | N | Y | Y | Y | Y | Y | Y |
| | hexanoate | | | | | | | | |
| By8 | 6-hydroxyhex-2-enoate | Y | Y | Y | Y | Y | Y | Y | Y |
| By9 | 3-hydroxyadipate | Y | Y | N | Y | Y | Y | Y | Y |
| By10 | 3-hydroxy-6-amino | Y | Y | N | Y | Y | Y | Y | Y |
| | hexanoate | | | | | | | | |
| By11 | 6-aminohex-2-enoate | Y | Y | N | Y | Y | Y | Y | Y |
| By12 | 4-hydroxypiperidin-2-one | Y | Y | N | Y | Y | Y | Y | Y |
| By13 | 5-carboxy-2-pentenoate | Y | N | N | Y | Y | Y | Y | Y |
| By14 | 6-hydroxy hex-4-enoate | Y | N | N | Y | Y | Y | Y | Y |
| By15 | 6-hydroxyhexanoate | Y | Y | N | Y | Y | Y | Y | Y |
| By16 | 6-aminohex 4-enoate | Y | N | N | Y | Y | Y | Y | Y |
| By17 | 6-aminocaproic acid (6- | Y | Y | N | N | N | N | N | N |
| | ACA) | | | | | | | | |
| By18 | adipate | Y | Y | N | Y | Y | Y | Y | Y |
| By19 | caprolactam (CPL) | Y | Y | N | N | N | N | Y | Y |
| By20 | 6-aminohexanol | Y | Y | N | N | N | N | N | N |
| By21 | N-hydroxy 6-ACA | Y | Y | N | N | N | N | Y | Y |
| By22 | N-hydroxy succinyl-6ACA | Y | Y | N | N | N | N | Y | Y |
| By23 | N-methyl 6-ACA | Y | Y | N | N | N | N | Y | Y |
| By24 | N-glutamyl-6-ACA | Y | Y | N | N | N | N | Y | Y |
| By25 | N-acetyl-6-amino caproic | Y | Y | N | N | N | N | Y | Y |
| | acid | | | | | | | | |
| By26 | N-carbamoyl-6ACA | Y | Y | N | N | N | N | Y | Y |
| By27 | N-acetyl-HMDA | Y | Y | N | N | N | N | N | N |
| By28 | N-carbamoyl-HMDA | Y | Y | N | N | N | N | N | N |
| By29 | Tetrahydroazepine | Y | Y | N | N | N | N | N | N |
| By30 | N-hydroxy HMDA | Y | Y | N | N | N | N | N | N |
| By31 | N-succinyl HMDA | Y | Y | N | N | N | N | N | N |
| By32 | N-hydroxy succinyl HMDA | Y | Y | N | N | N | N | N | N |
| By33 | N-methyl HMDA | Y | Y | N | N | N | N | N | N |
| By34 | N,N-dimethyl HMDA | Y | Y | N | N | N | N | N | N |
| By35 | Glutamyl-HMDA | Y | Y | N | N | N | N | N | N |
| By36 | 7-carboxy-3-oxohept-5- | Y | Y | N | Y | Y | Y | Y | Y |
| | enoate (or 3-oxo 5,6- | | | | | | | | |
| | didehydrosuberate) | | | | | | | | |
| By37 | N-acyl-HMDA or N1,N6- | Y | Y | N | N | N | N | N | N |
| | diacyl-HMDA | | | | | | | | |
| By38 | N-propylamine-HMDA | Y | Y | N | N | N | N | N | N |
| By39 | succinate | Y | N | Y | Y | Y | Y | Y | Y |
| By40 | 4-aminobutyrate | Y | Y | N | Y | Y | Y | Y | Y |
| By41 | N-acetyl- 4-amino | Y | Y | N | Y | Y | Y | Y | Y |
| | butyrate | | | | | | | | |
| By42 | methyl-4-amino butyrate | Y | Y | N | Y | Y | Y | Y | Y |
| By43 | 4-aminobutanol | Y | Y | N | Y | Y | Y | Y | Y |
| By44 | Glutamyl putrescine | Y | Y | N | Y | Y | Y | Y | Y |
| By45 | putrescine | Y | Y | N | Y | Y | Y | Y | Y |
| By46 | N-acetyl putrescine | Y | Y | N | Y | Y | Y | Y | Y |
| By47 | N-hydroxyputrescine | Y | Y | N | Y | Y | Y | Y | Y |
| By48 | methyl-putrescine | Y | Y | N | Y | Y | Y | Y | Y |
| By49 | Pyrroline | Y | Y | N | Y | Y | Y | Y | Y |
| By50 | Pyrrolidone | Y | Y | N | Y | Y | Y | Y | Y |
| By51 | 4-hydroxybutyrate | Y | Y | N | Y | Y | Y | Y | Y |
| By52 | N-Carbamoylputrescine | Y | Y | N | Y | Y | Y | Y | Y |
| By53 | N- | Y | Y | N | Y | Y | Y | Y | Y |
| | carbamoylaminobutyrate | | | | | | | | |
| By54 | N- | Y | Y | N | Y | Y | Y | Y | Y |
| | carbamoylaminobutanol | | | | | | | | |
| By55 | N1,N4-diacetylputrescine | Y | Y | N | Y | Y | Y | Y | Y |
| By56 | N1,N4-diacylputrescine or | Y | Y | N | Y | Y | Y | Y | Y |
| | N-acylputrescine | | | | | | | | |
| By57 | Spermidine | Y | Y | N | Y | Y | Y | Y | Y |
| By58 | N-acetyl-6-aminohexanol | Y | Y | N | N | N | N | N | N |
| By59 | N-hydroxy-6- | Y | Y | N | N | N | N | N | N |
| | aminohexanol | | | | | | | | |
| By60 | N-glutamyl-6- | Y | Y | N | N | N | N | N | N |
| | aminohexanol | | | | | | | | |
| By61 | 3,5-dioxooctanedioate | Y | N | Y | Y | Y | Y | Y | Y |
| By62 | 3-oxooctanedioate | Y | N | N | Y | Y | Y | Y | Y |
| By63 | 5-hydroxy-3- | Y | N | N | Y | Y | Y | Y | Y |
| | oxooctanedioate | | | | | | | | |
| By64 | 3-oxooct-4-enedioic acid | Y | N | N | Y | Y | Y | Y | Y |
| By65 | 8-amino-3-oxooctanoate | Y | N | N | N | N | N | N | N |
| By66 | N-p.ropylamine-6- | Y | N | N | N | N | N | Y | Y |
| | aminocaproate | | | | | | | | |
| By67 | 4-hydroxypentanoate | N | N | Y | N | N | N | N | N |

HMD produced using cells and methods described herein can include one or more byproducts selected from By2, By4, By7, By8, By11, By12, By15, By22, By23, By26, By28, By29, By31, By32, By33, By34, By37, By38, By42, By43, By46, By47, By48, By49, By52, By53, By54, By55, By56, By57, By58, By59, By60, or By66. HMD produced using the cells and methods described herein can include at least 2, 3, 4, 5, 6, or all of By2, By4, By7, By8, By11, By12, By15, By22, By23, By26, By28, By29, By31, By32, By33, By34, By37, By38, By42, By43, By46, By47, By48, By49, By52, By53, By54, By55, By56, By57, By58, By59, By60, and By66. HMD produced using the cells and methods described herein can include at least 2, 3, 4, 5, 6, or all of By2, By4, By7, By8, By11, By12, By15, By22, By23, By26, By28, By29, By31, By32, By33, By34, By37, By38, By42, By43, By46, By47, By48, By49, By52, By53, By54, By55, By56, By57, By58, By59, By60, and By66, where at least one of the byproducts is present at level lower than HMD produced in a cell lacking genetic modifications associated with reduction of the byproduct as described herein. HMD produced by cells and methods described herein can include at least 2, 3, 4, 5, 6, or all of By1, By9, By13, By14, By17, By18, By20, By 24, By25, By27, By35, By39, or By40 and By3, By5, By6, By10, By16, By19, By21, By30, By36, By41, By44, By45, By50, By51, By61, By62, By63, By64, or By65, and By2, By4, By7, By8, By11, By12, By15, By22, By23, By26, By28, By29, By31, By32, By33, By34, By37, By38, By42, By43, By46, By47, By48, By49, By52, By53, By54, By55, By56, By57, By58, By59, By60, and By66 where at least one of the byproducts is present at level lower than HMD produced in a cell lacking genetic modifications associated with reduction of the byproduct as described herein.

HMD produced by cells and methods described herein can include at least 2, 3, 4, 5, 6, or all of By1, By9, By13, By14, By17, By18, By20, By 24, By25, By27, By35, By39, or By40 and By2, By4, By7, By8, By11, By12, By15, By22, By23, By26, By28, By29, By31, By32, By33, By34, By37, By38, By42, By43, By46, By47, By48, By49, By52, By53, By54, By55, By56, By57, By58, By59, By60, or By66 where at least one of the byproducts is present at level lower than HMD produced in a cell lacking genetic modifications associated with reduction of the byproduct as described herein.

HMD produced by cells and methods described herein can include at least 2, 3, 4, 5, 6, or all of By3, By5, By6, By10, By16, By19, By21, By30, By36, By41, By44, By45, By50, By51, By61, By62, By63, By64, or By65, and By2, By4, By7, By8, By11, By12, By15, By22, By23, By26, By28, By29, By31, By32, By33, By34, By37, By38, By42, By43, By46, By47, By48, By49, By52, By53, By54, By55, By56, By57, By58, By59, By60, and By66 where at least one of the byproducts is present at level lower than HMD produced in a cell lacking genetic modifications associated with reduction of the byproduct as described herein.

Cells described herein can contain a LVA pathway described herein where such a cell is capable of producing LVA as a target product and has one or more genetic modifications described herein. Such cells have reduced levels of at least one of byproducts By1-By8, By39, By61, or By67 as set forth in Table 10 and Table 12. Cells expressing a LVA pathway described herein capable of producing LVA as a target product and having one or more genetic modifications described herein can include at least 2, 3, 4, 5, 6, 7, 8, 9, 10, or 11 byproducts selected from By1-By8, By39, By61, or By67 as set forth in Table 10 and Table 12 where at least one byproduct is present at a reduced level.

LVA produced by cells described herein can include one or more byproducts as described above. Particular byproducts may be desirable to reduce to lower levels than other byproducts produced by the same biosynthetic pathway. LVA produced using the cells and methods described herein can include one or more byproducts selected from By1 or By40 of Table 10 and 12 where at least one the byproducts when present is present at level lower than LVA produced in a cell lacking genetic modifications associated with reduction of the byproduct as described herein.

LVA produced using the cells and methods described herein can include one or more byproducts selected from By3, By5, By6, or By61 of Table 10 and 12. LVA produced using cells and methods described herein can include at least 2, 3, or all of By3, By5, By6, or By61. LVA produced using the cells and methods described herein can include at least 2, 3, or all of By3, By5, By6, or By61, where at least one of the byproducts when present is present at level lower than LVA produced in a cell lacking genetic modifications associated with reduction of the byproduct as described herein.

LVA produced using the cells and methods described herein can include one or more byproducts selected from By2, By4, By7, By8, or By67 of Table 10 and 12. LVA produced using cells and methods described herein can include at least 2, 3, 4, or all of By2, By4, By7, By8, or By67. LVA produced using the cells and methods described herein can include at least 2, 3, 4, or all of By2, By4, By7, By8, or By67, where at least one of the byproducts when present is present at level lower than LVA produced in a cell lacking genetic modifications associated with reduction of the byproduct as described herein.

Cells described herein can contain a HMD pathway described herein capable of producing ADA as a target product where such a cell is capable of producing ADA as a target product, and has one or more a genetic modifications described herein resulting in reduced levels of at least one of byproducts By1-By16, By18, By36, By39-By57, or By61-By64 as set forth in Table 10 and Table 12. Cells expressing a HMD pathway described herein capable of producing ADA as a target product, and having one or more genetic modifications described herein can have reduced levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 byproducts selected from By1-By16, By18, By36, By39-By57, or By61-By64 as set forth in Table 10 and Table 11.

ADA produced by cells described herein can include one or more byproducts as described above. Particular byproducts may be desirable to reduce to lower levels than other byproducts produced by the same biosynthetic pathway. ADA produced using the cells and methods described herein can include one or more byproducts selected from By1, By9, By13, By14, By18, By24, By25, By39, or By40 of Table 10 and 12 where at least one the byproducts when present is present at level lower than ADA produced in a cell lacking genetic modifications associated with reduction of the byproduct as described herein. ADA produced using cells and methods described herein can include at least 2, 3, 4, 5, 6, or all of By1, By9, By13, By14, By18, By24, By25, By39, or By40 of Table 10 and 12 where at least one the byproducts when present is present at level lower than ADA produced in a cell lacking genetic modifications associated with reduction of the byproduct as described herein.

ADA produced using the cells and methods described herein can include one or more byproducts selected from By3, By5, By6, By10, By16, By19, By21, By36, By41, By44, By45, By50, By51, By61, By62, By63 or By64 of Table 10 and 12. ADA produced using cells and methods described herein can include at least 2, 3, 4, 5, 6, or all of By3, By5, By6, By10, By16, By19, By21, By36, By41, By44, By45, By50, By51, By61, By62, By63 or By64. ADA produced using the cells and methods described herein can include at least 2, 3, 4, 5, 6, or all of By3, By5, By6, By10, By16, By19, By21, By36, By41, By44, By45, By50, By51, By61, By62, By63 or By64, where at least one of the byproducts when present is present at level lower than ADA produced in a cell lacking genetic modifications associated with reduction of the byproduct as described herein.

ADA produced using the cells and methods described herein can include one or more byproducts selected from By2, By4, By7, By8, By11, By12, By15, By22, By23, By26, By42, By43, By46, By47, By48, By49, By52, By53, By54, By55, By56, By57, and By66 of Table 10 and 12. ADA produced using cells and methods described herein can include at least 2, 3, 4, 5, 6, or all of By2, By4, By7, By8, By11, By12, By15, By22, By23, By26, By42, By43, By46, By47, By48, By49, By52, By53, By54, By55, By56, By57, and By66. ADA produced using the cells and methods described herein can include at least 2, 3, 4, 5, 6, or all of By2, By4, By7, By8, By11, By12, By15, By22, By23, By26, By42, By43, By46, By47, By48, By49, By52, By53, By54, By55, By56, By57, and By66, where at least one of the byproducts when present is present at level lower than ADA produced in a cell lacking genetic modifications associated with reduction of the byproduct as described herein.

Combinations of the above-referenced byproducts are possible for ADA produced using the cells and methods described herein where at least one of the byproducts is present at level lower than ADA produced in a cell lacking genetic modifications associated with reduction of the byproduct as described herein.

Cells described herein can contain a HMD pathway described herein capable of producing 6ACA as a target product where such a cell is capable of producing 6ACA as a target product, and where the cell has one or more a genetic modifications described herein resulting in reduced levels of at least one of byproducts By1-By16, By18-By19, By21-By26, By36, By39-By57, By61-By64, or By66 as set forth in Table 10 and Table 12. Cells expressing a HMD pathway described herein capable of producing 6ACA as a target product and having one or more genetic modifications described herein can have reduced levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47 byproducts selected from By1-By16, By18-By19, By21-By26, By36, By39-By57, By61-By64, or By66 as set forth in Table 10 and Table 12.

6ACA produced by cells described herein can include one or more byproducts as described above. Particular byproducts may be desirable to reduce to lower levels than other byproducts produced by the same biosynthetic pathway. 6ACA produced using the cells and methods described herein can include one or more byproducts selected from By1, By9, By13, By14, By18, By39, or By40 of Table 10 and 12 where at least one the byproducts when present is present at level lower than 6ACA produced in a cell lacking genetic modifications associated with reduction of the byproduct as described herein. GACA produced using cells and methods described herein can include at least 2, 3, 4, 5, 6, or all of By1, By9, By13, By14, By18, By39, or By40 of Table 10 and 12 where at least one the byproducts when present is present at level lower than 6ACA produced in a cell lacking genetic modifications associated with reduction of the byproduct as described herein.

6ACA produced using the cells and methods described herein can include one or more byproducts selected from By3, By5, By6, By10, By16, By36, By41, By44, By45, By50, By51, By61, By61, By62, By63 or By64 of Table 10 and 12. 6ACA produced using cells and methods described herein can include at least 2, 3, 4, 5, 6, or all of By3, By5, By6, By10, By16, By36, By41, By44, By45, By50, By51, By61, By61, By62, By63 or By64. 6ACA produced using the cells and methods described herein can include at least 2, 3, 4, 5, 6, or all of By3, By5, By6, or By61, where at least one of the byproducts when present is present at level lower than 6ACA produced in a cell lacking genetic modifications associated with reduction of the byproduct as described herein.

6ACA produced using the cells and methods described herein can include one or more byproducts selected from By2, By4, By7, By8, By11, By12, By15, By42, By43, By46, By47, By48, By49, By52, By53, By54, By55, By56, or By57 of Table 10 and 12. 6ACA produced using cells and methods described herein can include at least 2, 3, 4, 5, 6, or all of By2, By4, By7, By8, By11, By12, By15, By42, By43, By46, By47, By48, By49, By52, By53, By54, By55, By56, or By57. 6ACA produced using the cells and methods described herein can include at least 2, 3, 4, 5, 6, or all of By2, By4, By7, By8, By11, By12, By15, By42, By43, By46, By47, By48, By49, By52, By53, By54, By55, By56, or By57, where at least one of the byproducts when present is present at level lower than 6ACA produced in a cell lacking genetic modifications associated with reduction of the byproduct as described herein.

Combinations of the above-referenced byproducts are possible for 6ACA produced using the cells and methods described herein where at least one of the byproducts is present at level lower than 6ACA produced in a cell lacking genetic modifications associated with reduction of the byproduct as described herein.

Cells described herein can contain a HMD pathway described herein capable of producing CPL as a target product where such a cell is capable of producing CPL as a target product, and where the cell has one or more genetic modifications described herein resulting in reduced levels of at least one of byproducts By1-By16, By18-By19, By21-By26, By36, By39-By57, By61-By64, or By66 as set forth in Table 10 and Table 12. Cells expressing a HMD pathway described herein capable of producing CPL as a target product and having one or more genetic modifications described herein can have reduced levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 byproducts selected from By By18-By19, By21-By26, By36, By39-By57, By61-By64, or By66 as set forth in Table 10 and Table 12.

CPL produced by cells described herein can include one or more byproducts as described above. Particular byproducts may be desirable to reduce to lower levels than other byproducts produced by the same biosynthetic pathway. CPL produced using the cells and methods described herein can include one or more byproducts selected from By1, By9, By13, By14, By18, By24, By25, By39, or By40 of Table 10 and 12 where at least one the byproducts when present is present at level lower than CPL produced in a cell lacking genetic modifications associated with reduction of the byproduct as described herein. CPL produced using cells and methods described herein can include at least 2, 3, 4, 5, 6, or all of By1, By9, By13, By14, By18, By24, By25, By39, or By40 of Table 10 and 12 where at least one the byproducts when present is present at level lower than CPL produced in a cell lacking genetic modifications associated with reduction of the byproduct as described herein.

CPL produced using the cells and methods described herein can include one or more byproducts selected from By3, By5, By6, By10, By16, By19, By21, By36, By41, By44, By45, By50, By51, By61, By62, By63 or By64 of Table 10 and 12. CPL produced using cells and methods described herein can include at least 2, 3, 4, 5, 6, or all of By3, By5, By6, By10, By16, By19, By21, By36, By41, By44, By45, By50, By51, By61, By62, By63 or By64. CPL produced using the cells and methods described herein can include at least 2, 3, 4, 5, 6, or all of By3, By5, By6, By10, By16, By19, By21, By36, By41, By44, By45, By50, By51, By61, By62, By63 or By64, where at least one of the byproducts when present is present at level lower than CPL produced in a cell lacking genetic modifications associated with reduction of the byproduct as described herein.

CPL produced using the cells and methods described herein can include one or more byproducts selected from By2, By4, By7, By8, By11, By12, By15, By22, By23, By26, By42, By43, By46, By47, By48, By49, By52, By53, By54, By55, By56, By57, and By66 of Table 10 and 12. CPL produced using cells and methods described herein can include at least 2, 3, 4, 5, 6, or all of By2, By4, By7, By8, By11, By12, By15, By22, By23, By26, By42, By43, By46, By47, By48, By49, By52, By53, By54, By55, By56, By57, and By66. CPL produced using the cells and methods described herein can include at least 2, 3, 4, 5, 6, or all of By2, By4, By7, By8, By11, By12, By15, By22, By23, By26, By42, By43, By46, By47, By48, By49, By52, By53, By54, By55, By56, By57, and By66, where at least one of the byproducts when present is present at level lower than CPL produced in a cell lacking genetic modifications associated with reduction of the byproduct as described herein.

Combinations of the above-referenced byproducts are possible for CPL produced using the cells and methods described herein where at least one of the byproducts is present at level lower than CPL produced in a cell lacking genetic modifications associated with reduction of the byproduct as described herein.

Cells described herein can contain a CPO pathway as described herein and as shown in, for example, FIG. 5, where such a cell is capable of producing CPO as a target product, and where the cell has one or more a genetic modifications described herein resulting in reduced levels of at least one of byproducts By1-By16, By18, By36, By39-By57, or By61-By64 as set forth in Table 10 and Table 12. Cells expressing a CPO pathway described herein capable of producing CPO as a target product and having one or more a genetic modifications described herein can have reduced levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 byproducts selected from By1-By16, By18, By36, By39-By57, or By61-By64 as set forth in Table 10 and Table 12.

CPO produced by cells described herein can include one or more byproducts as described above. Particular byproducts may be desirable to reduce to lower levels than other byproducts produced by the same biosynthetic pathway. CPO produced using the cells and methods described herein can include one or more byproducts selected from By1, By9, By13, By14, By18, By39, and By40 of Table 10 and 12 where at least one the byproducts when present is present at level lower than CPO produced in a cell lacking genetic modifications associated with reduction of the byproduct as described herein. CPO produced using cells and methods described herein can include at least 2, 3, 4, 5, 6, or all of By1, By9, By13, By14, By18, By39, and By40 of Table 10 and 12 where at least one the byproducts when present is present at level lower than CPO produced in a cell lacking genetic modifications associated with reduction of the byproduct as described herein.

CPO produced using the cells and methods described herein can include one or more byproducts selected from By3, By5, By6, By10, By16, By36, By41, By44, By45, By50, By51, By61, By62, By63 and By64 of Table 10 and 12. CPO produced using cells and methods described herein can include at least 2, 3, 4, 5, 6, or all of By3, By5, By6, By10, By16, By36, By41, By44, By45, By50, By51, By61, By62, By63 and By64. CPO produced using the cells and methods described herein can include at least 2, 3, 4, 5, 6, or all of By3, By5, By6, By10, By16, By36, By41, By44, By45, By50, By51, By61, By62, By63 and By64, where at least one of the byproducts when present is present at level lower than CPO produced in a cell lacking genetic modifications associated with reduction of the byproduct as described herein.

CPO produced using the cells and methods described herein can include one or more byproducts selected from By2, By4, By7, By8, By11, By12, By15, By42, By43, By46, By47, By48, By49, By52, By53, By54, By55, By56, and By57 of Table 10 and 12. CPO produced using cells and methods described herein can include at least 2, 3, 4, 5, 6, or all of By2, By4, By7, By8, By11, By12, By15, By42, By43, By46, By47, By48, By49, By52, By53, By54, By55, By56, and By57. CPO produced using the cells and methods described herein can include at least 2, 3, 4, 5, 6, or all of By2, By4, By7, By8, By11, By12, By15, By42, By43, By46, By47, By48, By49, By52, By53, By54, By55, By56, and By57, where at least one of the byproducts when present is present at level lower than CPO produced in a cell lacking genetic modifications associated with reduction of the byproduct as described herein.

Combinations of the above-referenced byproducts are possible for CPO produced using the cells and methods described herein where at least one of the byproducts is present at level lower than CPO produced in a cell lacking genetic modifications associated with reduction of the byproduct as described herein.

Cells described herein can contain a HDO pathway as described herein and shown in, for example, FIG. 4, where such a cell is capable of producing HDO as a target product. In another embodiment, cells described herein can contain a HDO pathway as described herein and shown in, for example, FIG. 4, where such a cell is capable of producing HDO as a target product, and has one or more a genetic modifications described herein resulting in reduced levels of at least one of byproducts By1-By16, By18, By36, By39-By57, or By61-By64 as set forth in Table 10 and Table 12. Cells expressing a HDO pathway described herein capable of producing CPO as a target product and having one or more a genetic modifications described herein can have reduced levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 byproducts selected from By1-By16, By18, By36, By39-By57, or By61-By64 as set forth in Table 10 and Table 12.

HDO produced by cells described herein can include one or more byproducts as described above. Particular byproducts may be desirable to reduce to lower levels than other byproducts produced by the same biosynthetic pathway. HDO produced using the cells and methods described herein can include one or more byproducts selected from By1, By9, By13, By14, By18, By39, and By40 of Table 10 and 12 where at least one the byproducts when present is present at level lower than HDO produced in a cell lacking genetic modifications associated with reduction of the byproduct as described herein. HDO produced using cells and methods described herein can include at least 2, 3, 4, 5, 6, or all of By1, By9, By13, By14, By18, By39, and By40 of Table 10 and 12 where at least one the byproducts when present is present at level lower than HDO produced in a cell lacking genetic modifications associated with reduction of the byproduct as described herein.

HDO produced using the cells and methods described herein can include one or more byproducts selected from By3, By5, By6, By10, By16, By36, By41, By44, By45, By50, By51, By61, By62, By63 and By64 of Table 10 and 12. HDO produced using cells and methods described herein can include at least 2, 3, 4, 5, 6, or all of By3, By5, By6, By10, By16, By36, By41, By44, By45, By50, By51, By61, By62, By63 and By64. HDO produced using the cells and methods described herein can include at least 2, 3, 4, 5, 6, or all of By3, By5, By6, By10, By16, By36, By41, By44, By45, By50, By51, By61, By62, By63 and By64, where at least one of the byproducts when present is present at level lower than HDO produced in a cell lacking genetic modifications associated with reduction of the byproduct as described herein.

HDO produced using the cells and methods described herein can include one or more byproducts selected from By2, By4, By7, By8, By11, By12, By15, By42, By43, By46, By47, By48, By49, By52, By53, By54, By55, By56, and By57 of Table 10 and 12. HDO produced using cells and methods described herein can include at least 2, 3, 4, 5, 6, or all of By2, By4, By7, By8, By11, By12, By15, By42, By43, By46, By47, By48, By49, By52, By53, By54, By55, By56, and By57. HDO produced using the cells and methods described herein can include at least 2, 3, 4, 5, 6, or all of By2, By4, By7, By8, By11, By12, By15, By42, By43, By46, By47, By48, By49, By52, By53, By54, By55, By56, and By57, where at least one of the byproducts when present is present at level lower than HDO produced in a cell lacking genetic modifications associated with reduction of the byproduct as described herein.

Combinations of the above-referenced byproducts are possible for HDO produced using the cells and methods described herein where at least one of the byproducts is present at level lower than HDO produced in a cell lacking genetic modifications associated with reduction of the byproduct as described herein.

In certain instances, it may be desirable to genetically modify enzymes biosynthesizing intermediates in a pathway described herein when such genetic modification increases production of a target product or reduces another byproduct described herein. In such instances where an intermediate of a pathway is reduced using genetic modifications described herein the genetic modification can yield more favorable carbon flux along the pathway or use alternative co-factors which modify (e.g. increase) the yield of the desired target compound.

The invention also includes reduction of byproducts downstream of, and within a prescribed number of steps from the pathways described herein. Accordingly, in certain instances, a genetic modification described herein can reduce the level of a byproduct directly by reducing or eliminating enzymatic catalysis of the substrate necessary for catalysis to the byproduct. Alternatively, genetic modifications described herein can indirectly reduce the level of a byproduct by reducing or eliminating production of another byproduct which serves as substrate for enzymatic catalysis. Thus reduction or elimination of a particular byproduct can have a cascading effect to reduce or eliminate downstream byproducts of the particular byproduct such as those provided in Tables 3, 10 and 12. For example, reducing By5 of Table 10 can further reduce production of By6 and By8. By way of another example, reducing By40 of Table 10 can further reduce production of By45, By46, By47, By48, By52, By55, and By57. Such cascading is also set forth in Table 3, where the “By#” column corresponds to the Byproduct No. of Table 10.

The genetic modifications described herein of enzymes set forth in Table 3 can decrease activity of one or more of enzymes A1-A25, where the decreased activity results in reducing one or more byproducts of Table 10 or 11. Thus, provided herein are cells having a genetic modification of A1-A25 as described herein, where the genetic modification reduces one or more byproducts as indicated in Table 12. In certain instance, as described herein, a combination of two or more of A1-A25 can be genetically modified. In such instances, the byproducts indicated in Table 13 can be reduced or eliminated in additive fashion (e.g., genetic modification of A5A6 results in reduction of By15, By17, By18, and By39). Those skilled in the art also would readily recognize the combinations of A1-A25 and B1-B5 as described herein would result in additive reduction of the indicated byproducts set forth in Table 13. Thus, in embodiments, genetic modification of an enzyme set forth in the “Enzyme Number” column of Table 13 results in reduced production of the respective byproduct as indicated in the “Byproduct” column of Table 13.

Such genetic modification(s) can reduce or eliminate byproducts in a particular pathway described herein or across two or more pathways described herein, including a HMD pathway, a LVA pathway, a CPO pathway, or a HDO pathway as described herein. As set forth in Table 13, genetic modification of A1-A25 and B1-B5 as described herein alone and in the described combinations can result in decreased byproducts in the indicated pathway (where “Y” indicates genetic modification of the selected enzyme (e.g. A1-A25, B1-B5) reduces the byproducts indicated in the table in that pathway.

TABLE 13
Enzymes and Byproducts

| Enzyme | |
|---|---|
| Number | Byproduct No. |
| A1 | By5-By8, By10, By14, By15, By20, By43, |
| | By51, By54, By58-By60 |
| A2 | By5-By7, By10, By14-By15, By20, By43, |
| | By51, By54, By56, By58-By59 |
| A3 | By17, By18, By39 |
| A4 | By3, By4, By6-By8, By10-By12, By14-By16, |
| | By18, By39-By57 |
| A5 | By17, By18, By39 |
| A6 | By15 |
| A7 | By3, By4, By7, By8, By10-By12, By16, |
| | By40-By50, By53, By54 |
| A8 | By22, By33, By34, By42, By48 |
| A9 | By26, By28, By52-By54 |
| A10 | By36, By61-By65 |
| A11 | By22, By25, By27, By31, By32, By37, By41, |
| | By46, By55, By56, By58 |
| A12 | By38, By57, By66 |
| A13 | By3, By4, By7, By8, By10-By12, By16, |
| | By29, By40-By50, By53, By54 |
| A14 | By1, By2, By9, By12, By13, By18, By36, |
| | By39, By43-By50, By52, By54-By57, By61-By65 |
| A15 | By13, By18, By36, By39, By61-By65 |
| A16 | By2 |
| A17 | By8, By11 |
| A18 | By8 |
| A19 | By1, By2, By9, By12, By13, By18, By36, |
| | By39, By43-By50, By52, By54-By57, By61-By65 |
| A20 | By24, By35, By44, By60 |
| A21 | By21, By22, By30, By32, By47, By59 |
| B3 | By36 |
| B7 | By62 |

Genetic modification of A1 can reduce one or more byproducts selected from byproduct number By5, By6, By7, By8, By10, By14, By15, By20, By43, By51, By54, By58, By59, By60, or 67 of Table 10 or IB18, IB24, or IB15 of Table 11. Genetic modification of A2 can reduce one or more byproducts selected from byproduct number By5, By6, By7, By10, By14, By15, By20, By43, By51, By54, By56, By58, or By59 of Table 10 or IB15 or IB24 of Table 11. Genetic modification of A3 can reduce one or more byproducts selected from byproduct number By17, By18, or By39 of Table 10 or IB11 of Table 11. Genetic modification of A4 can reduce one or more byproducts selected from byproduct number By3, By4, By6, By7, By8, By10, By11, By12, By14, By15, By16, By18, or By39, By40, By41, By42, By43, By44, By45, By46, By47, By48, By49, By50, By51, By52, By53, By54, By55, By56, or By57 of Table 10 or IB18, IB24, IB25, IB11, or IB 15 of Table 11. Genetic modification of A5 can reduce one or more byproducts selected from byproduct number By17, By18, or By39 of Table 10 or IB11 of Table 11. Genetic modification of A6 can reduce one or more byproducts selected from at least byproduct number By15 of Table 10. Genetic modification of A7 can reduce one or more byproducts selected from byproduct number By3, By4, By7, By8, By10, By 11, By12, By16, By40, By41, By42, By43, By44, By45, By46, By47, By48, By49, By50, By53, or By54 of Table 10 or IB25, IB24, or IB11 of Table 11. Genetic modification of A8 can reduce one or more byproducts selected from byproduct number By22, By33, By34, By42, or By48 of Table 10. Genetic modification of A9 can reduce one or more byproducts selected from byproduct number By26, By28, By52, By 53, or By54 of Table 10. Genetic modification of A10 can reduce one or more byproducts selected from byproduct number By36, By61, By 62, By 63, By 64, or By65 of Table 10. Genetic modification of A11 can reduce one or more byproducts selected from byproduct number By22, By25, By27, By31, By32, By37, By41, By46, By55, By56, or By58 of Table 10. Genetic modification of A12 can reduce one or more byproducts selected from byproduct number By38, By57, or By66 of Table 10. Genetic modification of A13 can reduce one or more byproducts selected from byproduct number By3, By4, By7, By8, By10, By 11, By12, By16, By29, By40, By 41, By 42, By 43, By 44, By 45, By 46, By 47, By 48, By 49, By50, By53, or By54 of Table 10 or IB11, IB24 or IB26 of Table 11. Genetic modification of A14 can reduce one or more byproducts selected from byproduct number By1, By2, By9, By12, By13, By18, By36, By39, By43, By 44, By 45, By 46, By 47, By 48, By 49, By50, By52, By54, By 55, By 56, By57, By61, By 62, By 63, By 64, or By65 of Table 10 or IB26 or IB11 of Table 11. Genetic modification of A15 can reduce one or more byproducts selected from byproduct number By13, By18, By36, By39, or By61, By 62, By 63, By 64, or By65 of Table 10 or IB11 of Table 11. Genetic modification of A16 can reduce one or more byproducts selected from at least byproduct number By2 of Table 10. Genetic modification of A17 can reduce one or more byproducts selected from byproduct number By8 or By 11 of Table 10 or IB18 or IB25 of Table 11. Genetic modification of A18 can reduce one or more byproducts selected from at least byproduct number By8 of Table 10. Genetic modification of A19 can reduce one or more byproducts selected from byproduct number By 1, By2, By9, By12, By13, By18, By36, By39, By43, By 44, By 45, By 46, By 47, By 48, By 49, By50, By52, By54, By 55, By 56, By57, By 61, By 62, By 63, By 64, or By65 of Table 10 or IB11 of Table 11. Genetic modification of A20 can reduce one or more byproducts selected from byproduct number By24, By35, By44, or By60 of Table 10. Genetic modification of A21 can reduce one or more byproducts selected from byproduct number By21, By22, By30, By32, By47, or By59 of Table 10. Genetic modification of A22 can reduce one or more byproducts selected from byproduct number By1-26, By29, By36, By39-66 of Table 10 or IB 11, IB18, IB15, IB25 or IB25 of Table 11. Genetic modification of A23 can reduce one or more byproducts selected from byproduct number By1-26, By29, By36, By39-66 of Table 10 or IB 11, IB18, IB15, IB25 or IB25 of Table 11. Genetic modification of A24 can reduce one or more byproducts selected from byproduct number By43, By45, By47-50, By52, By55 of Table 10. Genetic modification of A25 can reduce one or more byproducts selected from byproduct number By43, By45, By47-50, By52, By55 of Table 10.

Genetic modification of B1 can reduce one or more byproducts selected from byproduct number By25-By28, By41, By46, By52-By55, By58 of Table 10. Genetic modification of B2 can reduce one or more byproducts selected from byproduct number By12, By19, By49, or By50 of Table 10, or IB24 or IB25 of Table 11. Genetic modification of B3 can reduce one or more byproducts selected from byproduct number By1-By11, By13-By18, By36, By39, By40, By61-By65 of Table 10, or IB11, IB18, IB24 or IB25 of Table 11. Genetic modification of B4 can reduce one or more byproducts selected from By45 of Table 10. Genetic modification of B5 can reduce one or more byproducts selected from By45 of Table 10

When genetic modifications of the above enzymes (A1-A25 and B1-B5) are included in a cell described herein, byproducts described above associated with each independent enzyme can be reduced in combination.

Thus, for example, genetic modification of A6 and A8 in combination reduces at least byproducts By15, By22, By33, By34, By42 and By48 in a pathway described for producing 6ACA, CPL, or HMD as a target product. One skilled in the art would recognize this applies to all enzymes A1-A25 and B1-B5 as set forth in Table 13 with respect to the pathways and byproducts indicated in the table and as set forth above. Accordingly, reduction of such byproduct(s) can increase the purity of the target product as described herein in other sections.

Cells described herein having at least one genetic modification of an enzyme selected from A1-A25 or B1-B5 may produce one or more target products described herein. For example, a cell described herein capable of producing HMD, CPL, or ACA can include genetic modification of one or more of A1-A25 and B1-B5. A cell described herein capable of producing HMD can include genetic modification of a subset of A1-A25 and B1-B5, where the subset of the enzymes A1-A15, A20-A25, and B1-B5.

A cell described herein capable of producing LVA can include genetic modification of a subset of A1-A25 and B1-B5, where the subset of the enzymes A1, A3, A4, A5, A7, A10, A14, A15, A17, A19, A22-A25, and B1-B5. A cell described herein capable of producing ADA can include genetic modification of a subset of A1-A25 and B1-B5, where the subset of the enzymes A1-A7, A10, A14-A17, A19, A22-A25, and B1-B5. A cell described herein capable of producing HDO can include genetic modification of a subset of A1-A25 and B1-B5, where the subset of the enzymes A1-A7, A10, A14-A17, A19, A22-A25, and B1-B5. A cell described herein capable of producing CPO can include genetic modification of a subset of A1-A25 and B1-B5, where the subset of the enzymes A1-A7, A10, A14-A17, A19, A22-A25, and B1-B5.

The cells described herein can include one or more gene modifications that confer production of a target product described herein having a decreased level of at least one byproduct described herein. The cells described herein can also include one or more gene disruptions that confer increased production of a target produced described herein. In one embodiment, such one or more gene disruptions confer growth-coupled production of target product, and can, for example, confer stable growth-coupled production of target product. In another embodiment, the one or more gene disruptions can confer obligatory coupling of target product production to growth of the microbial organism. Such one or more gene disruptions reduce the activity of the respective one or more encoded enzymes. The one or more genetic modifications described herein can reduce the levels of a byproduct described herein. Thus, in embodiments, genetic modifications described herein can increase the purity of a target product described herein.

The non-naturally occurring microbial organisms described herein can have one or more genetic modifications of an enzyme listed in Table 3 or 4. As disclosed herein, the one or more genetic modifications described herein can be a deletion of a gene encoding an enzyme described herein. Such non-naturally occurring microbial organisms of the invention include bacteria, yeast, fungus, or any of a variety of other microorganisms applicable to fermentation processes, as disclosed herein.

Thus, the invention provides a non-naturally occurring microbial organism that includes (a) one or more genetic modifications, where the one or more genetic modifications occur in genes encoding proteins or enzymes where the one or more gene modifications confer decreased production (e.g. levels of) byproducts in desired target product and (b) at least one or more gene disruptions, where the one or more gene disruptions occur in genes encoding proteins or enzymes set forth in a biosynthetic pathway described herein (e.g. a HMD pathway, a HDO pathway) where the one or more gene disruptions confer increased production of a target product in the organism. The production of target product can be growth-coupled or not growth-coupled. In a particular embodiment, the production of target product can be obligatorily coupled to growth of the organism, as disclosed herein.

The invention provides non-naturally occurring microbial organisms that have genetic alterations such as gene disruptions that increase production of a target product, for example, growth-coupled production of HMD, CPL, CPO, ADA, 6ACA, ADA, LVA, or HDO as described herein. Growth-coupled production can be linked to HMD, CPL, CPO, ADA, 6ACA, ADA, LVA, or HDO. Product production can be, for example, obligatorily linked to the exponential growth phase of the microorganism by genetically altering the biosynthetic pathways of the cell, as disclosed herein. Further, the cells include one or more genetic modifications as described herein of enzymes A1-A25 and B1-B5 which reduce the level of byproducts in the desired target product. Thus, the genetic modifications described herein increase the final purity of or increase the yield as described herein of the desired target product when compared to a cell lacking such genetic modifications. The purity of the desired target product can be 1, 2, 3, 4, 5, 6, 7, 8, 9, 10, 15, 20, 25, 30, 35, 40, 45, 50, 55, 60, 65, 70, 75, 80, 85, 90, 91, 92, 92, 93, 94, 95, 96, 97, 98, 99, or 100 percent greater than the same target product produced from a cell lacking the genetic modifications. The yield can be measured as described herein elsewhere and increased in accordance with description herein.

The genetic alterations can increase the production of the desired product or even make the desired product an obligatory product during the growth phase. Other sets of metabolic alterations or transformations that result in increased production and elevated levels of target product biosynthesis are exemplified in Table 14, FIG. 1, FIG. 2, FIG. 3, FIG. 4, and FIG. 5, are known in the art, and are exemplified by U.S. Pat. Nos. 8,377,680 and 8,940,509 which are herein incorporated in their entireties and for all purposes. Each alteration within a set corresponds to the requisite metabolic reaction that should be functionally disrupted. Functional disruption of all reactions within a given pathway described herein can result in the increased production of target product by the engineered strain during the growth phase. Further, genetic modifications described herein within such engineered strains increase the purity of the target product. Exemplary genes that encode enzymes or proteins useful in the biosynthetic pathways for decreasing byproducts described herein are set forth in for example Table 4. The genetic modification can be a gene mutation of a gene encoding the enzyme. Such gene mutations include those described herein, such as, for example a gene mutation of a transcriptional regulatory region of the gene encoding the enzyme, a gene mutation of a protein coding region of the gene encoding the enzyme, or a gene mutation of a gene encoding a transcriptional or translational regulator of the enzyme.

Given the teachings and guidance provided herein, those skilled in the art will understand that to introduce a metabolic alteration such as attenuation using genetic modifications described herein of an enzyme described herein, it can be necessary to disrupt the catalytic activity of the one or more enzymes involved in the reaction.

Alternatively, such genetic alteration can include disrupting expression of a regulatory protein or cofactor necessary for enzyme activity or maximal activity. Furthermore, genetic loss of a cofactor necessary for an enzymatic reaction can also have the same effect as a disruption of the gene encoding the enzyme. Disruption can occur by a variety of methods including, for example, deletion of an encoding gene or incorporation of a genetic alteration in one or more of the encoding gene sequences. The encoding genes targeted for disruption can be one, some, or all of the genes encoding enzymes involved in the catalytic activity. Thus in instances where genetic alteration of enzymes used in a biosynthetic pathway described herein to product a target product described herein, one or more enzymes in the pathway can be genetically disrupted as described herein. In instances where genetic modification of enzymes useful for decreasing byproducts described herein, genetic modifications described herein can be performed to alter activity of one or more of the enzymes catalyzing reaction to the byproduct. Such alteration is equally applicable to disruption of enzymes for catalysis in the biosynthetic pathways described herein.

TABLE 14
central metabolic byproducts

| Byproduct | | | Dissimilation | Steps from |
|---|---|---|---|---|
| No. | Byproduct Name | Mode of formation | Pathway | pathway |
| MB1 | acetate | From native enzymes (ackA-pta, | Pyr--> Ace | 1-2 |
| | | poxB and any | AcCoA --> Ace | |
| | | transferase/hydrolase acting on | AcCoA --> Acald --> | |
| | | acetyl-CoA), overflow | Ace | |
| | | metabolism product | | |
| MB2 | ethanol | From acetyl-CoA through non- | AcCoA--> Acald --> | 2 |
| | | native aid and rogue ADHs | EtOH | |
| | | | Pyr--> Acald --> | |
| | | | EtOH | |
| MB3 | ethanolamine | From transamination of | AcCoA--> Acald --> | 2 |
| | | acetaldehyde | EtAmine | |
| MB4 | pyruvate | Overflow product | — | 0 |
| MB5 | glutamate | Pathway, high likelihood in C. glutamicum | — | 0 |
| MB6 | lactate | Byproduct of central metabolism | Pyr --> Lac | 1 |
| | | | Mgx --> Lac | |
| MB7 | formate | If PfIB is used for converting | Pyr --> For; Fald--> | 1 |
| | | pyruvate to acetyl-CoA or via the | For | |
| | | methanol oxidation pathway | | |
| MB8 | aspartate | From pathway imbalance | — | 0 |
| MB9 | alanine | From pyruvate transamination | Pyr --> Ala | 1 |
| MB10 | acetaldehyde | From ald activity on acetyl-CoA | — | 0 |
| | | and from methanol | | |
| | | dehydrogenase activity on | | |
| | | ethanol | | |
| MB11 | formaldehyde | If methanol oxidation pathway is | — | 0 |
| | | not efficient | | |
| MB12 | 3- | From acetoacetyl-CoA that is | AcCoA --> AACoA --> | 3-4 |
| | hydroxybutyrate | formed by non-specific thiolase | 3HB-CoA --> 3HB | |
| | | activity on acetyl-CoA | AcCoA --> AACoA --> | |
| | | | 3HB-CoA --> 3HBald | |
| | | | --> 3HB | |
| MB13 | acetone | Decarboxylation of acetoacetate | AcCoA --> AACoA --> | 3 |
| | | | Aac --> Acetone | |
| MB14 | 4-hydroxy 2- | From acetoacetate | AcCoA --> AACoA --> | 3 |
| | butanone | | Aac --> 4H2B | |
| MB15 | butanol | 3-hydroxybutyryl-CoA can go | AcCoA --> AACoA --> | 6 |
| | | through a series of steps to form | 3HB-CoA --> CrtCoA | |
| | | butanol | --> BuCoA --> BuAld | |
| | | | --> BuOH | |
| MB16 | butyrate | 3-hydroxybutyryl-CoA can go | AcCoA --> AACoA --> | 6 |
| | | through a series of steps to form | 3HB-CoA --> CrtCoA | |
| | | butyrate | --> BuCoA --> BuAld | |
| | | | --> Butyrate | |
| | | | AcCoA --> AACoA --> | |
| | | | 3HB-CoA --> CrtCoA | |
| | | | --> BuCoA --> | |
| | | | Butyrate | |
| MB17 | 1,3-butanediol | 3-HB CoA formed from non- | AcCoA --> AACoA --> | 4 |
| | | specific activity of sec Adh can be | 3HB-CoA --> 3HBald | |
| | | converted to 13BDO via non | --> 13BDO | |
| | | specific ald and adh. | | |

Abbreviations: 1,3-butanediol=13BDO; 2-acetylputrescine=2Ac-Put; 3,6-diaminohexanoate=36DAhx; 3,6-dihydroxyhexanoate=36DHhx; 3-amino-6-hydroxyhexanoate=3A6Hhx; 3-hydroxy-6-aminohexanoyl-CoA=3H6AhexCoA; 3-hydroxy-6-aminohexanoate=3H6Ahx; 3-hydroxyadipyl-CoA=3hacoa; 3-hydroxyadipate=3HAdip; 3-hydroxyadipate=semialdehyde=3HAdipSA; 3-hydroxybutyrate=3HB; 3-hydroxybutyraldehyde=3HBald; 3-hydroxybutyryl-CoA=3HB-CoA; 3-keto-3-hydroxyhexanoate=3K3Hhx; 3-keto-6-aminohexanoate=3K6Hhx; 3-oxo-6-aminohexanoate=3O6Ahx; 3-oxoadipyl-CoA=3oacoa; 3-oxoadipate=3OAdip; 3-oxoadipate=semialdehyde=3OaSald; 4-aminobutyraldehyde=4ABal; 4-(hydroxyamino)butanol=4AB-OH; 4-aminobutanol=4ABol; 4-hydroxy-2-butanone=4H2B; 4-hydroxybutyrate=4HB; 4-hydroxypentanoate=4HP; 4-hydroxypiperidin-2-one=4Hpip2one; 4-oxopentanoate=4OPent; 5-carboxy-2-pentenoyl-CoA=5c2pcoa; 5-carboxy-2-pentenoate=5C2Pen; 5-carboxy-2-pentenal=5C2Penald; 6-aminocaproate=6aca; N-carbamoyl-ACA=6-ACA-Carb; 6-aminocaproyl-CoA=6-ACA-CoA; 6-aminohexanol=6-ACA-OH; 6-aminocaproate=semialdehyde=6acasa; 6-aminohex-4-enoate=6AH4en; 6-aminohexanol=6-AHexOH; 6-hydroxyhex-2-enoate=6H2HEN; 6-hydroxyhex-4-enoate=6HH4en; 6-hydroxyhexanoate=6HHex; 7-carboxy-3-oxooct-4-enoate=7-c-3-oxooct-4-enoate; 8-amino-3-oxooctanoate=8A3OOctate; 8-amino-3-oxooctanoyl-CoA=8A3OOct-CoA; acetoacetate=Aac=; acetoacetyl-CoA=AACoA=; acetaldehyde=Acald; acetyl-CoA=accoa; acetate=Ace; acetyl-ACA=acetyl-6-ACA; acetyl-HMDA=Acetyl-HMDA; acetyl-4-aminobutyrate=Ac-GABA; acetylputrescine=Ac-Put=; N-acyl-HMDA=acyl-HMDA; adipate=Adip; adipyl-CoA=adipcoa; adipate=semialdehyde=adipsa; alanine=Ala; butyraldehyde=BuAld; butyryl-CoA=BuCoA; butanol=BuOH; carbamoyl-4-aminobutyrate=Carb-GABA; carbamoyl-4-aminobutanol=Cm-4ABol; carbamoyl-putrescine=Cm-Put; crotonyl-CoA=CrtCoA; ethanolamine=EtAmine; ethanol=EtOH; formate=For; formaldehyde=Fald; 4-aminobutyrate=GABA; 4-aminobutyryl-CoA=GABA-CoA; N-glutamyl-HMDA=Glu-HMDA; glutamylputrescine=Glu-Put; hexamethylene=diamine=hmda; carbamoyl-HMDA=HMDA-Carb; lactate=Lac; N-methyl-4-aminobutyrate=Me-GABA; N-methyl-HMDA=ME-HMDA; N-methylputrescine=Me-Put; methylglyoxal=Mgx; N-acetyl-6-aminohexanol=N-acetyl-6-AHexOH; N-glutamyl-6-aminocaproate=Nglu-6ACA; N-glutamyl-6-aminohexanol=N-glu-6-AHexOH; N-hydroxy-6-aminohexanol=N-hydroxy-6-AHexOH; N-methyl-6-aminocaproate=Nme-6ACA; N,N-dimethyl-HMDA=NN-DM-HMDA; N-hydroxy-6-aminocaproate=NOH-6ACA; N-hydroxy-succinyl-aminocaproate=NOH-succ-6ACA; N-hydroxy-succinyl-HMDA=N—OH-succ-HMDA; N-hydroxy-HMDA=OH-HMDA; putrescine=Put=; N-hydroxy-putrescine=Put-OH; pyruvate=Pyr; succinate=Succ; succinyl-HMDA=Succ-HMDA; succinyl-CoA=succoa; succinate=semialdehyde=Sucsal; byprod=byproduct; intermed=intermediates; Exem.=exemplary.

For example, where a single enzyme is involved in a targeted catalytic activity, disruption can occur by a genetic alteration that reduces or eliminates the catalytic activity of the encoded gene product. Similarly, where the single enzyme is multimeric, including heteromeric, disruption can occur by a genetic alteration that reduces or destroys the function of one or all subunits of the encoded gene products. Destruction of activity can be accomplished by loss of the binding activity of one or more subunits required to form an active complex, by destruction of the catalytic subunit of the multimeric complex or by both. Other functions of multimeric protein association and activity also can be targeted in order to disrupt a metabolic reaction of the invention. Such other functions are well known to those skilled in the art. Similarly, a target enzyme activity can be reduced or eliminated by disrupting expression of a protein or enzyme that modifies and/or activates the target enzyme, for example, a molecule required to convert an apoenzyme to a holoenzyme. Further, some or all of the functions of a single polypeptide or multimeric complex can be disrupted according to the invention in order to reduce or abolish the catalytic activity of one or more enzymes involved in a reaction or metabolic modification of the invention. Similarly, some or all of enzymes involved in a reaction or metabolic modification of the invention can be disrupted so long as the targeted reaction is reduced or eliminated.

Given the teachings and guidance provided herein, those skilled in the art also will understand that an enzymatic reaction can be disrupted by reducing or eliminating reactions encoded by a common gene and/or by one or more orthologs of that gene exhibiting similar or substantially the same activity. Reduction of both the common gene and all orthologs can lead to complete abolishment of any catalytic activity of a targeted reaction. However, disruption of either the common gene or one or more orthologs can lead to a reduction in the catalytic activity of the targeted reaction sufficient to promote coupling of growth to product biosynthesis. Disruption of either the common gene or one or more orthologs (e.g. Table 4) of an enzyme described herein useful for decreasing byproducts described herein can lead to a reduction in the catalytic activity of the targeted reaction sufficient to reduce the levels of byproducts such as those set forth in Table 10 or 11. Exemplified herein are both the common genes encoding catalytic activities for a variety of enzymes as well as their orthologs. Those skilled in the art will understand that the genetic modifications described herein of some or all of the genes encoding enzyme(s) of a targeted enzymatic reaction to a byproduct described herein can be practiced in the methods of the invention and incorporated into the non-naturally occurring microbial organisms of the invention in order to achieve the reduced levels of byproducts described herein. Those skilled in the art will also understand that disruption of some or all of the genes encoding an enzyme of a targeted metabolic reaction can be practiced in the methods of the invention and incorporated into the non-naturally occurring microbial organisms of the invention in order to achieve the increased production of target product or growth-coupled product production.

Given the teachings and guidance provided herein, those skilled in the art also will understand that enzymatic activity or expression can be attenuated using well known methods. Reduction of the activity or amount of an enzyme can mimic complete disruption of a gene if the reduction causes activity of the enzyme to fall below a critical level that is normally required for a pathway to function. Reduction of enzymatic activity by various techniques rather than use of a gene disruption can be important for an organism's viability. Methods of reducing enzymatic activity that result in similar or identical effects of a gene disruption include, but are not limited to: reducing gene transcription or translation; destabilizing mRNA, protein or catalytic RNA; and mutating a gene that affects enzyme activity or kinetics (See, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999). Natural or imposed regulatory controls can also accomplish enzyme attenuation including: promoter replacement (See, Wang et al., Mol. Biotechnol. 52(2):300-308 (2012)); loss or alteration of transcription factors (Dietrick et al., Annu. Rev. Biochem. 79:563-590 (2010); and Simicevic et al., Mol. Biosyst. 6(3):462-468 (2010)); introduction of inhibitory RNAs or peptides such as siRNA, antisense RNA, RNA or peptide/small-molecule binding aptamers, ribozymes, aptazymes and riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); and addition of drugs or other chemicals that reduce or disrupt enzymatic activity such as an enzyme inhibitor, an antibiotic or a target-specific drug.

One skilled in the art will also understand and recognize that attenuation of an enzyme can be done at various levels. For example, at the gene level, a mutation causing a partial or complete null phenotype, such as a gene disruption, or a mutation causing epistatic genetic effects that mask the activity of a gene product (Miko, Nature Education 1(1) (2008)), can be used to attenuate an enzyme. At the gene expression level, methods for attenuation include: coupling transcription to an endogenous or exogenous inducer, such as isopropylthio-β-galactoside (IPTG), then adding low amounts of inducer or no inducer during the production phase (Donovan et al., J. Ind. Microbiol. 16(3):145-154 (1996); and Hansen et al., Curr. Microbiol. 36(6):341-347 (1998)); introducing or modifying a positive or a negative regulator of a gene; modify histone acetylation/deacetylation in a eukaryotic chromosomal region where a gene is integrated (Yang et al., Curr. Opin. Genet. Dev. 13(2):143-153 (2003) and Kurdistani et al., Nat. Rev. Mol. Cell Biol. 4(4):276-284 (2003)); introducing a transposition to disrupt a promoter or a regulatory gene (Bleykasten-Brosshans et al., C. R. Biol. 33(8-9):679-686 (2011); and McCue et al., PLoS Genet. 8(2):e1002474 (2012)); flipping the orientation of a transposable element or promoter region so as to modulate gene expression of an adjacent gene (Wang et al., Genetics 120(4):875-885 (1988); Hayes, Annu. Rev. Genet. 37:3-29 (2003); in a diploid organism, deleting one allele resulting in loss of heterozygosity (Daigaku et al., Mutation Research/Fundamental and Molecular Mechanisms of Mutagenesis 600(1-2)177-183 (2006)); introducing nucleic acids that increase RNA degradation (Houseley et al., Cell, 136(4):763-776 (2009); or in bacteria, for example, introduction of a transfer-messenger RNA (tmRNA) tag, which can lead to RNA degradation and ribosomal stalling (Sunohara et al., RNA 10(3):378-386 (2004); and Sunohara et al., J. Biol. Chem. 279:15368-15375 (2004)). At the translational level, attenuation can include: introducing rare codons to limit translation (Angov, Biotechnol. J. 6(6):650-659 (2011)); introducing RNA interference molecules that block translation (Castel et al., Nat. Rev. Genet. 14(2):100-112 (2013); and Kawasaki et al., Curr. Opin. Mol. Ther. 7(2):125-131 (2005); modifying regions outside the coding sequence, such as introducing secondary structure into an untranslated region (UTR) to block translation or reduce efficiency of translation (Ringner et al., PLoS Comput. Biol. 1(7):e72 (2005)); adding RNAase sites for rapid transcript degradation (Pasquinelli, Nat. Rev. Genet. 13(4):271-282 (2012); and Arraiano et al., FEMS Microbiol. Rev. 34(5):883-932 (2010); introducing antisense RNA oligomers or antisense transcripts (Nashizawa et al., Front. Biosci. 17:938-958 (2012)); introducing RNA or peptide aptamers, ribozymes, aptazymes, riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); or introducing translational regulatory elements involving RNA structure that can prevent or reduce translation that can be controlled by the presence or absence of small molecules (Araujo et al., Comparative and Functional Genomics, Article ID 475731, 8 pages (2012)). At the level of enzyme localization and/or longevity, enzyme attenuation can include: adding a degradation tag for faster protein turnover (Hochstrasser, Annual Rev. Genet. 30:405-439 (1996); and Yuan et al., PLoS One 8(4):e62529 (2013)); or adding a localization tag that results in the enzyme being secreted or localized to a subcellular compartment in a eukaryotic cell, where the enzyme would not be able to react with its normal substrate (Nakai et al. Genomics 14(4):897-911 (1992); and Russell et al., J. Bact. 189(21)7581-7585 (2007)). At the level of post-translational regulation, enzyme attenuation can include: increasing intracellular concentration of known inhibitors; or modifying post-translational modified sites (Mann et al., Nature Biotech. 21:255-261 (2003)). At the level of enzyme activity, enzyme attenuation can include: adding an endogenous or an exogenous inhibitor, such as an enzyme inhibitor, an antibiotic or a target-specific drug, to reduce enzyme activity; limiting availability of essential cofactors, such as vitamin B12, for an enzyme that requires the cofactor; chelating a metal ion that is required for enzyme activity; or introducing a dominant negative mutation. The applicability of a technique for attenuation described above can depend upon whether a given host microbial organism is prokaryotic or eukaryotic, and it is understand that a determination of what is the appropriate technique for a given host can be readily made by one skilled in the art.

In some embodiments, microaerobic designs can be used based on the growth-coupled formation of the desired product. To examine this, production cones can be constructed for each strategy by first maximizing and, subsequently minimizing the product yields at different rates of biomass formation feasible in the network. If the rightmost boundary of all possible phenotypes of the mutant network is a single point, it implies that there is a unique optimum yield of the product at the maximum biomass formation rate possible in the network. In other cases, the rightmost boundary of the feasible phenotypes is a vertical line, indicating that at the point of maximum biomass the network can make any amount of the product in the calculated range, including the lowest amount at the bottommost point of the vertical line. Such designs are given a low priority.

The target product-production strategies identified by the methods disclosed herein such as the OptKnock framework are generally ranked on the basis of their (i) theoretical yields, (ii) growth-coupled target product formation characteristics and (iii) reduction of specific byproducts identified for a respective pathway described herein to a target product such as a compound set forth in Table 12.

Accordingly, the invention also provides a non-naturally occurring microbial organism having a set of metabolic modifications coupling target product production to growth of the organism, where the set of metabolic modifications includes disruption of one or more genes selected from the set of genes encoding proteins as shown in Table 3, 4, 5, 6, 7, or FIG. 1, FIG. 2, FIG. 3, FIG. 4, or FIG. 5.

Each of the strains can be supplemented with additional deletions if it is determined that the strain designs do not sufficiently increase the production of target product and/or couple the formation of the product with biomass formation. Likewise, strains can be supplemented with additional genetic modifications described herein to decrease levels of byproducts described herein. Alternatively, some other enzymes not known to possess significant activity under the growth conditions can become active due to adaptive evolution or random mutagenesis. Such activities can also be knocked out. However, the list of gene deletion sets disclosed herein allows the construction of strains exhibiting high-yield production of target product with reduced levels of byproducts described herein, and such strains can include growth-coupled production of target product.

Target products can be harvested or isolated at any time point during the culturing of the microbial organism, for example, in a continuous and/or near-continuous culture period, as disclosed herein. Accordingly, and as discussed hereinabove, target products include intermediates (e.g. compounds) set forth in the described pathways disclosed herein where the non-naturally occurring microorganism includes a pathway described herein engineered in the cell for production of such intermediates. Generally, the longer the microorganisms are maintained in a continuous and/or near-continuous growth phase, the proportionally greater amount of target product can be produced. The genetic modifications described herein to reduce or eliminate the activity of enzymes producing byproducts described herein (e.g. A1-A25, B1-135, and orthologs and homologs thereof) can be proportionally greater with longer continuous and/or near-continuous growth phase. Longer continuous and/or near-continuous growth phase can therefore, in instances described herein, increase the purity of target products described herein (e.g. decrease levels of byproducts such as those of Table 10).

Therefore, the invention additionally provides a method for producing a target product having reduced levels of byproducts that includes culturing a non-naturally occurring microbial organism having one or more gene modifications, as disclosed herein. As described herein, such non-naturally occurring microorganisms can also include gene disruptions of enzymes in pathways described herein to increase production yield target products described herein. The genetic modifications and gene disruptions described herein can occur in one or more genes encoding an enzyme that increases production of target product, including optionally coupling target product production to growth of the microorganism when the gene disruption reduces or eliminates an activity of the enzyme. For example, the disruptions can confer stable growth-coupled production of target product onto the non-naturally microbial organism.

In some embodiments, the gene disruption can include a complete gene deletion as described herein using techniques known in the art and disclosed herein. In some embodiments other methods to disrupt or modify a gene include, for example, frameshifting by omission or addition of oligonucleotides or by mutations that render the gene inoperable. One skilled in the art will recognize the advantages of gene deletions, however, because of the stability it confers to the non-naturally occurring organism from reverting to a parental phenotype in which the gene disruption or genetic modification has not occurred. In particular, the gene disruptions and genetic modifications described herein are selected from the gene sets as disclosed herein.

Once computational predictions are made of gene sets for disruption to increase production of target product, and gene sets for modification to decrease levels of byproducts described herein, the strains can be constructed, evolved, and tested. Gene disruptions and genetic modifications, including gene deletions, are introduced into host organism by methods well known in the art. A particularly useful method for gene disruption is by homologous recombination, as disclosed herein.

The engineered strains can be characterized by measuring the growth rate, the substrate uptake rate, the product/byproduct secretion rate, and/or levels of byproducts produced. Such characterizations can be compared to cells lacking the gene disruptions and genetic modifications described herein. Cultures can be grown and used as inoculum for a fresh batch culture for which measurements are taken during exponential growth. The growth rate can be determined by measuring optical density using a spectrophotometer (A600). Concentrations of glucose and other organic acid byproducts in the culture supernatant can be determined by well known methods such as HPLC, GC-MS or other well known analytical methods suitable for the analysis of the desired product, as disclosed herein, and used to calculate uptake and secretion rates.

Strains containing gene disruptions and/or genetic modifications described herein can exhibit suboptimal growth rates until their metabolic networks have adjusted to their missing functionalities. To assist in this adjustment, the strains can be adaptively evolved. By subjecting the strains to adaptive evolution, cellular growth rate becomes the primary selection pressure and the mutant cells are compelled to reallocate their metabolic fluxes in order to enhance their rates of growth. This reprogramming of metabolism has been demonstrated for example for several E. coli mutants that had been adaptively evolved on various substrates to reach the growth rates predicted a priori by an in silico model (Fong and Palsson, Nat. Genet. 36:1056-1058 (2004)). The growth improvements and reduced levels of byproducts brought about by adaptive evolution can be accompanied by enhanced rates of target product production with reduced levels of byproducts such as those of Table 10 when compared to a cell lacking the genetic modifications. The strains are generally adaptively evolved in replicate, running in parallel, to account for differences in the evolutionary patterns that can be exhibited by a host organism (Fong and Palsson, Nat. Genet. 36:1056-1058 (2004); Fong et al., J. Bacteriol. 185:6400-6408 (2003); Ibarra et al., Nature 420:186-189 (2002)) that could potentially result in one strain having superior production qualities over the others. Evolutions can be run for a period of time, typically 2-6 weeks, depending upon the rate of growth improvement attained. In general, evolutions are stopped once a stable phenotype is obtained.

Following the adaptive evolution process, the new strains are characterized again by measuring the growth rate, the substrate uptake rate, the product/byproduct secretion rate, and the level of byproduct. These results are compared to the theoretical predictions by plotting actual growth and production yields alongside the production envelopes from metabolic modeling. The most successful design/evolution combinations are chosen to pursue further, and are characterized in lab-scale batch and continuous fermentations. The growth-coupled biochemical production concept behind the methods disclosed herein such as OptKnock approach should also result in the generation of genetically stable overproducers and strains demonstrated having reduced byproduct synthesis. Thus, the cultures are maintained in continuous mode for an extended period of time, for example, one month or more, to evaluate long-term stability. Periodic samples can be taken to ensure that yield and productivity are maintained.

Adaptive evolution is a powerful technique that can be used to increase growth rates of mutant or engineered microbial strains, or of wild-type strains growing under unnatural environmental conditions. It is especially useful for strains designed via methods such as OptKnock, which results in growth-coupled product formation. Therefore, evolution toward optimal growing strains will indirectly optimize production as well. Unique strains of E. coli K-12 MG1655 were created through gene knockouts and adaptive evolution. (Fong and Palsson, Nat. Genet. 36:1056-1058 (2004)). In this work, all adaptive evolutionary cultures were maintained in prolonged exponential growth by serial passage of batch cultures into fresh medium before the stationary phase was reached, thus rendering growth rate as the primary selection pressure. Knockout strains were constructed and evolved on minimal medium supplemented with different carbon substrates (four for each knockout strain). Evolution cultures were carried out in duplicate or triplicate, giving a total of 50 evolution knockout strains. The evolution cultures were maintained in exponential growth until a stable growth rate was reached. The computational predictions were accurate (within 10%) at predicting the post-evolution growth rate of the knockout strains in 38 out of the 50 cases examined. Furthermore, a combination of OptKnock design with adaptive evolution has led to improved lactic acid production strains. (Fong et al., Biotechnol. Bioeng. 91:643-648 (2005)). Similar methods can be applied to the strains disclosed herein and applied to various host strains.

There are a number of developed technologies for carrying out adaptive evolution. Exemplary methods are disclosed herein. In some embodiments, optimization of a non-naturally occurring organism of the present invention includes utilizing adaptive evolution techniques to increase target product production, reduced levels of byproducts described herein and/or stability of the producing strain described herein.

Serial culture involves repetitive transfer of a small volume of grown culture to a much larger vessel containing fresh growth medium. When the cultured organisms have grown to saturation in the new vessel, the process is repeated. This method has been used to achieve the longest demonstrations of sustained culture in the literature (Lenski and Travisano, Proc. Natl. Acad. Sci. USA 91:6808-6814 (1994)) in experiments which clearly demonstrated consistent improvement in reproductive rate over a period of years. Typically, transfer of cultures is usually performed during exponential phase, so each day the transfer volume is precisely calculated to maintain exponential growth through the next 24 hour period. Manual serial dilution is inexpensive and easy to parallelize.

In continuous culture the growth of cells in a chemostat represents an extreme case of dilution in which a very high fraction of the cell population remains. As a culture grows and becomes saturated, a small proportion of the grown culture is replaced with fresh media, allowing the culture to continually grow at close to its maximum population size. Chemostats have been used to demonstrate short periods of rapid improvement in reproductive rate (Dykhuizen, Methods Enzymol. 613-631 (1993)). The potential usefulness of these devices was recognized, but traditional chemostats were unable to sustain long periods of selection for increased reproduction rate, due to the unintended selection of dilution-resistant (static) variants. These variants are able to resist dilution by adhering to the surface of the chemostat, and by doing so, outcompete less adherent individuals, including those that have higher reproductive rates, thus obviating the intended purpose of the device (Chao and Ramsdell, J. Gen. Microbiol 20:132-138 (1985)). One possible way to overcome this drawback is the implementation of a device with two growth chambers, which periodically undergo transient phases of sterilization, as described previously (Marliere and Mutzel, U.S. Pat. No. 6,686,194).

Evolugator™ is a continuous culture device developed by Evolugate, LLC (Gainesville, Fla.) and exhibits significant time and effort savings over traditional evolution techniques (de Crecy et al., Appl. Microbiol. Biotechnol. 77:489-496 (2007)). The cells are maintained in prolonged exponential growth by the serial passage of batch cultures into fresh medium before the stationary phase is attained. By automating optical density measurement and liquid handling, the Evolugator™ can perform serial transfer at high rates using large culture volumes, thus approaching the efficiency of a chemostat in evolution of cell fitness. For example, a mutant of Acinetobacter sp ADP1 deficient in a component of the translation apparatus, and having severely hampered growth, was evolved in 200 generations to 80% of the wild-type growth rate. However, in contrast to the chemostat which maintains cells in a single vessel, the machine operates by moving from one “reactor” to the next in subdivided regions of a spool of tubing, thus eliminating any selection for wall-growth. The transfer volume is adjustable, and normally set to about 50%. A drawback to this device is that it is large and costly, thus running large numbers of evolutions in parallel is not practical. Furthermore, gas addition is not well regulated, and strict anaerobic conditions are not maintained with the current device configuration. Nevertheless, this is an alternative method to adaptively evolve a production strain.

As disclosed herein, a nucleic acid encoding a desired activity of a target product pathway can be introduced into a host organism. In some cases, it can be desirable to modify an activity of a target product pathway enzyme or protein to increase production of target product. Further, the non-naturally occurring microorganisms described herein include modified activity of enzymes that produce byproducts in the biosynthetic pathways described herein. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol. Eng 22:1-9 (2005); and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of a target product pathway enzyme or protein or an enzyme described herein associated with byproduct production. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

While generally described herein as a microbial organism that contains a one or more genetic modifications described herein that reduce at least one byproduct described herein (e.g. Table 10) and a target product (e.g., HMD, LVA, 6ACA, CPL, CPO, ADA, or HDO) pathway, it is understood that the invention additionally provides a non-naturally occurring microbial organism that includes one or more genetic modifications described herein that reduce at least one byproduct described herein (e.g. Table 10) and at least one exogenous nucleic acid encoding a target product (e.g., HMD, LVA, 6ACA, CPL, CPO, ADA, or HDO) pathway enzyme expressed in a sufficient amount to produce an intermediate of such a pathway. Thus, for example, in addition to a microbial organism containing a HMD pathway that produces HMD, 6ACA, ADA, CPL, or an intermediate described herein with less byproduct than a cell without the one or more genetic modifications described herein, the invention also provides a non-naturally occurring microbial organism that includes one or more genetic modifications described herein that reduce at least one byproduct described herein (e.g. Table 10) and at least one exogenous nucleic acid encoding a HMD pathway enzyme, where the microbial organism produces a HMD pathway intermediate, with less byproduct than a cell without the one or more genetic mutations described herein, where the intermediate for example, is a compound set forth in Table 10 or Table 11.

Likewise, in addition to a microbial organism described herein containing a LVA, CPO, or HDO pathway that produces LVA, CPO, or HDO respectively or an intermediate therein (e.g. 6ACA, ADA, CPL, LVA) with less byproduct than a cell without the one or more genetic mutations described herein, the invention additionally provides a non-naturally occurring microbial organism that includes one or more genetic modifications described herein that reduce at least one byproduct described herein (e.g. Table 10) and at least 2, 3, 4, 5, 6 or all exogenous nucleic acids encoding LVA, 6ACA, CPL, CPO, ADA, or HDO pathway enzymes respectively, where the microbial organism produces a LVA, 6ACA, CPL, CPO, ADA, or HDO pathway intermediate respectively, with less byproduct than a cell without the one or more genetic mutations described herein, where the intermediate for example, is a compound set forth in one of Table 10 or 11.

Accordingly, microorganisms having the pathways as described above for production of a pathway intermediate (e.g. a HMD, LVA, 6ACA, CPL, CPO, ADA, or HDO pathway intermediate) can produce such intermediates with less byproducts than a cell lacking the equivalent one or more genetic modifications.

It is understood that any of the pathways disclosed herein, as described in the Examples and exemplified in the Figures including the pathways of FIG. 1, FIG. 2, FIG. 3, FIG. 4, and FIG. 5, can be utilized to generate a non-naturally occurring microbial organism that produces any pathway intermediate or product, as desired and that such non-naturally occurring microbial organisms include one or more genetic modifications described herein which reduces byproducts in the respective pathway. As disclosed herein, such a microbial organism that produces an intermediate can be used in combination with another microbial organism expressing downstream pathway enzymes to produce a desired product. One such example as set forth herein is using a HMD pathway provided herein to biosynthesize intermediates for use in the HDO pathway as shown in FIG. 4. However, it is understood that a non-naturally occurring microbial organism that produces a pathway intermediate as described above can be utilized to produce the intermediate as a desired product.

The invention is described herein with general reference to the metabolic reaction, intermediates or target product thereof, or with specific reference to one or more nucleic acids or genes encoding an enzyme associated with or catalyzing, or a protein associated with, the referenced metabolic reaction, intermediate, target product or byproduct. Unless otherwise expressly stated herein, those skilled in the art will understand that reference to a reaction also constitutes reference to the reactants, intermediates and products of the reaction. Similarly, unless otherwise expressly stated herein, reference to a reactant, intermediate, target product or byproduct also references the reaction, and reference to any of these metabolic constituents also references the gene or genes encoding the enzymes that catalyze or proteins involved in the referenced reaction, reactant, intermediate, target product or byproduct. Likewise, given the well known fields of metabolic biochemistry, enzymology and genomics, reference herein to a gene or encoding nucleic acid also constitutes a reference to the corresponding gene alias, encoded enzyme and the reaction such an enzyme catalyzes or a protein associated with the reaction as well as the reactants, intermediates, target products and byproducts of the reaction.

The non-naturally occurring microbial organisms described herein can be produced by introducing genetic modifications described herein using technology known by those of skill in the art and disclosed herein to reduce activity of enzymes described herein and associated with production of byproducts in the biosynthesis of target products described herein. Further, non-naturally occurring microorganisms described herein can be produced by introducing expressible nucleic acids encoding one or more of the enzymes or proteins participating in one or more biosynthetic pathways described herein (e.g. a HMD, LVA, or HDO pathway). Depending on the host microbial organism chosen for biosynthesis, and the intended biosynthesized target product (e.g., HMD, LVA, 6ACA, CPL, CPO, ADA, or HDO), nucleic acids for some or all of a particular biosynthetic pathway described herein can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve HMD, LVA, 6ACA, CPL, CPO, ADA, or HDO biosynthesis. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as HMD, LVA, 6ACA, CPL, CPO, ADA, or HDO.

Host microbial organisms can be selected from, and the non-naturally occurring microbial organisms generated in, for example, bacteria, yeast, fungus or any of a variety of other microorganisms applicable or suitable to fermentation processes. Exemplary bacteria include any species selected from the order Enterobacteriales, family Enterobacteriaceae, including the genera Escherichia and Klebsiella; the order Aeromonadales, family Succinivibrionaceae, including the genus Anaerobiospirillum; the order Pasteurellales, family Pasteurellaceae, including the genera Actinobacillus and Mannheimia; the order Rhizobiales, family Bradyrhizobiaceae, including the genus Rhizobium; the order Bacillales, family Bacillaceae, including the genus Bacillus; the order Actinomycetales, families Corynebacteriaceae and Streptomycetaceae, including the genus Corynebacterium and the genus Streptomyces, respectively; order Rhodospirillales, family Acetobacteraceae, including the genus Gluconobacter; the order Sphingomonadales, family Sphingomonadaceae, including the genus Zymomonas; the order Lactobacillales, families Lactobacillaceae and Streptococcaceae, including the genus Lactobacillus and the genus Lactococcus, respectively; the order Clostridiales, family Clostridiaceae, genus Clostridium; and the order Pseudomonadales, family Pseudomonadaceae, including the genus Pseudomonas. Non-limiting species of host bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Bacillus methanolicus, Methylobacterium extorquens, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, Streptomyces coelicolor, and Pseudomonas putida.

Similarly, exemplary species of yeast or fungi species include any species selected from the order Saccharomycetales, family Saccaromycetaceae, including the genera Saccharomyces, Kluyveromyces and Pichia; the order Saccharomycetales, family Dipodascaceae, including the genus Yarrowia; the order Schizosaccharomycetales, family Schizosaccaromycetaceae, including the genus Schizosaccharomyces; the order Eurotiales, family Trichocomaceae, including the genus Aspergillus; and the order Mucorales, family Mucoraceae, including the genus Rhizopus. Non-limiting species of host yeast or fungi include Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Depending on the chosen biosynthetic pathway (e.g. a HMD, LVA, 6ACA, CPL, CPO, ADA, or HDO pathway) constituents of a selected host microbial organism, the non-naturally occurring microbial organisms of the invention will include at least one exogenously expressed pathway-encoding nucleic acid (e.g. a nucleic acid encoding an enzyme in a HMD, LVA, 6ACA, CPL, CPO, ADA, or HDO pathway) and up to all encoding nucleic acids for one or more biosynthetic pathways. For example, HMD biosynthesis can be established in a host deficient in a pathway enzyme or protein through exogenous expression of the corresponding encoding nucleic acid. In a host deficient in all enzymes or proteins of a HMD pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of HMD can be included, such as those set forth in Tables 3-6. Enzymes useful in a biosynthetic pathway for production of HDO can include those set forth in Tables 3 and 4 as well as in FIG. 4.

Biosynthesis of other target products described herein (e.g., HMD, LVA, 6ACA, CPL, CPO, ADA, or HDO) can be established in a similar manner and can include the respective enzymes as shown in FIG. 1, FIG. 2, and FIG. 5. Moreover, deepening on the biosynthetic pathway and target product, selected host microorganisms, the non-naturally occurring microorganism of the invention will include one or more genetic modifications described herein. In embodiments, the non-naturally occurring microorganism contains 1, 2, 3, 4, or more, including all combinations set forth in Tables 1 and 2 of genetic modifications described herein of enzymes A1-A25 and B1-B5. In hosts deficient in any one or more of enzymes A1-A25 and B1-B5 genetic modifications described herein may be unnecessary to reduce select byproducts of Table 12. One skilled in the art would understand that genetic modifications described herein of paralogs, homologs, and orthologs of enzymes described herein (e.g. A1-A25 and B1-B5) can be completed to reduce or eliminate byproducts produced from a biosynthetic pathway described herein.

Given the teachings and guidance provided herein, those skilled in the art will understand that the number of encoding nucleic acids to introduce in an expressible form will, at least, parallel the pathway to produce the desired target product (e.g. HMD, LVA, 6ACA, CPL, CPO, ADA, or HDO) pathway deficiencies of the selected host microbial organism. Therefore, a non-naturally occurring microbial organism of the invention can have one, two, three, four, or more or all nucleic acids encoding the enzymes or proteins constituting a biosynthetic pathway disclosed herein. Thus, for example, a non-naturally occurring microbial organism for biosynthesis of HMD can include 1, 2, 3, 4, 5, or more or all the nucleic acids encoding the enzymes that constituted a HMD pathway described herein.

A non-naturally occurring microbial organism for biosynthesis of HMD (including 6ACA, ADA, CPL and intermediates described herein) can include 1, 2, 3, 4, 5, or more or all nucleic acids encoding the enzymes that constituted a HMD pathway described herein. A non-naturally occurring microbial organism for biosynthesis of LVA can include 1, 2, 3, 4, 5, or more or all nucleic acids encoding the enzymes that constituted a LVA pathway described herein. A non-naturally occurring microbial organism for biosynthesis of CPO can include 1, 2, 3, 4, 5, or more or all nucleic acids encoding the enzymes that constituted a CPO pathway described herein. A non-naturally occurring microbial organism for biosynthesis of HDO can include 1, 2, 3, 4, 5, or more or all nucleic acids encoding the enzymes that constituted a HDO pathway described herein.

In some embodiments, the non-naturally occurring microbial organisms also can include other genetic modifications that facilitate or optimize HMD, LVA, 6ACA, CPL, CPO, ADA, or HDO biosynthesis or that confer other useful functions onto the host microbial organism. One such other functionality can include, for example, augmentation (e.g. attenuation) of the synthesis of one or more central metabolic byproducts such as those set forth in Table 14. In a similar manner, one skilled in the art would understand that the number of genetic modifications to reduce or eliminate specific byproducts from a biosynthetic pathway described herein is dependent in part upon the relationship of the byproduct of the given pathway. Thus, as shown in Table 3, an enzyme catalyzing reduction of a byproduct described herein may be 1, 2, 3, 4, 5, 6, or more steps from a given pathway intermediate or target product. Thus, one skilled in the art would understand in such instances a non-naturally occurring microorganism can contain at least 1, 2, 3, 4, 5, 6, or more or all genetic modifications of A1-A25 and B1-B5 as described herein to reduce byproducts in a target product.

In some embodiments, a non-naturally occurring microbial organism of the invention is generated from a host that contains the enzymatic capability to synthesize target product. In this specific embodiment it can be useful to increase the synthesis or accumulation of a target product to, for example, drive target product pathway reactions toward target product production. Increased synthesis or accumulation can be accomplished by, for example, overexpression of nucleic acids encoding one or more of the above-described target product pathway enzymes or proteins. Overexpression of the enzyme or enzymes and/or protein or proteins of the target product pathway can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing target product, through overexpression of one, two, three, four, or more, or all nucleic acids encoding target product biosynthetic pathway enzymes or proteins. In addition, a non-naturally occurring organism can be generated by mutagenesis of an endogenous gene that results in an increase in activity of an enzyme in the target product biosynthetic pathway.

In some embodiments, a non-naturally occurring microbial organism of the invention is generated from a host that contains the enzymatic capability to reduce levels of one or more byproducts described herein. In such embodiments it can be useful to decrease the synthesis or accumulation of a particular byproduct to, for example, by reducing its synthesis or synthesis of an intermediate compound which can be derived to the byproduct. Such reduction can be accomplished by, for example, deletion of genes encoding enzymes catalyzing such reactions. Alternatively, as in the instance of reducing byproduct levels where increased expression of an enzyme is desirable (e.g. B1-B5) overexpression of nucleic acids encoding one or more of enzymes or proteins can be completed. Overexpression of the enzyme or enzymes and/or protein or proteins can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing target product, having reduced levels of byproducts by overexpression of one, two, three, four, or more, or all nucleic acids encoding enzymes or proteins useful for reducing byproduct levels. In addition, a non-naturally occurring organism can be generated by mutagenesis of an endogenous gene that results in an decrease in activity of an enzyme catalyzing byproduct formation in a biosynthetic pathway described herein.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods described herein, any of the one or more exogenous nucleic acids can be introduced into a microbial organism to produce a non-naturally occurring microbial organism of the invention. The nucleic acids can be introduced so as to confer, for example, a HMD, LVA, 6ACA, CPL, CPO, ADA, or HDO biosynthetic pathway onto the microbial organism. Alternatively, encoding nucleic acids can be introduced to produce an intermediate microbial organism having the biosynthetic capability to catalyze some of the required reactions to confer HMD, LVA, 6ACA, CPL, CPO, ADA, or HDO biosynthetic capability. For example, a non-naturally occurring microbial organism having a HMD, LVA, 6ACA, CPL, CPO, ADA, or HDO biosynthetic pathway described herein can includes at least two exogenous nucleic acids encoding desired enzymes or proteins, such as the combination of enzymes set forth in FIG. 1, FIG. 2, FIG. 3, FIG. 4, FIG. 5, or Tables 3 or 4. Thus, it is understood that any combination of two or more enzymes or proteins of a biosynthetic pathway can be included in a non-naturally occurring microbial organism of the invention. Similarly, it is understood that any combination of three or more enzymes or proteins of a biosynthetic pathway can be included in a non-naturally occurring microbial organism of the invention, for example, enzymes set forth in FIG. 1, FIG. 2, FIG. 3, FIG. 4, FIG. 5, or Tables 3 or 4, and so forth, as desired, so long as the combination of enzymes and/or proteins of the desired biosynthetic pathway results in production of the corresponding desired product (e.g. HMD, LVA, 6ACA, CPL, CPO, ADA, or HDO). Similarly, any combination of four, or more enzymes or proteins of a biosynthetic pathway as disclosed herein can be included in a non-naturally occurring microbial organism of the invention, as desired, so long as the combination of enzymes and/or proteins of the desired biosynthetic pathway results in production of the corresponding desired product.

It is further understood that, in methods described herein, any of the one or more genetic modifications described herein can be introduced into a microbial organism to produce a non-naturally occurring microbial organism of the invention which biosynthesizes a target product described herein with reduced levels of byproduct. The genetic modifications can be introduced so as to confer, reduced production of a byproduct described herein in a biosynthetic pathway described herein. For example, a non-naturally occurring microbial organism having a HMD, LVA, 6ACA, CPL, CPO, ADA, or HDO biosynthetic pathway described herein can includes at least two genetic modifications described herein, such that the combination reduces a byproduct described herein. Thus, it is understood that any combination of two or more genetic modifications can be included in a non-naturally occurring microbial organism of the invention. Similarly, it is understood that any combination of three or more genetic modifications can be included in a non-naturally occurring microbial organism of the invention, for example, gene modification of an enzyme set forth in Table 3 or 4, and so forth, as desired, so long as the combination of genetic modifications results in production of the corresponding desired product (e.g. HMD, LVA, 6ACA, CPL, CPO, ADA, or HDO) with reduced levels of byproducts described herein. Similarly, any combination of four, or more genetic modifications as disclosed herein can be included in a non-naturally occurring microbial organism of the invention, as desired, so long as the combination of genetic modifications results in production of the corresponding desired target product with reduced levels of byproduct.

In addition to the biosynthesis of target product as described herein, the non-naturally occurring microbial organisms and methods of the invention also can be utilized in various combinations with each other and/or with other microbial organisms and methods well known in the art to achieve product biosynthesis by other routes. For example, one alternative to produce HDO other than use of a HDO pathway in a cell described herein is through addition of another microbial organism capable of converting a HDO product pathway intermediate to HDO. One such procedure includes, for example, the fermentation of a microbial organism that produces a target product pathway intermediate. The target product pathway intermediate can then be used as a substrate for a second microbial organism that converts the target product pathway intermediate to target product. The target product pathway intermediate can be added directly to another culture of the second organism or the original culture of the target product pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods of the invention can be assembled in a wide variety of subpathways to achieve biosynthesis of, for example, HDO, HMD, CPO, LVA, CPL, ADA, 6ACA or an intermediate of such pathways as described herein. In such embodiments, biosynthetic pathways for a desired product of the invention can be segregated into different microbial organisms, and the different microbial organisms where each microbial organism can separately contain one or more genetic modifications described herein that reduce levels of byproducts produced in biosynthetic pathways in such a cell. The different microbial organisms can be co-cultured to produce the final product. In such a biosynthetic scheme, the product of one microbial organism is the substrate for a second microbial organism until the final product is synthesized having reduced levels of byproducts described herein. For example, the biosynthesis of target product can be accomplished by constructing a microbial organism that contains biosynthetic pathways for conversion of one pathway intermediate to another pathway intermediate or the product. Alternatively, target product also can be biosynthetically produced from microbial organisms through co-culture or co-fermentation using two organisms in the same vessel, where the first microbial organism produces an intermediate of a biosynthetic pathway described herein and the second microbial organism converts the intermediate to target product.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce target product.

Similarly, it is understood by those skilled in the art that a host organism can be selected based on desired characteristics for introduction of one or more gene disruptions to increase production of target product. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods of the invention can be applied to any suitable host microorganism to identify the cognate metabolic alterations needed to construct an organism in a species of interest that will increase target product biosynthesis. In a particular embodiment, the increased production couples biosynthesis of target product to growth of the organism, and can obligatorily couple production of target product to growth of the organism if desired and as disclosed herein.

Similarly, it is understood by those skilled in the art that a host organism can be selected based on desired characteristics for introduction of one or more genetic modifications described herein which reduce levels of byproducts described herein. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired enzymatic reaction leading to a byproduct is also sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods of the invention can be applied to any suitable host microorganism to identify the cognate metabolic alterations needed to construct an organism in a species of interest that will reduce production of byproducts in a given target product biosynthetic pathway described herein.

Sources of encoding nucleic acids for a HMD, LVA, 6ACA, CPL, CPO, ADA, or HDO pathway enzyme can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, those species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite biosynthetic activity for producing a target product described herein (e.g. HMD, LVA, 6ACA, CPL, CPO, ADA, or HDO) for one or more genes in related or distant species, including for example, homologues, orthologs, paralogs and nonorthologous gene displacements of known genes, and the interchange of genetic alterations between organisms is well known in the art. Accordingly, the genetic modifications described herein which decrease levels of byproducts such as those of Table 12 in the biosynthesis of HMD, LVA, 6ACA, CPL, CPO, ADA, or HDO as described herein with reference to a particular organism such as, for example, E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative biosynthetic pathway exists for production of a target product described herein (e.g. HMD, LVA, 6ACA, CPL, CPO, ADA, or HDO) in an unrelated species, HMD, LVA, 6ACA, CPL, CPO, ADA, or HDO biosynthesis can be conferred onto the host species by, for example, exogenous expression of a paralog or paralogs from the unrelated species that catalyzes a similar, yet non-identical metabolic reaction to replace the referenced reaction. Similarly, in such instances genetic modifications of enzymes such as A1-A25 and B1-B5 may vary between species. One skilled in the art using the cells and methods described herein can readily identify paralogs, homologs, and orthologs of enzymes useful for genetic modification as described herein.

Because certain differences among metabolic networks exist between different organisms, those skilled in the art will understand that the actual gene usage between different organisms may differ. This gene usage is applicable to both the enzymes constituting the biosynthetic pathways described herein and to enzymes useful for reducing byproducts as described herein. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize HMD, LVA, 6ACA, CPL, CPO, ADA, or HDO.

A nucleic acid molecule encoding a target product pathway enzyme or protein of the invention can also include a nucleic acid molecule that hybridizes to a nucleic acid disclosed herein by SEQ ID NO, GenBank and/or GI number or a nucleic acid molecule that hybridizes to a nucleic acid molecule that encodes an amino acid sequence disclosed herein by SEQ ID NO, GenBank and/or GI number. Hybridization conditions can include highly stringent, moderately stringent, or low stringency hybridization conditions that are well known to one of skill in the art such as those described herein. Similarly, a nucleic acid molecule that can be used in the invention can be described as having a certain percent sequence identity to a nucleic acid disclosed herein by SEQ ID NO, GenBank and/or GI number or a nucleic acid molecule that hybridizes to a nucleic acid molecule that encodes an amino acid sequence disclosed herein by SEQ ID NO, GenBank and/or GI number. For example, the nucleic acid molecule can have at least 65%, 70%, 75%, 80%, 85%, 90%, 91%, 92%, 93%, 94%, 95%, 96%, 97%, 98% or 99% sequence identity, or be identical, to a nucleic acid described herein.

Stringent hybridization refers to conditions under which hybridized polynucleotides are stable. As known to those of skill in the art, the stability of hybridized polynucleotides is reflected in the melting temperature (Tₘ) of the hybrids. In general, the stability of hybridized polynucleotides is a function of the salt concentration, for example, the sodium ion concentration and temperature. A hybridization reaction can be performed under conditions of lower stringency, followed by washes of varying, but higher, stringency. Reference to hybridization stringency relates to such washing conditions. Highly stringent hybridization includes conditions that permit hybridization of only those nucleic acid sequences that form stable hybridized polynucleotides in 0.018 M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C. Hybridization conditions other than highly stringent hybridization conditions can also be used to describe the nucleic acid sequences disclosed herein. For example, the phrase moderately stringent hybridization refers to conditions equivalent to hybridization in 50% formamide, 5× Denhart's solution, 5×SSPE, 0.2% SDS at 42° C., followed by washing in 0.2×SSPE, 0.2% SDS, at 42° C. The phrase low stringency hybridization refers to conditions equivalent to hybridization in 10% formamide, 5× Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable low, moderate and high stringency hybridization buffers and conditions are well known to those of skill in the art and are described, for example, in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

A nucleic acid molecule encoding a HMD, LVA, 6ACA, CPL, CPO, ADA, or HDO pathway enzyme described herein can have at least a certain sequence identity to a nucleotide sequence disclosed herein. According, in some aspects of the invention, a nucleic acid molecule encoding a HMD, LVA, 6ACA, CPL, CPO, ADA, or HDO pathway enzyme described herein has a nucleotide sequence of at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid disclosed herein by SEQ ID NO, GenBank and/or GI number or a nucleic acid molecule that hybridizes to a nucleic acid molecule that encodes an amino acid sequence disclosed herein by SEQ ID NO, GenBank and/or GI number.

Sequence identity (also known as homology or similarity) refers to sequence similarity between two nucleic acid molecules or between two polypeptides. Identity can be determined by comparing a position in each sequence, which may be aligned for purposes of comparison. When a position in the compared sequence is occupied by the same base or amino acid, then the molecules are identical at that position. A degree of identity between sequences is a function of the number of matching or homologous positions shared by the sequences. The alignment of two sequences to determine their percent sequence identity can be done using software programs known in the art, such as, for example, those described in Ausubel et al., Current Protocols in Molecular Biology, John Wiley and Sons, Baltimore, Md. (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

It is understood that a nucleic acid described herein can exclude a wild type parental sequence. One skilled in the art will readily understand the meaning of a parental wild type sequence based on what is well known in the art. It is further understood that such a nucleic acid can exclude a naturally occurring amino acid sequence as found in nature. Thus, in a particular embodiment, the nucleic acid of the invention is as set forth above and herein, with the proviso that the encoded amino acid sequence is not the wild type parental sequence or a naturally occurring amino acid sequence and/or that the nucleic acid sequence is not a wild type or naturally occurring nucleic acid sequence. A naturally occurring amino acid or nucleic acid sequence is understood by those skilled in the art as relating to a sequence that is found in a naturally occurring organism. Thus, a nucleic acid or amino acid sequence that is not found in the same state or having the same nucleotide or encoded amino acid sequence as in a naturally occurring organism is included within the meaning of a nucleic acid and/or amino acid sequence of the invention. For example, a nucleic acid or amino acid sequence that has been altered at one or more nucleotide or amino acid positions from a parent sequence, including variants as described herein, are included within the meaning of a nucleic acid or amino acid sequence of the invention that is not naturally occurring. An isolated nucleic acid molecule of the invention excludes a naturally occurring chromosome that contains the nucleic acid sequence, and can further exclude other molecules as found in a naturally occurring cell such as DNA binding proteins, for example, proteins such as histones that bind to chromosomes with a eukaryotic cell.

Thus, an isolated nucleic acid sequence of the invention has physical and chemical differences compared to a naturally occurring nucleic acid sequence. An isolated or non-naturally occurring nucleic acid of the invention does not contain or does not necessarily have some or all of the chemical bonds, either covalent or non-covalent bonds, of a naturally occurring nucleic acid sequence as found in nature. An isolated nucleic acid of the invention thus differs from a naturally occurring nucleic acid, for example, by having a different chemical structure than a naturally occurring nucleic acid sequence as found in a chromosome. A different chemical structure can occur, for example, by cleavage of phosphodiester bonds that release an isolated nucleic acid sequence from a naturally occurring chromosome. An isolated nucleic acid of the invention can also differ from a naturally occurring nucleic acid by isolating or separating the nucleic acid from proteins that bind to chromosomal DNA in either prokaryotic or eukaryotic cells, thereby differing from a naturally occurring nucleic acid by different non-covalent bonds. With respect to nucleic acids of prokaryotic origin, a non-naturally occurring nucleic acid of the invention does not necessarily have some or all of the naturally occurring chemical bonds of a chromosome, for example, binding to DNA binding proteins such as polymerases or chromosome structural proteins, or is not in a higher order structure such as being supercoiled. With respect to nucleic acids of eukarytoic origin, a non-naturally occurring nucleic acid of the invention also does not contain the same internal nucleic acid chemical bonds or chemical bonds with structural proteins as found in chromatin. For example, a non-naturally occurring nucleic acid of the invention is not chemically bonded to histones or scaffold proteins and is not contained in a centromere or telomere. Thus, the non-naturally occurring nucleic acids of the invention are chemically distinct from a naturally occurring nucleic acid because they either lack or contain different van der Waals interactions, hydrogen bonds, ionic or electrostatic bonds, and/or covalent bonds from a nucleic acid as found in nature. Such differences in bonds can occur either internally within separate regions of the nucleic acid (that is cis) or such difference in bonds can occur in trans, for example, interactions with chromosomal proteins. In the case of a nucleic acid of eukaryotic origin, a cDNA is considered to be an isolated or non-naturally occurring nucleic acid since the chemical bonds within a cDNA differ from the covalent bonds that is the sequence, of a gene on chromosomal DNA. Thus, it is understood by those skilled in the art that an isolated or non-naturally occurring nucleic acid is distinct from a naturally occurring nucleic acid.

In some embodiments, the invention provides an isolated polypeptide having an amino acid sequence disclosed herein, where the amino acid sequence has at least 65%, 70%, 75%, 80%, 85%, 90%, 91%, 92%, 93%, 94%, 95%, 96%, 97%, 98% or 99% sequence identity, or is identical, to an amino acid sequence or GI number set forth in Tables 3-7. It is understood that a variant amino acid position can include any one of the 20 naturally occurring amino acids, a conservative substitution of a wild type or parental sequence at the corresponding position of the variant amino acid position, or a specific amino acid at the variant amino acid position. It is further understood that any of the variant amino acid positions can be combined to generate further variants. Variants with combinations of two or more variant amino acid positions can exhibit activities greater than wild type. Alternatively, combinations of two or more variant amino acid positions can decrease or nullify enzyme activity. One skilled in the art can readily generate polypeptides with single variant positions or combinations of variant positions using methods well known to those skilled in the art to generate polypeptides with desired properties, including increased enzyme activity and/or stability or loss of enzyme activity as described herein. One skilled in the art would also readily understand and identify conserved regions and invariable regions of which would be expected to have significant effect on enzyme activity. Such identification can be performed using sequence alignments as is well known in the art.

“Homology” or “identity” or “similarity” refers to sequence similarity between two polypeptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

A polypeptide or polypeptide region (or a polynucleotide or polynucleotide region) has a certain percentage (for example, 65%, 70%, 75%, 80%, 85%, 90%, 95%, 98% or 99%) of “sequence identity” to another sequence means that, when aligned, that percentage of amino acids (or nucleotide bases) are the same in comparing the two sequences. Sequence identity (also known as homology or similarity) refers to sequence similarity between two nucleic acid molecules or between two polypeptides. Identity can be determined by comparing a position in each sequence, which may be aligned for purposes of comparison. When a position in the compared sequence is occupied by the same base or amino acid, then the molecules are identical at that position. A degree of identity between sequences is a function of the number of matching or homologous positions shared by the sequences. This alignment and the percent homology or sequence identity can be determined using software programs known in the art, for example those described in Ausubel et al., supra. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information (NCBI).

It is understood that the variant polypeptides such as polypeptide variants of enzymes set forth in Tables 3 or 4 are designed in the case of enzymes A1-A25 to nullify activity or function. Polypeptide variants of enzymes useful in biosynthetic pathways described herein can include variants that provide a beneficial characteristic to the polypeptide, including but not limited to, improved catalytic activity, increased catalytic, turnover, increased substrate affinity, decreased product inhibition, and/or protein or enzyme stability. In a particular embodiment, such variants can have improved characteristics of stability while exhibiting similar activity to a wild type or parent polypeptide. In another particular embodiment, such enzyme variants can exhibit an activity that is at least the same or higher than a wild type or parent polypeptide, for example, 1.2, 1.5, 2, 2.5, 3, 3.5, 4, 4.5, 5, 5.5, 6, 6.5, 7, 7.5, 8, 8.5, 9, 9.5, 10, or even higher fold activity of the variant polypeptide over a wild type or parent polypeptide. Alternatively, polypeptide variants can include variants designed to decrease or nullify enzyme activity.

Methods for constructing and testing the expression levels of a non-naturally occurring microbial host capable of producing HMD, LVA, 6ACA, CPL, CPO, ADA, or HDO with less byproduct than a cell lacking one or more genetic modifications described herein can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of HMD, LVA, 6ACA, CPL, CPO, ADA, or HDO can be introduced stably or transiently into a host cell using techniques well known in the art including, but not limited to, conjugation, electroporation, chemical transformation, transduction, transfection, and ultrasound transformation. Likewise, the genetic modifications described herein can be introduced stably or transiently into a host cell using techniques well known in the art. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more biosynthetic pathway encoding nucleic acids as described herein operably linked to expression control sequences functional in the host organism. Such an expression vector is therefore capable of producing polypeptides described herein in a biosynthetic pathway for producing HMD, LVA, 6ACA, CPL, CPO, ADA, or HDO with less byproduct than a cell without one or more genetic modifications described herein. Expression vectors can also include nucleic acid encoding sequences for enzymes useful for reducing byproducts described herein. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

Suitable purification and/or assays to test for the production of HMD, LVA, 6ACA, CPL, CPO, ADA, or HDO can be performed using well known methods. Suitable replicates such as triplicate cultures can be grown for each engineered strain to be tested. For example, product and byproduct formation in the engineered production host can be monitored. The final product and intermediates, levels of byproducts described herein and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The target product (e.g. HMD, LVA, 6ACA, CPL, CPO, ADA, or HDO) can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. In certain instances target products described herein can be isolated using distillation. All of the above methods are well known in the art.

The target product can be purified by distillation, crystallization, ion exchange chromatography, and adsorption chromatography. In certain instances, target products described herein can be purified using distillation or crystallization. Such methods are well known in the art.

Any of the non-naturally occurring microbial organisms described herein can be cultured to produce and/or secrete the biosynthetic target products having reduced levels of byproducts described herein of the invention. For example, non-naturally occurring microbial organisms capable of producing HMD, LVA, 6ACA, CPL, CPO, ADA, or HDO can be cultured for the biosynthetic production of HMD, LVA, 6ACA, CPL, CPO, ADA, or HDO respectively. Accordingly, in some embodiments, the invention provides culture medium or fermentation broth containing HMD, LVA, 6ACA, CPL, CPO, ADA, or HDO or a HMD, LVA, 6ACA, CPL, CPO, ADA, or HDO pathway intermediate described herein, where the culture medium or fermentation broth includes less byproduct than a cell lacking one or more genetic modifications described herein. In some aspects, the culture medium can also be separated from the non-naturally occurring microbial organisms of the invention that produced target product or the pathway intermediate. Thus provided herein is a culture medium as described above where cells have been removed. Methods for separating a microbial organism from culture medium are well known in the art. Exemplary methods include filtration, flocculation, precipitation, centrifugation, sedimentation, distillation and the like.

For the production of a target product described herein, the recombinant strains of microbial organisms described herein are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by, for example, perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high target product yields with reduced levels of byproducts when compared to a cell lacking the genetic modifications.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microbial organism of the invention. Such sources include, for example, sugars such as glucose, xylose, arabinose, galactose, mannose, fructose, sucrose and starch; or glycerol, alone as the sole source of carbon or in combination with other carbon sources described herein or known in the art. In one embodiment, H₂, CO, CO₂ or any combination thereof can be supplied as the sole or supplemental feedstock to the other sources of carbon disclosed herein. In one embodiment, the carbon source is a sugar. In one embodiment, the carbon source is a sugar-containing biomass. In some embodiments, the sugar is glucose. In one embodiment, the sugar is xylose. In another embodiment, the sugar is arabinose. In one embodiment, the sugar is galactose. In another embodiment, the sugar is fructose. In other embodiments, the sugar is sucrose. In one embodiment, the sugar is starch. In certain embodiments, the carbon source is glycerol. In some embodiments, the carbon source is crude glycerol. In one embodiment, the carbon source is crude glycerol without treatment.

In other embodiments, the carbon source is glycerol and glucose. In another embodiment, the carbon source is methanol and glycerol. In one embodiment, the carbon source is carbon dioxide. In one embodiment, the carbon source is formate. In one embodiment, the carbon source is methane. In one embodiment, the carbon source is methanol. In one embodiment, the carbon source is chemoelectro-generated carbon (see, e.g., Liao et al. (2012) Science 335:1596). In one embodiment, the chemoelectro-generated carbon is methanol. In one embodiment, the chemoelectro-generated carbon is formate. In one embodiment, the carbon source is a sugar and methanol. In another embodiment, the carbon source is a sugar and glycerol. In other embodiments, the carbon source is a sugar and crude glycerol. In yet other embodiments, the carbon source is a sugar and crude glycerol without treatment. In one embodiment, the carbon source is a sugar-containing biomass and methanol. In another embodiment, the carbon source is a sugar-containing biomass and glycerol. In other embodiments, the carbon source is a sugar-containing biomass and crude glycerol. In other embodiments, the carbon source is a methanol and crude glycerol. In other embodiments, the carbon source is a methanol and glycerol. In yet other embodiments, the carbon source is a sugar-containing biomass and crude glycerol without treatment.

Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms provided herein for the production of HMD, 6ACA, ADA, CPL, CPO, LVA, or HDO, including intermediates in biosynthetic pathways described herein used to produce target products described herein.

In one embodiment, the carbon source is glycerol. In certain embodiments, the glycerol carbon source is crude glycerol or crude glycerol without further treatment. In a further embodiment, the carbon source can include glycerol or crude glycerol, and also sugar or a sugar-containing biomass, such as glucose. In a specific embodiment, the concentration of glycerol in the fermentation broth is maintained by feeding crude glycerol, or a mixture of crude glycerol and sugar (e.g., glucose). In certain embodiments, sugar is provided for sufficient strain growth. In some embodiments, the sugar (e.g., glucose) is provided at a molar concentration ratio of glycerol to sugar of from 200:1 to 1:200. In some embodiments, the sugar (e.g., glucose) is provided at a molar concentration ratio of glycerol to sugar of from 100:1 to 1:100. In some embodiments, the sugar (e.g., glucose) is provided at a molar concentration ratio of glycerol to sugar of from 100:1 to 5:1. In some embodiments, the sugar (e.g., glucose) is provided at a molar concentration ratio of glycerol to sugar of from 50:1 to 5:1.

In certain embodiments, the sugar (e.g., glucose) is provided at a molar concentration ratio of glycerol to sugar of 100:1. In one embodiment, the sugar (e.g., glucose) is provided at a molar concentration ratio of glycerol to sugar of 90:1. In one embodiment, the sugar (e.g., glucose) is provided at a molar concentration ratio of glycerol to sugar of 80:1. In one embodiment, the sugar (e.g., glucose) is provided at a molar concentration ratio of glycerol to sugar of 70:1. In one embodiment, the sugar (e.g., glucose) is provided at a molar concentration ratio of glycerol to sugar of 60:1. In one embodiment, the sugar (e.g., glucose) is provided at a molar concentration ratio of glycerol to sugar of 50:1. In one embodiment, the sugar (e.g., glucose) is provided at a molar concentration ratio of glycerol to sugar of 40:1. In one embodiment, the sugar (e.g., glucose) is provided at a molar concentration ratio of glycerol to sugar of 30:1. In one embodiment, the sugar (e.g., glucose) is provided at a molar concentration ratio of glycerol to sugar of 20:1. In one embodiment, the sugar (e.g., glucose) is provided at a molar concentration ratio of glycerol to sugar of 10:1. In one embodiment, the sugar (e.g., glucose) is provided at a molar concentration ratio of glycerol to sugar of 5:1. In one embodiment, the sugar (e.g., glucose) is provided at a molar concentration ratio of glycerol to sugar of 2:1. In one embodiment, the sugar (e.g., glucose) is provided at a molar concentration ratio of glycerol to sugar of 1:1.

In certain embodiments, the sugar (e.g., glucose) is provided at a molar concentration ratio of glycerol to sugar of 1:100. In one embodiment, the sugar (e.g., glucose) is provided at a molar concentration ratio of glycerol to sugar of 1:90. In one embodiment, the sugar (e.g., glucose) is provided at a molar concentration ratio of glycerol to sugar of 1:80. In one embodiment, the sugar (e.g., glucose) is provided at a molar concentration ratio of glycerol to sugar of 1:70. In one embodiment, the sugar (e.g., glucose) is provided at a molar concentration ratio of glycerol to sugar of 1:60. In one embodiment, the sugar (e.g., glucose) is provided at a molar concentration ratio of glycerol to sugar of 1:50. In one embodiment, the sugar (e.g., glucose) is provided at a molar concentration ratio of glycerol to sugar of 1:40. In one embodiment, the sugar (e.g., glucose) is provided at a molar concentration ratio of glycerol to sugar of 1:30. In one embodiment, the sugar (e.g., glucose) is provided at a molar concentration ratio of glycerol to sugar of 1:20. In one embodiment, the sugar (e.g., glucose) is provided at a molar concentration ratio of glycerol to sugar of 1:10. In one embodiment, the sugar (e.g., glucose) is provided at a molar concentration ratio of glycerol to sugar of 1:5. In one embodiment, the sugar (e.g., glucose) is provided at a molar concentration ratio of glycerol to sugar of 1:2. In certain embodiments of the ratios provided above, the sugar is a sugar-containing biomass. In certain other embodiments of the ratios provided above, the glycerol is a crude glycerol or a crude glycerol without further treatment. In other embodiments of the ratios provided above, the sugar is a sugar-containing biomass, and the glycerol is a crude glycerol or a crude glycerol without further treatment.

Crude glycerol can be a byproduct produced in the production of biodiesel, and can be used for fermentation without any further treatment. Biodiesel production methods include (1) a chemical method wherein the glycerol-group of vegetable oils or animal oils is substituted by low-carbon alcohols such as methanol or ethanol to produce a corresponding fatty acid methyl esters or fatty acid ethyl esters by transesterification in the presence of acidic or basic catalysts; (2) a biological method where biological enzymes or cells are used to catalyze transesterification reaction and the corresponding fatty acid methyl esters or fatty acid ethyl esters are produced; and (3) a supercritical method, wherein transesterification reaction is carried out in a supercritical solvent system without any catalysts. The chemical composition of crude glycerol can vary with the process used to produce biodiesel, the transesterification efficiency, recovery efficiency of the biodiesel, other impurities in the feedstock, and whether methanol and catalysts were recovered. For example, the chemical compositions of eleven crude glycerol collected from seven Australian biodiesel producers reported that glycerol content ranged between 38% and 96%, with some samples including more than 14% methanol and 29% ash. In certain embodiments, the crude glycerol can include from 5% to 99% glycerol. In some embodiments, the crude glycerol can include from 10% to 90% glycerol. In some embodiments, the crude glycerol can include from 10% to 80% glycerol. In some embodiments, the crude glycerol can include from 10% to 70% glycerol. In some embodiments, the crude glycerol can include from 10% to 60% glycerol. In some embodiments, the crude glycerol can include from 10% to 50% glycerol. In some embodiments, the crude glycerol can include from 10% to 40% glycerol. In some embodiments, the crude glycerol can include from 10% to 30% glycerol. In some embodiments, the crude glycerol can include from 10% to 20% glycerol. In some embodiments, the crude glycerol can include from 80% to 90% glycerol. In some embodiments, the crude glycerol can include from 70% to 90% glycerol. In some embodiments, the crude glycerol can include from 60% to 90% glycerol. In some embodiments, the crude glycerol can include from 50% to 90% glycerol. In some embodiments, the crude glycerol can include from 40% to 90% glycerol. In some embodiments, the crude glycerol can include from 30% to 90% glycerol. In some embodiments, the crude glycerol can include from 20% to 90% glycerol. In some embodiments, the crude glycerol can include from 20% to 40% glycerol. In some embodiments, the crude glycerol can include from 40% to 60% glycerol. In some embodiments, the crude glycerol can include from 60% to 80% glycerol. In some embodiments, the crude glycerol can include from 50% to 70% glycerol.

In one embodiment, the glycerol includes 5% glycerol. In one embodiment, the glycerol includes 10% glycerol. In one embodiment, the glycerol includes 15% glycerol. In one embodiment, the glycerol includes 20% glycerol. In one embodiment, the glycerol includes 25% glycerol. In one embodiment, the glycerol includes 30% glycerol. In one embodiment, the glycerol includes 35% glycerol. In one embodiment, the glycerol includes 40% glycerol. In one embodiment, the glycerol includes 45% glycerol. In one embodiment, the glycerol includes 50% glycerol. In one embodiment, the glycerol includes 55% glycerol. In one embodiment, the glycerol includes 60% glycerol. In one embodiment, the glycerol includes 65% glycerol. In one embodiment, the glycerol includes 70% glycerol. In one embodiment, the glycerol includes 75% glycerol. In one embodiment, the glycerol includes 80% glycerol. In one embodiment, the glycerol includes 85% glycerol. In one embodiment, the glycerol includes 90% glycerol. In one embodiment, the glycerol includes 95% glycerol. In one embodiment, the glycerol includes 99% glycerol.

In certain embodiments, methanol is used as a carbon source in biosynthetic pathways described herein. In certain embodiments, a sugar (e.g. glucose) is used as a carbon source in biosynthetic pathways described herein.

In one embodiment, the carbon source includes methanol, and sugar (e.g., glucose) or a sugar-containing biomass. In specific embodiments, methanol in the fermentation feed is provided as a mixture with sugar (e.g., glucose) or sugar-comprising biomass. In certain embodiments, sugar is provided for sufficient strain growth.

In certain embodiments, the carbon source includes methanol and a sugar (e.g., glucose). In some embodiments, the sugar (e.g., glucose) is provided at a molar concentration ratio of methanol to sugar of from 200:1 to 1:200. In some embodiments, the sugar (e.g., glucose) is provided at a molar concentration ratio of methanol to sugar of from 100:1 to 1:100. In some embodiments, the sugar (e.g., glucose) is provided at a molar concentration ratio of methanol to sugar of from 100:1 to 5:1. In some embodiments, the sugar (e.g., glucose) is provided at a molar concentration ratio of methanol to sugar of from 50:1 to 5:1. In certain embodiments, the sugar (e.g., glucose) is provided at a molar concentration ratio of methanol to sugar of 100:1. In one embodiment, the sugar (e.g., glucose) is provided at a molar concentration ratio of methanol to sugar of 90:1. In one embodiment, the sugar (e.g., glucose) is provided at a molar concentration ratio of methanol to sugar of 80:1. In one embodiment, the sugar (e.g., glucose) is provided at a molar concentration ratio of methanol to sugar of 70:1. In one embodiment, the sugar (e.g., glucose) is provided at a molar concentration ratio of methanol to sugar of 60:1. In one embodiment, the sugar (e.g., glucose) is provided at a molar concentration ratio of methanol to sugar of 50:1. In one embodiment, the sugar (e.g., glucose) is provided at a molar concentration ratio of methanol to sugar of 40:1. In one embodiment, the sugar (e.g., glucose) is provided at a molar concentration ratio of methanol to sugar of 30:1. In one embodiment, the sugar (e.g., glucose) is provided at a molar concentration ratio of methanol to sugar of 20:1. In one embodiment, the sugar (e.g., glucose) is provided at a molar concentration ratio of methanol to sugar of 10:1. In one embodiment, the sugar (e.g., glucose) is provided at a molar concentration ratio of methanol to sugar of 5:1. In one embodiment, the sugar (e.g., glucose) is provided at a molar concentration ratio of methanol to sugar of 2:1. In one embodiment, the sugar (e.g., glucose) is provided at a molar concentration ratio of methanol to sugar of 1:1.

In certain embodiments, the sugar (e.g., glucose) is provided at a molar concentration ratio of methanol to sugar of 1:100. In one embodiment, the sugar (e.g., glucose) is provided at a molar concentration ratio of methanol to sugar of 1:90. In one embodiment, the sugar (e.g., glucose) is provided at a molar concentration ratio of methanol to sugar of 1:80. In one embodiment, the sugar (e.g., glucose) is provided at a molar concentration ratio of methanol to sugar of 1:70. In one embodiment, the sugar (e.g., glucose) is provided at a molar concentration ratio of methanol to sugar of 1:60. In one embodiment, the sugar (e.g., glucose) is provided at a molar concentration ratio of methanol to sugar of 1:50. In one embodiment, the sugar (e.g., glucose) is provided at a molar concentration ratio of methanol to sugar of 1:40. In one embodiment, the sugar (e.g., glucose) is provided at a molar concentration ratio of methanol to sugar of 1:30. In one embodiment, the sugar (e.g., glucose) is provided at a molar concentration ratio of methanol to sugar of 1:20. In one embodiment, the sugar (e.g., glucose) is provided at a molar concentration ratio of methanol to sugar of 1:10. In one embodiment, the sugar (e.g., glucose) is provided at a molar concentration ratio of methanol to sugar of 1:5. In one embodiment, the sugar (e.g., glucose) is provided at a molar concentration ratio of methanol to sugar of 1:2. In certain embodiments of the ratios provided above, the sugar is a sugar-containing biomass.

In addition to renewable feedstocks such as those exemplified above, the non-naturally occurring microorganisms of the invention also can be modified for growth on syngas as its source of carbon. In one example, one or more proteins or enzymes are expressed in the microbial organisms described herein to provide a metabolic pathway for utilization of syngas or other gaseous carbon source to produce HMD, LVA, 6ACA, CPL, CPO, ADA, or HDO.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO₂+4H₂+n ADP+n Pi→CH₃COOH+2H₂O+n ATP

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a target product pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

The non-naturally occurring microbial organisms of the invention are constructed using methods well known in the art as exemplified herein to exogenously express at least one nucleic acid encoding a pathway enzyme as described in sufficient amounts to produce a particular target product (e.g. HMD, LVA, 6ACA, CPL, CPO, ADA, or HDO) having less byproducts than a cell producing the same target product and lacking the genetic modifications described herein. It is understood that the microbial organisms of the invention are cultured under conditions sufficient to produce HMD, LVA, 6ACA, CPL, CPO, ADA, or HDO. Following the teachings and guidance provided herein, the non-naturally occurring microbial organisms of the invention can achieve biosynthesis of a target product described herein resulting in intracellular concentrations between about 0.1-200 mM or more. Generally, the intracellular concentration of target product is between about 3-150 mM, particularly between about 5-125 mM and more particularly between about 8-100 mM, about 1-10 mM, including about 1 mM, 10 mM, 20 mM, 50 mM, 80 mM, or more. Intracellular concentrations between and above each of these exemplary ranges also can be achieved from the non-naturally occurring microbial organisms of the invention.

Target products described herein can be produced by cells described herein with less byproduct than production of such target products in cells lacking the genetic modifications described herein. Further target product described herein can be produced by cells described herein in greater amounts when the cells include one or more genetic modifications double stranded. Target products described herein can be produced in titers of 0.1 g/L to 300 g/L, 0.1 g/L to 250 g/L, 0.1 g/L to 200 g/L, 0.1 g/L to 150 g/L, 0.1 g/L to 120 g/L, 0.1 g/L to 100 g/L, 0.1 g/L to 50 g/L, 0.1 g/L to 25 g/L, 0.1 g/L to 10 g/L, or 0.1 g/L to 5 g/L. Target products described herein can be produced in titers greater than or equal to 0.1 g/L, 0.5 g/L, 1 g/L, 5 g/L, 10 g/L, 20 g/L, 25 g/L, 30 g/L, 40 g/L, 50 g/L, 60 g/L, 70 g/L, 80 g/L, 90 g/L, 100 g/L, 120 g/L, 150 g/L, 175 g/L, 200 g/L, 250 g/L, or 300 g/L. In certain instances a target product described herein is produced in titers of greater than or equal to 120 g/L. In certain instances a target product described herein is produced in titers of greater than or equal to 300 g/L. Thus, provided herein are non-naturally occurring microorganism capable of producing HMD at a titer described herein or a titer of >120 g/L where the HMD is produced by a cell having one or more of the genetic modifications described herein. Provided herein are non-naturally occurring microorganism capable of producing 6ACA at a titer described herein or a titer of >120 g/L where the 6ACA is produced by a cell having one or more of the genetic modifications described herein. Provided herein are non-naturally occurring microorganism capable of producing ADA at a titer described herein or a titer of >120 g/L where the ADA is produced by a cell having one or more of the genetic modifications described herein. Provided herein are non-naturally occurring microorganism capable of producing CPL at a titer described herein or a titer of >120 g/L where the CPL is produced by a cell having one or more of the genetic modifications described herein. Provided herein are non-naturally occurring microorganism capable of producing CPO at a titer described herein or a titer of >120 g/L where the CPO is produced by a cell having one or more of the genetic modifications described herein. Provided herein are non-naturally occurring microorganism capable of producing LVA at a titer described herein or a titer of >120 g/L where the LVA is produced by a cell having one or more of the genetic modifications described herein. Provided herein are non-naturally occurring microorganism capable of producing HDO at a titer described herein or a titer of >120 g/L where the HDO is produced by a cell having one or more of the genetic modifications described herein. Provided herein are non-naturally occurring microorganism capable of producing 6ACA at a titer described herein or a titer of >120 g/L where the 6ACA is produced by a cell having one or more of the genetic modifications described herein.

Target product can also be measured by the theoretical yield. The theoretical yield of a target product described herein is represented by the amount of a carbon feedstock (e.g. a sugar such as glucose, or methanol, or glycerol) used by the cell to biosynthesize the target compound. Theoretical yields for the target products described herein can be readily calculated by those of skill in the art. Target products herein can be produced at an amount of 1, 2, 3, 4, 5, 10, 15, 20, 25, 30, 35, 40, 45, 50, 55, 60, 70, 75, 80, 85, 90, 91, 92, 93, 94, 95, 96, 97, 98, 99, or 100 percent of the theoretical yield for the individual target product (e.g. HMD, 6ACA, ADA, CPO, CPL, LVA or HDO). In embodiments, target products described herein are produced at about 10%-50%, 30%-90%, 40%-80%, 60%-95%, 50%-70%, or 50%-100% of the theoretical yield for a given target product.

Theoretical yields described herein can be measured in a fermentation broth described herein which includes one or more carbon sources described herein (e.g., methanol, sugar, glycerol). A genetically modified cell described herein can produce a target product described herein at an amount greater than about 60%-95% theoretical yield in fermentation broth. A genetically modified cell described herein can produce a target product described herein at an amount greater than about 60%-95% theoretical yield in fermentation broth using a sugar (e.g. glucose). A genetically modified cell described herein can produce a target product described herein at an amount greater than about 60%-95% theoretical yield in fermentation broth using methanol. A genetically modified cell described herein can produce a target product described herein at an amount greater than about 60%-95% theoretical yield in fermentation broth using glycerol.

The amount of target product can also be measured as a rate of production from non-naturally occurring microorganisms described herein. Thus, in certain instances, it may be convenient to determine the amount of production of a target product described herein as a rate of grams of product per liter of fermentation per hour of fermentation time (g/L/hr). Target products described herein can be produced at rates of 1 g/L/hr to 10 g/L/hr, 1 g/L/hr to 8 g/L/hr, 1 g/L/hr to 6 g/L/hr, 1 g/L/hr to 5 g/L/hr, 1 g/L/hr to 4 g/L/hr, 1 g/L/hr to 3 g/L/hr, 2 g/L/hr to 10 g/L/hr, 2 g/L/hr to 8 g/L/hr, 2 g/L/hr to 6 g/L/hr or 2 g/L/hr to 4 g/L/hr. In certain instances target products described herein can be produced at a rate of production of about 4 g/L/hr to 5 g/L/hr.

The amount of target product can be produced at an amount greater than about 5, 10, 20, 30, 40, 50, 60, 70, 75, 80, 85, 90, 95, or 100 percent target product by weight (w/w) after processing or purification as described herein of such target products. The amount of target product described herein can be produced at an amount greater than about 99, 99.90, 99.92, 99.94, 99.96, 99.98, 99.99, or 100% target product by weight after processing or purification as described herein of such target products (e.g. distillation). Thus, provided herein are non-naturally occurring microorganisms having one or more genetic modifications described herein capable of producing a target product described herein according to the w/w production described above. Accordingly, provided herein are non-naturally occurring microorganisms having one or more genetic modifications described herein capable of producing HMD at an amount greater than about 5, 10, 20, 30, 40, 50, 60, 70, 75, 80, 85, 90, 95, or 100 percent by weight or about 99, 99.90, 99.92, 99.94, 99.96, 99.98, 99.99, or 100% HMD by weight after processing or purification as described herein of such target products. Such non-naturally occurring microorganisms capable of producing HMD can, in certain instances, produce HMD at an amount greater than 5, 10, 15, 20, 25, or 30% in the fermentation broth.

Provided herein are non-naturally occurring microorganisms having one or more genetic modifications described herein capable of producing 6ACA at an amount greater than about 5, 10, 20, 30, 40, 50, 60, 70, 75, 80, 85, 90, 95, or 100 percent by weight or about 99, 99.90, 99.92, 99.94, 99.96, 99.98, 99.99, or 100% 6ACA by weight after processing or purification as described herein of such target products. Such non-naturally occurring microorganisms capable of producing 6ACA can, in certain instances, produce 6ACA at an amount greater than 5, 10, 15, 20, 25, or 30% in the fermentation broth. Provided herein are non-naturally occurring microorganisms having one or more genetic modifications described herein capable of producing ADA at an amount greater than about 5, 10, 20, 30, 40, 50, 60, 70, 75, 80, 85, 90, 95, or 100 percent by weight or about 99, 99.90, 99.92, 99.94, 99.96, 99.98, 99.99, or 100% ADA by weight after processing or purification as described herein of such target products. Such non-naturally occurring microorganisms capable of producing ADA can, in certain instances, produce ADA at an amount greater than 5, 10, 15, 20, 25, or 30% in the fermentation broth. Provided herein are non-naturally occurring microorganisms having one or more genetic modifications described herein capable of producing CPL at an amount greater than about 5, 10, 20, 30, 40, 50, 60, 70, 75, 80, 85, 90, 95, or 100 percent by weight or about 99, 99.90, 99.92, 99.94, 99.96, 99.98, 99.99, or 100% CPL by weight after processing or purification as described herein of such target products. Such non-naturally occurring microorganisms capable of producing CPL can, in certain instances, produce CPL at an amount greater than 5, 10, 15, 20, 25, or 30% in the fermentation broth.

Provided herein are non-naturally occurring microorganisms having one or more genetic modifications described herein capable of producing CPO at an amount greater than about 5, 10, 20, 30, 40, 50, 60, 70, 75, 80, 85, 90, 95, or 100 percent by weight or about 99, 99.90, 99.92, 99.94, 99.96, 99.98, 99.99, or 100% CPO by weight after processing or purification as described herein of such target products. Such non-naturally occurring microorganisms capable of producing CPO can, in certain instances, produce CPO at an amount greater than 5, 10, 15, 20, 25, or 30% in the fermentation broth. Provided herein are non-naturally occurring microorganisms having one or more genetic modifications described herein capable of producing LVA at an amount greater than about 5, 10, 20, 30, 40, 50, 60, 70, 75, 80, 85, 90, 95, or 100 percent by weight or about 99, 99.90, 99.92, 99.94, 99.96, 99.98, 99.99, or 100% LVA by weight after processing or purification as described herein of such target products. Such non-naturally occurring microorganisms capable of producing LVA can, in certain instances, produce LVA at an amount greater than 5, 10, 15, 20, 25, or 30% in the fermentation broth. Provided herein are non-naturally occurring microorganisms having one or more genetic modifications described herein capable of producing HDO at an amount greater than about 5, 10, 20, 30, 40, 50, 60, 70, 75, 80, 85, 90, 95, or 100 percent by weight or about 99, 99.90, 99.92, 99.94, 99.96, 99.98, 99.99, or 100% HDO by weight after processing or purification as described herein of such target products. Such non-naturally occurring microorganisms capable of producing HDO can, in certain instances, produce HDO at an amount greater than 5, 10, 15, 20, 25, or 30% in the fermentation broth.

Target products can be further characterized by the level of byproducts described herein contained in the final target product yield. Accordingly, target products described herein can include less than threshold levels of byproducts described herein in ppm quantities set forth herein. Target products described herein can include less than about 10000 ppm to 1 ppm, 7500 ppm to 1 ppm, 5000 ppm to 1 ppm, 4000 pm to 1 ppm, 3000 ppm to 1 ppm, 2000 ppm to 1 ppm, 1000 ppm to 1 ppm, 500 ppm to 1 ppm, or 100 ppm to 1 ppm. Target products described herein can include less than about 10000, 7500, 5000, 4000, 3000, 2000, 1000, 500, 250, 125, 100, 90, 75, 50, 40, 30, 20, 10, 5, or 1 ppm of a byproduct selected from Table 10, 11 or 12. In certain instances, target products described herein can include less than about 10000, 7500, 5000, 4000, 3000, 2000, 1000, 500, 250, 125, 100, 90, 75, 50, 40, 30, 20, 10, 5, or 1 ppm of any combination of byproducts selected from Table 10, 11 or 12. Thus, target products provided herein can include less than a total amount of about 10000, 7500, 5000, 4000, 3000, 2000, 1000, 500, 250, 125, 100, 90, 75, 50, 40, 30, 20, 10, 5, or 1 ppm of byproducts selected from Table 10, 11 or 12. Provided herein are non-naturally occurring microorganisms capable of producing HMD, 6ACA, ADA, CPL, CPO, or LVA where the HMD, 6ACA, ADA, CPL, CPO, or LVA independently includes less than about 10000, 7500, 5000, 4000, 3000, 2000, 1000, 500, 250, 125, 100, 90, 75, 50, 40, 30, 20, 10, 5, or 1 ppm of a byproduct selected from Table 10 or 12. Also provided herein are non-naturally occurring microorganisms capable of producing HMD, 6ACA, ADA, CPL, CPO, or LVA where the HMD, 6ACA, ADA, CPL, CPO, or LVA independently includes less than a total amount of about 10000, 7500, 5000, 4000, 3000, 2000, 1000, 500, 250, 125, 100, 90, 75, 50, 40, 30, 20, 10, 5, or 1 ppm byproducts selected from Table 10, 11 or 12.

The level of a byproduct or combination of byproducts described herein can be reduced by 5, 10, 20, 25, 30, 35, 40, 45, 50, 60, 70, 80, 90. 95 or 100% compared to a control cell lacking the genetic modification. The level of a byproduct or combination of byproducts described herein can be reduced by 5%-10%, 5%-20%, 5%-30%, 5%-40%, 5%-50%, 10%-20%, 10%-30%, 10%-40%, 10%-50%, 25%-50%, 25%-75%, 30%-60%, 30%-90%, 30%-95%, 50%-75%, 50%-95%, 60%-95%, 75%-95%, 80%-90%, 80%-95%, or 80%-100% compared to a control lacking the genetic modification.

Target products described herein can also be characterized by the percent weight of a byproduct described herein present in the target product. Thus, target products described herein can include less than about 20, 10, 5, 1, or 0.5 percent by weight of a byproduct described herein (e.g. Table 10, 11 or 12) or a combination of byproducts described herein. Accordingly, provided herein are non-naturally occurring microorganisms capable of producing HMD, 6ACA, ADA, CPL, CPO, or LVA where the HMD, 6ACA, ADA, CPL, CPO, or LVA independently includes less than about 20, 10, 5, 1, or 0.5 percent by weight of a byproduct described herein (e.g. Table 10, 11 or 12) or a combination of byproducts described herein.

Target products described herein can also be produced as a base, salt, or carbamate. HMD can be produced herein as a HMD base, a HMD salt (e.g. carbonate or bicarbonate), or HMD carbamate. 6ACA can be produced herein as a 6ACA base, a 6ACA salt (e.g. carbonate or bicarbonate), or 6ACA carbamate. ADA can be produced herein as an ADA base, an ADA salt (e.g. carbonate or bicarbonate), or ADA carbamate. CPL can be produced herein as a CPL base, a CPL salt (e.g. carbonate or bicarbonate), or CPL carbamate. CPO can be produced herein as a CPO base, a CPO salt (e.g. carbonate or bicarbonate), or CPO carbamate. LVA can be produced herein as a LVA base, a LVA salt (e.g. carbonate or bicarbonate), or LVA carbamate. HDO can be produced herein as a HDO base, a HDO salt (e.g. carbonate or bicarbonate), or HDO carbamate.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the non-naturally occurring microbial organisms described herein can synthesize HMD, LVA, 6ACA, CPL, CPO, ADA, or HDO at intracellular concentrations of 1-10 mM, 5-10 mM or more as well as all other concentrations exemplified herein having less byproduct than a comparable cell lacking the one or more genetic modifications described herein. It is understood that, even though the above description refers to intracellular concentrations, such microbial organisms can produce [HMD, LVA, 6ACA, CPL, CPO, ADA, or HDO intracellularly and/or secrete the product into the culture medium. The rate or percentage of product secreted into the culture media can be about 1, 5, 10, 15, 20, 25, 30, 35, 40, 45, 50, 55, 60, 65, 70, 75, 80, 85, 90, 95, 96, 97. 98. 99, or 100% product secreted out of the cell.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

In addition to the culturing and fermentation conditions disclosed herein, growth condition for achieving biosynthesis of HMD, LVA, 6ACA, CPL, CPO, ADA, or HDO can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

In some embodiments, the carbon feedstock and other cellular uptake sources such as phosphate, ammonia, sulfate, chloride and other halogens can be chosen to alter the isotopic distribution of the atoms present in target product or any intermediate described or set forth in a biosynthetic pathway described herein. The various carbon feedstock and other uptake sources enumerated above will be referred to herein, collectively, as “uptake sources.” Uptake sources can provide isotopic enrichment for any atom present in the target product or biosynthetic pathway intermediate described herein, or for side products generated in reactions diverging away from a biosynthetic pathway described herein. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S−B)/(M−B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950) ¹⁴C/¹²C ratio of 1.176±0.010×10⁻¹² (Karlen et al., Arkiv Geoftsik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³. In certain instances target products described herein can be characterized by calculating the isotopic ratios described herein.

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mil. ASTM D6866-11 suggests use of the available Oxalic Acid II standard SRM 4990 C (Ho2) for the modern standard (see discussion of original vs. currently available oxalic acid standards in Mann, Radiocarbon, 25(2):519-527 (1983)). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content of a compound or material and/or prepared downstream products that utilize a compound or material of the invention having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, the present invention provides target product or a target product pathway intermediate having a reduced level of byproducts described herein that has a carbon-12, carbon-13, and carbon-14 ratio that reflects an atmospheric carbon, also referred to as environmental carbon, uptake source. For example, in some aspects the target product or a target product pathway intermediate having a reduced level of byproducts described herein can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, the present invention provides target product or a target product pathway intermediate having a reduced level of byproducts described herein that has a carbon-12, carbon-13, and carbon-14 ratio that reflects petroleum-based carbon uptake source. In this aspect, the target product or a target product pathway intermediate having a reduced level of byproducts described herein can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, the present invention provides target product or a target product pathway intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present invention relates to the biologically produced target product or target product pathway intermediate as disclosed herein, and to the products derived there from, wherein the target product or a target product pathway intermediate has a carbon-12, carbon-13, and carbon-14 isotope ratio of about the same value as the CO₂ that occurs in the environment. For example, in some aspects the invention provides target product or a target product intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from target product or a target product pathway intermediate as disclosed herein, wherein the product is chemically modified to generate a final product. Methods of chemically modifying a product of target product, or an intermediate thereof, to generate a desired product are well known to those skilled in the art, as described herein. The invention further provides polyamides having a carbon-12 versus carbon-13 versus carbon-14 isotope ratio of about the same value as the CO₂ that occurs in the environment, wherein such polyamides are generated directly from or in combination with target product or a target product pathway intermediate as disclosed herein.

The invention further provides a composition comprising a target product, and a compound other than the target product. The compound other than the target product can be a cellular portion, for example, a trace amount of a cellular portion of, or can be fermentation broth or culture medium or a purified or partially purified fraction thereof produced in the presence of, a non-naturally occurring microbial organism of the invention having a target product pathway. The composition can comprise, for example, a reduced level of a byproduct when produced by an organism having reduced byproduct formation, as disclosed herein. The composition can comprise, for example, target product, or a cell lysate or culture supernatant of a microbial organism of the invention.

Target products described herein can be useful a chemicals for commercial and industrial applications. Non-limiting examples of such applications include production of polyamides (PA), polymers, precursors to polymers, resins, molded products, film, textiles, fibers, and solvents. In certain instances, target products described herein can be useful as solvents. In other instances, target products described herein can be useful chemical for production of resins or polymers Moreover, target product is also used as a raw material in the production of a wide range of products including PAs such as PA6 and PA6,6. Accordingly, provided herein are biobased PA products comprising one or more target products or target product pathway intermediates produced by a non-naturally occurring microorganism of the invention or produced using a method disclosed herein. A biobased product produced from a target product described herein can be molded or otherwise manipulated into a molded product.

As used herein, the term “bioderived” means derived from or synthesized by a biological organism and can be considered a renewable resource since it can be generated by a biological organism. Such a biological organism, in particular the microbial organisms of the invention disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a target product described herein having reduced levels of byproduct and produced using the cells and methods described herein. A biobased or product is in contrast to a petroleum derived product, wherein such a product is derived from or synthesized from petroleum or a petrochemical feedstock.

In some embodiments, the invention provides a PA biobased product comprising target product or target product pathway intermediate, wherein the target product or target product pathway intermediate includes all or part of the target product or target product pathway intermediate used in the production of a PA. For example, the final PA biobased product can contain the target product, target product pathway intermediate, or a portion thereof that is the result of the manufacturing of PAs. Such manufacturing can include chemically reacting the target product or target product pathway intermediate (e.g. chemical conversion, chemical functionalization, chemical coupling, oxidation, reduction, polymerization, copolymerization and the like) into the final PA compound or product. Thus, in some aspects, the invention provides a biobased PA product comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% target product or a target product pathway intermediate as disclosed herein.

Provided herein are methods of producing polyamide (PA) from renewable sources. In one aspect is a method of producing PA by using the cells described herein to produce a PA. In such a method, polymerization of a target product described herein is initiated and allowed to continue to produce the desired PA. The polymerization is terminated and the PA is isolated, thereby producing PA from a renewable source. The target product can be one described herein (e.g. HMD, ADA, or CPL) where the starting composition includes, in whole or in part, a target product described herein e.g. HMD, ADA, or CPL) produced from cells described herein (e.g. bioderived). The starting composition can be 1, 2, 3, 4, 5, 10, 20, 30, 40, 50, 60, 70. 80, 90, 91, 92, 93, 94, 95, 96, 97, 98, 99, or 100 percent target product described herein (e.g. HMD, ADA, or CPL). The renewable source can be a cell as described herein. The polyamide can be PA6, PA6,6, PA6,9, PA6,10, PA6,12 or PA6T.

Polyamides are generally synthesized from diamines and dibasic (dicarboxylic) acids, amino acids or lactams. Different polyamide (PA) types are identified by numbers denoting the number of carbon atoms in the monomers (generally diamine first). Exemplary commercial polyamides produced using the compounds produced by the invention include: polyamide 6 (polycaprolactam) made by the polycondensation of caprolactam; polyamide 66 (polyhexamethylene adipamide) made by condensing hexamethylenediamine with adipic acid; polyamide 69 (polyhexamethylene azelaamide) made by condensing hexamethylenediamine with azelaic acid [COOH(CH₂)₇COOH]; polyamide 6,10 made by condensing hexamethylenediamine with sebacic acid; polyamide 6/12 made from hexamethylenediamine and a 12-carbon dibasic acid; and PA6T made with HMD and terephthalic acid.

The starting composition can further include one or more byproducts described herein at a reduced level as described herein. The starting composition can also include non-target product compounds useful for polymerization to PA. Those of skill in the art readily understand that the exemplary target products described herein, e.g. HMD, 6ACA, ADA, CPL, CPO, LVA, and HDO, can be combined together in combination with each other and with other known chemicals (e.g. terephthalic acid) to arrive at useful polyamide polymers and products. Exemplary polyamide products (e.g. biobased products) which can be derived from using target products described herein include PA6, PA6,6, PA6,9, PA6,10, PA 6,12 or PA6T.

Polyamides are generally synthesized from diamines and dibasic (dicarboxylic) acids, amino acids or lactams. Different polyamide (PA) types are identified by numbers denoting the number of carbon atoms in the monomers (generally diamine first). Exemplary commercial polyamides produced using the compounds produced by the invention include: polyamide 6 (polycaprolactam)—made by the polycondensation of caprolactam; polyamide 66 (polyhexamethylene adipamide)—made by condensing hexamethylenediamine with adipic acid; polyamide 69 (polyhexamethylene azelaamide)—made by condensing hexamethylenediamine with azelaic acid [COOH(CH₂)₇COOH]; polyamide 6,10-made by condensing hexamethylenediamine with sebacic acid; polyamide 6/12-made from hexamethylenediamine and a 12-carbon dibasic acid; and and PA6T made with HMD and terephthalic acid.

Levulinic acid uses include for example its dehydrogenation to gamma-valerolactone (GVL) which is a prodrug to gamma-hydroxyvaleric acid (GHV) (see for example US20130296579A1) or a biofuel, use as a solvent or excipient, and so on.

Caprolactone (ε-Caprolactone) is a colorless liquid is miscible with most organic solvents. It is produced as a precursor to caprolactam. The caprolactone monomer is used in the manufacture of highly specialized polymers because of its ring-opening potential. Ring-opening polymerization, for example, results in the production of polycaprolactone. Caprolactone is typically prepared by oxidation of cyclohexanone with peracetic acid. Caprolactone undergoes reactions typical for primary alcohols. Downstream applications of these product groups include protective and industrial coatings, polyurethanes, cast elastomers, adhesives, colorants, pharmaceuticals and many more. Other useful properties of caprolactone include high resistance to hydrolysis, excellent mechanical properties, and low glass transition temperature.

6ACA is an analog of the amino acid lysine, which makes it an effective inhibitor for enzymes that bind that particular residue. Such enzymes include proteolytic enzymes like plasmin, the enzyme responsible for fibrinolysis. For this reason it is effective in treatment of certain bleeding disorders, and it is marketed as Amicar. 6ACA is also an intermediate in the polymerization of PA6 and is a precursor to caprolactam.

1,6-Hexanediol uses include production of polyester and polyurethane where it improves hardness and flexibility of polyesters. For polyurethanes it finds use as a chain extender. 16HDO is an intermediate to acrylics, adhesives, dyestuffs, styrene, maleic anhydride and fumaric acid.

Thus provided herein are biobased products derived at least in part using one or more target products described herein. The biobased product can be a PA described herein. Biobased products derived at least in part using target products described herein can include at least 5%, 10%, 20%, 30%, 40%, or at least 50% of HMD, 6ACA, ADA, CPL, CPO, LVA, or HDO produced according to the methods described herein. Such biobased products can be molded into molded products.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of target product includes anaerobic culture or fermentation conditions. In certain embodiments, the non-naturally occurring microbial organisms of the invention can be sustained, cultured or fermented under anaerobic or substantially anaerobic conditions. Briefly, an anaerobic condition refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of target product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of target product. Generally, and as with non-continuous culture procedures, the continuous and/or near-continuous production of target product will include culturing a non-naturally occurring target product producing organism of the invention in sufficient nutrients and medium to sustain and/or nearly sustain growth in an exponential phase. Continuous culture under such conditions can include, for example, growth or culturing for 1 day, 2, 3, 4, 5, 6 or 7 days or more. Additionally, continuous culture can include longer time periods of 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of target product can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the target product producers of the invention for continuous production of substantial quantities of target product, the target product producers also can be, for example, simultaneously subjected to chemical synthesis and/or enzymatic procedures to convert the product to other compounds or the product can be separated from the fermentation culture and sequentially subjected to chemical and/or enzymatic conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of target product.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

Employing the methods exemplified above, the methods of the invention allow the construction of cells and organisms that increase production of a desired product, for example, by coupling the production of a desired product to growth of the cell or organism engineered to harbor the identified genetic alterations. As disclosed herein, metabolic alterations have been identified that couple the production of HDO to growth of the organism. Microbial organism strains constructed with the identified metabolic alterations produce elevated levels, relative to the absence of the metabolic alterations, of HDO during the exponential growth phase. These strains can be beneficially used for the commercial production of HDO in continuous fermentation process without being subjected to the negative selective pressures described previously. Although exemplified herein as metabolic alterations, in particular one or more gene disruptions, that confer growth coupled production of HDO, it is understood that any gene disruption that increases the production of HDO can be introduced into a host microbial organism, as desired.

Therefore, the methods of the invention provide a set of metabolic modifications that are identified by an in silico method such as OptKnock. The set of metabolic modifications can include functional disruption of one or more metabolic reactions including, for example, disruption by gene deletion. For target product production, genetic modifications can be selected from the set of metabolic modifications listed in Table 3 or 4.

Also provided is a method of producing a non-naturally occurring microbial organisms having stable growth-coupled production of HDO. The method can include identifying in silico a set of metabolic modifications that increase production of HDO, for example, increase production during exponential growth; genetically modifying an organism to contain the set of metabolic modifications that increase production of HDO, and culturing the genetically modified organism. If desired, culturing can include adaptively evolving the genetically modified organism under conditions requiring production of target product. The methods of the invention are applicable to bacterium, yeast and fungus as well as a variety of other cells and microorganism, as disclosed herein.

Thus, the invention provides a non-naturally occurring microbial organism comprising one or more gene disruptions that confer increased production of HDO. In one embodiment, the one or more gene disruptions confer growth-coupled production of HDO, and can, for example, confer stable growth-coupled production of HDO. In another embodiment, the one or more gene disruptions can confer obligatory coupling of HDO production to growth of the microbial organism. Such one or more gene disruptions reduce the activity of the respective one or more encoded enzymes.

The non-naturally occurring microbial organism can have one or more gene disruptions included in a metabolic modification listed in Tables described herein such as, for example, Tables 3 and 4. The non-naturally occurring microorganism also include a genetic modification as described herein. As disclosed herein, the one or more gene disruptions can be a deletion. Such non-naturally occurring microbial organisms of the invention include bacteria, yeast, fungus, or any of a variety of other microorganisms applicable to fermentation processes, as disclosed herein.

Thus, the invention provides a non-naturally occurring microbial organism, comprising one or more gene disruptions, where the one or more gene disruptions occur in genes encoding proteins or enzymes where the one or more gene disruptions confer increased production of target product in the organism. The production of target product can be growth-coupled or not growth-coupled. In a particular embodiment, the production of target product can be obligatorily coupled to growth of the organism, as disclosed herein.

Also provided herein are compositions of target products described herein where the target product is produced from cells or methods described herein and can include a byproduct selected from Table 10 or 11. Such a byproduct can be present at a reduced level in the isolated target product when compared to isolated target product from a cell lacking one or more genetic modifications described herein. Compositions described herein can also include a reduced amount of metabolic byproducts (MB) such as those set forth in Table 14. Such disruption of MBs are known in the art and can redirect carbon flux along a given pathway when gene disruptions such as those described herein are introduced. Accordingly, provided herein is a composition of HMD, where the HMD is produced by a method described herein or a non-naturally occurring microorganism described herein having one or more genetic modifications described herein. The titer, yield, and rates of production of HMD produced by non-naturally occurring microorganisms described herein is as described herein. The HMD produced using the cells and methods described herein can include one or more byproducts described herein where the level of the one or more byproducts is reduced compared to production from a cell lacking such genetic modifications. Further, as described herein the yield of HMD may be increased as a result of reducing one or more byproducts described herein in a HMD pathway capable of producing HMD as described herein.

Also provided herein is a composition of 6ACA, where the 6ACA is produced by a method described herein or a non-naturally occurring microorganism described herein having one or more genetic modifications described herein. The titer, yield, and rates of production of 6ACA produced by non-naturally occurring microorganisms described herein is as described herein. The 6ACA produced using the cells and methods described herein can include one or more byproducts described herein where the level of the one or more byproducts is reduced compared to production from a cell lacking such genetic modifications. Further, as described herein the yield of 6ACA may be increased as a result of reducing one or more byproducts described herein in a 6ACA pathway capable of producing 6ACA as described herein.

Also provided herein is a composition of ADA, where the ADA is produced by a method described herein or a non-naturally occurring microorganism described herein having one or more genetic modifications described herein. The titer, yield, and rates of production of ADA produced by non-naturally occurring microorganisms described herein is as described herein. The ADA produced using the cells and methods described herein can include one or more byproducts described herein where the level of the one or more byproducts is reduced compared to production from a cell lacking such genetic modifications. Further, as described herein the yield of ADA may be increased as a result of reducing one or more byproducts described herein in a ADA pathway capable of producing ADA as described herein.

Also provided herein is a composition of CPL, where the CPL is produced by a method described herein or a non-naturally occurring microorganism described herein having one or more genetic modifications described herein. The titer, yield, and rates of production of CPL produced by non-naturally occurring microorganisms described herein is as described herein. The CPL produced using the cells and methods described herein can include one or more byproducts described herein where the level of the one or more byproducts is reduced compared to production from a cell lacking such genetic modifications. Further, as described herein the yield of CPL may be increased as a result of reducing one or more byproducts described herein in a CPL pathway capable of producing CPL as described herein.

Also provided herein is a composition of CPO, where the CPO is produced by a method described herein or a non-naturally occurring microorganism described herein having one or more genetic modifications described herein. The titer, yield, and rates of production of CPO produced by non-naturally occurring microorganisms described herein is as described herein. The CPO produced using the cells and methods described herein can include one or more byproducts described herein where the level of the one or more byproducts is reduced compared to production from a cell lacking such genetic modifications. Further, as described herein the yield of CPO may be increased as a result of reducing one or more byproducts described herein in a CPO pathway capable of producing CPO as described herein.

Also provided herein is a composition of LVA, where the LVA is produced by a method described herein or a non-naturally occurring microorganism described herein having one or more genetic modifications described herein. The titer, yield, and rates of production of LVA produced by non-naturally occurring microorganisms described herein is as described herein. The LVA produced using the cells and methods described herein can include one or more byproducts described herein where the level of the one or more byproducts is reduced compared to production from a cell lacking such genetic modifications. Further, as described herein the yield of LVA may be increased as a result of reducing one or more byproducts described herein in a LVA pathway capable of producing LVA as described herein.

Also provided herein is a composition of HDO, where the HDO is produced by a method described herein or a non-naturally occurring microorganism described herein having one or more genetic modifications described herein. The titer, yield, and rates of production of HDO produced by non-naturally occurring microorganisms described herein is as described herein. The HDO produced using the cells and methods described herein can include one or more byproducts described herein where the level of the one or more byproducts is reduced compared to production from a cell lacking such genetic modifications. Further, as described herein the yield of HDO may be increased as a result of reducing one or more byproducts described herein in a HDO pathway capable of producing HDO as described herein.

Compositions described herein can include byproduct present at a reduced amount in the composition when compared to target product produced from a cell lacking a genetic modification of one or more enzymes selected from A1-A25 or B1-B5. Such reduced amounts of byproduct can increase target product yield as described herein.

The composition can be any form of the fermentation, growth, and purification process of target product. Accordingly, in certain instances the composition is a fermentation broth. The fermentation broth can be as described herein. In certain instances the composition is a fermentation broth isolated from the cells (e.g. cells removed from the fermentation broth). Target product can be present in such compositions at an amount of at least 5, 10, 15, 20, 25 or 30% by weight (e.g. 50 g/L to about 300 g/L).

The composition can be a purified fermentation broth or downstream solution/solvent following fermentation with cells described herein. In such instances, when a target product is included in a processed or purified composition, the target product can be present in the composition at an amount of at least 50, 60, 70, 75, 80, 90, 95, or 99% by weight of the composition. Compositions described herein can include target products described herein at an amount greater than about 99, 99.90, 99.92, 99.94, 99.96, 99.98, 99.99, or 100% target product by weight after processing or purification.

Compositions of target products described herein can be produced in amounts as described herein. As such, compositions described herein can include target product produced in a cell described herein at about 60%-95% theoretical yield. Compositions described herein including target product produced in a cell described herein can be produced at a titer of about 0.1 g/L to about 300 g/L or about 0.1 g/L to about 120 g/L fermentation.

The compositions described herein also contain reduced amounts of one or more byproducts described herein (e.g. Table 10 or 11). The compositions described herein therefore can include one or more byproducts described herein at an amount of less than 10000, 7500, 5000, 4000, 3000, 2000, 1000, 500, 250, 125, 100, 90, 75, 50, 40, 30, 20, 10, 5, or 1 ppm. Reduced amounts of byproducts described herein are relative to production of the same target product in a cell lacking the genetic modifications described herein.

The compositions described herein can include HMD. The HMD can include byproducts described herein as described above. Compositions described herein can include 6ACA, where the 6ACA can include byproducts described herein as described above.

Compositions described herein can include ADA, where the ADA can include byproducts described herein as described above. Compositions described herein can include CPL, where the CPL can include byproducts described herein as described above. Compositions described herein can include CPO, where the CPO can include byproducts described herein as described above. Compositions described herein can include LVA, where the LVA can include byproducts described herein as described above. Compositions described herein can include HDO, where the HDO can include byproducts described herein as described above. In certain instances, compositions described herein include one or more target products described herein.

Provided herein are methods of producing target products described herein. In one aspect is a method of producing a target product described herein (e.g., 6ACA, ADA, CPL, CPO, LVA, and HDO) by culturing cells described herein under conditions and for a sufficient period of time to produce the desired target product(s). Such methods can further include isolation the target product. Isolation can be from either the cells or from the broth. Methods for producing target products described herein can also include purification of the target product using techniques known in the art and described herein. In a particular example, target products can be purified using distillation techniques or crystallization (e.g. as a salt).

Accordingly, provided herein is HMD produced according the methods described above. Also provided herein is 6ACA produced according to the methods described above. Further provided herein is ADA produced according to the methods described above. Provided herein is CPL produced according to the methods described above. Provided herein is CPO produced according to the methods described above. Provided herein is LVA produced according to the methods described above. Provided herein is HDO produced according to the methods described above.

It is understood that modifications which do not substantially affect the activity of the various embodiments of this invention are also included within the definition of the invention provided herein. Accordingly, the following examples are intended to illustrate but not limit the present invention

### EXAMPLES

### Example 1

Described below are various pathways leading to the production of HMD, or 6-aminocaproate from common central metabolites. The first described pathway entails the activation of 6-aminocaproate to 6-aminocaproyl-CoA by a transferase or synthase enzyme (FIG. 1, Step Q or R) followed by the spontaneous cyclization of 6-aminocaproyl-CoA to form caprolactam (FIG. 1, Step T). The second described pathway entails the activation of 6-aminocaproate to 6-aminocaproyl-CoA (FIG. 1, Step Q or R), followed by a reduction (FIG. 1, Step U) and amination (FIG. 1, Step V or W) to form HMD. 6-Aminocaproic acid can alternatively be activated to 6-aminocaproyl-phosphate instead of 6-aminocaproyl-CoA. 6-Aminocaproyl-phosphate can spontaneously cyclize to form caprolactam. Alternatively, 6-aminocaproyl-phosphate can be reduced to 6-aminocaproate semialdehye, which can be then converted to HMD as depicted in FIG. 1. In either this case, the amination reaction must occur relatively quickly to minimize the spontaneous formation of the cyclic imine of 6-aminocaproate semialdehyde. Linking or scaffolding the participating enzymes represents a potentially powerful option for ensuring that the 6-aminocaproate semialdehyde intermediate is efficiently channeled from the reductase enzyme to the amination enzyme. Note that 6-aminocaproate can be formed from various starting molecules. For example, the carbon backbone of 6-aminocaproate can be derived from succinyl-CoA and acetyl-CoA as depicted in FIG. 1.

1.1.1 Oxidoreductases.

Four transformations depicted in FIG. 1 require oxidoreductases that convert a ketone functionality to a hydroxyl group. Step B in FIG. 1 involves converting a 3-oxoacyl-CoA to a 3-hydroxyacyl-CoA.

Exemplary enzymes that can convert 3-oxoacyl-CoA molecules such as 3-oxoadipyl-CoA and 3-oxo-6-aminohexanoyl-CoA into 3-hydroxyacyl-CoA molecules such as 3-hydroxyadipyl-CoA and 3-hydroxy-6-aminohexanoyl-CoA, respectively, include enzymes whose natural physiological roles are in fatty acid beta-oxidation or phenylacetate catabolism. For example, subunits of two fatty acid oxidation complexes in E. coli, encoded by fadB and fadJ, function as 3-hydroxyacyl-CoA dehydrogenases (Binstock et al., Methods Enzymol. 71:403-411 (1981)). Furthermore, the gene products encoded by phaC in Pseudomonas putida U (Olivera et al., Proc. Natl. Acad. Sci. USA 95:6419-6424 (1998)) and paaC in Pseudomonas fluorescens ST (Di Gennaro et al., Arch. Microbiol. 188:117-125 (2007)) catalyze the reverse reaction of step B in FIG. 1, that is, the oxidation of 3-hydroxyadipyl-CoA to form 3-oxoadipyl-CoA, during the catabolism of phenylacetate or styrene. Note that the reactions catalyzed by such enzymes are reversible. In addition, given the proximity in E. coli of paaH to other genes in the phenylacetate degradation operon (Nogales et al., Microbiology 153:357-365 (2007)) and the fact that paaH mutants cannot grow on phenylacetate (Ismail et al., Eur. J Biochem. 270:3047-3054 (2003)), it is expected that the E. coli paaH gene encodes a 3-hydroxyacyl-CoA dehydrogenase. Additional exemplary oxidoreductases capable of converting 3-oxoacyl-CoA molecules to their corresponding 3-hydroxyacyl-CoA molecules include those exemplified in U.S. Pat. No. 8,377,680 which is herein incorporated in its entirety and for all purposes.

Several of these alcohol hyderogenases have been shown to demonstrate activity on 3-oxoadipyl-CoA and convert it to 3-hydroxyadipyl-CoA.

| Gene | | GenBank | |
|---|---|---|---|
| name | GI# | Accession # | Organism |
| fadB | 119811 | P21177.2 | Escherichia coli |
| fadJ | 3334437 | P77399.1 | Escherichia coli |
| paaH | 16129356 | NP_415913.1 | Escherichia coli |
| paaH1 | 113866312 | YP_724801.1 | Ralstonia eutropha |
| | | | H16 (Cupriavidus necator) |
| dcaH | 15812039 | AAL09091.1 | Acinetobacter sp. ADP1 |
| hbd | 15895965 | 15 NP_349314.1 | Clostridium acetobutylicum |
| paaC | 26990000 | NP_745425.1 | Pseudomonas putida KT2240 |
| paaC | 106636095 | ABF82235.1 | Pseudomonas fluorescens |

Various alcohol dehydrogenases represent good candidates for converting 3-oxoadipate to 3-hydroxyadipate (step H, FIG. 1). Two such enzymes capable of converting an oxoacid to a hydroxyacid are encoded by the malate dehydrogenase (mdh) and lactate dehydrogenase (ldhA) genes in E. coli. In addition, lactate dehydrogenase from Ralstonia eutropha has been shown to demonstrate high activities on substrates of various chain lengths such as lactate, 2-oxobutyrate, 2-oxopentanoate and 2-oxoglutarate (Steinbuchel et al., Eur. J. Biochem. 130:329-334 (1983)). Conversion of alpha-ketoadipate into alpha-hydroxyadipate can be catalyzed by 2-ketoadipate reductase, an enzyme reported to be found in rat and in human placenta (Suda et al., Arch. Biochem. Biophys. 176:610-620 (1976); Suda et al., Biochem. Biophys. Res. Commun. 77:586-591 (1977)). An additional candidate for these steps is the mitochondrial 3-hydroxybutyrate dehydrogenase (bdh) from the human heart which has been cloned and characterized (Marks et al., J. Biol. Chem. 267:15459-15463 (1992)). This enzyme is a dehydrogenase that operates on a 3-hydroxyacid. Another exemplary alcohol dehydrogenase converts acetone to isopropanol as was shown in C. beijerinckii (Ismaiel et al., J. Bacteriol. 175:5097-5105 (1993) and T. brockii (Lamed et al., Biochem. J. 195:183-190 (1981); Peretz et al., Biochemistry 28:6549-6555 (1989)).

| Gene | | GenBank | |
|---|---|---|---|
| name | GI# | Accession # | Organism |
| mdh | 1789632 | AAC76268.1 | Escherichia coli |
| ldhA | 16129341 | NP_415898.1 | Escherichia coli |
| ldh | 113866693 | YP_725182.1 | Ralstonia eutropha |
| bdh | 177198 | AAA58352.1 | Homo sapiens |
| adh | 60592974 | AAA23199.2 | Clostridium beijerinckii |
| adh | 113443 | P14941.1 | Thermoanaerobacter brockii |

1.2.1 Oxidoreductase (acyl-CoA to aldehyde).

The transformations of adipyl-CoA to adipate semialdehyde (Step N, FIG. 1) and 6-aminocaproyl-CoA to 6-aminocaproate semialdehyde (Step U, FIG. 1) require acyl-CoA dehydrogenases capable of reducing an acyl-CoA to its corresponding aldehyde. Exemplary genes that encode such enzymes include the Acinetobacter calcoaceticus acr1 encoding a fatty acyl-CoA reductase (Reiser et al., J. Bacteriology 179:2969-2975 (1997)), the Acinetobacter sp. M-1 fatty acyl-CoA reductase (Ishige et al., Appl. Environ. Microbiol. 68:1192-1195 (2002)), and a CoA- and NADP-dependent succinate semialdehyde dehydrogenase encoded by the sucD gene in Clostridium kluyveri (Sohling et al., J. Bacteriol. 178:871-880 (1996)). SucD of P. gingivalis is another succinate semialdehyde dehydrogenase (Takahashi et al., J. Bacteriol. 182:4704-4710 (2000)). The enzyme acylating acetaldehyde dehydrogenase in Pseudomonas sp, encoded by bphG, is yet another candidate as it has been demonstrated to oxidize and acylate acetaldehyde, propionaldehyde, butyraldehyde, isobutyraldehyde and formaldehyde (Powlowski et al., J Bacteriol. 175:377-385 (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Kazahaya et al., J. Gen. Appl. Microbiol. 18:43-55 (1972); Koo et al., Biotechnol Lett. 27:505-510 (2005)). An additional enzyme type that converts an acyl-CoA to its corresponding aldehyde is malonyl-CoA reductase which transforms malonyl-CoA to malonic semialdehyde. Such enzymes are known in the art and exemplified in for example U.S. Pat. No. 8,377,680 which is herein incorporated in its entirety and for all purposes.

| Gene | | GenBank | |
|---|---|---|---|
| name | GI# | Accession # | Organism |
| acr1 | 50086359 | YP_047869.1 | Acinetobacter calcoaceticus |
| acr1 | 1684886 | AAC45217 | Acinetobacter baylyi |
| acr1 | 18857901 | BAB885476.1 | Acinetobacter sp. Strain M-1 |
| sucD | 172046062 | P38947.1 | Clostridium kluyveri |
| sucD | 34540484 | NP_904963.1 | Porphyromonas gingivalis |
| bphG | 425213 | BAA03892.1 | Pseudomonas sp |
| adhE | 55818563 | AAV66076.1 | Leuconostoc mesenteroides |

1.3.1 Oxidoreductase Operating on CH—CH Donors.

Referring to FIG. 1, step D refers to the conversion of 5-carboxy-2-pentenoyl-CoA to adipyl-CoA by 5-carboxy-2-pentenoyl-CoA reductase. One exemplary enoyl-CoA reductase is the gene product of bcd from C. acetobutylicum (Boynton et al., J Bacteriol. 178:3015-3024 (1996); Atsumi et al., Metab. Eng. 2008 10(6):305-311 (2008)(Epub Sep. 14, 2007), which naturally catalyzes the reduction of crotonyl-CoA to butyryl-CoA. Activity of this enzyme can be enhanced by expressing bcd in conjunction with expression of the C. acetobutylicum etfAB genes, which encode an electron transfer flavoprotein. An additional candidate for the enoyl-CoA reductase step is the mitochondrial enoyl-CoA reductase from E. gracilis (Hoffmeister et al., J Biol. Chem. 280:4329-4338 (2005)). A construct derived from this sequence following the removal of its mitochondrial targeting leader sequence was cloned in E. coli resulting in an active enzyme (Hoffmeister et al., supra). This approach is well known to those skilled in the art of expressing eukaryotic genes, particularly those with leader sequences that may target the gene product to a specific intracellular compartment, in prokaryotic organisms. A close homolog of this gene, TDE0597, from the prokaryote Treponema denticola represents a third enoyl-CoA reductase which has been cloned and expressed in E. coli (Tucci et al., FEBS Letters 581:1561-1566 (2007)).

| Gene name | GI# | GenBank Accession # | Organism |
|---|---|---|---|
| bcd | 15895968 | NP_349317.1 | Clostridium acetobutylicum |
| etfA | 15895966 | NP_349315.1 | Clostridium acetobutylicum |
| etfB | 15895967 | NP_349316.1 | Clostridium acetobutylicum |
| dcaA | 15812042 | AAL09094.1 | Acinetobacter sp. ADP1 |
| TER | 62287512 | Q5EU90.1 | Euglena gracilis |
| TER | 150016955 | YP_001309209.1 | Clostridium beijerinckii |
| | | | NCIMB 8052 |
| TDE0597 | 42526113 | NP_971211.1 | Treponema denticola |
| ETR1 | 51316051 | Q8WZM3.1 | Candida tropicalis |
| CTRG_06166 | 255723510 | XP_002546688.1 | Candida tropicalis MYA-3404 |
| YALI0C19624 | 50549095 | XP_502018.1 | Yarrowia lipolytica CLIB122 |

Several of the gene candidates listed here have been checked in house for activity to convert 5-carboxy 2-pentenoyl-CoA to adipyl-CoA and have been shown to be active.

Step J of FIG. 1 requires a 2-enoate reductase enzyme. 2-Enoate reductases (EC 1.3.1.31) are known to catalyze the NAD(P)H-dependent reduction of a wide variety of α, β-unsaturated carboxylic acids and aldehydes (Rohdich et al., J. Biol. Chem. 276:5779-5787 (2001)). 2-Enoate reductase is encoded by enr in several species of Clostridia (Giesel et al., Arch Microbiol 135:51-57 (1983)) including C. tyrobutyricum, and C. thermoaceticum (now called Moorella thermoaceticum) (Rohdich et al., supra). In the published genome sequence of C. kluyveri, 9 coding sequences for enoate reductases have been reported, out of which one has been characterized (Seedorf et al., Proc. Natl. Acad. Sci. USA, 105:2128-2133 (2008)). The enr genes from both C. tyrobutyricum and C. thermoaceticum have been cloned and sequenced and show 59% identity to each other. The former gene is also found to have approximately 75% similarity to the characterized gene in C. kluyveri (Giesel et al., supra). It has been reported based on these sequence results that enr is very similar to the dienoyl CoA reductase in E. coli (fadH) (Rohdich et al., supra). The C. thermoaceticum enr gene has also been expressed in an enzymatically active form in E. coli (Rohdich et al., supra).

| | | GenBank | |
|---|---|---|---|
| Gene name | GI# | Accession # | Organism |
| fadH | 16130976 | NP_417552.1 | Escherichia coli |
| enr | 169405742 | ACA54153.1 | Clostridium botulinum A3 |
| | | | str |
| enr | 2765041 | CAA71086.1 | Clostridium tyrobutyricum |
| enr | 3402834 | CAA76083.1 | Clostridium kluyveri |
| enr | 83590886 | YP_430895.1 | Moorella thermoacetica |

1.4.1 Oxidoreductase Operating on Amino Acids.

FIG. 1 depicts two reductive aminations. Specifically, step P of FIG. 1 involves the conversion of adipate semialdehyde to 6-aminocaproate and step W of FIG. 1 entails the conversion of 6-aminocaproate semialdehyde to hexamethylenediamine.

Most oxidoreductases operating on amino acids catalyze the oxidative deamination of alpha-amino acids with NAD+ or NADP+ as acceptor, though the reactions are typically reversible. Exemplary oxidoreductases operating on amino acids include glutamate dehydrogenase (deaminating), encoded by gdhA, leucine dehydrogenase (deaminating), encoded by ldh, and aspartate dehydrogenase (deaminating), encoded by nadX. The gdhA gene product from Escherichia coli (McPherson et al., Nucleic. Acids Res. 11:5257-5266 (1983); Korber et al., J. Mol. Biol. 234:1270-1273 (1993)), gdh from Thermotoga maritima (Kort et al., Extremophiles 1:52-60 (1997); Lebbink et al., J. Mol. Biol. 280:287-296 (1998); Lebbink et al., J. Mol. Biol. 289:357-369 (1999)), and gdhA1 from Halobacterium salinarum (Ingoldsby et al., Gene. 349:237-244 (2005)) catalyze the reversible interconversion of glutamate to 2-oxoglutarate and ammonia, while favoring NADP(H), NAD(H), or both, respectively. The ldh gene of Bacillus cereus encodes the LeuDH protein that has a wide of range of substrates including leucine, isoleucine, valine, and 2-aminobutanoate (Stoyan et al., J. Biotechnol 54:77-80 (1997); Ansorge et al., Biotechnol Bioeng. 68:557-562 (2000)). The nadX gene from Thermotoga maritime encoding for the aspartate dehydrogenase is involved in the biosynthesis of NAD (Yang et al., J. Biol. Chem. 278:8804-8808 (2003)). Additional useful enzymes for steps P and W of FIG. 1 are known in the art and exemplified for example in U.S. Pat. No. 8,377,680 which is herein incorporated in its entirety and for all purposes.

| Gene | | | |
|---|---|---|---|
| name | GI# | GenBank Accession # | Organism |
| gdhA | 118547 | P00370 | Escherichia coli |
| gdh | 6226595 | P96110.4 | Thermotoga maritima |
| gdhA1 | 15789827 | NP_279651.1 | Halobacterium salinarum |
| ldh | 61222614 | P0A393 | Bacillus cereus |
| nadX | 15644391 | NP_229443.1 | Thermotoga maritima |

2.3.1 Acyl Transferase.

Referring to FIG. 1, step A involves 3-oxoadipyl-CoA thiolase, or equivalently, succinyl CoA:acetyl CoA acyl transferase (β-ketothiolase). The gene products encoded by pcaF in Pseudomonas strain B13 (Kaschabek et al., J. Bacteriol. 184:207-215 (2002)), phaD in Pseudomonas putida U (Olivera et al., supra), paaE in Pseudomonas fluorescens ST (Di Gennaro et al., supra), and paaf from E. coli (Nogales et al., supra) catalyze the conversion of 3-oxoadipyl-CoA into succinyl-CoA and acetyl-CoA during the degradation of aromatic compounds such as phenylacetate or styrene. Since β-ketothiolase enzymes catalyze reversible transformations, these enzymes can be employed for the synthesis of 3-oxoadipyl-CoA. For example, the ketothiolase phaA from R. eutropha combines two molecules of acetyl-CoA to form acetoacetyl-CoA (Sato et al., J Biosci Bioeng 103:38-44 (2007)). Similarly, a β-keto thiolase (bktB) has been reported to catalyze the condensation of acetyl-CoA and propionyl-CoA to form β-ketovaleryl-CoA (Slater et al., J. Bacteria 180:1979-1987 (1998)) in R. eutropha. In addition to the likelihood of possessing 3-oxoadipyl-CoA thiolase activity, all such enzymes represent good candidates for condensing 4-aminobutyryl-CoA and acetyl-CoA to form 3-oxo-6-aminohexanoyl-CoA either in their native forms or once they have been appropriately engineered. Other acyl transferases are known in the art to catalyze step A and are exemplified for example in U.S. Pat. No. 8,377,680 which is herein incorporated in its entirety and for all purposes. Several thiolases candidates have been shown inhouse to combine acetyl-CoA with succinyl-CoA and convert it to 3-oxoadipyl-CoA.

| | | GenBank | |
|---|---|---|---|
| Gene name | GI# | Accession # | Organism |
| paaJ | 16129358 | NP_415915.1 | Escherichia coli |
| pcaF | 17736947 | AAL02407 | Pseudomonas knackmussii |
| | | | (B13) |
| phaD | 3253200 | AAC24332.1 | Pseudomonas putida |
| paaE | 106636097 | ABF82237.1 | Pseudomonas fluorescens |
| dcaF | 50084844 | YP_046354.1 | Acinetobacter |
| | | | sp. strain ADP1 |

2.6.1 Aminotransferase.

Step O and V of FIG. 1 require transamination of a 6-aldehyde to an amine. These transformations can be catalyzed by gamma-aminobutyrate transaminase (GABA transaminase). One E. coli GABA transaminase is encoded by gabT and transfers an amino group from glutamate to the terminal aldehyde of succinyl semialdehyde (Bartsch et al., J. Bacteriol. 172:7035-7042 (1990)). The gene product of puuE catalyzes another 4-aminobutyrate transaminase in E. coli (Kurihara et al., J. Biol. Chem. 280:4602-4608 (2005)). GABA transaminases in Mus musculus, Pseudomonasfluorescens, and Sus scrofa have been shown to react with 6-aminocaproic acid (Cooper, Methods Enzymol. 113:80-82 (1985); Scott et al., J. Biol. Chem. 234:932-936 (1959)).

| | | GenBank | |
|---|---|---|---|
| Gene name | GI# | Accession # | Organism |
| gabT | 16130576 | NP_417148.1 | Escherichia coli |
| puuE | 16129263 | NP_415818.1 | Escherichia coli |
| abat | 37202121 | NP_766549.2 | Mus musculus |
| gabT | 70733692 | YP_257332.1 | Pseudomonas fluorescens |
| abat | 47523600 | NP_999428.1 | Sus scrofa |

Additional enzyme candidates are known in the art and include putrescine aminotransferases, beta-alanine/alpha-ketoglutarate aminotransferases or other diamine aminotransferases such as those exemplified by U.S. Pat. No. 8,377,680 which is herein incorporated in its entirety and for all purposes. Such enzymes are particularly well suited for carrying out the conversion of 6-aminocaproate semialdehyde to hexamethylenediamine. The E. coli putrescine aminotransferase is encoded by the ygjG gene and the purified enzyme also was able to transaminate cadaverine and spermidine (Samsonova et al., BMC Microbiol 3:2 (2003)). In addition, activity of this enzyme on 1,7-diaminoheptane and with amino acceptors other than 2-oxoglutarate (e.g., pyruvate, 2-oxobutanoate) has been reported (Samsonova et al., supra; Kim, K. H., J Biol Chem 239:783-786 (1964)). A putrescine aminotransferase with higher activity with pyruvate as the amino acceptor than alpha-ketoglutarate is the spuC gene of Pseudomonas aeruginosa (Lu et al., J Bacteriol 184:3765-3773 (2002)).

| | | GenBank | |
|---|---|---|---|
| Gene name | GI# | Accession # | Organism |
| ygjG | 145698310 | NP_417544 | Escherichia coli |
| spuC | 9946143 | AAG03688 | Pseudomonas aeruginosa |
| FG99_15380 | 664810528 | KES23458 | Pseudomonas sp. AAC |
| FG99_14885 | 664810430 | KES23360 | Pseudomonas sp. AAC |
| FG99_07980 | 664811586 | KES24511.1 | Pseudomonas sp. AAC |

Gene candidates listed here have been tested for activity to convert 6-aminocaproic acid to adipate semialdehyde and hexamethylenediamine to 6-aminocaproate semialdehyde and have been shown to be active.

2.8.3 Coenzyme-A Transferase.

CoA transferases catalyze reversible reactions that involve the transfer of a CoA moiety from one molecule to another. For example, step E of FIG. 1 is catalyzed by a 3-oxoadipyl-CoA transferase. In this step, 3-oxoadipate is formed by the transfer of the CoA group from 3-oxoadipyl-CoA to succinate, acetate, or another CoA acceptor. One candidate enzyme for these steps is the two-unit enzyme encoded by pad and pcaJ in Pseudomonas, which has been shown to have 3-oxoadipyl-CoA/succinate transferase activity (Kaschabek et al., supra). Similar enzymes based on homology exist in Acinetobacter sp. ADP1 (Kowalchuk et al., Gene 146:23-30 (1994)) and Streptomyces coelicolor. Additional exemplary succinyl-CoA:3:oxoacid-CoA transferases are present in Helicobacter pylori (Corthesy-Theulaz et al., J. Biol. Chem. 272:25659-25667 (1997)) and Bacillus subtilis (Stols et al., Protein. Expr. Purif. 53:396-403 (2007)).

| | | GenBank | |
|---|---|---|---|
| Gene name | GI# | Accession # | Organism |
| pcaI | 24985644 | AAN69545.1 | Pseudomonas putida |
| pcaJ | 26990657 | NP_746082.1 | Pseudomonas putida |
| pcaI | 50084858 | YP_046368.1 | Acinetobacter sp. ADP1 |
| pcaJ | 141776 | AAC37147.1 | Acinetobacter sp. ADP1 |
| pcaI | 21224997 | NP_630776.1 | Streptomyces coelicolor |
| pcaJ | 21224996 | NP_630775.1 | Streptomyces coelicolor |
| HPAG1_0676 | 108563101 | YP_627417 | Helicobacter pylori |
| HPAG1_0677 | 108563102 | YP_627418 | Helicobacter pylori |
| ScoA | 16080950 | NP_391778 | Bacillus subtilis |
| ScoB | 16080949 | NP_391777 | Bacillus subtilis |
| dcaI | 15812044 | AAL09096.1 | Acinetobacter sp. ADP1 |
| dcaJ | 15812045 | AAL09097.1 | Acinetobacter sp. ADP1 |
| catI | 631779821 | CDF84299 | Pseudomonas |
| | | | knackmussii B13 |
| catJ | 631779820 | CDF84298 | Pseudomonas |
| | | | knackmussii B13 |

A 3-oxoacyl-CoA transferase that can utilize acetate as the CoA acceptor is acetoacetyl-CoA transferase, encoded by the E. coli atoA (alpha subunit) and atoD (beta subunit) genes (Vanderwinkel et al., Biochem. Biophys. Res Commun. 33:902-908 (1968); Korolev et al., Acta Crystallogr. D Biol Crystallogr. 58:2116-2121 (2002)). This enzyme has also been shown to transfer the CoA moiety to acetate from a variety of branched and linear acyl-CoA substrates, including isobutyrate (Matthies et al., Appl Environ Microbiol 58:1435-1439 (1992)), valerate (Vanderwinkel et al., supra) and butanoate (Vanderwinkel et al., supra). Similar enzymes exist in Corynebacterium glutamicum ATCC 13032 (Duncan et al., Appl Environ Microbiol 68:5186-5190 (2002)), Clostridium acetobutylicum (Cary et al., Appl Environ Microbiol 56:1576-1583 (1990)), and Clostridium saccharoperbutylacetonicum (Kosaka et al., Biosci. Biotechnol Biochem. 71:58-68 (2007)).

| | | GenBank | |
|---|---|---|---|
| Gene name | GI# | Accession # | Organism |
| atoA | 2492994 | P76459.1 | Escherichia coli K12 |
| atoD | 2492990 | P76458.1 | Escherichia coli K12 |
| actA | 62391407 | YP_226809.1 | Corynebacterium glutamicum |
| | | | ATCC 13032 |
| cg0592 | 62389399 | YP_224801.1 | Corynebacterium glutamicum |
| | | | ATCC 13032 |
| ctfA | 15004866 | NP_149326.1 | Clostridium acetobutylicum |
| ctfB | 15004867 | NP_149327.1 | Clostridium acetobutylicum |
| ctfA | 31075384 | AAP42564.1 | Clostridium |
| | | | saccharoperbutylacetonicum |
| ctfB | 31075385 | AAP42565.1 | Clostridium |
| | | | saccharoperbutylacetonicum |

The above enzymes may also exhibit the desired activities on adipyl-CoA and adipate (FIG. 1, step K) or 6-aminocaproate and 6-aminocaproyl-CoA (FIG. 11, step Q). Nevertheless, additional exemplary transferase candidates are catalyzed by the gene products of cat1, cat2, and cat3 of Clostridium kluyveri which have been shown to exhibit succinyl-CoA, 4-hydroxybutyryl-CoA, and butyryl-CoA transferase activity, respectively (Seedorf et al., supra; Sohling et al., Eur. J Biochem. 212:121-127 (1993); Sohling et al., J Bacteria 178:871-880 (1996)).

| Gene name | GI# | GenBank Accession # | Organism |
|---|---|---|---|
| cat1 | 729048 | P38946.1 | Clostridium kluyveri |
| cat2 | 172046066 | P38942.2 | Clostridium kluyveri |
| cat3 | 146349050 | EDK35586.1 | Clostridium kluyveri |

The glutaconate-CoA-transferase (EC 2.8.3.12) enzyme from anaerobic bacterium Acidaminococcus fermentans reacts with diacid glutaconyl-CoA and 3-butenoyl-CoA (Mack et al., FEBS Lett. 405:209-212 (1997)). The genes encoding this enzyme are gctA and gctB. This enzyme has reduced but detectable activity with other CoA derivatives including glutaryl-CoA, 2-hydroxyglutaryl-CoA, adipyl-CoA and acrylyl-CoA (Buckel et al., Eur. J. Biochem. 118:315-321 (1981)). The enzyme has been cloned and expressed in E. coli (Mack et al., Eur. J. Biochem. 226:41-51 (1994)).

| | | GenBank | |
|---|---|---|---|
| Gene name | GI# | Accession # | Organism |
| gctA | 559392 | CAA57199.1 | Acidaminococcus fermentans |
| gctB | 559393 | CAA57200.1 | Acidaminococcus fermentans |
| gctA | 542983069 | ERI79632.1 | Clostridium symbiosum |
| | | | ATCC 14940 |
| gctB | 542983070 | ERI79633.1 | Clostridium symbiosum |
| | | | ATCC 14940 |

Several of these exemplary gene candidates listed above have been tested inhouse for CoA transferase activity on adipate, 3-oxoadipate, 6 aminocaproate and 2,3-dehydroadipate and activity has been demonstrated.

3.1.2 Thiolester Hydrolase (CoA Specific).

Several eukaryotic acetyl-CoA hydrolases have broad substrate specificity and thus represent suitable candidate enzymes for hydrolyzing 3-oxoadipyl-CoA, adipyl-CoA, 3-oxo-6-aminohexanoyl-CoA, or 6-aminocaproyl-CoA (Steps G and M of FIG. 1). For example, the enzyme from Rattus norvegicus brain (Robinson et al., Biochem. Biophys. Res. Commun. 71:959-965 (1976)) can react with butyryl-CoA, hexanoyl-CoA and malonyl-CoA.

| Gene name | GI# | GenBank Accession # | Organism |
|---|---|---|---|
| acot12 | 18543355 | NP_570103.1 | Rattus norvegicus |

Additional hydrolase enzymes include 3-hydroxyisobutyryl-CoA hydrolase which has been described to efficiently catalyze the conversion of 3-hydroxyisobutyryl-CoA to 3-hydroxyisobutyrate during valine degradation (Shimomura et al., J Biol Chem. 269:14248-14253 (1994)). Genes encoding this enzyme include hibch of Rattus norvegicus (Shimomura et al., supra; Shimomura et al., Methods Enzymol. 324:229-240 (2000)) and Homo sapiens (Shimomura et al., supra). Candidate genes by sequence homology include hibch of Saccharomyces cerevisiae and BC 2292 of Bacillus cereus.

| | | GenBank | |
|---|---|---|---|
| Gene name | GI# | Accession # | Organism |
| hibch | 146324906 | Q5XIE6.2 | Rattus norvegicus |
| hibch | 146324905 | Q6NVY1.2 | Homo sapiens |
| hibch | 2506374 | P28817.2 | Saccharomyces cerevisiae |
| BC_2292 | 29895975 | AP09256 | Bacillus cereus |

Yet another candidate hydrolase is the human dicarboxylic acid thioesterase, acot8, which exhibits activity on glutaryl-CoA, adipyl-CoA, suberyl-CoA, sebacyl-CoA, and dodecanedioyl-CoA (Westin et al., J. Biol. Chem. 280:38125-38132 (2005)) and the closest E. coli homolog, tesB, which can also hydrolyze a broad range of CoA thiolesters (Naggert et al., J Biol Chem 266:11044-11050 (1991)). A similar enzyme has also been characterized in the rat liver (Deana R., Biochem Int 26:767-773 (1992)).

| Gene name | GI# | GenBank Accession # | Organism |
|---|---|---|---|
| tesB | 16128437 | NP_414986 | Escherichia coli |
| acot8 | 3191970 | CAA15502 | Homo sapiens |
| acot8 | 51036669 | NP_570112 | Rattus norvegicus |

Other potential E. coli thiolester hydrolases include the gene products of tesA (Bonner et al., J Biol Chem 247:3123-3133 (1972)), ybgC (Kuznetsova et al., FEMS Microbiol Rev 29:263-279 (2005); Zhuang et al., FEBS Lett 516:161-163 (2002)), paaI (Song et al., J Blot Chem 281:11028-11038 (2006)), and ybdB (Leduc et al., J Bacteriol 189:7112-7126 (2007)).

| Gene name | GI# | GenBank Accession # | Organism |
|---|---|---|---|
| tesA | 16128478 | NP_415027 | Escherichia coli |
| ybgC | 16128711 | NP_415264 | Escherichia coli |
| paaI | 16129357 | NP_415914 | Escherichia coli |
| yciA | 1787506 | AAC74335.1 | Escherichia coli |
| ybdB | 16128580 | NP_415129 | Escherichia coli |

6.3.1/6.3.2 Amide Synthases/Peptide Synthases.

The direct conversion of 6-caprolactam (Step S, FIG. 1) requires the formation of an intramolecular peptide bond. Ribosomes, which assemble amino acids into proteins during translation, are nature's most abundant peptide bond-forming catalysts. Nonribosomal peptide synthetases are peptide bond forming catalysts that do not involve messenger mRNA (Schwarzer et al., Nat Prod. Rep. 20:275-287 (2003)). Additional enzymes capable of forming peptide bonds include acyl-CoA synthetase from Pseudomonas chlororaphis (Abe et al., J Biol Chem 283:11312-11321 (2008)), gamma-Glutamylputrescine synthetase from E. coli (Kurihara et al., J Biol Chem 283:19981-19990 (2008)), and beta-lactam synthetase from Streptomyces clavuligerus (Bachmann et al., Proc Natl Acad Sci USA 95:9082-9086 (1998); Bachmann et al., Biochemistry 39:11187-11193 (2000); Miller et al., Nat Struct. Biol 8:684-689 (2001); Miller et al., Proc Natl Acad Sci USA 99:14752-14757 (2002); Tahlan et al., Antimicrob. Agents. Chemother. 48:930-939 (2004)).

| | | GenBank | |
|---|---|---|---|
| Gene name | GI# | Accession # | Organism |
| acsA | 60650089 | BAD90933 | Pseudomonas chlororaphis |
| puuA | 87081870 | AAC74379 | Escherichia coli |
| bls | 41016784 | Q9R8E3 | Streptomyces clavuligerus |

4.2.1 Hydrolyase.

Most dehydratases catalyze the α, β-elimination of water. This involves activation of the α-hydrogen by an electron-withdrawing carbonyl, carboxylate, or CoA-thiol ester group and removal of the hydroxyl group from the β-position. Enzymes exhibiting activity on substrates with an electron-withdrawing carboxylate group are excellent candidates for dehydrating 3-hydroxyadipate (FIG. 1, Step I).

For example, fumarase enzymes naturally catalyze the reversible dehydration of malate to fumarate. E. coli has three fumarases: FumA, FumB, and FumC that are regulated by growth conditions. FumB is oxygen sensitive and only active under anaerobic conditions. FumA is active under microanaerobic conditions, and FumC is the only active enzyme in aerobic growth (Tseng et al., J Bacteriol 183:461-467 (2001); Woods et al., Biochim Biophys Acta 954:14-26 (1988); Guest et al., J Gen Microbiol 131:2971-2984 (1985)). Additional enzyme candidates are found in Campylobacter jejuni (Smith et al., Int. J Biochem. Cell Biol 31:961-975 (1999)), Thermus thermophilus (Mizobata et al., Arch. Biochem. Biophys. 355:49-55 (1998)) and Rattus norvegicus (Kobayashi et al., J Biochem. 89:1923-1931 (1981)). Similar enzymes with high sequence homology include fum1 from Arabidopsis thaliana and fumC from Corynebacterium glutamicum. The MmcBC fumarase from Pelotomaculum thermopropionicum is another class of fumarase with two subunits (Shimoyama et al., FEMS Microbiol Lett 270:207-213 (2007)). Additional dehydratase candidates are known in the art and include those of U.S. Pat. No. 8,377,680 which is herein incorporated in its entirety and for all purposes.

| Gene | | GenBank | |
|---|---|---|---|
| name | GI# | Accession # | Organism |
| fumA | 81175318 | P0AC33 | Escherichia coli |
| fumB | 33112655 | P14407 | Escherichia coli |
| fumC | 120601 | P05042 | Escherichia coli |
| fumC | 9789756 | O69294 | Campylobacter jejuni |
| fumC | 3062847 | BAA25700 | Thermus thermophilus |
| fumH | 120605 | P14408 | Rattus norvegicus |
| fumI | 39931311 | P93033 | Arabidopsis thaliana |
| fumC | 39931596 | Q8NRN8 | Corynebacterium glutamicum |
| MmcB | 147677691 | YP_001211906 | Pelotomaculum |
| | | | thermopropionicum |
| MmcC | 147677692 | YP_001211907 | Pelotomaculum |
| | | | thermopropionicum |

Enzymes exhibiting activity on substrates with an electron-withdrawing CoA-thiol ester group adjacent to the α-hydrogen are excellent candidates for dehydrating 3-hydroxyadipyl-CoA (FIG. 1, Step C). The enoyl-CoA hydratases, phaA and phaB, of P. putida are believed to carry out the hydroxylation of double bonds during phenylacetate catabolism (Olivera et al., Proc. Natl. Acad. Sci. USA 95:6419-6424 (1998)). The paaA and paaB from P. fluorescens catalyze analogous transformations (Olivera et al., Proc. Natl. Acad. Sci. USA 95:6419-6424 (1998)). Lastly, a number of Escherichia coli genes have been shown to demonstrate enoyl-CoA hydratase functionality including maoC (Park et al., J Bacteriol. 185:5391-5397 (2003)), paaF (Ismail et al., supra; Park et al., Appl. Biochem. Biotechnol 113-116:335-346 (2004); Park et al., Biotechnol Bioeng 86:681-686 (2004)) and paaG (Ismail et al., supra; Park et al., Appl. Biochem. Biotechnol 113-116:335-346 (2004); Park et al., Biotechnol Bioeng 86:681-686 (2004)). Crotonase enzymes are additional candidates for dehydrating the required 3-hydroxyacyl-CoA molecules depicted in FIG. 1. These enzymes are required for n-butanol formation in some organisms, particularly Clostridial species, and also comprise one step of the 3-hydroxypropionate/4-hydroxybutyrate cycle in thermoacidophilic Archaea of the genera Sulfolobus, Acidianus, and Metallosphaera. Exemplary genes encoding crotonase enzymes can be found in C. acetobutylicum (Boynton et al., supra), C. kluyveri (Hillmer et al., FEBS Lett. 21:351-354 (1972)), and Metallosphaera sedula (Berg et al., supra) though the sequence of the latter gene is not known. Enoyl-CoA hydratases, which are involved in fatty acid beta-oxidation and/or the metabolism of various amino acids, can also catalyze the hydration of crotonyl-CoA to form 3-hydroxybutyryl-CoA (Roberts et al., Arch. Microbiol 117:99-108 (1978); Agnihotri et al., Bioorg. Med. Chem. 11:9-20 (2003); Conrad et al., J Bacteriol. 118:103-111 (1974)).

| Gene | | GenBank | |
|---|---|---|---|
| name | GI# | Accession # | Organism |
| PP_3284 | 26990002 | NP_745427.1 | Pseudomonas putida |
| | | | KT2440 |
| phaB | 26990001 | NP_745426.1 | Pseudomonas putida |
| | | | KT2440 |
| paaA | 106636093 | ABF82233.1 | Pseudomonas putida |
| paaB | 106636094 | ABF82234.1 | Pseudomonas putida |
| maoC | 16129348 | NP_415905.1 | Escherichia coli |
| paaF | 16129354 | NP_415911.1 | Escherichia coli |
| paaG | 16129355 | NP_415912.1 | Escherichia coli |
| crt | 15895969 | NP_349318.1 | Clostridium |
| | | | acetobutylicum |
| crt1 | 153953091 | YP_001393856 | Clostridium kluyveri |
| | | | DSM 555 |
| h16_A3307 | 113869255 | YP_727744.1 | Ralstonia eutropha H16 |
| | | | (Cupriavidus necator) |
| dcaE | 50084847 | YP_046357.1 | Acinetobacter sp. ADP1 |

Several of these candidates have been tested inhouse for activity on 3-hydroxy adipyl-CoA and have demonstrated activity.

6.2.1 Acid-Thiol Ligase.

Steps F, L, and R of FIG. 1 require acid-thiol ligase or synthetase functionality (the terms ligase, synthetase, and synthase are used herein interchangeably and refer to the same enzyme class). Exemplary genes encoding enzymes likely to carry out these transformations include the sucCD genes of E. coli which naturally form a succinyl-CoA synthetase complex. This enzyme complex naturally catalyzes the formation of succinyl-CoA from succinate with the concaminant consumption of one ATP, a reaction which is reversible in vivo (Buck et al., Biochem. 24:6245-6252 (1985)). Given the structural similarity between succinate and adipate, that is, both are straight chain dicarboxylic acids, it is reasonable to expect some activity of the sucCD enzyme on adipyl-CoA. Additional exemplary CoA-ligases are known in the art and exemplified for example in U.S. Pat. No. 8,377,680 which is herein incorporated in its entirety and for all purposes.

| Gene name | GI# | GenBank Accession # | Organism |
|---|---|---|---|
| sucC | 16128703 | NP_415256.1 | Escherichia coli |
| sucD | 1786949 | AAC73823.1 | Escherichia coli |

No enzyme required—Spontaneous cyclization. 6-Aminocaproyl-CoA will cyclize spontaneously to caprolactam, thus eliminating the need for a dedicated enzyme for this step. A similar spontaneous cyclization is observed with 4-aminobutyryl-CoA which forms pyrrolidinone (Ohsugi et al., J Biol Chem 256:7642-7651 (1981)).

### Example 2: Production of Caprolactone

Pathways for producing caprolactone are depicted in FIG. 5. FIG. 5 shows pathways for converting adipate or adipyl-CoA to caprolactone. Adipate is an intermediate produced during the degradation of aromatic and aliphatic ring containing compounds such as cyclohexanol. Biosynthetic pathways for forming adipate and adipyl-CoA are well known in the art (for example, see U.S. Pat. No. 7,799,545). In the pathway shown in FIG. 5, adipate semialdehyde is formed either from adipate via an adipate reductase (Step E) or adipyl-CoA via adipyl-CoA reductase (Step A). Adipate semialdehyde is then reduced to 5-hydroxyhexanoate in Step B. The 6-hydroxyhexanoate intermediate is converted to caprolactone by one of several alternate routes. In one route, 6-hydroxyhexanoate is directly converted to caprolactone by a caprolactone hydrolase (step G). In yet another route, 6-hydroxyhexanoate is activated to its corresponding acyl-CoA, which then cyclizes to caprolactone (step C/D), or cyclizes via a 6-hydroxyhexanoyl-phosphate intermediate (steps J/I). In an alternate route, 6-hydroxyhexanote is activated to 6-hydroxyhexanoyl-phosphate, which is then cyclized to caprolactone (step H/I).

1.1.1 Alcohol Dehydrogenase.

Alcohol dehydrogenase enzymes catalyze Step B of FIG. 5. Exemplary alcohol dehydrogenase enzymes are described in further detail below.

6-Hydroxyhexanoate dehydrogenase (adipate semialdehyde reductase) catalyzes the reduction of adipate semialdehyde to 6-hydroxyhexanoate. Such an enzyme is required in Step B of FIG. 5. Enzymes with this activity are found in organisms that degrade cyclohexanone, and are encoded by chnD of Acinetobacter sp. NCIMB9871 (Iwaki et al, AEM 65:5158-62 (1999)), Rhodococcus sp. Phi2 and Arthrobacter sp. BP2 (Brzostowicz et al, AEM 69:334-42 (2003)).

| Gene | GenBank ID | GI Number | Organism |
|---|---|---|---|
| chnD | BAC80217.1 | 33284997 | Acinetobacter sp. NCIMB9871 |
| chnD | AAN37477.1 | 27657618 | Arthrobacter sp. BP2 |
| chnD | AAN37489.1 | 27657631 | Rhodococcus sp. Phi2 |

Additional aldehyde reductase enzymes are shown in the table below. AlrA encodes a medium-chain alcohol dehydrogenase for C2-C14 compounds (Tani et al., Appl. Environ. Microbiol. 66:5231-5235 (2000)). Other candidates are yqhD and fucO from E. coli (Sulzenbacher et al., 342:489-502 (2004)), and bdh I and bdh II from C. acetobutylicum (Walter et al., 174:7149-7158 (1992)). YqhD catalyzes the reduction of a wide range of aldehydes using NADPH as the cofactor, with a preference for chain lengths longer than C(3) (Sulzenbacher et al., 342:489-502 (2004); Perez et al., J Biol. Chem. 283:7346-7353 (2008)). The adhA gene product from Zymomonas mobilis has been demonstrated to have activity on a number of aldehydes including formaldehyde, acetaldehyde, propionaldehyde, butyraldehyde, and acrolein (Kinoshita et al., Appl Microbiol Biotechnol 22:249-254 (1985)). Additional aldehyde reductase candidates are encoded by bdh in C. saccharoperbutylacetonicum and Cbei_1722, Cbei_2181 and Cbei_2421 in C. beijerinckii.

| Protein | GenBank ID | GI number | Organism |
|---|---|---|---|
| alrA | BAB12273.1 | 9967138 | Acinetobacter sp. strain M-1 |
| ADH2 | NP_014032.1 | 6323961 | Saccharomyces cerevisiae |
| yqhD | NP_417484.1 | 16130909 | Escherichia coli |
| fucO | NP_417279.1 | 16130706 | Escherichia coli |
| bdh I | NP_349892.1 | 15896543 | Clostridium acetobutylicum |
| bdh II | NP_349891.1 | 15896542 | Clostridium acetobutylicum |
| adhA | YP_162971.1 | 56552132 | Zymomonas mobilis |
| bdh | BAF45463.1 | 124221917 | Clostridium saccharoperbutylacetonicum |
| Cbei_1722 | YP_001308850 | 150016596 | Clostridium beijerinckii |
| Cbei_2181 | YP_001309304 | 150017050 | Clostridium beijerinckii |
| Cbei_2421 | YP_001309535 | 150017281 | Clostridium beijerinckii |

Other enzymes performing similar catalysis are known in the art and useful for Step B of FIG. 5. Such enzymes include those exemplified in U.S. Pat. No. 8,940,509 which are herein incorporated in its entirety and for all purposes.

1.2.1 Oxidoreductase (acyl-CoA to Aldehyde).

An adipyl-CoA reductase converts adipyl-CoA to adipate semialdehyde in Step A of FIG. 5. Several acyl-CoA reductase enzymes are found in EC class 1.2.1. Exemplary enzymes include fatty acyl-CoA reductase, succinyl-CoA reductase (EC 1.2.1.76), acetyl-CoA reductase, butyryl-CoA reductase and propionyl-CoA reductase (EC 1.2.1.3). Exemplary fatty acyl-CoA reductases enzymes are encoded by acr1 of Acinetobacter calcoaceticus (Reiser, Journal of Bacteriology 179:2969-2975 (1997)) and Acinetobacter sp. M-1 (Ishige et al., Appl. Environ. Microbiol. 68:1192-1195 (2002)). Enzymes with succinyl-CoA reductase activity are encoded by sucD of Clostridium kluyveri (Sohling, J. Bacteriol. 178:871-880 (1996)) and sucD of P. gingivalis (Takahashi, J. Bacteriol 182:4704-4710 (2000)). Additional succinyl-CoA reductase enzymes participate in the 3-hydroxypropionate/4-hydroxybutyrate cycle of thermophilic archaea including Metallosphaera sedula (Berg et al., Science 318:1782-1786 (2007)) and Thermoproteus neutrophilus (Ramos-Vera et al., J Bacteriol, 191:4286-4297 (2009)). The M. sedula enzyme, encoded by Msed_0709, is strictly NADPH-dependent and also has malonyl-CoA reductase activity. The T. neutrophilus enzyme is active with both NADPH and NADH. The enzyme acylating acetaldehyde dehydrogenase in Pseudomonas sp, encoded by bphG, is yet another as it has been demonstrated to oxidize and acylate acetaldehyde, propionaldehyde, butyraldehyde, isobutyraldehyde and formaldehyde (Powlowski, J. Bacteriol. 175:377-385 (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Kazahaya, J. Gen. Appl. Microbiol. 18:43-55 (1972); and Koo et al., Biotechnol Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci Biotechnol Biochem., 71:58-68 (2007)). Exemplary propionyl-CoA reductase enzymes include pduP of Salmonella typhimurium LT2 (Leal, Arch. Microbiol. 180:353-361 (2003)) and eutE from E. coli (Skraly, WO Patent No. 2004/024876). The propionyl-CoA reductase of Salmonella typhimurium LT2, which naturally converts propionyl-CoA to propionaldehyde, also catalyzes the reduction of 5-hydroxyvaleryl-CoA to 5-hydroxypentanal (WO 2010/068953A2).

| Protein | GenBank ID | GI Number | Organism |
|---|---|---|---|
| acr1 | YP_047869.1 | 50086359 | Acinetobacter calcoaceticus |
| acr1 | AAC45217 | 1684886 | Acinetobacter baylyi |
| acr1 | BAB85476.1 | 18857901 | Acinetobacter sp. Strain M-1 |
| Msed_0709 | YP_001190808.1 | 146303492 | Metallosphaera sedula |
| Tneu_0421 | ACB39369.1 | 170934108 | Thermoproteus neutrophilus |
| sucD | P38947.1 | 172046062 | Clostridium kluyveri |
| sucD | NP_904963.1 | 34540484 | Porphyromonas gingivalis |
| bphG | BAA03892.1 | 425213 | Pseudomonas sp |
| adhE | AAV66076.1 | 55818563 | Leuconostoc mesenteroides |
| bld | AAP42563.1 | 31075383 | Clostridium |
| | | | saccharoperbutylacetonicum |
| pduP | NP_460996 | 16765381 | Salmonella typhimurium LT2 |
| eutE | NP_416950 | 16130380 | Escherichia coli |

Additional enzyme types and enzymes that convert an acyl-CoA to its corresponding aldehyde are known in the art and exemplified in for example U.S. Pat. No. 8,940,509 which are herein incorporated in its entirety and for all purposes.

1.2.1 (CAR).

The conversion of an acid to an aldehyde is thermodynamically unfavorable and typically requires energy-rich cofactors and multiple enzymatic steps. For example, in butanol biosynthesis conversion of butyrate to butyraldehyde is catalyzed by activation of butyrate to its corresponding acyl-CoA by a CoA transferase or ligase, followed by reduction to butyraldehyde by a CoA-dependent aldehyde dehydrogenase. Alternately, an acid can be activated to an acyl-phosphate and subsequently reduced by a phosphate reductase. Direct conversion of the acid to aldehyde by a single enzyme is catalyzed by a bifunctional enzyme in the 1.2.1 family. Exemplary enzymes that catalyze these transformations include carboxylic acid reductase, alpha-aminoadipate reductase and retinoic acid reductase.

Carboxylic acid reductase (CAR), found in Nocardia iowensis, catalyzes the magnesium, ATP and NADPH-dependent reduction of carboxylic acids to their corresponding aldehydes (Venkitasubramanian et al., J Biol. Chem. 282:478-485 (2007)). The natural substrate of this enzyme is benzoic acid and the enzyme exhibits broad acceptance of aromatic and aliphatic substrates (Venkitasubramanian et al., Biocatalysis in Pharmaceutical and Biotechnology Industries. CRC press (2006)). This enzyme, encoded by car, was cloned and functionally expressed in E. coli (Venkitasubramanian et al., J Biol. Chem. 282:478-485 (2007)). CAR requires post-translational activation by a phosphopantetheine transferase (PPTase) that converts the inactive apo-enzyme to the active holo-enzyme (Hansen et al., Appl. Environ. Microbiol 75:2765-2774 (2009)). Expression of the npt gene, encoding a specific PPTase, product improved activity of the enzyme. An enzyme with similar characteristics, alpha-aminoadipate reductase (AAR, EC 1.2.1.31), participates in lysine biosynthesis pathways in some fungal species. This enzyme naturally reduces alpha-aminoadipate to alpha-aminoadipate semialdehyde. The carboxyl group is first activated through the ATP-dependent formation of an adenylate that is then reduced by NAD(P)H to yield the aldehyde and AMP. Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279 (2003)), and Schizosaccharomyces pombe (Ford et al., Curr. Genet. 28:131-137 (1995)). The AAR from S. pombe exhibited significant activity when expressed in E. coli (Guo et al., Yeast 21:1279-1288 (2004)). The AAR from Penicillium chrysogenum accepts S-carboxymethyl-L-cysteine as an alternate substrate, but did not react with adipate, L-glutamate or diaminopimelate (Hijarrubia et al., J Biol. Chem. 278:8250-8256 (2003)). The gene encoding the P. chrysogenum PPTase has not been identified to date and no high-confidence hits were identified by sequence comparison homology searching.

| Protein | GenBank ID | GI Number | Organism |
|---|---|---|---|
| car | AAR91681.1 | 40796035 | Nocardia iowensis |
| npt | ABI83656.1 | 114848891 | Nocardia iowensis |
| LYS2 | AAA34747.1 | 171867 | Saccharomyces cerevisiae |
| LYSS | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Penicillium chrysogenum |

2.3.1 Acyltransferase (Transferring Phosphate Group).

An enzyme with phosphotrans-6-hydroxyhexanoylase activity is required to convert 6-hydroxyhexanoyl-CoA to 6-hydroxyhexanoyl phosphate (Step J of FIG. 5). Exemplary phosphate-transferring acyltransferases include phosphotransacetylase (EC 2.3.1.8) and phosphotransbutyrylase (EC 2.3.1.19). The pta gene from E. coli encodes a phosphotransacetylase that reversibly converts acetyl-CoA into acetyl-phosphate (Suzuki, Biochim. Biophys. Acta 191:559-569 (1969)). This enzyme can also utilize propionyl-CoA as a substrate, forming propionate in the process (Hesslinger et al., Mol. Microbiol 27:477-492 (1998)). Other phosphate acetyltransferases that exhibit activity on propionyl-CoA are found in Bacillus subtilis (Rado et al., Biochim. Biophys. Acta 321:114-125 (1973)), Clostridium kluyveri (Stadtman, Methods Enzymol 1:596-599 (1955)), and Thermotoga maritima (Bock et al., J Bacteriol. 181:1861-1867 (1999)). Similarly, the ptb gene from C. acetobutylicum encodes phosphotransbutyrylase, an enzyme that reversibly converts butyryl-CoA into butyryl-phosphate (Wiesenborn et al., Appl Environ. Microbiol 55:317-322 (1989); Walter et al., Gene 134:107-111 (1993)). Additional ptb genes are found in butyrate-producing bacterium L2-50 (Louis et al., J. Bacteriol. 186:2099-2106 (2004)) and Bacillus megaterium (Vazquez et al., Curr. Microbiol 42:345-349 (2001)).

| Protein | GenBank ID | GI Number | Organism |
|---|---|---|---|
| pta | NP_416800.1 | 71152910 | Escherichia coli |
| pta | P39646 | 730415 | Bacillus subtilis |
| pta | A5N801 | 146346896 | Clostridium kluyveri |
| pta | Q9X0L4 | 6685776 | Thermotoga maritima |
| ptb | NP_349676 | 34540484 | Clostridium acetobutylicum |
| ptb | AAR19757.1 | 38425288 | butyrate-producing bacterium L2-50 |
| ptb | CAC07932.1 | 10046659 | Bacillus megaterium |

2.7.2 Phosphotransferase (Carboxy Group Acceptor).

Kinase or phosphotransferase enzymes in the EC class 2.7.2 transform carboxylic acids to phosphonic acids with concurrent hydrolysis of one ATP. Such an enzyme is required for the phosphorylation of 6-hydroxyhexanoate depicted in Step H of FIG. 5. Exemplary enzyme candidates include butyrate kinase (EC 2.7.2.7), isobutyrate kinase (EC 2.7.2.14), aspartokinase (EC 2.7.2.4), acetate kinase (EC 2.7.2.1), glycerate kinase (EC 2.7.1.31) and gamma-glutamyl kinase (EC 2.7.2.11). Butyrate kinase catalyzes the reversible conversion of butyryl-phosphate to butyrate during acidogenesis in Clostridial species (Cary et al., Appl Environ Microbiol 56:1576-1583 (1990)). The Clostridium acetobutylicum enzyme is encoded by either of the two buk gene products (Huang et al., J Mol. Microbiol Biotechnol 2:33-38 (2000)). Other butyrate kinase enzymes are found in C. butyricum and C. tetanomorphum (Twarog et al., J Bacteriol. 86:112-117 (1963)). A related enzyme, isobutyrate kinase from Thermotoga maritima, was expressed in E. coli and crystallized (Diao et al., J Bacteriol. 191:2521-2529 (2009); Diao et al., Acta Crystallogr. D. Biol. Crystallogr. 59:1100-1102 (2003)). Aspartokinase catalyzes the ATP-dependent phosphorylation of aspartate and participates in the synthesis of several amino acids. The aspartokinase III enzyme in E. coli, encoded by lysC, has a broad substrate range and the catalytic residues involved in substrate specificity have been elucidated (Keng et al., Arch Biochem Biophys 335:73-81 (1996)). Two additional kinases in E. coli are also acetate kinase and gamma-glutamyl kinase. The E. coli acetate kinase, encoded by ackA (Skarstedt et al., J. Biol. Chem. 251:6775-6783 (1976)), phosphorylates propionate in addition to acetate (Hesslinger et al., Mol. Microbiol 27:477-492 (1998)). The E. coli gamma-glutamyl kinase, encoded by proB (Smith et al., J. Bacteriol. 157:545-551 (1984)), phosphorylates the gamma carbonic acid group of glutamate.

| Protein | GenBank ID | GI Number | Organism |
|---|---|---|---|
| buk1 | NP_349675 | 15896326 | Clostridium acetobutylicum |
| buk2 | Q97II1 | 20137415 | Clostridium acetobutylicum |
| buk2 | Q9278.1 | 6685256 | Thermotoya maritima |
| lysC | NP_418448.1 | 16131850 | Escherichia coli |
| ackA | NP_416799.1 | 16130231 | Escherichia coli |
| proB | NP_414777.1 | 16128228 | Escherichia coli |

Acetylglutamate kinase phosphorylates acetylated glutamate during arginine biosynthesis. This enzyme is not known to accept alternate substrates; however, several residues of the E. coli enzyme involved in substrate binding and phosphorylation have been elucidated by site-directed mutagenesis (Marco-Marin et al., 334:459-476 (2003); Ramon-Maiques et al., Structure. 10:329-342 (2002)). The enzyme is encoded by argB in Bacillus subtilis and E. coli (Parsot et al., Gene 68:275-283 (1988)), and ARG5,6 in S. cerevisiae (Pauwels et al., Eur. J Biochem. 270:1014-1024 (2003)). The ARG5,6 gene of S. cerevisiae encodes a polyprotein precursor that is matured in the mitochondrial matrix to become acetylglutamate kinase and acetylglutamylphosphate reductase.

| Protein | GenBank ID | GI Number | Organism |
|---|---|---|---|
| argB | NP_418394.3 | 145698337 | Escherichia coli |
| argB | NP_389003.1 | 16078186 | Bacillus subtilis |
| ARG5,6 | NP_010992.1 | 6320913 | Saccharomyces cerevisiae |

Glycerate kinase (EC 2.7.1.31) activates glycerate to glycerate-2-phosphate or glycerate-3-phosphate. Three classes of glycerate kinase have been identified. Enzymes in class I and II produce glycerate-2-phosphate, whereas the class III enzymes found in plants and yeast produce glycerate-3-phosphate (Bartsch et al., FEBS Lett. 582:3025-3028 (2008)). In a recent study, class III glycerate kinase enzymes from Saccharomyces cerevisiae, Oryza sativa and Arabidopsis thaliana were heterologously expressed in E. coli and characterized (Bartsch et al., FEBS Lett. 582:3025-3028 (2008)). This study also assayed the glxK gene product of E. coli for ability to form glycerate-3-phosphate and found that the enzyme can only catalyze the formation of glycerate-2-phosphate, in contrast to previous work (Doughty et al., J Biol. Chem. 241:568-572 (1966)).

| Protein | GenBank ID | GI Number | Organism |
|---|---|---|---|
| glxK | AAC73616.1 | 1786724 | Escherichia coli |
| YGR205W | AAS56599.1 | 45270436 | Saccharomyces cerevisiae |
| Os01g0682500 | BAF05800.1 | 113533417 | Oryza sativa |
| At1g80380 | BAH57057.1 | 227204411 | Arabidopsis thaliana |

2.8.3 CoA Transferase.

CoA transferases catalyze the reversible transfer of a CoA moiety from one molecule to another. Several transformations require a CoA transferase to interconvert carboxylic acids and their corresponding acyl-CoA derivatives, including steps C and F of FIG. 5. CoA transferase enzymes have been described in the open literature and represent suitable candidates for these steps. Exemplary candidates are described below.

Many transferases have broad specificity and thus can utilize CoA acceptors as diverse as acetate, succinate, propionate, butyrate, 2-methylacetoacetate, 3-ketohexanoate, 3-ketopentanoate, valerate, crotonate, 3-mercaptopropionate, propionate, vinylacetate, butyrate, among others. For example, an enzyme from Roseburia sp. A2-183 was shown to have butyryl-CoA:acetate:CoA transferase and propionyl-CoA:acetate:CoA transferase activity (Charrier et al., Microbiology 152, 179-185 (2006)). Close homologs can be found in, for example, Roseburia intestinalis L1-82, Roseburia inulinivorans DSM 16841, Eubacterium rectale ATCC 33656. Another enzyme with propionyl-CoA transferase activity can be found in Clostridium propionicum (Selmer et al., Eur J Biochem 269, 372-380 (2002)). This enzyme can use acetate, (R)-lactate, (S)-lactate, acrylate, and butyrate as the CoA acceptor (Selmer et al., Eur J Biochem 269, 372-380 (2002); Schweiger and Buckel, FEBS Letters, 171(1) 79-84 (1984)). Close homologs can be found in, for example, Clostridium novyi NT, Clostridium beijerinckii NCIMB 8052, and Clostridium botulinum C str. Eklund. YgfH encodes a propionyl CoA: succinate CoA transferase in E. coli (Haller et al., Biochemistry, 39(16) 4622-4629). Close homologs can be found in, for example, Citrobacter youngae ATCC 29220, Salmonella enterica subsp. arizonae serovar, and Yersinia intermedia ATCC 29909. These proteins are identified below. Other candidates are well known and discussed in the art and include those exemplified for example by U.S. Pat. No. 8,940,509 which are herein incorporated in its entirety and for all purposes.

| Protein | GenBank ID | GI Number | Organism |
|---|---|---|---|
| Ach1 | AAX19660.1 | 60396828 | Roseburia sp. A2-183 |
| ROSINTL182_07121 | ZP_04743841.2 | 257413684 | Roseburia intestinalis |
| ROSEINA2194_03642 | ZP_03755203.1 | 225377982 | Roseburia inulinivorans |
| EUBREC_3075 | YP_002938937.1 | 238925420 | Eubacterium rectale |
| pct | CAB77207.1 | 7242549 | Clostridium propionicum |
| NT01CX_2372 | YP_878445.1 | 118444712 | Clostridium novyi NT |
| Cbei_4543 | YP_001311608.1 | 150019354 | Clostridium beijerinckii |
| CBC_A0889 | ZP_02621218.1 | 168186583 | Clostridium botulinum |
| ygfH | NP_417395.1 | 16130821 | Escherichia coli |
| CIT292_04485 | ZP_03838384.1 | 227334728 | Citrobacter youngae |
| SARI_04582 | YP _001573497.1 | 161506385 | Salmonella enterica |
| yinte0001_14430 | ZP _04635364.1 | 238791727 | Yersinia intermedia |

3.1.1 Esterase/Lipase.

Enzymes in the EC class 3.1.1 catalyze the hydrolysis and synthesis of ester bonds. Caprolactone hydrolase enzymes required for step G of FIG. 5 are found in organisms that degrade cyclohexanone. The chnC gene product of Acinetobacter sp. NCIMB9871 was found to hydrolyze the ester bond of caprolactone, forming 6-hydroxyhexanote (Iwaki et al, AEM 65:5158-62 (1999)). Similar enzymes were identified in Arthrobacter sp. BP2 and Rhodococcus sp. Phi2 (Brzostowicz et al, AEM 69:334-42 (2003)).

| Gene | GenBank ID | GI Number | Organism |
|---|---|---|---|
| chnC | BAC80218.1 | 33284998 | Acinetobacter sp. NCIMB9871 |
| chnC | AAN37478.1 | 27657619 | Arthrobacter sp. BP2 |
| chnC | AAN37490.1 | 27657632 | Rhodococcus sp. Phi2 |

Formation of caprolactone may also be catalyzed by enzymes that catalyze the interconversion of cyclic lactones and open chain hydroxycarboxylic acids. The L-lactonase from Fusarium proliferatum ECU2002 exhibits lactonase and esterase activities on a variety of lactone substrates (Zhang et al., Appl. Microbiol. Biotechnol. 75:1087-1094 (2007)). The 1,4-lactone hydroxyacylhydrolase (EC 3.1.1.25), also known as 1,4-lactonase or gamma-lactonase, is specific for 1,4-lactones with 4-8 carbon atoms. The gamma lactonase in human blood and rat liver microsomes was purified (Fishbein et al., J Biol Chem 241:4835-4841 (1966)) and the lactonase activity was activated and stabilized by calcium ions (Fishbein et al., J Biol Chem 241:4842-4847 (1966)). The optimal lactonase activities were observed at pH 6.0, whereas high pH resulted in hydrolytic activities (Fishbein and Bessman, J Biol Chem 241:4842-4847 (1966)). Genes from Xanthomonas campestris, Aspergillus niger and Fusarium oxysporum have been annotated as 1,4-lactonase and can be utilized to catalyze the transformation of 4-hydroxybutyrate to GBL (Zhang et al., Appl Microbiol Biotechnol 75:1087-1094 (2007)).

| Gene | Accession No. | GI No. | Organism |
|---|---|---|---|
| EU596535.1: 1 . . . 1206 | ACC61057.1 | 183238971 | Fusarium proliferation |
| xccb100_2516 | YP_001903921.1 | 188991911 | Xanthomonas campestris |
| An16g06620 | CAK46996.1 | 134083519 | Aspergillus niger |
| BAA34062 | BAA34062.1 | 3810873 | Fusarium oxysporum |

Other enzyme candidates for converting 6-hydroxyhexanoate to caprolactone are well known in the art (including for example lipases and esterases) and include those exemplified in for example U.S. Pat. No. 8,940,509 which are herein incorporated in its entirety and for all purposes.

3.1.2 CoA Hydrolase.

Enzymes in the 3.1.2 family hydrolyze acyl-CoA molecules to their corresponding acids. Such an enzyme is depicted in Step F of FIG. 5. Several CoA hydrolases have been demonstrated to hydrolyze adipyl-CoA, or alternately accept a broad range of substrates. For example, the enzyme encoded by acot12 from Rattus norvegicus brain (Robinson et al., Biochem. Biophys. Res. Commun. 71:959-965 (1976)) can react with butyryl-CoA, hexanoyl-CoA and malonyl-CoA. The human dicarboxylic acid thioesterase, encoded by acot8, exhibits activity on glutaryl-CoA, adipyl-CoA, suberyl-CoA, sebacyl-CoA, and dodecanedioyl-CoA (Westin et al., J. Biol. Chem. 280:38125-38132 (2005)). The closest E. coli homolog to this enzyme, tesB, can also hydrolyze a range of CoA thiolesters (Naggert et al., J Biol Chem 266:11044-11050 (1991)). A similar enzyme has also been characterized in the rat liver (Deana R., Biochem Int 26:767-773 (1992)). Additional enzymes with hydrolase activity in E. coli include ybgC, paaI, and ybdB (Kuznetsova, et al., FEMS Microbiol Rev, 2005, 29(2):263-279; Song et al., J Biol Chem, 2006, 281(16):11028-38). Though its sequence has not been reported, the enzyme from the mitochondrion of the pea leaf has a broad substrate specificity, with demonstrated activity on acetyl-CoA, propionyl-CoA, butyryl-CoA, palmitoyl-CoA, oleoyl-CoA, succinyl-CoA, and crotonyl-CoA (Zeiher et al., Plant. Physiol. 94:20-27 (1990)) The acetyl-CoA hydrolase, ACH1, from S. cerevisiae represents another candidate hydrolase (Buu et al., J. Biol. Chem. 278:17203-17209 (2003)).

| Gene name | GenBank ID | GI number | Organism |
|---|---|---|---|
| acot12 | NP_570103.1 | 18543355 | Rattus norvegicus |
| tesB | NP_414986 | 16128437 | Escherichia coli |
| acot8 | CAA15502 | 3191970 | Homo sapiens |
| acot8 | NP_570112 | 51036669 | Rattus norvegicus |
| tesA | NP_415027 | 16128478 | Escherichia coli |
| ybgC | NP_415264 | 16128711 | Escherichia coli |
| paaI | NP_415914 | 16129357 | Escherichia coli |
| ybdB | NP_415129 | 16128580 | Escherichia coli |
| ACH1 | NP_009538 | 6319456 | Saccharomyces cerevisiae |

Other candidate hydrolases useful for Step F of FIG. 5 include those known in the art and described in U.S. Pat. No. 8,940,509 which are herein incorporated in its entirety and for all purposes

6.2.1 CoA Synthetase.

The conversion of acyl-CoA substrates to their acid products can be catalyzed by a CoA acid-thiol ligase or CoA synthetase in the 6.2.1 family of enzymes. Several transformations require a CoA synthetase to interconvert carboxylic acids and their corresponding acyl-CoA derivatives, including steps C and F of FIG. 5. Enzymes catalyzing these exact transformations have not been characterized to date; however, several enzymes with broad substrate specificities have been described in the literature.

ADP-forming acetyl-CoA synthetase (ACD, EC 6.2.1.13) is an enzyme that couples the conversion of acyl-CoA esters to their corresponding acids with the concomitant synthesis of ATP. ACD I from Archaeoglobus fulgidus, encoded by AF1211, was shown to operate on a variety of linear and branched-chain substrates including isobutyrate, isopentanoate, and fumarate (Musfeldt et al., J Bacteriol. 184:636-644 (2002)). A second reversible ACD in Archaeoglobus fulgidus, encoded by AF1983, was also shown to have a broad substrate range with high activity on cyclic compounds phenylacetate and indoleacetate (Musfeldt and Schonheit, J Bacteriol. 184:636-644 (2002)). The enzyme from Haloarcula marismortui (annotated as a succinyl-CoA synthetase) accepts propionate, butyrate, and branched-chain acids (isovalerate and isobutyrate) as substrates, and was shown to operate in the forward and reverse directions (Brasen et al., Arch Microbiol 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen et al, supra). Directed evolution or engineering can be used to modify this enzyme to operate at the physiological temperature of the host organism. The enzymes from A. fulgidus, H. marismortui and P. aerophilum have all been cloned, functionally expressed, and characterized in E. coli (Brasen and Schonheit, supra; Musfeldt and Schonheit, J Bacteriol. 184:636-644 (2002)). An additional candidate is succinyl-CoA synthetase, encoded by sucCD of E. coli and LSC1 and LSC2 genes of Saccharomyces cerevisiae. These enzymes catalyze the formation of succinyl-CoA from succinate with the concomitant consumption of one ATP in a reaction which is reversible in vivo (Buck et al., Biochemistry 24:6245-6252 (1985)). The acyl CoA ligase from Pseudomonas putida has been demonstrated to work on several aliphatic substrates including acetic, propionic, butyric, valeric, hexanoic, heptanoic, and octanoic acids and on aromatic compounds such as phenylacetic and phenoxyacetic acids (Fernandez-Valverde et al., Appl. Environ. Microbiol. 59:1149-1154 (1993)). A related enzyme, malonyl CoA synthetase (6.3.4.9) from Rhizobium leguminosarum could convert several diacids, namely, ethyl-, propyl-, allyl-, isopropyl-, dimethyl-, cyclopropyl-, cyclopropylmethylene-, cyclobutyl-, and benzyl-malonate into their corresponding monothioesters (Pohl et al., J. Am. Chem. Soc. 123:5822-5823 (2001)).

| Protein | GenBank ID | GI Number | Organism |
|---|---|---|---|
| AF1211 | NP_070039.1 | 11498810 | Archaeoglobus fulgidus |
| AF1983 | NP_070807.1 | 11499565 | Archaeoglobus fulgidus |
| scs | YP_135572.1 | 55377722 | Haloarcula marismortui |
| PAE3250 | NP_560604.1 | 18313937 | Pyrobaculum aerophilum str. |
| | | | IM2 |
| sucC | NP_415256.1 | 16128703 | Escherichia coli |
| sucD | AAC73823.1 | 1786949 | Escherichia coli |
| LSC1 | NP_014785 | 6324716 | Saccharomyces cerevisiae |
| LSC2 | NP_011760 | 6321683 | Saccharomyces cerevisiae |
| paaF | AAC24333.2 | 22711873 | Pseudomonas putida |
| matB | AAC83455.1 | 3982573 | Rhizobium leguminosarum |

Another candidate enzymes include those known in the art and described by U.S. Pat. No. 8,940,509 which are herein incorporated in its entirety and for all purposes

No EC.

Formation of caprolactone from 6-hydroxyhexanoyl-CoA (step D of FIG. 5) either occurs spontaneously or is catalyzed by enzymes having 6-hydroxyhexanoyl-CoA cyclase or alcohol transferase activity. Several enzymes with alcohol transferase activity were demonstrated in Examples 1-10 of U.S. Pat. No. 7,901,915. These include Novozyme 435 (immobilized lipase B from Candida antarctica, Sigma), Lipase C2 from Candida cylindracea (Alphamerix Ltd), lipase from Pseudomonas fluorescens (Alphamerix Ltd), L-aminoacylase ex Aspergillus spp., and protease ex Aspergillus oryzae. Such enzymes were shown to form methyl acrylate and ethyl acrylate from acrylyl-CoA and methanol or ethanol, respectively. Similar alcohol transferase enzymes can also be used to form cyclic esters such as caprolactone. Other suitable candidates include esterase enzymes in EC class 3.1.1, described above. Additional candidates include O-acyltransferases that transfer acyl groups from acyl-CoA to alcohols. Suitable O-acyltransferases include serine O-acetyltransferase (EC 2.3.1.30) such as cysE of E. coli, homoserine O-acetyltransferase (EC 2.3.1.31) enzymes such as met2 of Saccharomyces cerevisiae, or carnitine O-acyltransferases (EC 2.3.1.21) such as Cpt1a of Rattus norvegicus (Langin et al Gene 49:283-93 (1986); Denk et al, J Gen Microbiol 133:515-25 (1987); de Vries et al, Biochem 36:5285-92 (1997)).

| Gene | Accession No. | GI No. | Organism |
|---|---|---|---|
| Met2 | NP_014122.1 | 6324052 | Saccharomyces cerevisiae |
| cysE | NP_418064.1 | 16131478 | Escherichia coli |
| Cpt1a | NP_113747.2 | 162287173 | Rattus norvegicus |

Cyclization of 6-hydroxyhexanoyl-phosphate to caprolactone (Step I of FIG. 5) can either occur spontaneously or by an enzyme with 6-hydroxyhexanoyl phosphate cyclase activity. An exemplary enzyme for this transformation is acyl-phosphate:glycerol-3-phosphate acyltransferase, encoded by plsY of Streptococcus pneumoniae (Lu et al, J Biol Chem 282:11339-46 (2007)). Although this enzyme catalyzes an intermolecular reaction, it could also catalyze the intramolecular ester-forming reaction to caprolactone. Genes encoding similar enzymes are listed in the table below. Alcohol transferase enzymes and esterase enzymes described above are also suitable candidates.

| Gene | Accession No. | GI No. | Organism |
|---|---|---|---|
| plsY | P0A4P9.1 | 61250558 | Streptococcus pneumoniae |
| plsY | YP_001035186.1 | 125718053 | Streptococcus sanguinis |
| ykaC | NP_267134.1 | 15672960 | Lactococcus lactis |
| plsY | NP_721591.1 | 24379636 | Streptococcus mucans |

### Example 3 HDO

Pathways for producing HDO from ACA, adipyl-CoA or adipate are depicted in FIG. 4. Biosynthetic pathways for forming ACA, adipate and adipyl-CoA are well known in the art (for example, see U.S. Pat. No. 7,799,545) and are also described above. Pathways for HDO formation include the those pathways exemplified in Table 9.

Adipyl-CoA and adipate are converted to HDO by several alternate pathways pathways shown in FIG. 4. Adipyl-CoA is reduced to adipate semialdehyde by adipyl-CoA dehydrogenase (Step E, FIG. 4). Alternately, adipyl-CoA is hydrolyzed to adipate, which is further reduced to adipate semialdehyde by a carboxylic acid reductase (Steps M; L, FIG. 4). An alcohol dehydrogenase further reduces adipate semialdehyde to its corresponding alcohol (Step F, FIG. 4). The 6-hydroxyhexanoate intermediate is reduced to 6-hydroxyhexanal by either a carboxylic acid reductase (Step K, FIG. 4), or by CoA activation (Step G, FIG. 4) followed by reduction by a CoA-dependent aldehyde dehydrogenase (Step H, FIG. 4). Further reduction of 6-hydroxyhexanal by an HDO dehydrogenase yields HDO (Step I, FIG. 4). Adipate to HDO pathways entail either reduction of adipate to adipate semialdehyde by a CAR enzyme (Step L, FIG. 4) or by an adipyl-CoA transferase or synthase (Step M, FIG. 4) combined with an acylating adipate semialdehyde dehydrogenase (Step E, FIG. 4).

6-Aminocaprote to HDO pathways entail reduction of 6-aminocaproate to 6-aminocaproate semialdehyde. This transformation is catalyzed directly by a carboxylic acid reductase (Step D, FIG. 4). Alternately the 6-aminocaproate semialdehyde is formed in two steps by a CoA synthetase or transferase (Step A, FIG. 4) and a 6-aminocaproyl-CoA reductase (Step B, FIG. 4). 6-aminocaproate semialdehyde reductase converts the aldehyde to 6-aminohexanol intermediate (Step C, FIG. 4). An aminotransferase or dehydrogenase converts 6-aminohexanol to 6-hydroxyhexanal (Step J, FIG. 4), which is subsequently reduced to HDO by an alcohol dehydrogenase (Step I, FIG. 4).

In addition to the pathways shown in FIG. 4 and described herein, HDO can be biosynthesized from other PAI intermediates such as HMD, CPL and CPO. For example, aminotransferase and alcohol dehydrogenase enzymes can convert the two amine groups of HMD to their corresponding alcohols. CPL can be converted to 6ACA, and subsequently to HDO, by a CPL amidase in combination with any of the HDO pathways shown in FIG. 4. Hydrolysis of CPO by a lipase or esterase yields HDO pathway intermediate, 6-hydroxyhexanoate. Exemplary aminotransferase, alcohol dehydrogenase, amidase and esterase enzymes candidates are listed herein.

In the pathway shown in FIG. 4, adipate semialdehyde is formed either from adipate via an adipate reductase (Step E) or adipyl-CoA via adipyl-CoA reductase (Step A). Adipate semialdehyde is then reduced to 5-hydroxyhexanoate in Step B. The 6-hydroxyhexanoate intermediate is converted to caprolactone by one of several alternate routes. In one route, 6-hydroxyhexanoate is directly converted to caprolactone by a caprolactone hydrolase (step G). In yet another route, 6-hydroxyhexanoate is activated to its corresponding acyl-CoA, which then cyclizes to caprolactone (step C/D), or cyclizes via a 6-hydroxyhexanoyl-phosphate intermediate (steps J/I). In an alternate route, 6-hydroxyhexanote is activated to 6-hydroxyhexanoyl-phoshphate, which is then cyclized to caprolactone (step H/I).

The transformations of adipyl-CoA to adipate semialdehyde (Step E, FIG. 4), 6-aminocaproyl-CoA to 6-aminocaproate semialdehyde (Step B, FIG. 4) and 6-hydroxyhexanoyl-CoA to 6-hydroxyhexanal (Step H, FIG. 4) require acyl-CoA dehydrogenases such as those described herein above in Example 1. Additional candidates are listed in Tables 3 and 4.

The transformation of adipate to adipyl-CoA to adipate (Step M, FIG. 4), 6-aminocaproate to 6-aminocaproyl-CoA (Step A, FIG. 4) and 6-hydroxyhexanoate to 6-hydroxyhexanoyl-CoA (Step G, FIG. 4) can be performed by CoA hydrolase, transferases or ligases such as those described above in Example 1 that have broad substrate specificity. Additional candidates are found in the EC classes 3.2.1, 2.8.3 and 6.2.1 and are listed in the Tables 3 and 4.

Carboxylic acid reductase enzymes are required to convert adipate to adipate semialdehyde (Step L, FIG. 4), 6-aminocaproate to 6-aminocapropate semialdehyde (Step D, FIG. 4) and 6-hydroxyhexanoate to 6-hydroxyhexanal (Step K, FIG. 4). Exemplary enzymes include carboxylic acid reductase (CAR), alpha-aminoadipate reductase, hydroxybenzoic acid reductase and retinoic acid reductase. Carboxylic acid reductase (CAR) catalyzes the magnesium, ATP and NADPH-dependent reduction of carboxylic acids to their corresponding aldehydes. The CAR enzyme from Nocardia iowensis exhibits activity on a broad range of substrates (Venkitasubramanian et al., Biocatalysis in Pharmaceutical and Biotechnology Industries. CRC press (2006)). The enzyme from Nocardia iowensis, encoded by car, was cloned and functionally expressed in E. coli (Venkitasubramanian et al., J Biol. Chem. 282:478-485 (2007)). CAR requires post-translational activation by a phosphopantetheine transferase (PPTase) encoded by npt that converts the inactive apo-enzyme to the active holo-enzyme (Hansen et al., Appl. Environ. Microbiol 75:2765-2774 (2009)). An additional enzyme candidate found in Streptomyces griseus is encoded by the griC and griD genes. This enzyme is believed to convert 3-amino-4-hydroxybenzoic acid to 3-amino-4-hydroxybenzaldehyde as deletion of either griC or griD led to accumulation of extracellular 3-acetylamino-4-hydroxybenzoic acid, a shunt product of 3-amino-4-hydroxybenzoic acid metabolism (Suzuki, et al., J. Antibiot. 60(6):380-387 (2007)). Co-expression of griC and griD with SGR_665, an enzyme similar in sequence to the Nocardia iowensis npt, can be beneficial. Alpha-aminoadipate reductase (AAR, EC 1.2.1.31), participates in lysine biosynthesis pathways in some fungal species. This enzyme naturally reduces alpha-aminoadipate to alpha-aminoadipate semialdehyde. The carboxyl group is first activated through the ATP-dependent formation of an adenylate that is then reduced by NAD(P)H to yield the aldehyde and AMP. Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279 (2003)), and Schizosaccharomyces pombe (Ford et al., Curr. Genet. 28:131-137 (1995)). The AAR from S. pombe exhibited significant activity when expressed in E. coli (Guo et al., Yeast 21:1279-1288 (2004)).

| | GenBank | | |
|---|---|---|---|
| Gene | Accession No. | GI No. | Organism |
| car | AAR91681.1 | 40796035 | Nocardia iowensis |
| npt | ABI83656.1 | 114848891 | Nocardia iowensis |
| griC | YP_001825755.1 | 182438036 | Streptomyces griseus |
| griD | YP_001825756.1 | 182438037 | Streptomyces griseus |
| LYS2 | AAA34747.1 | 171867 | Saccharomyces cerevisiae |
| LYS5 | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Penicillium chrysogenum |

The transformations of 6-aminocaproate semialdehyde to 6-aminohexanol (Step C, FIG. 4), adipate semialdehyde to 6-hydroxyhexanoate (Step F, FIG. 4) and 6-hydroxyhexanal to HDO (Step I, FIG. 4) are catalyzed by alcohol dehydrogenase enzymes. Exemplary alcohol dehydrogenase enzymes for catalyzing these transformations include alrA encoding a medium-chain alcohol dehydrogenase active on a range fo C2-C14 compounds (Tani et al., Appl. Environ. Microbiol. 66:5231-5235 (2000)), yqhD, yahK, adhP and fucO from E. coli (Sulzenbacher et al., J Mol Biol 342:489-502 (2004)), and butanol dehydrogenase enyzmes from Clostridial species (Walter et al, J. Bacteriol 174:7149-7158 (1992)). YqhD of E. coli catalyzes the reduction of a wide range of aldehydes using NADPH as the cofactor, with a preference for chain lengths longer than C(3) (Sulzenbacher et al, J Mol Biol 342:489-502 (2004); Perez et al., J Biol. Chem. 283:7346-7353 (2008)).

| Protein | GenBank ID | GI Number | Organism |
|---|---|---|---|
| alrA | BAB12273.1 | 9967138 | Acinetobacter sp. strain M-1 |
| ADH2 | NP_014032.1 | 6323961 | Saccharomyces cerevisiae |
| fucO | NP_417279.1 | 16130706 | Escherichia coli |
| yqhD | NP_417484.1 | 16130909 | Escherichia coli |
| yahK | P75691 | 2492774 | Escherichia coli |
| adhP | NP_415995 | 90111280 | Escherichia coli |
| bdh I | NP_349892.1 | 15896543 | Clostridium acetobutylicum |
| bdh II | NP_349891.1 | 15896542 | Clostridium acetobutylicum |
| bdh | BAF45463.1 | 124221917 | Clostridium |
| | | | saccharoperbutylacetonicum |
| Cbei_1722 | YP_001308850 | 150016596 | Clostridium beijerinckii |
| Cbei_2181 | YP_001309304 | 150017050 | Clostridium beijerinckii |
| Cbei_2421 | YP_001309535 | 150017281 | Clostridium beijerinckii |

The transformation of 6-aminohexanol to 6-hydroxyhexanal (Step J, FIG. 4) is catalyzed by an aminotransferase such as those described above in Example 1 that have broad substrate specificity. Additional candidates include aminotransferase and oxidoreductase enzymes found in the EC classes 2.6.1 and 1.4.1, listed in Tables 3 and 4.

Throughout this application various publications have been referenced. The disclosures of these publications in their entireties, including GenBank and GI number publications, are hereby incorporated by reference in this application in order to more fully describe the state of the art to which this invention pertains. Although the invention has been described with reference to the examples provided above, it should be understood that various modifications can be made without departing from the spirit of the invention.

1. A genetically modified cell capable of producing a target product, said target product comprising hexamethylenediamine (HMD), levulinic acid (LVA), 6-aminocaproic acid (6ACA), caprolactam (CPL), caprolactone (CPO), adipic acid (ADA), or 1,6-hexanediol (HDO) or a combination thereof, wherein said genetically modified cell comprises one or more genetic modifications selected from:
(a) a genetic modification that decreases activity of an enzyme selected from an Oxidoreductase acting on an aldehyde or oxo moiety (A1); Oxidoreductase acting on a acyl-CoA moiety (A2); Oxidoreductase acting on an aldehyde moiety (A3); Oxidoreductase acting on an aldehyde or acyl-CoA moiety (A4); Aldehyde oxidase acting on an aldehyde moiety (A5); Oxidoreductase acting on an alkene or alkane moiety (A6); Oxidoreductase acting on an amine moiety (A7); Amine N-methyltransferase acting on an amine moiety (A8); Carbamoyl transferase acting on an amine moiety (A9); Acyltransferase acting on an acyl-CoA moiety (A10); Acyltransferase acting on an amine or acyl-CoA moiety (A11); N-propylamine synthase acting on an amine moiety (A12); Aminotransferase acting on an amine or aldehyde moiety (A13); CoA transferase acting on an acyl-CoA or an acid moiety (A14); Thioester hydrolase acting on an acyl-CoA moiety (A15); Decarboxylase acting on an oxoacid moiety (A16); Dehydratase acting on a hydroxyacid moiety (A17); Ammonia-lyase acting on an amine moiety (A18); CoA ligase acting on an acyl-CoA or acid moiety (A19); glutamyl:amine ligase acting on an amine moiety (A20); Amine hydroxylase acting on an amine moiety (A21); Oxidoreductase acting on an acyl-CoA moiety (A22); Amine oxidase acting on an amine moiety (A23); short chain diamine exporter acting on a diamine moiety (A24); and putrescine permease acting on a diamine moiety (A25);
(b) a genetic modification that increases activity of an enzyme selected from Amide hydrolase or amidase acting on an amide moiety (B1); Cyclic amide hydrolase or lactamase acting on a cyclic amide moiety (B2); CoA ligase acting on an acid moiety (B3); Diamine transporter (longer chain diamines) acting on an amine moiety (B4); and diamine permease acting on an amine moiety (B5); and
(c) a combination of two or more, three or more, four or more, five or more, six or more, seven or more, eight or more, nine or more, ten or more, or all of the genetic modifications of (a) and (b);
wherein said cell produces a reduced amount of one or more byproducts when compared to a cell without said one or more genetic modifications.

2.-24. (canceled)

25. The genetically modified cell of claim 1, wherein said cell produces HMD, ADA, 6ACA, CPO, CPL, LVA, or HDO comprising a reduced level of one or more byproducts of Table 10 or Table 11.

26.-53. (canceled)

54. The genetically modified cell of claim 1, wherein reducing the amount of said byproduct increases yield of target product.

55. The genetically modified cell of claim 1, wherein said byproduct decreases yield of said target product.

56. The genetically modified cell of claim 1, wherein said byproduct increases the degradation of a polymer comprising said target product.

57. The genetically modified cell of claim 1, wherein said byproduct inhibits polymerization of target product to a polymer in a polymerization reaction.

58. The genetically modified cell of claim 56 or 57, wherein said polymer is a polyamide (PA).

59. The genetically modified cell of claim 58, wherein said PA is selected from PA6, PA6,6, PA6,9, PA6,10, PA6,12 or PA6T.

60. The genetically modified cell of claim 1, wherein said byproduct inhibits polymerization of HMD, ADA, 6ACA, CPL, CPO, LVA, or HDO in a polymerization reaction.

61. The genetically modified cell of claim 1, wherein said cell produces HMD, 6ACA, ADA, CPL, CPO, LVA, or HDO that comprises greater than about 5, 10, 15, 20, 25, or 30% HMD, 6ACA, ADA, CPL, CPO, LVA, or HDO respectively by weight in fermentation broth.

62. The genetically modified cell of claim 1, wherein said cell produces HMD, 6ACA, ADA, CPL, CPO, LVA, or HDO that comprises greater than about 5, 10, 20, 30, 40, 50, 60, 70, 75, 80, 85, 90, 95, or 100% HMD, 6ACA, ADA, CPL, CPO, LVA, or HDO respectively by weight after processing or purification.

63. The genetically modified cell of claim 1, wherein said cell produces HMD, 6ACA, ADA, CPL, CPO, LVA, or HDO that comprises greater than about 99, 99.90, 99.92, 99.94, 99.96, 99.98, 99.99, or 100% HMD, 6ACA, ADA, CPL, CPO, LVA, or HDO respectively by weight after processing or purification.

64. The genetically modified cell of claim 1, wherein said cell produces HMD, 6ACA, ADA, CPL, CPO, LVA, or HDO that comprises less than 10000, 7500, 5000, 4000, 3000, 2000, 1000, 500, 250, 125, 100, 90, 75, 50, 40, 30, 20, 10, 5, or 1 ppm of one or more byproducts selected from Table 10 or Table 11.

65. The genetically modified cell of claim 1, wherein said cell produces HMD, 6ACA, ADA, CPL, CPO, LVA, or HDO that comprises less than 20, 10, 5, 1, 0.5% by weight of one or more byproducts selected from Table 10 or Table 11.

66. (canceled)

67. The genetically modified cell of claim 1, wherein said level of said byproduct is reduced by 5, 10, 20, 25, 30, 35, 40, 45, 50, 60, 70, 80, 90. 95 or 100% compared to a control cell lacking said genetic modification.

68.-107. (canceled)

108. A composition comprising a target product selected from LVA, 6ACA, CPL, CPO, ADA, HMD or HDO and a byproduct selected from Table 10 or Table 11.

109.-112. (canceled)

113. The composition of claim 108, wherein said composition comprises at least 5, 10, 15, 20, 25, or 30% by weight of said target product in said fermentation broth.

114.-118. (canceled)

119. The composition of claim 108, wherein said target product comprises less than 20, 10, 5, 1, 0.5% by weight a byproduct or combination of byproducts selected from Table 10 or Table 11.

120. The composition of claim 119, wherein said composition comprises HMD.

121.-160. (canceled)

161. The genetically modified cell of claim 1, wherein said cell comprises a target product pathway comprising at least one exogenous nucleic acid encoding a target product pathway enzyme expressed in a sufficient amount to produce the target product, wherein said target product pathway comprises a pathway selected from FIG. 1, 2, 3, 4 or 5.
